(19) United States
(12) Patent Application Publication
(10) Pub. No.: US 2011/0224204 A1
(43) Pub. Date: Sep. 15, 2011

(54) DI-SUBSTITUTED PHENYL COMPOUNDS

(76) Inventors: Richard Chesworth, Boston, MA (US); Gideon Shapiro, Gainsville, FL (US); Amy Ripka, Reading, MA (US)

(21) Appl. No.: 13/001,361

(22) PCT Filed: Jun. 25, 2009
(86) PCT No.: PCT/US2009/048608

§ 371 Date: May 18, 2011

Related U.S. Application Data
(60) Provisional application No. 61/075,599, filed on Jun. 25, 2008.
(60) Provisional application No. 61/138,856, filed on Dec. 18, 2008.

(51) Int. Cl. A61K 31/5377; C07D 401/10; A61K 31/4439; C07D 413/10; C07D 417/10; A61K 31/444; A61K 31/517; A61K 31/4709; A61K 31/498; A61P 3/10; A61P 15/00; A61P 25/00

(52) U.S. Cl. 514/233.8; 546/273.4; 514/338; 546/271.7; 546/270.1; 546/262; 514/335; 544/284; 514/266.21; 546/152; 514/314; 544/353; 514/249; 544/137; 544/139; 544/124; 514/235.5

(57) ABSTRACT

Di-substituted phenyl compounds which are inhibitors of phosphodiesterase 10 are described as are processes, pharmaceutical compositions, pharmaceutical preparations and pharmaceutical use of the compounds in the treatment of mammals, including human(s) for central nervous system (CNS) disorders and other disorders which may affect CNS function. The disclosure also relates to methods for treating neurological, neurodegenerative and psychiatric disorders including but not limited to those comprising cognitive deficits or schizophrenic symptoms.

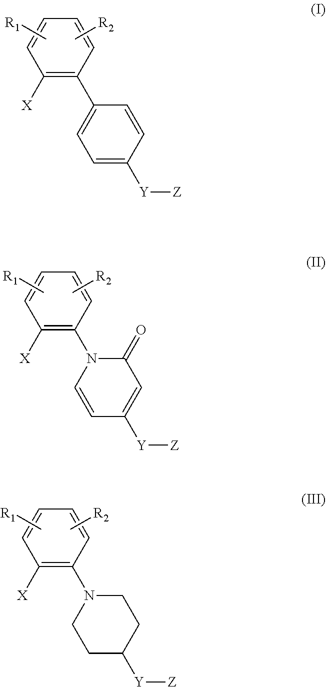

The disclosure relates to di-substituted phenyl compounds which are inhibitors of phosphodiesterase 10. The disclosure further relates to processes, pharmaceutical compositions, pharmaceutical preparations and pharmaceutical use of the compounds in the treatment of mammals, including human(s) for central nervous system (CNS) disorders and other disorders which may affect CNS function. The disclosure also relates to methods for treating neurological, neurodegenerative and psychiatric disorders including but not limited to those comprising cognitive deficits or schizophrenic symptoms.

### BACKGROUND

Cyclic phosphodiesterases are intracellular enzymes which, through the hydrolysis of cyclic nucleotides cAMP and cGMP, regulate the levels of these mono phosphate nucleotides which serve as second messengers in the signaling cascade of G-protein coupled receptors. In neurons, PDEs also play a role in the regulation of downstream cGMP and cAMP dependent kinases which phosphorylate proteins involved in the regulation of synaptic transmission and homeostasis. To date, eleven different PDE families have been identified which are encoded by 21 genes. The PDEs contain a variable N-terminal regulatory domain and a highly conserved C-terminal catalytic domain and differ in their substrate specificity, expression and localization in cellular and tissue compartments, including the CNS.

The discovery of a new PDE family, PDE10, was reported simultaneously by three groups in 1999 (Soderling et al. “Isolation and characterization of a dual-substrate phosphodiesterase gene family: PDE10A” Proc. Natl. Sci. 1999, 96, 7071-7076; Loughney et al. “Isolation and characterization of PDE10A, a novel human 3′,5′-cyclic nucleotide phosphodiesterase” Gene 1999, 234, 109-117; Fujishige et al. “Cloning and characterization of a novel human phosphodiesterase that hydrolyzes both cAMP and cGMP (PDE10A)” J. Biol. Chem. 1999, 274, 18438-18445). The human PDE10 sequence is highly homologous to both the rat and mouse variants with 95% amino acid identity overall, and 98% identity conserved in the catalytic region.

PDE10 is primarily expressed in the brain (caudate nucleus and putamen) and is highly localized in the medium spiny neurons of the striatum, which is one of the principal inputs to the basal ganglia. This localization of PDE10 has led to speculation that it may influence the dopaminergic and glutamatergic pathways both which play roles in the pathology of various psychotic and neurodegenerative disorders.

PDE10 hydrolyzes both cAMP (Kₘ=0.05 uM) and cGMP (Kₘ=3 uM) (Soderling et al. “Isolation and Characterization of a dual-substrate phosphodiesterase gene family: PDE10.” Proc. Natl. Sci. USA 1999, 96(12), 7071-7076). In addition, PDE10 has a five-fold greater Vₘₐₓ for cGMP than for cAMP and these in vitro kinetic data have lead to the speculation that PDE10 may act as a cAMP-inhibited cGMP phosphodiesterase in vivo (Soderling and Beavo “Regulation of cAMP and cGMP signaling: New phosphodiesterases and new functions,” Curr. Opin. Cell Biol., 2000, 12, 174-179).

PDE10 is also one of five phosphodiesterase members to contain a tandem GAF domain at their N-terminus. It is differentiated by the fact that the other GAF containing PDEs (PDE2, 5, 6, and 11) bind cGMP while recent data points to the tight binding of cAMP to the GAF domain of PDE10 (Handa et al. “Crystal structure of the GAF-B domain from human phosphodiesterase 10A complexed with its ligand, cAMP” J. Biol. Chem. 2008, May 13^{th}, ePub).

PDE10 inhibitors have been disclosed for the treatment of a variety of neurological and psychiatric disorders including Parkinson's disease, schizophrenia, Huntington's disease, delusional disorders, drug-induced psychoses, obsessive compulsive and panic disorders (US Patent Application 2003/0032579). Studies in rats (Kostowski et. al “Papaverine drug induced stereotypy and catalepsy and biogenic amines in the brain of the rat” Pharmacol. Biochem. Behay. 1976, 5, 15-17) have showed that papaverine, a selective PDE10 inhibitor, reduces apomorphine induced stereotypies and rat brain dopamine levels and increases haloperidol induced catalepsy. This experiment lends support to the use of a PDE10 inhibitor as an antipsychotic since similar trends are seen with known, marketed antipsychotics.

Antipsychotic medications are the mainstay of current treatment for schizophrenia. Conventional or classic antipsychotics, typified by haloperidol, were introduced in the mid-1950s and have a proven track record over the last half century in the treatment of schizophrenia. While these drugs are effective against the positive, psychotic symptoms of schizophrenia, they show little benefit in alleviating negative symptoms or the cognitive impairment associated with the disease. In addition, drugs such as haloperidol have extreme side effects such as extrapyramidal symptoms (EPS) due to their specific dopamine D2 receptor interaction. An even more severe condition characterized by significant, prolonged, abnormal motor movements known as tardive dyskinesia also may emerge with prolonged classic antipsychotic treatment.

The 1990s saw the development of several new drugs for schizophrenia, referred to as atypical antipsychotics, typified by risperidone and olanzapine and most effectively, clozapine. These atypical antipsychotics are generally characterized by effectiveness against both the positive and negative symptoms associated with schizophrenia, but have little effectiveness against cognitive deficiencies and persisting cognitive impairment remain a serious public health concern (Davis, J. M et al. “Dose response and dose equivalence of antipsychotics.” Journal of Clinical Psychopharmacology, 2004, 24 (2), 192-208; Friedman, J. H. et al “Treatment of psychosis in Parkinson's disease: Safety considerations.” Drug Safety, 2003, 26 (9), 643-659). In addition, the atypical antipsychotic agents, while effective in treating the positive and, to some degree, negative symptoms of schizophrenia, have significant side effects. For example, clozapine which is one of the most clinically effective antipsychotic drugs shows agranulocytosis in approximately 1.5% of patients with fatalities due to this side effect being observed. Other atypical antipsychotic drugs have significant side effects including metabolic side effects (type 2 diabetes, significant weight gain, and dyslipidemia), sexual dysfunction, sedation, and potential cardiovascular side effects that compromise their clinically effectiveness. In the large, recently published NIH sponsored CATIE study, (Lieberman et al “The Clinical Antipsychotic Trials Of Intervention Effectiveness (CATIE) Schizophrenia Trial: clinical comparison of subgroups with and without the metabolic syndrome.” Schizophrenia Research, 2005, 80 (1), 9-43) 74% of patients discontinued use of their antipsychotic medication within 18 months due to a number of factors including poor tolerability or incomplete efficacy. Therefore, a substantial clinical need still exists for more effective and better tolerated antipsychotic mediations possibly through the use of PDE10 inhibitors.

### Brief Summary

Described herein are di-substituted phenyl compounds which are inhibitors of phosphodiesterase 10 of Formulas (I), (II) and (III):

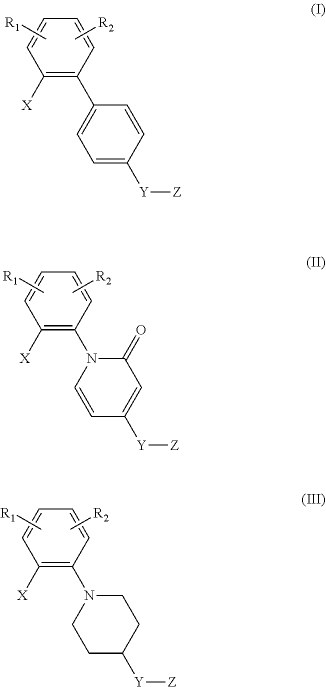

Wherein:

X is selected from C₃-C₈ alkyl, optionally substituted cycloalkyl, optionally substituted cycloalkyloxy, optionally substituted cycloalkylalkyl, optionally substituted cycloalkylalkoxy, optionally substituted heterocycloalkyl, optionally substituted heterocycloalkyloxy, optionally substituted heterocycloalkylalkyl, optionally substituted aryl, optionally substituted arylalkyl, optionally substituted aryloxy, optionally substituted arylalkoxy, optionally substituted heteroaryl, optionally substituted heteroarylalkyl, optionally substituted heteroaryloxy and optionally substituted heteroarylalkoxy;

Y is a bond or a divalent linker group selected from —CH₂—, —O—, —SO₂—, —CH₂O—, —OCH₂— and —CH₂CH₂— with the rightmost radical of the Y group connected to the Z substituent;

Z is optionally substituted heteroaryl;

R₁ is selected from hydrogen, alkyl, CF₃, alkoxy, alkoxyalkyl, optionally substituted cycloalkyl, optionally substituted cycloalkyloxy, optionally substituted cycloalkylalkyl, optionally substituted cycloalkylalkoxy, optionally substituted heterocycloalkyl, optionally substituted heterocycloalkylalkyl, optionally substituted heteroaryl, optionally substituted heteroarylalkyl, halogen, alkylthio, alkylsulfonyl, cyano, amino, alkylamino, dialkylamino, amido, alkylamido, dialkylamido and nitro; and

R₂ is selected from hydrogen, C₁-C₄ alkyl, CF₃, optionally substituted cycloalkyl, halogen, alkoxy, alkylthio, alkylsulfonyl, cyano and nitro.

In some embodiments, alkyl groups are fully saturated whether present on their own or as part of another group (e.g., alkylamino).

In certain embodiments, substituent groups are not further substituted.

In various embodiments, any group that is defined as being optionally substituted is independently singly or multiply substituted.

In various embodiments, any group that is defined as being optionally substituted not substituted.

In one embodiment, X is selected from C₃-C₈ alkyl, cycloalkyl, cycloalkyloxy, cycloalkylalkyl and cycloalkylalkoxy.

In a further embodiment X is selected from cycloalkyl and cycloalkylalkyl. Examples include but are not limited to cyclohexyl and cyclohexylmethyl.

In another embodiment X is selected from cycloalkyloxy and cycloalkylalkyloxy. Examples include but are not limited to cyclohexyloxy and cyclohexylmethyloxy

In another embodiment X is C₃-C₈ alkyl. Examples include but are not limited to isopropyl, t-butyl and isopentyl.

In another embodiment X is heteroaryl.

In another embodiment, X is selected from a monocyclic aromatic ring having 5 ring atoms selected from C, O, S and N provided the total number of ring heteroatoms is less than or equal to four and where no more than one of the total number of heteroatoms is oxygen or sulfur, and a monocyclic aromatic ring having 6 atoms selected from C and N provided that not more than 3 ring atoms are N, and where said ring may be optionally and independently substituted with up to two groups selected from C₁-C₄ alkyl, cycloalkyl, cycloalkyloxy, C₁-C₄ alkoxy, CF₃, carboxy, alkoxyalkyl, cycloalkylalkoxy, amino, alkylamino, dialkylamino, amido, alkylamido, dialkylamido, thioalkyl, halogen, cyano, and nitro. Examples include but are not limited to 1H-pyrrolyl, furanyl, thiophenyl, imidazolyl, pyrazolyl, isothiazolyl, isoxazolyl, oxazolyl, thiazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, 1,2,3,4-oxatriazolyl, 1,2,3,5-oxatriazolyl, 1,2,3,4-thiatriazolyl, 1,2,3,5-thiatriazolyl, 1,2,3-triazinyl, 1,2,4-triazinyl, 1,3,5-triazinyl, pyridinyl, pyrazinyl, pyridazinyl and pyrimidinyl.

In a further embodiment, X is a monocyclic aromatic ring having 6 ring atoms selected from C and N provided that not more than 3 ring atoms are N, and where said ring may be optionally and independently substituted with up to two groups selected from C₁-C₄ alkyl, cycloalkyl, cycloalkyloxy, C₁-C₄ alkoxy, CF₃, carboxy, alkoxyalkyl, cycloalkylalkoxy, amino, alkylamino, dialkylamino, amido, alkylamido, dialkylamido, thioalkyl, halogen, cyano, and nitro. Examples include but are not limited to 1,2,3-triazinyl, 1,2,4-triazinyl, 1,3,5-triazinyl, pyridinyl, pyrazinyl, pyridazinyl and pyrimidinyl.

In a further embodiment, X is a monocyclic aromatic ring having 5 ring atoms selected from C, O, S, and N, provided the total number of ring heteroatoms is less than or equal to four and where no more than one of the total number of heteroatoms is oxygen or sulfur and where said ring may be optionally and independently substituted with up to two groups selected from C₁-C₄ alkyl, cycloalkyl, cycloalkyloxy, C₁-C₄ alkoxy, CF₃, carboxy, alkoxyalkyl, cycloalkylalkoxy, amino, alkylamino, dialkylamino, amido, alkylamido, dialkylamido, thioalkyl, halogen, cyano, and nitro. Examples include but are not limited to 1H-pyrrolyl, furanyl, thiophenyl, imidazolyl, pyrazolyl, isothiazolyl, isoxazolyl, oxazolyl, thiazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, 1,2,3,4-oxatriazolyl, 1,2,3,5-oxatriazolyl, 1,2,3,4-thiatriazolyl, 1,2,3,5-thiatriazolyl.

In a further embodiment, X is selected from 2-pyridinyl, 3-pyridinyl or 4-pyridinyl optionally substituted with one group selected from C₁-C₄ alkyl, cyclopropyl, cyclopropyloxy, cyclopropylmethyl, C₁-C₄ alkoxy, CF₃, amino, alkylamino, dialkylamino, thioalkyl, halogen or cyano.

In a further embodiment, X is 3-pyridinyl optionally substituted with one group selected from C₁-C₄ alkyl, cyclopropyl, cyclopropyloxy, cyclopropylmethyl, C₁-C₄ alkoxy, CF₃, amino, alkylamino, dialkylamino, thioalkyl, halogen or cyano.

In a further embodiment, X is 4-pyridinyl optionally substituted with one group selected from C₁-C₄ alkyl, cyclopropyl, cyclopropyloxy, cyclopropylmethyl, C₁-C₄ alkoxy, CF₃, amino, alkylamino, dialkylamino, thioalkyl, halogen or cyano.

In a further embodiment, X is selected from 3-pyridinyl or 4-pyridinyl.

In a further embodiment, X is 3-pyridinyl.

In another embodiment, X is 2-methoxy-5-pyridinyl

In a further embodiment, X is 4-pyridinyl.

In another embodiment, X is 2-methoxy-4-pyridinyl

In a further embodiment X is a heterobicyclic ring system.

In another embodiment X is a heterobicyclic ring system where one ring is aromatic.

In a further embodiment, X is a heterobicyclic ring system where both rings are aromatic.

In another embodiment, X is a heterobicyclic ring system containing exactly 9 ring atoms.

In another embodiment, X is a heterobicyclic ring system containing exactly 10 ring atoms.

In another embodiment X is selected from benzo[d]oxazoyl, benzo[c][1,2,5]oxadiazyl, benzo[c][1,2,5]thiadiazolyl, benzo[c/]isoxazolyl, 1H-benzo[c]imidazoyl, benzo[d]thiazoyl, benzo[c]isothiazolyl, benzo[c/]isothiazolyl, benzo[c]isoxazolyl, imidazo[1,2-a]pyridinyl and imidazo[1,5-a]pyridinyl

In another embodiment X is selected from benzo[c][1,2,5]oxadiazyl and benzo[c][1,2,5]thiadiazolyl.

In a further embodiment, X is selected from benzo[d]oxazoyl, 1H-benzo[d]imidazoyl and benzo[d]thiazoyl.

In a further embodiment, X is benzo[d]oxazoyl.

In a further embodiment, X is 1H-benzo[d]imidazoyl.

In a further embodiment, X is benzo[d]thiazoyl.

In another embodiment X is benzo[c][1,2,5]oxadiazoyl.

In a further embodiment X is benzo[c][1,2,5]thiadiazolyl

In a further embodiment, X is benzo[d]isoxazolyl.

In another embodiment, X is benzo[d]isothiazolyl.

In another embodiment, X is benzo[c]isothiazolyl.

In another embodiment, X is benzo[c]isoxazolyl.

In another embodiment, X is imidazo[1,2-a]pyridinyl.

In another embodiment, X is imidazo[1,5-a]pyridinyl.

In an additional embodiment, X is selected from heterocycloalkyl or heterocycloalkyloxy.

In a further embodiment X is heterocycloalkyl consisting of 6 ring atoms. Examples include but are not limited to morpholino, piperidinyl, piperazinyl N-Me-piperazinyl and pyranyl.

In another embodiment X is heterocycloalkyl consisting of 5 ring atoms. Examples include but are not limited to tetrahydrofuranyl and pyrrolidinyl.

In another embodiment, X is a heterocycloalkyl group selected from Formulas A1-A16 depicted below:

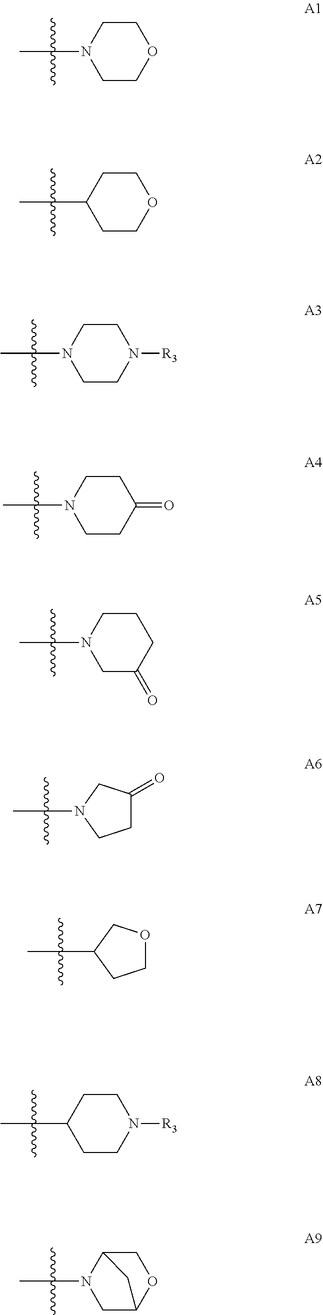

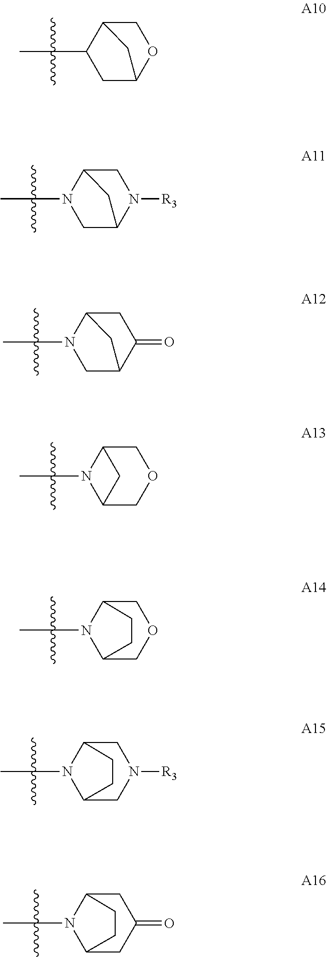

Where R₃ is selected from hydrogen, C₁-C₆ alkyl, C₃-C₆ cycloalkyl and C₃-C₆ cycloalkylalkyl, all of which can be optionally substituted.

In another embodiment X is selected from morpholino, pyranyl or tetrahydrofuranyl.

In another embodiment X is selected from morpholino (having formula A1) or 4-pyranyl (having Formula A2).

In an additional embodiment X is heterocycloalkyloxy.

In a further embodiment X is heterocycloalkyloxy consisting of 6 ring atoms. Examples include but are not limited to piperidin-4-oxy-yl, and tetrahydro-2H-pyran-4-oxy-yl.

In another embodiment X is heterocycloalkyloxy consisting of 5 ring atoms. Examples include but are not limited to tetrahydrofuran-3-oxy-yland pyrrolidin-3-oxy-yl.

In another embodiment, X is a heterocycloalkyloxy group selected from Formulas B1-B3 depicted below

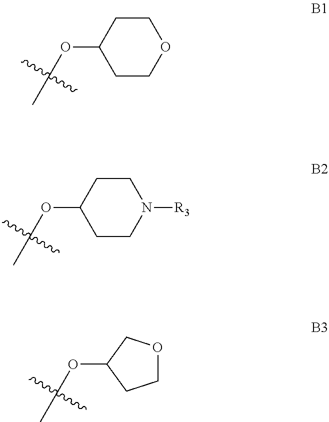

Where R₃ is selected from hydrogen, C₁-C₆ alkyl, C₃-C₆ cycloalkyl and C₃-C₆ cycloalkylalkyl

In an additional embodiment, X is aryl.

In another embodiment, X is selected from phenyl or pyridinyl.

In a further embodiment, X is phenyl.

In another embodiment, X is phenyl optionally substituted with one or more substituents selected from F, Cl, CN, NO₂, CF₃, OCF₃, OCHF₂, CH₂CF₃ and OMe.

In another embodiment, X is restricted phenyl.

In a further embodiment, X is selected from a 3,4-disubstituted phenyl, 3-substituted phenyl and 4-substituted phenyl.

In another embodiment, X is selected from 3,4-disubstituted phenyl and 4-substituted phenyl.

In another embodiment, X is 3-chloro-4-methoxyphenyl

In another embodiment, X is 3-cyano-4-methoxyphenyl

In a further embodiment, X is 3-chloro-4-difluoromethoxyphenyl

In a further embodiment, X is 3-cyano-4-difluoromethoxyphenyl

In an additional embodiment, X is 4-substituted phenyl.

In a further embodiment, X is 4-methoxyphenyl.

In another embodiment, X is 4-nitrophenyl.

In another embodiment, X is 4-chlorophenyl.

In another embodiment, X is 4-cyanophenyl.

In another embodiment, X is 4-trifluoroethylphenyl.

In a further embodiment, X is 4-trifluoromethoxyphenyl.

In a further embodiment, X is 3-substituted phenyl.

In another embodiment, X is 3-nitrophenyl.

In another embodiment, X is 3-trifluoromethoxyphenyl.

In a further embodiment, X is 3-methoxyphenyl.

In another embodiment, X is 3-chlorophenyl.

In another embodiment, X is 3-cyanophenyl.

In another embodiment, X is 3-trifluoroethylphenyl.

In a further embodiment, X is 3-trifluoromethoxyphenyl.

In one embodiment, Y is —CH₂O— or —OCH₂— with the rightmost radical connected to the Z substituent.

In another embodiment, Y is —CH₂CH₂— with the rightmost radical connected to the Z substituent.

In an additional embodiment, Y is —CH₂O— with the rightmost radical connected to the Z substituent.

In a further embodiment, Y is —OCH₂— with the rightmost radical connected to the Z substituent.

In one embodiment, Z is selected from heteroaryl consisting of 6 ring atoms and a heterobicyclic ring system

In another embodiment, Z is a heterobicyclic ring system.

In another embodiment, Z is a heterobicyclic ring system where one ring is aromatic.

In a further embodiment, Z is a heterobicyclic ring system where both rings are aromatic.

In another embodiment, Z is a heterobicyclic ring system containing exactly 9 ring atoms.

In another embodiment, Z is a heterobicyclic ring system containing exactly 10 ring atoms.

In an additional embodiment, Z is selected from benzimidazolyl, quinolinyl, tetrahydroquinolyl, imidazo[1,2-a]pyridin-2-yl, tetrahydroisoquinolyl, 5-methylpyridin-2-yl, 3,5-dimethylpyridin-2-yl, 6-fluoroquinolyl and isoquinolinyl, all of which may be optionally substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In an additional embodiment, Z is selected from benzimiazolyl, quinolinyl, tetrahydroquinolyl, tetrahydroisoquinolyl or isoquinolinyl, all of which may be optionally substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In an additional embodiment, Z is selected from quinolinyl, imidazo[1,2-a]pyridin-2-yl, 5-methylpyridin-2-yl, 3,5-dimethylpyridin-2-yl and 6-fluoroquinolin-2-yl, all of which may be optionally substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In an additional embodiment, Z is selected from quinolinyl and isoquinolinyl, both of which may be optionally substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In an further embodiment, Z is selected from 2-quinolinyl and 2-benzimidazolyl, both of which may be optionally substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In a further embodiment, Z is 2-quinolinyl substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In a further embodiment, Z is 6-fluoroquinolin-2-yl substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In a further embodiment, Z is 3,5-dimethylpyridin-2-yl substituted with up to 1 substituent independently selected from alkyl, alkoxy, cycloalkyl, cycloalkyloxy, cycloalkylalkyl, cycloalkylalkoxy, halogen, alkylsulfonyl and cyano and nitro.

In a further embodiment, Z is 5-methylpyridin-2-yl substituted with up to 3 substituents independently selected from alkyl, alkoxy, cycloalkyl, cycloalkyloxy, cycloalkylalkyl, cycloalkylalkoxy, halogen, alkylsulfonyl and cyano and nitro.

In an additional embodiment, Z is selected from 2-quinolinyl and 2-benzimidazolyl.

In an additional embodiment, Z is selected from 2-quinolinyl and 5-methylpyridin-2-yl.

In an additional embodiment, Z is selected from 2-quinolinyl and 3,5-dimethylpyridin-2-yl.

In an additional embodiment, Z is selected from 2-quinolinyl and 6-fluoroquinolin-2-yl.

In an additional embodiment, Z is 2-quinolinyl.

In another embodiment, Z is heteroaryl consisting of 6 ring atoms selected from C and N provided the total number of ring nitrogens is less than or equal to two; said ring is optionally substituted with up to 2 substituents independently selected from alkyl, alkoxy, cycloalkyl, cycloalkyloxy, cycloalkylalkyl, cycloalkylalkoxy, halogen, alkylsulfonyl and cyano and nitro.

In another embodiment, Z is heteroaryl consisting of 6 ring atoms selected from C and N provided the total number of ring nitrogens is less than or equal to two.

In a further embodiment, Z is pyridinyl optionally substituted with up to 2 substituents independently selected from alkyl, alkoxy, cycloalkyl, cycloalkyloxy, cycloalkylalkyl, cycloalkylalkoxy, halogen, alkylsulfonyl and cyano and nitro.

In a further embodiment, any Z is substituent may be unsubstituted.

In one embodiment, R₁ is selected from alkyl, CF₃, cycloalkyl, cycloalkyloxy, cycloalkylalkyl, cycloalkylalkoxy, alkoxyalkyl, halogen, alkoxy, thioalkyl, alkylsulfonyl, cyano, amino, alkylamino, dialkylamino, amido, alkylamido, dialkylamido and nitro

In another embodiment, R₁ is selected from halogen, CF₃, cyano, alkoxy, cycloalkoxy and alkoxyalkyl

In another embodiment, R₁ is selected from halogen, CF₃, cyano and alkoxy.

In a further embodiment, R₁ is selected from halogen, CF₃ and cyano.

In another embodiment, R₁ is halogen.

In an additional embodiment, R₁ is cyano.

In another embodiment, R₁ is methoxy.

In another embodiment, R₁ is CF₃;

In one embodiment R₁ is attached as follows:

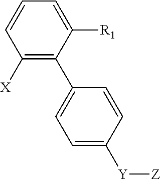

In another embodiment R₁ is attached as follows:

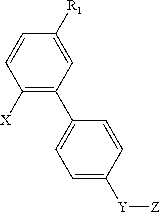

In one embodiment, R₂ is selected from hydrogen, C₁-C₄ alkyl, halogen, alkoxy, alkylthio, alkylsulfonyl, cyano or nitro.

In another embodiment, R₂ is selected from hydrogen, C₁-C₄ alkyl, halogen, alkoxy and cyano.

In another embodiment, R₂ is selected from hydrogen, halogen, alkoxy and cyano.

In another embodiment, R₂ is hydrogen.

In one embodiment R₂ is attached as follows in relationship to R₁:

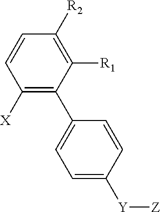

Compounds of the disclosure may contain asymmetric centers and exist as different enantiomers or diastereomers or a combination of these therein. All enantiomeric, diastereomeric forms of Formulas (I), (II) and (III) are embodied herein.

Compounds in the disclosure may be in the form of pharmaceutically acceptable salts. The phrase “pharmaceutically acceptable” refers to salts prepared from pharmaceutically acceptable non-toxic bases and acids, including inorganic and organic bases and inorganic and organic acids. Salts derived from inorganic bases include lithium, sodium, potassium, magnesium, calcium and zinc. Salts derived from organic bases include ammonia, primary, secondary and tertiary amines, and amino acids. Salts derived from inorganic acids include sulfuric, hydrochloric, phosphoric, hydrobromic. Salts derived from organic acids include C₁₋₆ alkyl carboxylic acids, di-carboxylic acids and tricarboxylic acids such as acetic acid, proprionic acid, fumaric acid, maleic acid, succinic acid, tartaric acid, adipic acid and citric acid, and alkylsulfonic acids such as methanesulphonic, and aryl sulfonic acids such as para-tolouene sulfonic acid and benzene sulfonic acid.

Compounds in the disclosure may be in the form of a solvate. This occurs when a compound of Formulas (I) or (II) or (III) has an energetically favorable interaction with a solvent, crystallizes in a manner that it incorporates solvent molecules into the crystal lattice or a complex is formed with solvent molecules in the solid or liquid state. Examples of solvents forming solvates are water (hydrates), MeOH, EtOH, iPrOH, and acetone.

Compounds in the disclosure may exist in different crystal forms known as polymorphs. Polymorphism is the ability of a substance to exist in two or more crystalline phases that have different arrangements and/or conformations of the molecule in the crystal lattice.

Compounds in the disclosure may exist as isotopically labeled compounds of Formulas (I) or (II) or (III) where one or more atoms are replaced by atoms having the same atomic number but a different atomic mass from the atomic mass which is predominantly seen in nature. Examples of isotopes include, but are not limited to hydrogen isotopes (deuterium, tritium), carbon isotopes (¹¹C, ¹³C, ¹⁴C) and nitrogen isotopes (¹³N, ¹⁵N). For example, substitution with heavier isotopes such as deuterium (²H) may offer certain therapeutic advantages resulting from greater metabolic stability which could be preferable and lead to longer in vivo half-life or dose reduction in a mammal or human.

Prodrugs of compounds embodied by Formulas (I) or (II) or (III) are also within the scope of this disclosure. Particular derivatives of compounds of Formulas (I) or (II) or (III) which may have little to negligible pharmacological activity themselves, can, when administered to a mammal or human, be converted into compounds of Formulas (I) or (II) or (III) having the desired biological activity.

Compounds in the disclosure and their pharmaceutically acceptable salts, prodrugs, as well as metabolites of the compounds, may also be used to treat certain eating disorders, obesity, compulsive gambling, sexual disorders, narcolepsy, sleep disorders, diabetes, metabolic syndrome, neurodegenerative disorders and CNS disorders/conditions as well as in smoking cessation treatment.

In one embodiment the treatment of CNS disorders and conditions by the compounds of the disclosure can include Huntington's disease, schizophrenia and schizo-affective conditions, delusional disorders, drug-induced psychoses, panic and obsessive compulsive disorders, post-traumatic stress disorders, age-related cognitive decline, attention deficit/hyperactivity disorder, bipolar disorders, personality disorders of the paranoid type, personality disorders of the schizoid type, psychosis induced by alcohol, amphetamines, phencyclidine, opioids hallucinogens or other drug-induced psychosis, dyskinesia or choreiform conditions including dyskinesia induced by dopamine agonists, dopaminergic therapies, psychosis associated with Parkinson's disease, psychotic symptoms associated with other neurodegenerative disorders including Alzheimer's disease, dystonic conditions such as idiopathic dystonia, drug-induced dystonia, torsion dystonia, and tardive dyskinesia, mood disorders including major depressive episodes, post-stroke depression, minor depressive disorder, premenstrual dysphoric disorder, dementia including but not limited to multi-infarct dementia, AIDS-related dementia, and neurodegenerative dementia,

In another embodiment, compounds of the disclosure may be used for the treatment of eating disorders, obesity, compulsive gambling, sexual disorders, narcolepsy, sleep disorders as well as in smoking cessation treatment.

In a further embodiment, compounds of the disclosure may be used for the treatment of obesity, schizophrenia, schizo-affective conditions, Huntington's disease, dystonic conditions and tardive dyskinesia.

In another embodiment, compounds of the disclosure may be used for the treatment of schizophrenia, schizo-affective conditions, Huntington's disease and obesity.

In a further embodiment, compounds of the disclosure may be used for the treatment of schizophrenia and schizo-affective conditions.

In an additional embodiment, compounds of the disclosure may be used for the treatment of Huntington's disease.

In another embodiment, compounds of the disclosure may be used for the treatment of obesity and metabolic syndrome.

Compounds of the disclosure may also be used in mammals and humans in conjuction with conventional antipsychotic medications including but not limited to Clozapine, Olanzapine, Risperidone, Ziprasidone, Haloperidol, Aripiprazole, Sertindole and Quetiapine. The combination of a compound of Formula (I) or (II) or (III) with a subtherapeutic dose of an aforementioned conventional antipsychotic medication may afford certain treatment advantages including improved side effect profiles and lower dosing requirements.

### DEFINITIONS

Alkyl is meant to denote a linear or branched saturated or unsaturated aliphatic C₁-C₈ hydrocarbon which can be optionally substituted with up to 3 fluorine atoms. Unsaturation in the form of a double or triple carbon-carbon bond may be internal or terminally located and in the case of a double bond both cis and trans isomers are included. Examples of alkyl groups include but are not limited to methyl, trifluoromethyl, ethyl, trifluoroethyl, isobutyl, neopentyl, cis- and trans-2-butenyl, isobutenyl, propargyl. C₁-C₄ alkyl is the subset of alkyl limited to a total of up to 4 carbon atoms.

In each case in which a size range for the number of atoms in a ring or chain is disclosed, all subsets are disclosed. Thus, Cₓ-C_{y} includes all subsets, e.g., C₁-C₄ includes C₁-C₂, C₂-C₄, C₁-C₃ etc.

Acyl is an alkyl-C(O)— group wherein alkyl is as defined above. Examples of acyl groups include acetyl and proprionyl.

Alkoxy is an alkyl-O— group wherein alkyl is as defined above. C₁-C₄ alkoxy is the subset of alkyl-O— where the subset of alkyl is limited to a total of up to 4 carbon atoms. Examples of alkoxy groups include methoxy, trifluoromethoxy, ethoxy, trifluoroethoxy, and propoxy

Alkoxyalkyl is an alkyl-O—(C₁-C₄alkyl)-group wherein alkyl is as defined above. Examples of alkoxyalkyl groups include methoxymethyl and ethoxymethyl.

Alkoxyalkyloxy is an alkoxy-alkyl-O— group wherein alkoxy and alkyl are as defined above. Examples of alkoxyalkyloxy groups include methoxymethyloxy (CH₃OCH₂O—) and methoxyethyloxy (CH₃OCH₂CH₂O—) groups.

Alkylthio is alkyl-S— group wherein alkyl is as defined above.

Alkylsulfonyl is alkyl-SO₂— wherein alkyl is as defined above.

Alkylamino is alkyl-NH— wherein alkyl is as defined above.

Dialkylamino is (alkyl)₂—N— wherein alkyl is as defined above.

Amido is H₂NC(O)—

Alkylamido is alkyl-NHC(O)— wherein alkyl is as defined above.

Dialkylamido is (alkyl)₂—NC(O)— wherein alkyl is as defined above.

Aromatic is heteroaryl or aryl wherein heteroaryl and aryl are as defined below.

Aryl is a phenyl or napthyl group. Aryl groups may be optionally and independently substituted with up to three groups selected from halogen, CF₃, CN, NO₂, OH, alkyl, cycloalkyl, cycloalkylalkyl, alkoxy, alkoxyalkyl, aryloxy, alkoxyalkyloxy, heterocycloalkyl, heterocycloalkylalkyl, heterocycloalkyloxy, heteroaryl, heteroaryloxy, —OCH₂CH₂OCH₃, —OC(O)Rₐ, —OC(O)ORₐ, —OC(O)NHRₐ, —OC(O)N(Rₐ), —SRₐ, —S(O)Rₐ, —NH₂, —NHRₐ, —N(Rₐ)(R_{b}), —NHC(O)Rₐ, —N(Rₐ)C(O)R_{b}, —NHC(O)ORₐ, —N(Rₐ)C(O)OR_{b}, —N(Rₐ)C(O)NH(R_{b}), —N(Rₐ)C(O)NH(R_{b})₂, —C(O)NH₂, —C(O)NHRₐ, —C(O)N(Rₐ)(R_{b}), —CO₂H, —CO₂Rₐ, —CORₐ wherein Rₐ and R_{b} are independently chosen from alkyl, alkoxyalkyl, —CH₂CH₂OH, —CH₂CH₂OMe, cycloalkyl, cycloalkylalkyl, aryl, arylalkyl, heteroaryl, heteroarylalkyl, heterocycloalkyl, and heterocycloalkylalkyl, each of which is optionally and independently substituted with up to three groups selected from only halogen, Me, Et, ⁱPr, ^{t}Bu, unsubstituted cyclopropyl, unsubstituted cyclobutyl, CN, NO₂, NH₂, CF₃, NHMe, NMe₂, OMe, OCF₃, each of which are attached via carbon-carbon or carbon-nitrogen or carbon-oxygen single bonds; or Rₐ and R_{b} taken together with the atom(s) to which they are attached form a 5-6 membered ring.

Arylalkyl is an aryl-alkyl-group wherein aryl and alkyl are as defined above.

Aryloxy is an aryl-O— group wherein aryl is as defined above.

Arylalkoxy is an aryl-(C₁-C₄ alkyl)-O— group wherein aryl is as defined above.

Carboxy is a CO₂H or CO₂R_{c} group wherein R_{c} is independently chosen from, alkyl, C₁-C₄ alkyl, cycloalkyl, arylalkyl, cycloalkylalkyl, CF₃, and alkoxyalkyl, wherein alkyl is as defined above.

Cycloalkyl is a C₃-C₇ cyclic non-aromatic hydrocarbon which may contain a single double bond and is optionally and independently substituted with up to three groups selected from alkyl, alkoxy, hydroxyl and oxo. Examples of cycloalkyl groups include cyclopropyl, cyclobutyl, cyclopentyl, cyclohexyl, cyclopentenyl and cyclohexanonyl.

Cycloalkyloxy is a cycloalkyl-O— group wherein cycloalkyl is as defined above. Examples include cyclopropyloxy, cyclobutyloxy and cyclopentyloxy. C₃-C₆ cycloalkyloxy is the subset of cycloalkyl-O— where cycloalkyl contains 3-6 carbon atoms.

Cycloalkylalkyl is a cycloalkyl-(C₁-C₄ alkyl)-group. Examples include cyclopropylmethyl, cyclopropylethyl, cyclohexylmethyl and cyclohexylethyl.

Cycloalkylalkoxy is a cycloalkyl-(C₁-C₄ alkyl)-O— group wherein cycloalkyl and alkyl are as defined above. Examples of cycloalkylalkoxy groups include cyclopropylmethoxy, cyclopentylmethoxy and cyclohexylmethoxy.

Halogen is F, Cl, Br or I.

Heteroaryl is a tetrazole, 1,2,3,4-oxatriazole, 1,2,3,5-oxatriazole, a mono or bicyclic aromatic ring system, or a heterobicyclic ring system with one aromatic ring having 5 to 10 ring atoms independently selected from C, N, O and S, provided that not more than 3 ring atoms in any single ring are other than C. Examples of heteroaryl groups include but are not limited to thiophenyl, furanyl, thiazolyl, isothiazolyl, oxazolyl, isoxazolyl, 1,2,4-oxadiazolyl, 1,3,4-oxadiazolyl, pyrrazolyl, imidazolyl, 1,2,3-triazolyl, 1,3,4-triazolyl, pyrimidinyl, pyrazinyl, indolyl, quinolyl, tetrahydroquinolyl, isoquinolyl, tetrahydroisoquinolyl, indazolyl, benzthiadiazololyl, benzoxadiazolyl and benzimidazolyl. Heteroaryl groups may be optionally and independently substituted with up to 3 substituents independently selected from halogen, CF₃, CN, NO₂, OH, alkyl, cycloalkyl, cycloalkylalkyl, alkoxy, alkoxyalkyl, aryloxy, alkoxyalkyloxy, heterocycloalkyl, heterocycloalkylalkyl, heterocycloalkyloxy, heteroaryl, heteroaryloxy, —OCH₂CH₂OCH₃, —OC(O)Rₐ, —OC(O)ORₐ, —OC(O)NHRₐ, —OC(O)N(Rₐ), —SRₐ, —S(O)Rₐ, —NH₂, —NHRₐ, —N(Rₐ)(R_{b}), —NHC(O)Rₐ, —N(Rₐ)C(O)R_{b}, —NHC(O)ORₐ, —N(Rₐ)C(O)OR_{b}, —N(Rₐ)C(O)NH(R_{b}), —N(Rₐ)C(O)NH(R_{b})₂, —C(O)NH₂, —C(O)NHRₐ, —C(O)N(Rₐ)(R_{b}), —CO₂H, —CO₂Rₐ, —CORₐ wherein Rₐ and R_{b} are independently chosen from alkyl, alkoxyalkyl, —CH₂CH₂OH, —CH₂CH₂OMe, cycloalkyl, cycloalkylalkyl, aryl, arylalkyl, heteroaryl, heteroarylalkyl, heterocycloalkyl, and heterocycloalkylalkyl, each of which is optionally and independently substituted with up to three groups selected from only halogen, Me, Et, ⁱPr, ^{t}Bu, unsubstituted cyclopropyl, unsubstituted cyclobutyl, CN, NO₂, NH₂, CF₃, NHMe, NMe₂, OMe, OCF₃, each of which are attached via carbon-carbon or carbon-nitrogen or carbon-oxygen single bonds; or Rₐ and R_{b} taken together with the atom(s) to which they are attached form a 5-6 membered ring.

Heteroarylalkyl is a heteroaryl-(C₁-C₄ alkyl)-group wherein heteroaryl and alkyl are as defined above. Examples of heteroarylalkyl groups include 4-pyridinylmethyl and 4-pyridinylethyl.

Heteroaryloxy is a heteroaryl-O group wherein heteroaryl is as defined above.

Heteroarylalkoxy is a heteroaryl-(C₁-C₄ alkyl)-O— group wherein heteroaryl and alkoxy are as defined above. Examples of heteroarylalkyl groups include 4-pyridinylmethoxy and 4-pyridinylethoxy.

Heterobicyclic ring system is a ring system having 8-10 atoms independently selected from C, N, O and S, provided that not more than 3 ring atoms in any single ring are other than carbon and provided that at least one of the rings is aromatic; said bicyclic ring may be optionally and independently substituted with up to 3 substituents independently selected from alkyl, alkoxy, cycloalkyl, C₃-C₆ cycloalkyloxy, cycloalkylalkyl, halogen, nitro, alkylsulfonyl and cyano. Examples of 8-10 membered heterobicyclic ring systems include but are not limited to 1,5-naphthyridyl, 1,2,3,4-tetrahydro-1,5-naphthyridyl 1,6-naphthyridyl, 1,2,3,4-tetrahydro-1,6-naphthyridyl 1,7-naphthyridyl, 1,2,3,4-tetrahydro-1,7-naphthyridinyl 1,8-naphthyridyl, 1,2,3,4-tetrahydro-1,8-naphthyridyl, 2,6-naphthyridyl, 2,7-naphthyridyl, cinnolyl, isoquinolyl, tetrahydroisoquinolinyl, phthalazyl, quinazolyl, 1,2,3,4-tetrahydroquinazolinyl, quinolyl, tetrahydroquinolinyl, quinoxalyl, tetrahydroquinoxalinyl, benzo[d][1,2,3]triazyl, benzo[e][1,2,4]triazyl, pyrido[2,3-b]pyrazyl, pyrido[2,3-c]pyridazyl, pyrido[2,3-d]pyrimidyl, pyrido[3,2-b]pyrazyl, pyrido[3,2-c]pyridazyl, pyrido[3,2-c]pyrimidyl, pyrido[3,4-b]pyrazyl, pyrido[3,4-c]pyridazyl, pyrido[3,4-d]pyrimidyl, pyrido[4,3-b]pyrazyl, pyrido[4,3-c]pyridazyl, pyrido[4,3-c]pyrimidyl, quinazolyl, 1H-benzo[d][1,2,3]triazoyl, 1H-benzo[d]imidazoyl, 1H-indazoyl, 1H-indoyl, 2H-benzo[d][1,2,3]triazoyl, 2H-pyrazolo[3,4-b]pyridinyl, 2H-pyrazolo[4,3-b]pyridinyl, [1,2,3]triazolo[1,5-a]pyridinyl, [1,2,4]triazolo[1,5-a]pyridinyl, [1,2,4]triazolo[4,3-a]pyridinyl, benzo[b]thienyl, benzo[c][1,2,5]oxadiazyl, benzo[c][1,2,5]thiadiazolyl, benzo[d]isothiazoyl, benzo[d]isoxazoyl, benzo[d]oxazoyl, benzo[d]thiazoyl, benzofuryl, imidazo[1,2-a]pyrazyl, imidazo[1,2-a]pyridinyl, imidazo[1,2-a]pyrimidyl, imidazo[1,2-b]pyridazyl, imidazo[1,2-c]pyrimidyl, imidazo[1,5-a]pyrazyl, imidazo[1,5-a]pyridinyl, imidazo[1,5-a]pyrimidyl, imidazo[1,5-b]pyridazyl, imidazo[1,5-c]pyrimidyl, indolizyl, pyrazolo[1,5-a]pyrazyl, pyrazolo[1,5-a]pyridinyl, pyrazolo[1,5-a]pyrimidyl, pyrazolo[1,5-b]pyridazine, pyrazolo[1,5-c]pyrimidine, pyrrolo[1,2-a]pyrazine, pyrrolo[1,2-a]pyrimidyl, pyrrolo[1,2-b]pyridazyl, pyrrolo[1,2-c]pyrimidyl, 1H-imidazo[4,5-b]pyridinyl, 1H-imidazo[4,5-c]pyridinyl, 1H-pyrazolo[3,4-b]pyridinyl, 1H-pyrazolo[3,4-c]pyridinyl, 1H-pyrazolo[4,3-b]pyridinyl, 1H-pyrazolo[4,3-c]pyridinyl, 1H-pyrrolo[2,3-b]pyridinyl, 1H-pyrrolo[2,3-c]pyridinyl, 1H-pyrrolo[3,2-b]pyridinyl, 1H-pyrrolo[3,2-c]pyridinyl, 2H-indazoyl, 3H-imidazo[4,5-b]pyridinyl, 3H-imidazo[4,5-c]pyridinyl, benzo[c]isothiazyl, benzo[c]isoxazyl, furo[2,3-b]pyridinyl, furo[2,3-c]pyridinyl, furo[3,2-b]pyridinyl, furo[3,2-c]pyridiyl, isothiazolo[4,5-b]pyridinyl, isothiazolo[4,5-c]pyridinyl, isothiazolo[5,4-b]pyridinyl, isothiazolo[5,4-c]pyridinyl, isoxazolo[4,5-b]pyridinyl, isoxazolo[4,5-c]pyridinyl, isoxazolo[5,4-b]pyridinyl, isoxazolo[5,4-c]pyridinyl, oxazolo[4,5-b]pyridinyl, oxazolo[4,5-c]pyridinyl, oxazolo[5,4-b]pyridinyl, oxazolo[5,4-c]pyridinyl, thiazolo[4,5-b]pyridiyl, thiazolo[4,5-c]pyridinyl, thiazolo[5,4-b]pyridinyl, thiazolo[5,4-c]pyridinyl, thieno[2,3-b]pyridinyl, thieno[2,3-c]pyridinyl, thieno[3,2-b]pyridinyl and thieno[3,2-c]pyridinyl.

Heterocycloalkyl is a non-aromatic, monocyclic or bicyclic saturated or partially unsaturated ring system comprising 5-10 ring atoms selected from C, N, O and S, provided that not more than 2 ring atoms in any single ring are other than C. In the case where the heterocycloalkyl group contains a nitrogen atom the nitrogen may be substituted with an alkyl, acyl, —C(O)O-alkyl, —C(O)NH(alkyl) or a —C(O)N(alkyl)₂ group. Heterocycloalkyl groups may be optionally and independently substituted with hydroxy, alkyl and alkoxy groups and may contain up to two oxo groups. Heterocycloalkyl groups may be linked to the rest of the molecule via either carbon or nitrogen ring atoms. Examples of heterocycloalkyl groups include tetrahydrofuranyl, tetrahydrothienyl, tetrahydro-2H-pyran, tetrahydro-2H-thiopyranyl, pyrrolidinyl, pyrrolidonyl, succinimidyl, piperidinyl, piperazinyl, N-methylpiperazinyl, morpholinyl, morpholin-3-one, thiomorpholinyl, thiomorpholin-3-one, 2,5-diazabicyclo[2.2.2]octanyl, 2,5-diazabicyclo[2.2.1]heptanyl, octahydro-1H-pyrido[1,2-a]pyrazine, 3-thia-6-azabicyclo[3.1.1]heptane and 3-oxa-6-azabicyclo[3.1.1]heptanyl

Heterocycloalkylalkyl is a heterocycloalkyl-(C₁-C₄ alkyl)-group wherein heterocycloalkyl is as defined above.

Heterocycloalkyloxy is a heterocycloalkyl-O— group wherein heterocycloalkyl is as defined above.

Heterocycloalkylalkoxy is a heterocycloalkyl-(C₁-C₄ alkyl)-O— group wherein heterocycloalkyl is as defined above.

Oxo is a —C(O)— group.

Phenyl is a benzene ring which may be optionally and independently substituted with up to three groups selected from halogen, CF₃, CN, NO₂, OH, alkyl, cycloalkyl, cycloalkylalkyl, alkoxy, alkoxyalkyl, aryloxy, alkoxyalkyloxy, heterocycloalkyl, heterocycloalkylalkyl, heterocycloalkyloxy, heteroaryl, heteroaryloxy, —OCH₂CH₂OCH₃, —OC(O)Rₐ, —OC(O)ORₐ, —OC(O)NHRₐ, —OC(O)N(Rₐ), —SRₐ, —S(O)Rₐ, —NH₂, —NHRₐ, —N(Rₐ)(R_{b}), —NHC(O)Rₐ, —N(Rₐ)C(O)R_{b}, —NHC(O)ORₐ, —N(Rₐ)C(O)OR_{b}, —N(Rₐ)C(O)NH(R_{b}), —N(Rₐ)C(O)NH(R_{b})₂, —C(O)NH₂, —C(O)NHRₐ, —C(O)N(Rₐ)(R_{b}), —CO₂H, —CO₂Rₐ, —CORₐ wherein Rₐ and R_{b} are independently chosen from alkyl, alkoxyalkyl, —CH₂CH₂OH, —CH₂CH₂OMe, cycloalkyl, cycloalkylalkyl, aryl, arylalkyl, heteroaryl, heteroarylalkyl, heterocycloalkyl, and heterocycloalkylalkyl, each of which is optionally and independently substituted with up to three groups selected from only halogen, Me, Et, ⁱPr, ^{t}Bu, unsubstituted cyclopropyl, unsubstituted cyclobutyl, CN, NO₂, NH₂, CF₃, NHMe, NMe₂, OMe, OCF₃, each of which are attached via carbon-carbon or carbon-nitrogen or carbon-oxygen single bonds; or Rₐ and R_{b} taken together with the atom(s) to which they are attached form a 5-6 membered ring.

Restricted phenyl is a benzene ring which may be optionally and independently substituted with up to three groups selected from halogen, CF₃, CN, alkoxy, alkoxyalkyl, aryloxy, alkoxyalkyloxy, heterocycloalkyl, heterocycloalkyloxy, heteroaryl, heteroaryloxy, —OCH₂CH₂OCH₃, —OC(O)Rₐ, —OC(O)ORₐ, —OC(O)N(Rₐ), —N(Rₐ)(R_{b}), —NHC(O)Rₐ, —N(Rₐ)C(O)R_{b}, —NHC(O)ORₐ, —N(Rₐ)C(O)OR_{b}, —C(O)N(Rₐ)(R_{b}), —CORₐ wherein Rₐ and R_{b} are independently chosen from alkyl, alkoxyalkyl, —CH₂CH₂OH, —CH₂CH₂OMe, cycloalkyl, cycloalkylalkyl, aryl, arylalkyl, heteroaryl, heteroarylalkyl, heterocycloalkyl, and heterocycloalkylalkyl, each of which is optionally and independently substituted with up to three groups selected from only halogen, Me, Et, ⁱPr, ^{t}Bu, unsubstituted cyclopropyl, unsubstituted cyclobutyl, CN, NO₂, NH₂, CF₃, NHMe, NMe₂, OMe, OCF₃, each of which are attached via carbon-carbon or carbon-nitrogen or carbon-oxygen single bonds; or Rₐ and R_{b} taken together with the atom(s) to which they are attached form a 5-6 membered ring.

The position of R₁ (or the position of R₂) on the central phenyl ring is defined as follows:

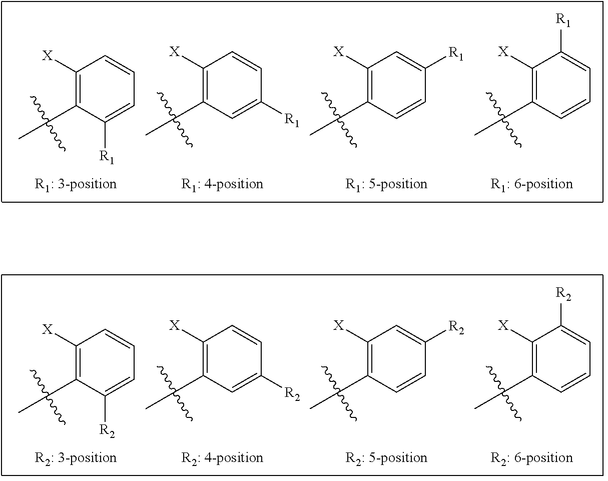

Abbreviations used in the following examples and preparations include:
Ac Acyl (Me-C(O)—)
AcN Acetonitrile
BINAP 2,2′-bis(diphenylphosphino)-1,1′-binaphthyl
Bn Benzyl
Celite® Diatomaceous earth
DBU 1,8-Diazabicyclo[5.4.0]undec-7-ene
DCC N,N′, Dicyclohexylcarbodiimide
DCM Dichloromethane
DIEA Di-isopropylethyl amine
DIPEA Di-isopropylethyl amine
DMAP 4-Dimethylaminopyridine
DMF Dimethylformamide
DMP Dess Martin Periodinane
DMSO Dimethyl sulfoxide
Dppf 1,4-Bis(diphenylphosphino) ferrocene
EDC 1-(3-Dimethylaminopropyl)-3-ethylcarbodiimide Hydrochloride
Et₃N Triethylamine
g gram(s)
h Hour(s)
hr Hour(s)
HATU 2-(7-Aza-1H-benzotriazole-1-yl)-1,1,3,3-tetramethyluronium hexafluorophosphate
HMDS Hexamethyldisilazide
HOBt 1-Hydroxybenzotriazole
HPLC High Pressure Liquid Chromatography
HRMS High resolution mass spectrometry
i.v. Intravenous
KHMDS Potassium Hexamethydisilazide
LDA Lithium Di-isopropylamide
m Multiplet
m-meta
MEM Methoxyethoxymethyl
MeOH Methyl Alcohol or Methanol
min Minute(s)
mmol millimoles
mmole millimoles
Ms Mesylate
MS Mass Spectrometry
MW Molecular Weight
NBS N-Bromosuccinamide
NIS N-Iodosuccinamide
NMR Nuclear Magnetic Resonance
NMM N-Methyl Morpholine
NMP N-Methyl-2-pyrrolidone
o ortho
o/n overnight
p para
PCC Pyridinium Chlorochromate
PEPPSI 1,3-Bis(2,6-diisopropylphenyl)imidazolidene)(3-chloropyridinyl)palladium(II) dichloride
PhNTf₂ 1,1,1-trifluoro-N-phenyl-N-(trifluoromethylsulfonyl)methanesulfonamide
POPd Dihydrogen dichlorobis(di-tert-butylphosphinito-kp) palladate (2-)
p.s.i. Pounds per square inch
PPA Polyphosphoric acid
PPAA 1-Propanephosphonic Acid Cyclic Anhydride
PTSA p-Toluenesulfonic acid
PyBOP® Benzotriazol-1-yl-oxytripyrrolidinophosphonium hexafluorophosphate
RT (or rt) room temperature (about 20-25° C.)
s Singlet
sat. Saturated
t Triplet
TBAF Tetra-butyl ammonium fluoride
TEA Triethylamine
TFA Trifluoroacetic Acid
THF Tetrahydrofuran
TLC Thin layer chromatography
TMS Trimethylsilyl
Tf Triflate
Tof-MS Time of Flight Mass Spectrometry
Ts Tosylate
v/v volume/volume
wt/v weight/volume

### DETAILED DESCRIPTION OF THE DISCLOSURE

The di-substituted phenyl compounds of Formulas (I), (II) and (III) may be prepared from multi-step organic synthesis routes from known diiodo- or dibromobenzenes, or alternatively from nitrophenol or bromophenol starting materials by one skilled in the art of organic synthesis using established organic synthesis procedures.

Compounds of the disclosure of Formula (I) in which R₁=R₂ and X=phenyl or heteroaryl are as described previously and thus having general Formula XII may be prepared generally as depicted in Scheme 1.

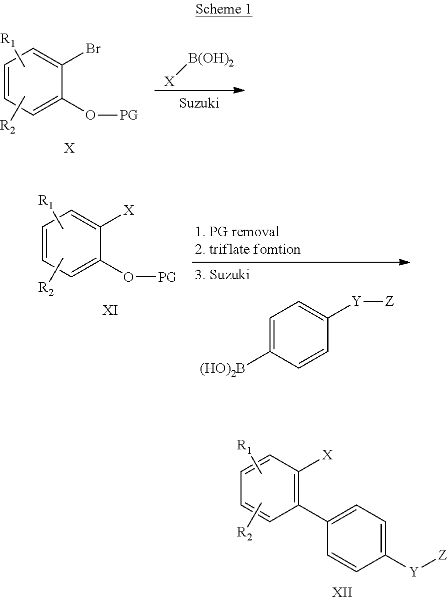

Compounds of the disclosure of Formula (I) in which X=C₃-C₈ alkyl, cycloalkyl, cycloalkylalkyl, cycloalkylalkoxy, heterocycloalkyl, heterocycloalkyloxy and R₁=R₂=H are as described previously and thus having general Formula XXI may be prepared generally as depicted in Scheme 2.

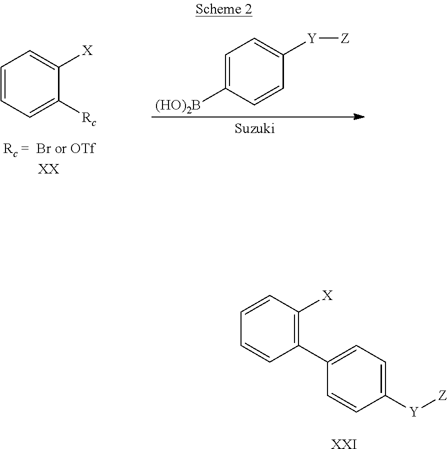

Compounds of the disclosure of Formula (I) in which X=phenyl or heteroaryl and R₁≠R₂ are as described previously and thus having general Formula XXXIV may be prepared generally as depicted in Scheme 3.

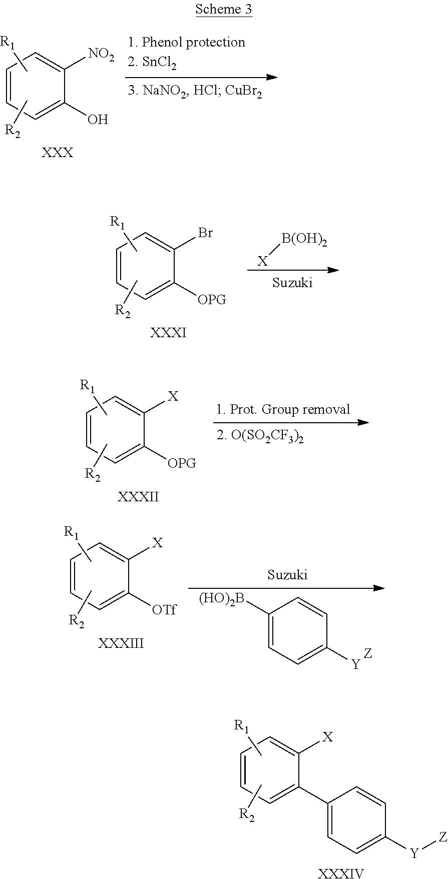

Compounds of the disclosure of Formula (II) in which X=phenyl or heteroaryl are as described previously and thus having general Formula XLIII may be prepared generally as depicted in Scheme 4.

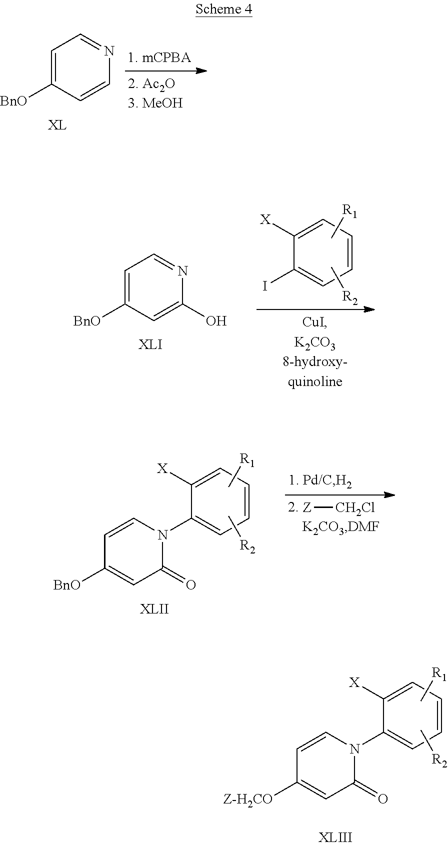

Compounds of the disclosure of Formula (III) in which X=phenyl or heteroaryl are as described previously and thus having general Formula LII may be prepared generally as depicted in Scheme 5.

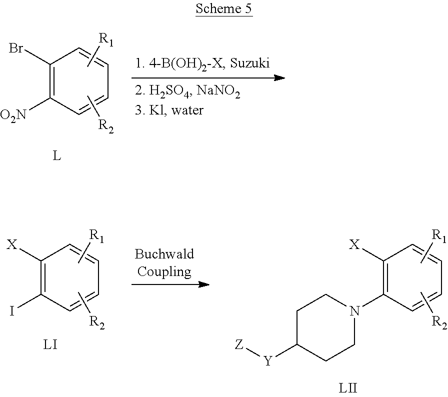

Reactive groups not involved in the above processes can be protected with standard protecting groups (PG) during the reactions and removed by standard procedures (T. W. Greene & P. G. M. Wuts, Protecting Groups in Organic Synthesis, Third Edition, Wiley-Interscience) known to those of ordinary skill in the art. Presently preferred protecting groups include methyl, MEM, benzyl, acetate and tetrahydropyranyl for the hydroxyl moiety, and BOC, Cbz, trifluoroacetamide and benzyl for the amino moiety, methyl, ethyl, tert-butyl and benzyl esters for the carboxylic acid moiety.

### EXPERIMENTAL PROCEDURES

### Synthesis of 2-(4′-Methyl-2′-pyridin-4-yl-biphenyl-4-yloxymethyl)-quinoline

### Example 1867

### 2-(2-Bromo-4-methyl-phenoxy)-tetrahydropyran

### Error! Objects cannot be created from editing field codes

To a stirred solution of 2-bromo-4-methylphenol (5.050 g) in CH₂Cl₂ (30 mL) was added pyridinium p-toluenesulfonate (PPTS, 0.068 g), followed by 3,4-dihydro-2H-pyran (2.730 g) at room temperature under an argon atmosphere and the reaction mixture was stirred at room temperature for 20 h. The solvent was removed under reduced pressure and the residue was purified by silica gel chromatography eluting with 0-20% EtOAc/heptane to provide the title compound 2-(2-bromo-4-methylphenoxy)tetrahydro-2H-pyran as a colorless oil (6.9 g). ¹H NMR (300 MHz, CDCl₃/TMS) δ 7.35 (s, 1H), 7.03 (s, 2H), 5.45 (s, 1H), 3.92 (dt, J=10.9, 2.4 Hz, 1H), 3.59 (d, J=10.8 Hz, 1H), 2.27 (s, 3H), 2.20-1.80 (m, 3H), 1.80-1.56 (m, 3H); ¹³C NMR (75 MHz, CDCl₃/TMS) δ 150.9, 133.3, 132.4, 128.6, 116.5, 112.7, 96.7, 61.7, 30.1, 25.2, 20.2, 18.3.

4-(5-Methyl-2-(tetrahydro-pyran-2-yloxy)-phenyl)-pyridine

### Error! Objects cannot be created from editing field codes

A mixture of 2-(2-bromo-4-methyl-phenoxy)-tetrahydropyran (1.98 g), pyridine-4-boronic acid (1.080 g) and Cs₂CO₃ (7.14 g) in dry DMF (20 mL) was purged with argon. Pd(dppf)Cl₂ (0.270 g) was added and the mixture was purged again with argon. The reaction mixture was heated to 110° C. for 24 h. The mixture was cooled to room temperature and the solvent was removed under reduced pressure. The residue was suspended in EtOAc and filtered through a silica gel plug eluting with EtOAc. Evaporation and purification by chromatography eluting with 0-70% EtOAc/heptane produced the title compound 4-(5-Methyl-2-(tetrahydro-pyran-2-yloxy)-phenyl)-pyridine (0.970 g) as a brown oil. ¹H NMR (300 MHz, CDCl₃/TMS) δ 8.62 (dd, J=4.8, 1.5 Hz, 2H), 7.50 (dd, J=4.5, 1.5 Hz, 2H), 7.16 (s, 3H), 5.39 (s, 1H), 3.76 (t, J=10.3 Hz, 1H), 3.57 (d, J=11.1 Hz, 1H), 2.34 (s, 3H), 1.88-1.70 (m, 3H), 1.70-1.46 (m, 3H); ¹³C NMR (75 MHz, CDCl₃/TMS) δ 151.5, 149.1, 146.4, 131.2, 130.6, 130.3, 128.1, 124.2, 115.6, 96.7, 61.8, 30.2, 25.1, 20.5, 18.5.

### 4-Methyl-2-pyridin-4-yl-phenol

### Error! Objects cannot be created from editing field codes

To a solution of 4-(5-methyl-2-(tetrahydropyran-2-yloxy)-phenyl)-pyridine (0.750 g) in MeOH (20 mL) was added trifluoroacetic acid (0.950 g) and the reaction mixture was stirred at room temperature for 20 h. The solvent was removed under reduced pressure. The residue was suspended in EtOAc (50 mL) and neutralized with saturated aqueous NaHCO₃ solution. The organic phase was separated and washed with brine, and dried over MgSO₄. Filtration and concentration produced the title compound 4-methyl-2-pyridin-4-yl-phenol (0.510 g) as a yellow solid. ¹H NMR (300 MHz, CD₃OD/CDCl₃/TMS) δ 8.52 (b s, 2H), 7.71 (d, J=5.1 Hz, 2H), 7.15 (br s, 1H), 7.08 (d, J=9.3 Hz, 1H), 6.87 (d, J=8.4 Hz, 1H), 2.32 (s, 3H); ¹³C NMR (75 MHz, CD₃OD/CDCl₃/TMS) δ 152.4, 149.1, 147.5, 131.2, 130.6, 129.4, 124.8, 124.4, 116.4, 20.4.

### Trifluoromethanesulfonic acid 4-methyl-2-pyridin-4-yl-phenyl ester

### Error! Objects cannot be created from editing field codes

A solution of 4-methyl-2-pyridin-4-yl-phenol (0.590 g) in dry pyridine (10 mL) was treated with trifluoromethanesulfonic anhydride (0.990 g) at 0° C. under argon. The resulting mixture was stirred at 0° C. for 0.5 h, then allowed to warm to room temperature and stirred for 16 h. The solvent was removed under reduced pressure, and the residue was dissolved in CH₂Cl₂ (100 mL), washed with cold saturated NaHCO₃ aqueous solution (2×50 mL), and dried over MgSO₄. Filtration, evaporation and purification by chromatography eluting with 0-40% EtOAc/heptane provided title compound trifluoromethanesulfonic acid 4-methyl-2-pyridin-4-yl-phenyl ester (0.780 g) as a colorless oil. ¹H NMR (300 MHz, CDCl₃/TMS) δ 8.70 (dd, J=4.7, 1.5 Hz, 2H), 7.39 (dd, J=4.5, 1.5 Hz, 2H), 7.30 (br s, 2H), 7.27 (br s, 1H), 2.44 (s, 3H); ¹³C NMR (75 MHz, CDCl₃/TMS) δ 149.8, 144.1, 143.4, 138.9, 132.2, 131.7, 130.7, 123.7, 121.9, 118.1 (J=318 Hz), 20.9.

### 2-(4′-Methyl-2′-pyridin-4-yl-biphenyl-4-yloxymethyl)-quinoline

### Example 1867

### Error! Objects cannot be created from editing field codes

A suspension of trifluoromethanesulfonic acid 4-methyl-2-pyridin-4-yl-phenyl ester (0.390 g), 2-(4-(4,4,5,5-tetramethyl(1,3,2)dioxaborolan-2-yl)-phenoxymethyl)-quinoline (0.490 g) and Cs₂CO₃ (1.200 g) in dry DMF (10 mL) was purged with argon. Pd(dppf)Cl₂ (0.045 g) was added and the mixture was purged again with argon. The reaction mixture was heated to 110° C. for 24 h. The mixture was cooled to room temperature and the solvent was removed under reduced pressure. The residue was suspended in EtOAc and filtered through a silica gel plug eluting with EtOAc. Evaporation and purification by chromatography eluting with 10-50% EtOAc/heptane produced the title compound 2-(4′-methyl-2′-pyridin-4-yl-biphenyl-4-yloxymethyl)-quinoline (0.038 g) as a yellow wax. ¹H NMR (300 MHz, CDCl₃/TMS) δ 8.43 (d, J=2.1 Hz, 2H), 8.19 (d, J=8.4 Hz, 1H), 8.08 (d, J=8.4 Hz, 1H), 7.83 (d, J=7.8 Hz, 1H), 7.73 (t, J=7.2 Hz, 1H), 7.66 (d, J=8.4 Hz, 1H), 7.54 (t, J=7.2 Hz, 1H), 7.34-7.22 (m, 2H), 7.20 (b s, 1H), 7.08-6.97 (m, 4H), 6.89 (d, J=8.4 Hz, 2H), 5.35 (s, 2H), 2.43 (s, 3H); ¹³C NMR (75 MHz, CDCl₃/TMS) δ 157.5, 157.2, 149.5, 149.1, 147.3, 137.2, 137.1, 137.0, 136.7, 133.2, 130.7, 130.6, 130.5, 129.6, 129.2, 128.7, 127.5, 127.4, 126.3, 124.5, 118.9, 114.4, 71.1, 21.0; HRMS: M⁺H m/z=403.1838.

### Synthesis of 2-(5′-methyl-2′-pyridin-4-yl-biphenyl-4-yloxymethyl)-quinoline

### Example 408

### 2-(5′-Methyl-2′-(tetrahydropyran-2-yloxy)-biphenyl-4-yloxymethyl)-quinoline

### Error! Objects cannot be created from editing field codes

A suspension of 2-(2-bromo-4-methyl-phenoxy)-tetrahydropyran (1.380 g), 2-(4-(4,4,5,5-tetramethyl(1,3,2)dioxaborolan-2-yl)-phenoxymethyl)-quinoline (2.020 g) and Cs₂CO₃ (4.970 g) in dry DMF (20 mL) was purged with argon. Pd(dppf)Cl₂ (0.190 g) was added and the mixture was purged again with argon. The reaction mixture was heated to 110° C. for 24 h. The mixture was cooled to room temperature and the solvent was removed under reduced pressure. The residue was suspended in EtOAc and filtered through a silica gel plug eluting with EtOAc Evaporation and purification by chromatography eluting with 10-70% EtOAc/heptane produced the title compound 2-(5′-Methyl-2′-(tetrahydropyran-2-yloxy)-biphenyl-4-yloxymethyl)-quinoline (1.320 g) as a white solid. ¹H NMR (300 MHz, CDCl₃/TMS) δ 8.19 (d, J=8.7 Hz, 1H), 8.09 (d, J=8.4 Hz, 1H), 7.83 (d, J=8.1 Hz, 1H), 7.78-7.62 (m, 2H), 7.60-7.40 (m, 3H), 7.15-6.82 (m, 5H), 5.43 (s, 2H), 5.31 (s, 1H), 3.76 (t, J=10.7 Hz, 1H), 3.52 (d, J=11.4 Hz, 1H), 2.31 (s, 3H), 1.82-1.40 (m, 6H); ¹³C NMR (75 MHz, CDCl₃/TMS) δ 157.8, 157.1, 151.4, 147.3, 136.7, 131.5, 131.04, 130.96, 130.8, 130.5, 129.5, 128.7, 128.3, 127.5, 127.4, 126.3, 119.0, 116.0, 114.0, 96.7, 71.2, 61.6, 30.2, 25.2, 20.6, 18.5.

### 5-Methyl-4′-(quinolin-2-ylmethoxy)-biphenyl-2-ol

### Error! Objects cannot be created from editing field codes

To a suspension of 2-(5′-methyl-2′-(tetrahydro-pyran-2-yloxy)-biphenyl-4-yloxymethyl)-quinoline (0.790 g) in a mixture of MeOH (30 mL) and CH₂Cl₂ (5 mL) was added pyridinium p-toluenesulfonate (PPTS, 0.009 g) and the reaction mixture was stirred and heated to 60° C. for 19 h. The solvent was removed under reduced pressure. The residue was purified by chromatography eluting with 0-2% MeOH/CH₂Cl₂ to produce the title compound 5-methyl-4′-(quinolin-2-ylmethoxy)-biphenyl-2-ol (0.600 g) as a white solid. ¹H NMR (300 MHz, CD₃OD/CDCl₃/TMS) δ 8.33 (d, J=8.4 Hz, 1H), 8.07 (d, J=8.4 Hz, 1H), 7.91 (d, J=8.4 Hz, 1H), 7.82-7.72 (m, 2H), 7.60 (t, J=7.5 Hz, 1H), 7.50 (d, J=8.7 Hz, 2H), 7.12-7.01 (m, 3H), 6.93 (dd, J=6.3, 0.6 Hz, 1H), 6.78 (d, J=8.1 Hz, 1H), 5.40 (s, 2H), 2.27 (s, 3H); ¹³C NMR (75 MHz, CD₃OD/CDCl₃/TMS) δ 158.6, 157.9, 152.0, 147.7, 138.3, 132.7, 131.4, 131.0, 130.6, 129.5, 129.0, 128.42, 128.40, 127.3, 120.0, 116.3, 115.1, 71.4, 20.5.

### Trifluoro-methanesulfonic acid 5-methyl-4′-(quinolin-2-ylmethoxy)-2-yl ester

### Error! Objects cannot be created from editing field codes

A solution of 5-methyl-4′-(quinolin-2-ylmethoxy)-biphenyl-2-ol (0.410 g) in dry pyridine (10 mL) was treated with trifluoromethanesulfonic anhydride (0.370 g) at 0° C. under argon. The resulting mixture was stirred at 0° C. for 0.5 h, then allowed to warm to room temperature and stirred for 7 h. The solvent was removed under reduced pressure, and the residue was dissolved in CH₂Cl₂ (100 mL), washed with cold saturated aqueous NaHCO₃ solution (2×50 mL), and dried over MgSO₄. Filtration, evaporation and purification by chromatography eluting with 0-2% MeOH/CH₂Cl₂ provided trifluoro-methanesulfonic acid 5-methyl-4′-(quinolin-2-ylmethoxy)-2-yl ester (0.350 g) as a colorless oily wax. ¹H NMR (300 MHz, CDCl₃/TMS) δ 8.14 (d, J=8.4 Hz, 1H), 8.09 (d, J=8.4 Hz, 1H), 7.78 (d, J=8.4 Hz, 1H), 7.74-7.62 (m, 2H), 7.51 (t, J=7.5 Hz, 1H), 7.37 (d, J=8.4 Hz, 2H), 7.25-7.16 (m, 2H), 7.16-7.05 (m, 3H), 5.40 (s, 2H), 2.34 (s, 3H); ¹³C NMR (75 MHz, CDCl₃/TMS) δ 158.2, 157.3, 147.3, 144.6, 138.3, 136.8, 134.4, 132.1, 130.4, 129.6, 128.9, 128.7, 128.4, 127.5, 127.4, 126.3, 121.5, 118.9, 118.2 (J=318 Hz), 114.7, 71.2, 20.8.

### 2-(5′-Methyl-2′-pyridin-4-yl-biphenyl-4-yloxymethyl)-quinoline

### Example 408

### Error! Objects cannot be created from editing field codes

A mixture of trifluoromethanesulfonic acid 5-methyl-4′-(quinolin-2-ylmethoxy)-2-yl ester (0.350 g), pyridine-4-boronic acid (0.136 g) and 2M aqueous Na₂CO₃ solution (2 mL) in dioxane (10 mL) was purged with argon. Pd(dppf)Cl₂ (0.027 g) was added and the mixture was purged again with argon. The reaction mixture was heated to reflux for 20 h. The mixture was then cooled to room temperature and the solvent was removed under reduced pressure. The residue was suspended in EtOAc and filtered through a silica gel plug. Evaporation and purification by silica gel flash chromatography eluting with 0-2% MeOH/CH₂Cl₂ provided 2-(5′-methyl-2′-pyridin-4-yl-biphenyl-4-yloxymethyl)-quinoline (0.035 g) as a colorless oily wax. ¹H NMR (300 MHz, CDCl₃/TMS) δ 8.43 (b s, 2H), 8.19 (d, J=8.7 Hz, 1H), 8.08 (d, J=8.1 Hz, 1H), 7.83 (d, J=7.8 Hz, 1H), 7.73 (t, J=7.4 Hz, 1H), 7.66 (d, J=8.7 Hz, 1H), 7.55 (t, J=7.4 Hz, 1H), 7.32-7.19 (m, 3H), 7.08-6.97 (m, 4H), 6.90 (d, J=8.4 Hz, 2H), 5.36 (s, 2H), 2.42 (s, 3H); ¹³C NMR (75 MHz, CDCl₃/TMS) δ 157.5, 157.3, 149.3, 149.0, 147.3, 139.8, 138.4, 136.7, 134.6, 133.4, 131.3, 130.7, 129.9, 129.6, 128.7, 128.0, 127.5, 127.4, 126.3, 124.6, 118.9, 114.4, 71.2, 21.1; HRMS: M⁺H m/z=403.1817.

### Synthesis of 2-(6′-Methyl-2′-pyridin-4-yl-biphenyl-4-yloxymethyl)-quinoline

### Example 387

### 2-(2-Bromo-6-methyl-phenoxy)-tetrahydro-pyran

### Error! Objects cannot be created from editing field codes

To a stirred solution of 2-bromo-6-methylphenol (2.500 g) in CH₂Cl₂ (25 mL) was added pyridinium p-toluenesulfonate (PPTS, 0.067 g), followed by 3,4-dihydro-2H-pyran (2.25 g) at room temperature under argon and the reaction mixture was stirred at room temperature for 66 h. The solvent was removed under reduced pressure and the residue was purified by chromatography eluting with 0-20% EtOAc/heptane to provided 2-(2-bromo-6-methyl-phenoxy)-tetrahydro-pyran (1.510 g) as a colorless oil. ¹H NMR (300 MHz, CDCl₃/TMS) δ 7.36 (d, J=8.1 Hz, 1H), 7.08 (d, J=7.2 Hz, 1H), 6.85 (t, J=7.8 Hz, 1H), 5.09 (t, J=2.1 Hz, 1H), 4.20-4.05 (m, 1H), 3.59-3.48 (m, 1H), 2.37 (s, 3H), 2.10-1.90 (m, 3H), 1.70-1.50 (m, 3H); ¹³C NMR (75 MHz, CDCl₃/TMS) δ 153.2, 134.2, 130.9, 130.1, 124.9, 117.0, 103.0, 64.2, 30.8, 25.1, 20.1, 18.0.

### 4-(3-Methyl-2-(tetrahydro-pyran-2-yloxy)-phenyl)-pyridine

### Error! Objects cannot be created from editing field codes

A mixture of 2-(2-bromo-6-methyl-phenoxy)-tetrahydropyran (1.570 g), pyridine-4-boronic acid (1.070 g) and Cs₂CO₃ (5.670 g) in dry dioxane (20 mL) was purged with argon. Pd(PPh₃)₄ (0.347 g) was added and the mixture was purged again with argon. The reaction mixture was then heated to reflux for 18 h. The cooled mixture was filtered through a silica gel plug eluting with EtOAc. Evaporation and purification by chromatography eluting with 0-50% EtOAc/heptane produced 4-(3-methyl-2-(tetrahydro-pyran-2-yloxy)-phenyl)-pyridine (1.320 g) as a yellow oil. ¹H NMR (300 MHz, CDCl₃/TMS) δ 8.63 (dd, J=4.5, 1.2 Hz, 2H), 7.45 (dd, J=4.4, 1.5 Hz, 2H), 7.28-7.20 (m, 1H), 7.16-7.06 (m, 2H), 4.56 (br s, 1H), 3.66-3.56 (m, 1H), 3.27-3.15 (m, 1H), 2.40 (s, 3H), 1.78-1.64 (m, 1H), 1.62-1.48 (m, 2H), 1.48-1.28 (m, 3H); ¹³C NMR (75 MHz, CDCl₃/TMS) δ 153.1, 149.3, 147.3, 132.6, 132.5, 131.5, 128.0, 124.2, 124.1, 102.4, 63.5, 30.5, 24.9, 19.6, 17.4.

### 2-Methyl-6-pyridin-4-yl-phenol

### Error! Objects cannot be created from editing field codes

To a solution of 4-(5-methyl-2-(tetrahydropyran-2-yloxy)-phenyl)-pyridine (1.320 g) in MeOH (30 mL) was added trifluoroacetic acid (1.680 g) and the reaction mixture was stirred at room temperature for 16 h. The solvent was removed under reduced pressure. The residue was then partitioned between EtOAc (40 mL) and water (40 mL), and neutralized with an aqueous saturated NaHCO₃ solution. The organic phase was separated and the aqueous layer was extracted with EtOAc (2×40 mL). The combined organic phases were washed with brine and dried over MgSO₄. Filtration and concentration in vacuo produced 2-methyl-6-pyridin-4-yl-phenol (0.820 g) as a light yellow solid. ¹H NMR (300 MHz, CD₃OD/TMS) δ 8.50 (dd, J=4.8, 1.5 Hz, 2H), 7.61 (dd, J=4.5, 1.5 Hz, 2H), 7.15 (t, J=6.3 Hz, 2H), 6.88 (t, J=7.6 Hz, 1H), 2.29 (s, 3H); ¹³C NMR (75 MHz, CD₃OD/TMS) δ 153.2, 149.8, 149.4, 132.5, 128.8, 127.4, 127.1, 125.8, 121.4, 16.8.

### Trifluoro-methanesulfonic acid 2-methyl-6-pyridin-4-yl-phenyl ester

### Error! Objects cannot be created from editing field codes

A solution of the 6-methyl-2-pyridin-4-yl-phenol (0.810 g) in dry pyridine (15 mL) was treated with trifluoromethanesulfonic anhydride (1.850 g) at 0° C. under argon. The resulting mixture was stirred at 0° C. for 0.5 h, and then allowed to warm to room temperature and stirred for an additional 18 h. The solvent was removed under reduced pressure, and the residue was dissolved in CH₂Cl₂ (100 mL), washed with cold saturated aqueous NaHCO₃ solution (2×50 mL), and dried over MgSO₄. Filtration, evaporation and purification by chromatography eluting with 0-40% EtOAc/heptane provided trifluoro-methanesulfonic acid 2-methyl-6-pyridin-4-yl-phenyl ester (1.31 g) as light yellow wax. ¹H NMR (300 MHz, CDCl₃/TMS) δ 8.68 (d, J=8.7 Hz, 2H), 7.40-7.32 (m, 4H), 7.26 (d, J=8.1 Hz, 1H), 2.49 (s, 3H); ¹³C NMR (75 MHz, CDCl₃/TMS) δ 149.8, 144.8, 144.2, 133.4, 132.6, 132.5, 129.2, 128.4, 124.0, 118.0 (J=318 Hz), 17.3.

### 2-(6′-Methyl-2′-pyridin-4-yl-biphenyl-4-yloxymethyl)-quinoline

### Example 387

### Error! Objects cannot be created from editing field codes

A suspension of trifluoromethanesulfonic acid 6-methyl-2-pyridin-4-yl-phenyl ester (0.317 g), 4-(quinolin-2′-ylmethylenoxy)-phenylboronic acid (0.335 g) and 2 M Na₂CO₃ solution (1.5 mL) in dioxane (10 mL) was purged with argon. Pd(PPh₃)₄ (0.058 g) was added and the mixture was purged again with argon. The reaction mixture was heated to reflux for 22 h. More Pd(PPh₃)₄ (0.058 g) was added and the mixture was refluxed for another 23 h. The mixture was cooled to room temperature and the solvent was removed under reduced pressure. The residue was dissolved in EtOAc and filtered through a silica gel plug eluting with EtOAc. Evaporation and purification by chromatography eluting with 0-50% EtOAc/heptane produced 2-(6′-methyl-2′-pyridin-4-yl-biphenyl-4-yloxymethyl)-quinoline (0.310 g) as a colorless oily wax. ¹H NMR (300 MHz, CDCl₃/TMS) δ 8.33 (d, J=5.7 Hz, 2H), 8.19 (d, J=8.7 Hz, 1H), 8.08 (d, J=8.4 Hz, 1H), 7.83 (d, J=7.8 Hz, 1H), 7.73 (dt, J=7.4, 1.2 Hz, 1H), 7.65 (d, J=8.4 Hz, 1H), 7.54 (t, J=7.5 Hz, 1H), 7.32 (d, J=4.5 Hz, 2H), 7.21 (d, J=4.4 Hz, 1H), 7.02-6.86 (m, 6H), 5.34 (s, 2H), 2.18 (s, 3H); ¹³C NMR (75 MHz, CDCl₃/TMS) δ 157.5, 157.0, 149.8, 148.6, 147.3, 139.5, 138.8, 137.0, 136.7, 131.9, 131.1, 130.1, 129.5, 128.7, 127.5, 127.4, 127.1, 126.9, 126.3, 124.5, 118.9, 114.2, 71.1, 21.0; HRMS: M⁺H m/z=403.1816.

### Synthesis of 2-(3′-Methyl-2′-pyridin-4-yl-biphenyl-4-yloxymethyl)-quinoline

### Example 1886

### 2-(3′-Methyl-2′-(tetrahydro-pyran-2-yloxy)-biphenyl-4-yloxymethyl)-quinoline

### Error! Objects cannot be created from editing field codes

To a solution of 2-(2-bromo-6-methylphenoxy)-tetrahydro-pyran (0.920 g) and 2-(4-(4,4,5,5-tetramethyl(1,3,2)dioxaborolan-2-yl)-phenoxymethyl)-quinoline (1.350 g) in dioxane (20 mL) was added 2M aqueous Na₂CO₃ solution (5.1 mL), and the mixture was purged with argon. Pd(PPh₃)₄ (0.196 g) was added and the mixture was purged again with argon. The reaction mixture was heated to reflux for 18 h. The mixture was cooled to room temperature and the solvent was removed under reduced pressure. The residue was passed through a silica gel plug eluting with EtOAc. Evaporation and purification by chromatography eluting with 0-2% MeOH/CH₂Cl₂ produced 2-(3′-methyl-2′-(tetrahydro-pyran-2-yloxy)-biphenyl-4-yloxymethyl)-quinoline (1.250 g) as a yellow wax. ¹H NMR (300 MHz, CDCl₃/TMS) δ 8.19 (d, J=8.4 Hz, 1H), 8.10 (d, J=8.7 Hz, 1H), 7.83 (d, J=8.4 Hz, 1H), 7.78-7.64 (m, 2H), 7.55 (t, J=7.4 Hz, 1H), 7.43 (d, J=9.0 Hz, 2H), 7.16-6.94 (m, 5H), 5.42 (s, 2H), 4.55 (br s, 1H), 3.74-3.60 (m, 1H), 3.28-3.16 (m, 1H), 2.38 (s, 3H), 1.74-1.60 (m, 1H), 1.52-1.18 (m, 5H); ¹³C NMR (75 MHz, CDCl₃/TMS) δ 157.6, 157.1, 153.2, 147.3, 136.7, 134.5, 132.3, 132.1, 130.5, 129.8, 129.6, 128.7, 128.5, 127.5, 127.4, 126.3, 123.7, 119.0, 114.4, 102.0, 71.2, 63.5, 30.5, 25.0, 19.7, 17.5.

### 3-Methyl-4′-(quinolin-2-ylmethoxy)-biphenyl-2-ol

### Error! Objects cannot be created from editing field codes

To a solution of 2-(3′-methyl-2′-(tetrahydro-pyran-2-yloxy)-biphenyl-4-yloxymethyl)-quinoline (1.250 g) in a mixture of MeOH (40 mL) and CH₂Cl₂ (10 mL) was added pyridinium p-toluenesulfonate (PPTS, 0.015 g) and the reaction mixture was stirred and heated to 60° C. for 23 h. The solvent was removed under reduced pressure. The residue was purified by chromatography eluting with 0-2% MeOH/CH₂Cl₂ to produce the title compound 3-methyl-4′-(quinolin-2-ylmethoxy)-biphenyl-2-ol (0.96 g) as a yellow solid. ¹H NMR (300 MHz, CD₃OD/CDCl₃/TMS) δ 8.32 (d, J=8.4 Hz, 1H), 8.05 (d, J=8.7 Hz, 1H), 7.90 (d, J=8.4 Hz, 1H), 7.80-7.68 (m, 2H), 7.59 (t, J=7.7 Hz, 1H), 7.42 (d, J=8.7 Hz, 2H), 7.08 (d, J=87. Hz, 2H), 7.01 (t, J=8.6 Hz, 2H), 6.80 (t, J=7.7 Hz, 1H), 5.37 (s, 2H), 2.26 (s, 3H); ¹³C NMR (75 MHz, CD₃OD/CDCl₃/TMS) δ 158.7, 158.3, 152.1, 147.8, 138.5, 132.8, 131.2, 130.8, 130.3, 129.5, 128.8, 128.6, 128.5, 127.5, 126.2, 120.7, 120.2, 115.4, 71.4, 16.7.

### Trifluoro-methanesulfonic acid 3-methyl-4′-(quinolin-2-ylmethoxy)-2-yl ester

### Error! Objects cannot be created from editing field codes

A solution of 3-methyl-4′-(quinolin-2-ylmethoxy)-biphenyl-2-ol (0.550 g) in dry pyridine (10 mL) was treated with trifluoromethanesulfonic anhydride (0.590 g) at 0° C. under argon. The resulting mixture was stirred at 0° C. for 0.5 h, and then allowed to warm to room temperature and stirred for another 16 h. The solvent was removed under reduced pressure, and the residue was dissolved in CH₂Cl₂ (100 mL), washed with cold saturated NaHCO₃ aqueous solution (2×50 mL), and dried over MgSO₄. Filtration, evaporation and purification by chromatography eluting with 0-2% MeOH/CH₂Cl₂ provided trifluoro-methanesulfonic acid 3-methyl-4′-(quinolin-2-ylmethoxy)-2-yl ester (0.480 g) as a light yellow wax. ¹H NMR (300 MHz, CDCl₃/TMS) δ 8.15 (d, J=8.4 Hz, 1H), 8.09 (d, J=8.4 Hz, 1H), 7.79 (d, J=8.1 Hz, 1H), 7.71 (dt, J=8.1, 1.3 Hz, 1H), 7.65 (d, J=8.7 Hz, 1H), 7.51 (t, J=7.4 Hz, 1H), 7.34 (d, J=8.7 Hz, 2H), 7.25-7.15 (m, 3H), 7.08 (d, J=8.4 Hz, 2H), 5.41 (s, 2H), 2.45 (s, 3H); ¹³C NMR (75 MHz, CDCl₃/TMS) δ 158.1, 157.4, 147.3, 145.5, 136.7, 135.4, 131.9, 130.6, 130.5, 129.6, 129.1, 128.7, 127.8, 127.5, 127.4, 126.3, 118.9, 117.8 (J=318 Hz), 114.7, 71.2, 17.4.

### 2-(3′-Methyl-2′-pyridin-4-yl-biphenyl-4-yloxymethyl)-quinoline

### Example 1886

### Error! Objects cannot be created from editing field codes

A suspension of trifluoro-methanesulfonic acid 3-methyl-4′-(quinolin-2-ylmethoxy)-2-yl ester (0.480 g), pyridine-4-boronic acid (0.187 g) and 2M aqueous Na₂CO₃ solution (1.5 mL) in dioxane (15 mL) was purged with argon. Pd(PPh₃)₄ (0.059 g) was added and the mixture was purged again with argon. The reaction mixture was heated to reflux for 21 h. The mixture was cooled to room temperature and the solvent was removed under reduced pressure. The residue was suspended in EtOAc and filtered through a silica gel plug eluting with EtOAc. Evaporation and purification by chromatography eluting with 0-50% EtOAc/heptane provided 2-(3′-methyl-2′-pyridin-4-yl-biphenyl-4-yloxymethyl)-quinoline (0.13 g) as a light yellow solid. ¹H NMR (300 MHz, CDCl₃/TMS) δ 8.46 (d, J=6.0 Hz, 2H), 8.16 (d, J=8.7 Hz, 1H), 8.07 (d, J=8.4 Hz, 1H), 7.81 (d, J=7.8 Hz, 1H), 7.72 (t, J=7.2 Hz, 1H), 7.61 (d, J=8.4 Hz, 1H), 7.53 (t, J=7.1 Hz, 1H), 7.36-7.21 (m, 3H), 7.02-6.90 (m, 4H), 6.81 (d, J=9.0 Hz, 2H), 5.30 (s, 2H), 2.14 (s, 3H); ¹³C NMR (75 MHz, CDCl₃/TMS) δ 157.5, 156.9, 149.0, 148.7, 147.3, 140.4, 137.4, 136.7, 135.4, 133.8, 130.6, 129.5, 128.9, 128.7, 127.8, 127.7, 127.5, 127.3, 126.3, 125.4, 118.9, 114.0, 71.1, 21.0; HRMS: M⁺H m/z=403.1811.

### Synthesis of 2-(4′-Fluoro-2′-pyridin-4-yl-biphenyl-4-yloxymethyl)-quinoline

### Example 1856

### 2-(2-Bromo-4-fluorophenoxy)-tetrahydropyran

### Error! Objects cannot be created from editing field codes

To a solution of 2-bromo-4-fluoro-phenol (4.260 g) in CH₂Cl₂ (30 mL) was added pyridinium p-toluenesulfonate (PPTS, 0.112 g) followed by 3,4-dihydro-2H-pyran (2.25 g) at room temperature under argon and the reaction mixture was stirred at room temperature for 64 h. The solvent was removed under reduced pressure and the residue was purified by silica gel chromatography eluting with 0.5-7% EtOAc/heptane to provide the title compound 2-(2-bromo-4-fluorophenoxy)-tetrahydropyran (5.230 g) as a colorless oil. ¹H NMR (300 MHz, CDCl₃/TMS) δ 7.28 (dd, J=8.1, 3.0 Hz, 1H), 7.11 (dd, J=9.0, 5.1 Hz, 1H), 7.00-6.90 (m, 1H), 5.40 (s, 1H), 3.90 (dt, J=10.2, 2.7 Hz, 1H), 3.65-3.54 (m, 1H), 2.18-1.80 (m, 3H), 1.80-1.56 (m, 3H); ¹³C NMR (75 MHz, CDCl₃/TMS) δ 156.9 (J=242 Hz), 149.8, 119.9 (J=26 Hz), 117.3 (J=8 Hz), 114.6 (J=22 Hz), 113.1 (J=10 Hz), 97.3, 61.7, 30.1, 25.1, 18.3.

### 4-(5-Fluoro-2-(tetrahydropyran-2-yloxy)-phenyl)-pyridine

### Error! Objects cannot be created from editing field codes

A mixture of 2-(2-bromo-4-fluorophenoxy)-tetrahydro-pyran (1.560 g), pyridine-4-boronic acid (1.050 g) and Cs₂CO₃ (5.540 g) in dioxane (20 mL) was purged with argon. Pd(PPh₃)₄ (0.270 g) was added and the mixture was purged again with argon. The reaction mixture was heated to reflux for 20 h. The mixture was cooled to room temperature, passed through a silica gel plug eluting with EtOAc, and the filtrate was evaporated to dryness. The residue was purified by chromatography eluting with 0-50% EtOAc/heptane to produce 4-(5-fluoro-2-(tetrahydropyran-2-yloxy)-phenyl)-pyridine (1.15 g) as a yellow oil. ¹H NMR (300 MHz, CDCl₃/TMS) δ 8.65 (dd, J=6.5, 1.7 Hz, 2H), 7.48 (dd, J=4.5, 1.7 Hz, 2H), 7.22 (dd, J=8.7, 4.6 Hz, 1H), 7.10-6.98 (m, 2H), 5.35 (s, 1H), 3.75 (dt, J=10.2, 2.7 Hz, 1H), 3.63-3.52 (m, 1H), 1.86-1.46 (m, 6H); ¹³C NMR (75 MHz, CDCl₃/TMS) δ 157.4 (J=238 Hz), 149.9, 149.3, 145.1, 129.6 (J=7 Hz), 124.0, 117.1 (J=8 Hz), 116.5 (J=23 Hz), 116.0 (J=22 Hz), 97.2, 61.9, 30.1, 25.0, 18.5.

### 4-Fluoro-2-pyridin-4-yl-phenol

### Error! Objects cannot be created from editing field codes

To a solution of 4-(5-fluoro-2-(tetrahydropyran-2-yloxy)-phenyl)-pyridine (1.150 g) in MeOH (30 mL) was added trifluoroacetic acid (1.440 g) and the reaction mixture was stirred at room temperature for 18 h. The solvent was removed under reduced pressure. The residue was partitioned between EtOAc (30 mL) and water (30 mL), and neutralized with saturated aqueous NaHCO₃ solution. The organic phase was separated from the aqueous phase, and the aqueous phase was extracted with EtOAc (2×30 mL). The combined organic layers were washed with brine and dried over MgSO₄. Filtration and concentration produced title compound 4-fluoro-2-pyridin-4-yl-phenol (0.770 g) as a light yellow solid. ¹H NMR (300 MHz, CD₃OD/TMS) δ 8.53 (d, J=5.7 Hz, 2H), 7.69 (dd, J=4.8, 1.5 Hz, 2H), 7.14 (dd, J=9.3, 3.0 Hz, 1H), 7.00 (dt, J=8.7, 3.0 Hz, 1H), 6.91 (dd, J=9.0, 4.8 Hz, 1H); ¹³C NMR (75 MHz, CD₃OD/TMS) δ 157.7 (J=234 Hz), 152.1, 149.5, 148.0, 126.8 (J=7 Hz), 125.5, 118.1 (J=8 Hz), 117.4 (J=23 Hz), 116.9 (J=24 Hz).

### Trifluoro-methanesulfonic acid 4-fluoro-2-pyridin-4-yl-phenyl ester

### Error! Objects cannot be created from editing field codes

A solution of 4-fluoro-2-pyridin-4-yl-phenol (0.770 g) in dry pyridine (15 mL) was treated with trifluoromethanesulfonic anhydride (1.720 g) at 0° C. under argon. The resulting mixture was stirred at 0° C. for 0.5 h, then was allowed to warm to room temperature and stirred for an additional 18 h. The solvent was removed under reduced pressure, and the residue was dissolved in CH₂Cl₂ (100 mL), washed with cold saturated aqueous NaHCO₃ solution (2×50 mL), and dried over MgSO₄. Filtration, evaporation and purification by silica gel chromatography eluting with 0-50% EtOAc/heptane provided trifluoro-methanesulfonic acid 4-fluoro-2-pyridin-4-yl-phenyl ester (1.170 g) as a light yellow oil. ¹H NMR (300 MHz, CDCl₃/TMS) δ 8.74 (dd, J=8.7, 1.5 Hz, 2H), 7.48-7.30 (m, 3H), 7.26-7.12 (m, 2H); ¹³C NMR (75 MHz, CDCl₃/TMS) δ 161.3 (J=248 Hz), 150.1, 142.2, 141.9, 134.7 (J=8 Hz), 124.1 (J=9 Hz), 123.5, 118.1 (J=318 Hz), 118.0 (J=24 Hz), 116.9 (J=24 Hz).

### 2-(4′-Fluoro-2′-pyridin-4-yl-biphenyl-4-yloxymethyl)-quinoline

### Example 1856

### Error! Objects cannot be created from editing field codes

A suspension of trifluoromethanesulfonic acid 4-fluoro-2-pyridin-4-yl-phenyl ester (0.205 g), 4-(quinolin-2′-ylmethylenoxy)-phenylboronic acid (0.214 g) and 2M aqueous Na₂CO₃ solution (0.96 mL) in dioxane (10 mL) was purged with argon. Pd(PPh₃)₄ (0.037 g) was added and the mixture was purged again with argon. The reaction mixture was heated to reflux for 26 h. The mixture was cooled to room temperature and the solvent was removed under reduced pressure. The residue was passed through a silica gel plug eluting with EtOAc. Concentration and purification by chromatography eluting with 0-40% EtOAc/heptane produced 2-(4′-fluoro-2′-pyridin-4-yl-biphenyl-4-yloxymethyl)-quinoline (0.182 g) as a colorless oily wax. ¹H NMR (300 MHz, CDCl₃/TMS) δ 8.45 (b s, 2H), 8.18 (d, J=8.4 Hz, 1H), 8.07 (d, J=8.7 Hz, 1H), 7.82 (d, J=7.5 Hz, 1H), 7.73 (t, J=7.1 Hz, 1H), 7.65 (d, J=8.4 Hz, 1H), 7.54 (t, J=7.1 Hz, 1H), 7.36 (dd, J=8.1, 5.7 Hz, 1H), 7.18-7.05 (m, 2H), 7.05-6.93 (m, 4H), 6.93-6.80 (m, 2H), 5.35 (s, 2H); ¹³C NMR (75 MHz, CDCl₃/TMS) δ 161.7 (J=245 Hz), 157.4, 157.3, 149.3, 148.2, 147.3, 139.1 (J=8 Hz), 136.7, 136.0 (J=3 Hz), 132.2, 132.1, 130.7, 129.6, 128.7, 127.5, 127.4, 126.3, 124.2, 118.9, 116.6 (J=22 Hz), 115.3 (J=21 Hz), 114.5, 71.2; HRMS: M⁺H m/z=407.1554.

### Synthesis of 2-(5′-fluoro-2′-pyridin-4-yl-biphenyl-4-yloxymethyl)-quinoline

### Example 1112

### 2-(5′-Fluoro-2′-(tetrahydropyran-2-yloxy)-biphenyl-4-yloxymethyl)-quinoline

### Error! Objects cannot be created from editing field codes

A suspension of 2-(2-bromo-4-fluorophenoxy)-tetrahydropyran (1.000 g), 2-(4-(4,4,5,5-tetramethyl(1,3,2)dioxaborolan-2-yl)-phenoxymethyl)-quinoline (1.450 g) and 2 M aqueous Na₂CO₃ solution (5.5 mL) in dioxane (20 mL) was purged with argon. Pd(PPh₃)₄ (0.210 g) was added and the mixture was purged again with argon. The reaction mixture was heated to reflux for 18 h. The mixture was cooled to room temperature and the solvent was removed under reduced pressure. The residue was passed through a silica gel plug eluting with EtOAc. Concentration and purification by chromatography eluting with 1.5-30% EtOAc/heptane produced the title compound 2-(5′-fluoro-2′-(tetrahydropyran-2-yloxy)-biphenyl-4-yloxymethyl)-quinoline (1.400 g) as a yellow wax. ¹H NMR (300 MHz, CDCl₃/TMS) δ 8.20 (d, J=8.4 Hz, 1H), 8.10 (d, J=8.4 Hz, 1H), 7.83 (d, J=8.1 Hz, 1H), 7.78-7.65 (m, 2H), 7.55 (t, J=7.5 Hz, 1H), 7.49 (d, J=8.4 Hz, 2H), 7.14 (dd, J=8.7, 5.0 Hz, 1H), 7.07 (d, J=9.0 Hz, 2H), 7.02 (dd, J=9.5, 3.0 Hz, 1H), 6.92 (dt, J=8.4, 2.7 Hz, 1H), 5.43 (s, 2H), 5.25 (s, 1H), 3.75 (dt, J=10.5, 2.7 Hz, 1H), 3.53 (d, J=11.1 Hz, 1H), 1.84-1.42 (m, 6H); ¹³C NMR (75 MHz, CDCl₃/TMS) δ 157.60, 157.59 (J=238 Hz), 157.48, 149.7, 147.3, 136.8, 132.6 (J=7 Hz), 130.5, 130.3, 129.6, 128.7, 127.5, 127.4, 126.3, 119.0, 117.4 (J=8 Hz), 116.6 (J=23 Hz), 114.2, 113.9 (J=23 Hz), 97.3, 71.2, 61.7, 30.2, 25.1, 18.5.

### 5-Fluoro-4′-(quinolin-2-ylmethoxy)-biphenyl-2-ol

### Error! Objects cannot be created from editing field codes

To a solution of 2-(5′-fluoro-2′-(tetrahydropyran-2-yloxy)-biphenyl-4-yloxymethyl)-quinoline (1.400 g) in a mixture of MeOH (40 mL) and CH₂Cl₂ (8 mL) was added pyridinium p-toluenesulfonate (PPTS, 0.016 g) and the reaction mixture was stirred and heated to 60° C. for 20 h. The solvent was removed under reduced pressure. The residue was washed with MeOH to produce the title compound 5-fluoro-4′-(quinolin-2-ylmethoxy)-biphenyl-2-ol (1.040 g) as a white solid. ¹H NMR (300 MHz, CD₃OD/CDCl₃/TMS) δ 8.32 (d, J=8.4 Hz, 1H), 8.07 (d, J=8.4 Hz, 1H), 7.91 (d, J=7.5 Hz, 1H), 7.84-7.70 (m, 2H), 7.60 (t, J=7.5 Hz, 1H), 7.52 (d, J=8.1 Hz, 2H), 7.09 (d, J=8.4 Hz, 2H), 6.95 (d, J=9.0 Hz, 1H), 6.83 (d, J=4.5 Hz, 1H), 5.41 (s, 2H); ¹³C NMR (75 MHz, CD₃OD/CDCl₃/TMS) δ 158.3, 158.0, 157.0 (J=234 Hz), 150.3, 147.5, 138.2, 131.4, 130.8, 130.5, 129.6 (J=8 Hz), 128.2, 127.2, 119.8, 117.0, 116.9 (J=4 Hz), 116.5, 115.0, 114.3 (J=22 Hz), 71.2.

### 5-Fluoro-4′-(quinolin-2-ylmethoxy)biphenyl-2-yl trifluoromethanesulfonate

### Error! Objects cannot be created from editing field codes

A solution of 5-fluoro-4′-(quinolin-2-ylmethoxy)-biphenyl-2-ol (0.595 g) in dry pyridine (10 mL) was treated with trifluoromethanesulfonic anhydride (0.632 g) at 0° C. under argon. The resulting mixture was stirred at 0° C. for 0.5 h, then was allowed to warm to room temperature and stirred for an additional 16 h. The solvent was removed under reduced pressure, and the residue was dissolved in CH₂Cl₂ (100 mL), washed with cold saturated aqueous NaHCO₃ solution (2×50 mL), and dried over MgSO₄. Filtration, evaporation and purification by silica gel chromatography eluting with 0-2% MeOH/CH₂Cl₂ provided title compound 5-fluoro-4′-(quinolin-2-ylmethoxy)biphenyl-2-yl trifluoromethanesulfonate (0.780 g) as an off-white solid. ¹H NMR (300 MHz, CDCl₃/TMS) δ 8.20 (d, J=8.7 Hz, 1H), 8.10 (d, J=8.7 Hz, 1H), 7.84 (d, J=7.8 Hz, 1H), 7.74 (dt, J=7.2, 1.8 Hz, 1H), 7.68 (d, J=8.4 Hz, 1H), 7.56 (t, J=6.9 Hz, 1H), 7.42-7.35 (m, 3H), 7.18-7.00 (m, 4H), 5.43 (s, 2H); ¹³C NMR (75 MHz, CDCl₃/TMS) δ 161.1 (J=247 Hz), 158.6, 157.2, 147.4, 142.3, 137.0 (J=8 Hz), 136.9, 130.4, 129.6, 128.8, 127.5, 127.4, 127.3, 126.4, 123.5 (J=9 Hz), 118.9, 118.12 (J=318 Hz), 118.10 (J=24 Hz), 115.0 (J=23 Hz), 114.9, 71.3.

### 2-(5′-Fluoro-2′-pyridin-4-yl-biphenyl-4-yloxymethyl)-quinoline

### Example 1112

### Error! Objects cannot be created from editing field codes

A mixture of 5-fluoro-4′-(quinolin-2-ylmethoxy)biphenyl-2-yl trifluoromethanesulfonate (0.477 g), pyridine-4-boronic acid (0.184 g) and 2M aqueous Na₂CO₃ solution (1.5 mL) in dioxane (15 mL) was purged with argon. Pd(PPh₃)₄ (0.058 g) was added and the mixture was purged again with argon. The reaction mixture was heated to reflux for 23 h. The mixture was cooled to room temperature, passed through a silica gel plug eluting with EtOAc. Concentration and purification by chromatography eluting with 0-1.5% MeOH/CH₂Cl₂ produced the title compound 2-(5′-fluoro-2′-pyridin-4-yl-biphenyl-4-yloxymethyl)-quinoline (0.330 g). ¹H NMR (300 MHz, CDCl₃/TMS) δ 8.44 (dd, J=4.5, 1.5 Hz, 2H), 8.19 (d, J=8.4 Hz, 1H), 8.08 (d, J=8.4 Hz, 1H), 7.83 (d, J=8.4 Hz, 1H), 7.73 (dt, J=6.9, 1.2 Hz, 1H), 7.65 (d, J=8.4 Hz, 1H), 7.55 (dt, J=7.5, 1.2 Hz, 1H), 7.34 (dd, J=7.7, 6.1 Hz, 1H), 7.12 (d, J=8.7 Hz, 2H), 7.06-6.98 (m, 4H), 6.91 (d, J=8.7 Hz, 2H), 5.35 (s, 2H); ¹³C NMR (75 MHz, CDCl₃/TMS) δ 162.3 (J=247 Hz), 157.7, 157.2, 149.2, 148.4, 147.3, 142.5 (J=8 Hz), 136.7, 133.4 (J=3 Hz), 132.1, 131.6 (J=8 Hz), 130.6, 129.6, 128.7, 127.5, 127.3, 126.3, 124.4, 118.9, 117.2 (J=21 Hz), 114.6, 114.1 (J=21 Hz), 71.2; HRMS: M⁺H m/z=407.1540.

### Synthesis of 2-(6′-fluoro-2′-pyridin-4-yl-biphenyl-4-yloxymethyl)-quinoline

### Example 381

### 2-(2-Bromo-6-fluorophenoxy)-tetrahydro-pyran

### Error! Objects cannot be created from editing field codes

To a stirred solution of 2-bromo-6-fluorophenol (5.020 g) in CH₂Cl₂ (30 mL) was added pyridinium p-toluenesulfonate (PPTS, 0.066 g), followed by 3,4-dihydro-2H-pyran (4.420 g) at room temperature under argon and the reaction mixture was stirred at room temperature for 64 h. The solvent was removed under reduced pressure and the residue was purified by silica gel chromatography eluting with 0-5% EtOAc/heptane to provide the title compound 2-(2-bromo-6-fluorophenoxy)-tetrahydro-pyran (6.410 g) as a colorless oil. ¹H NMR (300 MHz, CDCl₃/TMS) δ 7.28 (dd, J=10.4, 2.3 Hz, 1H), 7.20-7.15 (m, 1H), 7.09 (t, J=8.6 Hz, 1H), 5.40 (s, 1H), 3.90 (dt, J=10.7, 2.7 Hz, 1H), 3.66-3.46 (m, 1H), 2.10-1.78 (m, 3H), 1.78-1.50 (m, 3H); ¹³C NMR (75 MHz, CDCl₃/TMS) δ 152.9 (J=248 Hz), 144.0 (J=10 Hz), 127.1 (J=4 Hz), 119.6 (J=22 Hz), 119.5, 113.2 (J=8 Hz), 97.5, 61.8, 30.0, 25.0, 18.2.

### 4-(3-Fluoro-2-(tetrahydropyran-2-yloxy)-phenyl)-pyridine

### Error! Objects cannot be created from editing field codes

A mixture of 2-(2-bromo-6-fluorophenoxy)-tetrahydropyran (1.110 g), pyridine-4-boronic acid (0.740 g) and 2 M aqueous Na₂CO₃ solution (6.0 mL) in dioxane (25 mL) was purged with argon. Pd(PPh₃)₄ (0.230 g) was added and the mixture was purged again with argon. The reaction mixture was heated to reflux for 18 h. The cooled mixture was evaporated to dryness and the residue was filtered through a silica gel plug eluting with EtOAc. Concentration and purification by silica gel chromatography eluting with 0-50% EtOAc/heptane produced the title compound 4-(3-fluoro-2-(tetrahydropyran-2-yloxy)-phenyl)-pyridine (0.880 g) as a light yellow oily wax. ¹H NMR (300 MHz, CDCl₃/TMS) δ 8.63 (dd, J=4.2, 1.5 Hz, 2H), 7.48-7.38 (m, 2H), 7.36 (d, J=9.3 Hz, 2H), 7.29 (d, J=10.5 Hz, 1H), 5.53 (s, 1H), 3.94 (t, J=10.2 Hz, 1H), 3.65 (d, J=10.5 Hz, 1H), 2.20-1.83 (m, 3H), 1.83-1.55 (m, 3H); ¹³C NMR (75 MHz, CDCl₃/TMS) δ 153.2 (J=245 Hz), 150.1, 146.5, 145.4 (J=11 Hz), 131.9 (J=7 Hz), 112.6 (J=3 Hz), 120.9, 118.5, 114.6 (J=20 Hz), 97.3, 61.9, 30.0, 25.0, 18.3.

### 2-Fluoro-6-pyridin-4-yl-phenol

### Error! Objects cannot be created from editing field codes

To a solution of 4-(3-fluoro-2-(tetrahydropyran-2-yloxy)-phenyl)-pyridine (0.880 g) in MeOH (30 mL) was added trifluoroacetic acid (1.100 g) and the reaction mixture was stirred at room temperature for 16 h. The solvent was removed under reduced pressure. The residue was suspended in a mixture of EtOAc (30 mL) and water (30 mL), neutralized with saturated NaHCO₃ solution. The resulting yellow precipitate was filtered, washed with water, and dried over high vacuum to give title compound 2-fluoro-6-pyridin-4-yl-phenol (0.520 g) as a yellow solid. ¹H NMR (300 MHz, CD₃OD/TMS) δ 8.52 (d, J=4.5 Hz, 2H), 7.57 (d, J=6.0 Hz, 2H), 7.48-7.33 (m, 2H), 7.06 (t, J=8.6 Hz, 1H); ¹³C NMR (75 MHz, CD₃OD/TMS) δ 152.3 (J=240 Hz), 149.5, 148.5, 146.7 (J=13 Hz), 129.5 (J=7 Hz), 123.5 (J=3 Hz), 121.6, 118.7 (J=3 Hz), 114.8 (J=20 Hz).

### Trifluoromethanesulfonic acid 2-fluoro-6-pyridin-4-yl-phenyl ester

### Error! Objects cannot be created from editing field codes

A solution of the 6-fluoro-2-pyridin-4-yl-phenol (0.430 g) in dry pyridine (10 mL) was treated with trifluoromethanesulfonic anhydride (0.960 g) at 0° C. under argon. The resulting mixture stirred at 0° C. for 0.5 h, then allowed to warm to room temperature and stirred for 18 h. The solvent was removed under reduced pressure, and the residue was dissolved in CH₂Cl₂ (50 mL), washed with cold saturated aqueous NaHCO₃ solution (2×25 mL), and dried over MgSO₄. Filtration, evaporation and purification by silica gel chromatography eluting with 0-1.0% MeOH/CH₂Cl₂ provided title compound trifluoromethanesulfonic acid 2-fluoro-6-pyridin-4-yl-phenyl ester (0.700 g) as a light yellow oil. ¹H NMR (300 MHz, CDCl₃/TMS) δ 8.73 (dd, J=5.4, 1.2 Hz, 2H), 7.60-7.44 (m, 5H); ¹³C NMR (75 MHz, CDCl₃/TMS) δ 153.7 (J=253 Hz), 150.4, 145.2, 140.1 (J=6 Hz), 136.9 (J=14 Hz), 124.1, 123.4 (J=4 Hz), 121.3, 118.5 (J=318 Hz), 116.1 (J=19 Hz).

### 2-(6′-Fluoro-2′-pyridin-4-yl-biphenyl-4-yloxymethyl)-quinoline

### Example 381

### Error! Objects cannot be created from editing field codes

A suspension of trifluoromethanesulfonic acid 6-fluoro-2-pyridin-4-yl-phenyl ester (0.210 g), 2-(4-(4,4,5,5-tetramethyl(1,3,2)dioxaborolan-2-yl)-phenoxymethyl)-quinoline (0.260 g) and Cs₂CO₃ (0.639 g) in dioxane (10 mL) was purged with argon. Pd(dppf)Cl₂.CH₂Cl₂ (0.027 g) was added and the mixture was purged again with argon. The reaction mixture was heated to reflux for 20 h. The mixture was cooled to room temperature, the resulting precipitate was filtered, and the filtrate was concentrated to dryness. The residue was combined with the collected precipitate and purified by silica gel chromatography eluting with 0-3% MeOH/CH₂Cl₂ to produce the title compound 2-(6′-fluoro-2′-pyridin-4-yl-biphenyl-4-yloxymethyl)-quinoline (0.150 g) as a white solid. ¹H NMR (300 MHz, CD₃OD/CDCl₃/TMS) δ 8.61 (d, J=6.0 Hz, 2H), 8.34 (d, J=8.4 Hz, 1H), 8.09 (d, J=8.4 Hz, 1H), 7.92 (d, J=8.4 Hz, 1H), 7.85-7.72 (m, 2H), 7.70-7.48 (m, 8H), 7.16 (d, J=8.4 Hz, 2H), 5.44 (s, 2H); ¹³C NMR (75 MHz, CD₃OD/CDCl₃/TMS) δ 160.5 (J=244 Hz), 158.8, 158.1, 149.9, 148.1, 147.5, 138.5 (J=8 Hz), 138.1, 131.6 (J=4 Hz), 130.59, 130.55, 129.9 (J=14 Hz), 128.5, 128.4, 128.2, 127.2, 123.3, 123.2, 122.1, 119.8, 115.4, 114.9 (J=24 Hz), 71.3; HRMS: M⁺H m/z=407.1566.

### Synthesis of 2-(3′-Fluoro-2′-pyridin-4-ylbiphenyl-4-yloxymethyl)-quinoline

### Example 1946

### 2-(3′-Fluoro-2′-(tetrahydropyran-2-yloxy)-biphenyl-4-yloxymethyl)-quinoline

### Error! Objects cannot be created from editing field codes

To a solution of 2-(2-bromo-6-fluoro-phenoxy)-tetrahydropyran (1.000 g) and 2-(4-(4,4,5,5-tetramethyl(1,3,2)dioxaborolan-2-yl)-phenoxymethyl)-quinoline (1.450 g) in dioxane (20 mL) was added 2M aqueous Na₂CO₃ solution (5.5 mL), and the mixture was purged with argon. Pd(PPh₃)₄ (0.210 g) was added and the mixture was purged again with argon. The reaction mixture was heated to reflux for 17 h. The mixture was then cooled to room temperature and the solvent was removed under reduced pressure. The residue was passed through a silica gel plug eluting with EtOAc. Concentration and purification by chromatography eluting with 0-1.5% MeOH/CH₂Cl₂ produced the title compound 2-(3′-fluoro-2′-(tetrahydropyran-2-yloxy)-biphenyl-4-yloxymethyl)-quinoline (1.370 g) as a red solid. ¹H NMR (300 MHz, CDCl₃/TMS) δ 8.18 (d, J=8.7 Hz, 1H), 8.09 (d, J=8.7 Hz, 1H), 7.82 (d, J=8.1 Hz, 1H), 7.74 (dt, J=7.8, 1.2 Hz, 1H), 7.68 (d, J=8.4 Hz, 1H), 7.54 (t, J=7.7 Hz, 1H), 7.44 (d, J=8.7 Hz, 2H), 7.30-7.14 (m, 3H), 7.06 (d, J=8.7 Hz, 2H), 5.46 (b s, 1H), 5.41 (s, 2H), 3.97 (dt, J=10.8, 2.7 Hz, 1H), 3.63 (d, J=11.4 Hz, 1H), 2.14-1.80 (m, 3H), 1.80-1.50 (m, 3H); ¹³C NMR (75 MHz, CDCl₃/TMS) δ 157.7, 157.6, 153.2 (J=244 Hz), 147.3, 143.4 (J=11 Hz), 136.8, 135.1 (J=7 Hz), 132.6, 129.6, 128.7, 127.7, 127.5, 127.4, 126.3, 122.0 (J=3 Hz), 118.9, 118.6, 115.0, 114.3 (J=20 Hz), 97.5, 71.2, 61.8, 30.1, 25.1, 18.4.

### 3-Fluoro-4′-(quinolin-2-ylmethoxy)-biphenyl-2-ol

### Error! Objects cannot be created from editing field codes

To a solution of 2-(3′-fluoro-2′-(tetrahydropyran-2-yloxy)-biphenyl-4-yloxymethyl)-quinoline (1.340 g) in a mixture of MeOH (45 mL) and CH₂Cl₂ (10 mL) was added pyridinium p-toluenesulfonate (PPTS, 0.016 g) and the reaction mixture was stirred and heated to 60° C. for 20 h. The solvent was then removed under reduced pressure. The residue was purified by chromatography eluting with 0-2% MeOH/CH₂Cl₂ to produce the title compound 3-fluoro-4′-(quinolin-2-ylmethoxy)-biphenyl-2-ol (1.010 g) as an off-white solid. ¹H NMR (300 MHz, CD₃OD/CDCl₃/TMS) δ 8.28 (d, J=8.4 Hz, 1H), 8.08 (d, J=8.7 Hz, 1H), 7.88 (d, J=8.1 Hz, 1H), 7.82-7.70 (m, 2H), 7.59 (t, J=7.4 Hz, 1H), 7.45 (d, J=8.4 Hz, 2H), 7.22 (d, J=12.3 Hz, 1H), 7.16 (d, J=9.0 Hz, 1H), 7.08 (d, J=9.0 Hz, 2H), 6.97 (t, J=8.7 Hz, 1H), 5.40 (s, 2H); ¹³C NMR (75 MHz, CD₃OD/CDCl₃/TMS) δ 157.9, 157.7, 151.8 (J=238 Hz), 147.2, 143.7 (J=13 Hz), 137.8, 133.5, 133.1 (J=6 Hz), 130.3, 128.2, 127.93, 127.86, 127.80, 126.9, 122.6 (J=3 Hz), 119.5, 118.0 (J=2.4 Hz), 115.3, 114.2 (J=19 Hz), 71.1.

### 3-Fluoro-4′-(quinolin-2-ylmethoxy)biphenyl-2-yl trifluoromethanesulfonate

### Error! Objects cannot be created from editing field codes

A solution of 3-fluoro-4′-(quinolin-2-ylmethoxy)-biphenyl-2-ol (0.538 g) in dry pyridine (10 mL) was treated with trifluoromethanesulfonic anhydride (0.571 g) at 0° C. under argon. The resulting mixture was stirred at 0° C. for 0.5 h, then allowed to warm to room temperature and stirred for 19 h. The solvent was removed under reduced pressure, and the residue was dissolved in CH₂Cl₂ (100 mL), washed with cold saturated aqueous NaHCO₃ solution (2×50 mL), and dried over MgSO₄. Filtration, evaporation and purification by chromatography eluting with 0-1% MeOH/CH₂Cl₂ provided the title compound 3-fluoro-4′-(quinolin-2-ylmethoxy)biphenyl-2-yl trifluoromethanesulfonate (0.540 g) as a white solid. ¹H NMR (300 MHz, CDCl₃/TMS) δ 8.20 (d, J=8.7 Hz, 1H), 8.10 (d, J=8.7 Hz, 1H), 7.84 (d, J=7.8 Hz, 1H), 7.74 (dt, J=7.2, 1.8 Hz, 1H), 7.68 (d, J=8.4 Hz, 1H), 7.56 (t, J=6.9 Hz, 1H), 7.42-7.35 (m, 3H), 7.18-7.00 (m, 4H), 5.43 (s, 2H); ¹³C NMR (75 MHz, CDCl₃/TMS) δ 161.1 (J=247 Hz), 158.6, 157.2, 147.4, 142.3, 137.0 (J=8 Hz), 136.9, 130.4, 129.6, 128.8, 127.5, 127.4, 127.3, 126.4, 123.5 (J=9 Hz), 118.9, 118.12 (J=318 Hz), 118.10 (J=24 Hz), 115.0 (J=23 Hz), 114.9, 71.3.

### 2-(3′-Fluoro-2′-pyridin-4-ylbiphenyl-4-yloxymethyl)-quinoline

### Example 1946

### Error! Objects cannot be created from editing field codes

To a suspension of 3-fluoro-4′-(quinolin-2-ylmethoxy)biphenyl-2-yl trifluoromethanesulfonate (0.360 g) and pyridine-4-boronic acid (0.139 g) in dioxane (12 mL) was added 2M aqueous Na₂CO₃ solution (1.13 mL), and the mixture was purged with argon. Pd(PPh₃)₄ (0.044 g) was added and the mixture was purged again with argon. The reaction mixture was then heated to reflux for 23 h. The mixture was cooled to room temperature and the solvent was removed under reduced pressure. The residue was suspended in a mixture of EtOAc (30 mL) and water (10 mL), and neutralized with 2N aqueous HCl solution. The insoluble materials were filtered off and the filtrate was separated. The organic phase was washed with brine and dried over MgSO₄. Concentration and purification by chromatography eluting with 0-60% EtOAc/heptane provided the title compound 2-(3′-fluoro-2′-pyridin-4-ylbiphenyl-4-yloxymethyl)-quinoline (0.130 g) as a light yellow solid. ¹H NMR (300 MHz, CDCl₃/TMS) δ 8.68 (b s, 2H), 8.21 (d, J=8.4 Hz, 1H), 8.10 (d, J=8.7 Hz, 1H), 7.84 (d, J=8.1 Hz, 1H), 7.75 (t, J=7.5 Hz, 1H), 7.69 (m, 1H), 7.62-7.30 (m, 8H), 7.13 (d, J=8.7 Hz, 2H), 5.45 (s, 2H); ¹³C NMR (75 MHz, CDCl₃/TMS) δ 160.0 (J=248 Hz), 158.6, 157.4, 149.8, 147.5, 143.2 (J=26 Hz), 136.8, 134.7 (J=11 Hz), 132.0, 131.9, 130.2 (J=4 Hz), 129.6, 128.9, 128.3 (J=13 Hz), 128.0, 127.5, 126.4, 123.2, 122.6, 118.9, 115.4, 114.2 (J=23 Hz), 71.5; HRMS: M⁺H m/z=407.1575.

### Synthesis of 2-Pyridin-4-yl-4′-(quinolin-2-ylmethoxy)-biphenyl-3-carbonitrile

### Example 1870

### 3-Bromo-2-hydroxybenzonitrile

### Error! Objects cannot be created from editing field codes

To a solution of o-cyanophenol (5.960 g) and diisopropylamine (0.400 g) in toluene (500 mL) at 70° C. was added NBS (9.790 g) in one portion under argon and the reaction mixture was stirred for 2 h at the same temperature. An additional portion of NBS (0.890 g) was added and heating continued until disappearance of starting material (4 h). The reaction mixture was cooled, diluted with EtOAc (250 mL), washed with water (2×100 mL) and brine (100 mL), and dried over MgSO₄. Concentration and purification by silica gel chromatography eluting with 0-5% MeOH/CH₂Cl₂ gave 9.330 g of crude product as a yellow solid. NMR showed a mixture of 3-bromo-2-hydroxybenzonitrile and 3,5-dibromo-2-hydroxybenzonitrile with a molar ratio of 1:0.3. This mixture was used directly in the next step without further purification. ¹H NMR (300 MHz, CD₃OD/TMS) δ 7.77 (dd, J=8.2, 1.6 Hz, 1H), 7.54 (dd, J=7.8, 1.5 Hz, 1H), 6.89 (t, J=8.0 Hz, 1H); ¹³C NMR (75 MHz, CD₃OD/TMS) δ 157.6, 138.9, 133.6, 122.3, 116.7, 112.3, 103.2.

### 3-Bromo-2-(tert-butyldimethylsilanyloxy)-benzonitrile

### Error! Objects cannot be created from editing field codes

To a solution of a mixture of 3-bromo-2-hydroxybenzonitrile and 3,5-dibromo-2-hydroxybenzonitrile (2.180 g, molar ratio: 1:0.3) in DMF (20 mL) were added imidazole (1.680 g), DMAP (0.130 g), and tert-butyldimethylsilyl chloride (2.230 g) at room temperature and the reaction mixture was stirred for 19 h at the same temperature. The reaction mixture was then diluted with water (200 mL) and brine (20 mL), and extracted with EtOAc (3×60 mL). The combined organic phases were washed with 1 N NaOH (30 mL), water (30 mL) and brine (30 mL), and dried over MgSO₄. Concentration gave 2.8 g crude product as light yellow oil. Chromatography eluting with 1-5% EtOAc/heptane provided pure title compound 3-bromo-2-(tert-butyldimethylsilanyloxy)-benzonitrile (1.9 g) as a colorless oil. ¹H NMR (300 MHz, CDCl₃/TMS) δ 7.75 (dd, J=7.8, 1.5 Hz, 1H), 7.50 (dd, J=7.8, 1.5 Hz, 1H), 6.92 (t, J=8.0 Hz, 1H), 1.09 (s, 9H), 0.38 (s, 6H); ¹³C NMR (75 MHz, CDCl₃/TMS) δ 154.7, 138.1, 132.7, 122.5, 116.6, 116.2, 106.7, 25.8, 18.6, −2.8.

### 2-Hydroxy-4′-(quinolin-2-ylmethoxy)-biphenyl-3-carbonitrile

### Error! Objects cannot be created from editing field codes

To a solution of 3-bromo-2-(tert-butyldimethylsilanyloxy)-benzonitrile (0.880 g), 2-(4-(4,4,5,5-tetramethyl(1,3,2)dioxaborolan-2-yl)-phenoxymethyl)-quinoline (1.120 g) in dioxane (15 mL) was added 2M aqueous Na₂CO₃ solution (4.2 mL) and the mixture was purged with argon. Pd(PPh₃)₄ (0.160 g) was added and the mixture was purged again with argon. The reaction mixture was heated to reflux for 21 h. The cooled mixture was evaporated to dryness and the residue was suspended in EtOAc (60 mL) and neutralized with 2 N aqueous HCl solution. The black precipitate was filtered. The organic phase of the filtrate was separated, washed with brine (20 mL), and dried over MgSO₄. Concentration and purification by chromatography eluting with 0-3% MeOH/CH₂Cl₂ provided the title compound 2-hydroxy-4′-(quinolin-2-ylmethoxy)-biphenyl-3-carbonitrile (0.4 g) as a yellow wax. ¹H NMR (300 MHz, CD₃OD/CDCl₃/TMS) δ 8.26 (d, J=8.4 Hz, 1H), 8.05 (d, J=8.4 Hz, 1H), 7.86 (d, J=8.1 Hz, 1H), 7.76 (t, J=7.7 Hz, 1H), 7.69 (d, J=8.4 Hz, 1H), 7.57 (t, J=7.5 Hz, 1H), 7.48-7.30 (m, 4H), 7.09 (d, J=9.0 Hz, 2H), 6.97 (t, J=7.8 Hz, 1H), 5.36 (s, 2H), 4.70 (b s, 1H); ¹³C NMR (75 MHz, CD₃OD/CDCl₃/TMS) δ 158.3, 157.7, 156.7, 147.2, 138.0, 135.6, 132.2, 130.8, 130.7, 130.4, 129.7, 128.12, 128.06, 127.9, 127.1, 120.8, 119.6, 117.3, 115.3, 115.2, 70.9.

### Trifluoromethanesulfonic acid 3-cyano-4′-(quinolin-2-ylmethoxy)-biphenyl-2-yl ester

### Error! Objects cannot be created from editing field codes

To a solution of 2-hydroxy-4′-(quinolin-2-ylmethoxy)-biphenyl-3-carbonitrile (0.460 g) in dry pyridine (10 mL) was added DMAP (0.016 g) followed by trifluoromethanesulfonic anhydride (0.552 g) at room temperature and the mixture was stirred for 24 h under argon at the same temperature. The solvent was removed under reduced pressure and the residue was dissolved in CH₂Cl₂ (80 mL), washed with cold saturated NaHCO₃ (2×40 mL), and dried over MgSO₄. Concentration and purification by chromatography eluting with 0-2% MeOH/CH₂Cl₂ provided the title compound trifluoromethanesulfonic acid 3-cyano-4′-(quinolin-2-ylmethoxy)-biphenyl-2-yl ester (0.610 g) as a white solid. ¹H NMR (300 MHz, CDCl₃/TMS) δ 8.21 (d, J=8.1 Hz, 1H), 8.09 (d, J=8.4 Hz, 1H), 7.84 (d, J=7.8 Hz, 1H), 7.75 (t, J=7.7 Hz, 1H), 7.72-7.60 (m, 4H), 7.56 (t, J=7.2 Hz, 1H), 7.51 (t, J=7.8 Hz, 1H), 7.39 (d, J=8.4 Hz, 2H), 7.15 (d, J=8.7 Hz, 1H), 5.44 (s, 2H); ¹³C NMR (75 MHz, CDCl₃/TMS) δ 158.9, 157.0, 147.3, 146.5, 137.2, 136.8, 136.4, 132.7, 130.5, 129.7, 128.7, 128.6, 127.5, 127.4, 126.8, 126.4, 117.9 (J=318 Hz), 114.0, 108.5, 71.3.

2-Pyridin-4-yl-4′-(quinolin-2-ylmethoxy)-biphenyl-3-carbonitrile

### Example 1870

### Error! Objects cannot be created from editing field codes

To a suspension of trifluoromethanesulfonic acid 3-cyano-4′-(quinolin-2-ylmethoxy)-biphenyl-2-yl ester (0.128 g) in dioxane (5 mL) and pyridine-4-boronic acid (0.049 g) was added 2M aqueous Na₂CO₃ solution (0.39 mL), and the mixture was purged with argon. Pd(dppf)Cl₂.CH₂Cl₂ (0.011 g) was added and the mixture was purged again with argon. The reaction mixture was heated to reflux for 17 h and then cooled to room temperature and the solvent was removed under reduced pressure. The residue was partitioned between EtOAc (25 mL) and water (25 mL), and neutralized with a 2N aqueous HCl solution. The organic phase was separated from the aqueous phase, and the aqueous phase was extracted with EtOAc (2×15 mL). The combined organic phases were washed with brine (10 mL), and dried over MgSO₄. Concentration and purification by chromatography eluting with 0-70% EtOAc/heptane provided 2-yridin-4-yl-4′-(quinolin-2-ylmethoxy)-biphenyl-3-carbonitrile (0.051 g) as a white solid. ¹H NMR (300 MHz, CDCl₃/TMS) δ 8.55 (d, J=5.7 Hz, 2H), 8.19 (d, J=8.4 Hz, 1H), 8.07 (d, J=8.4 Hz, 1H), 7.83 (d, J=7.8 Hz, 1H), 7.74 (t, J=8.4 Hz, 2H), 7.63 (t, J=7.1 Hz, 2H), 7.60-7.45 (m, 2H), 7.11 (d, J=5.7 Hz, 2H), 6.95 (d, J=9.0 Hz, 2H), 6.87 (d, J=8.4, 2H), 5.33 (s, 2H); ¹³C NMR (75 MHz, CDCl₃/TMS) δ 157.7, 157.1, 149.5, 147.3, 145.1, 141.7, 140.6, 136.8, 134.6, 132.0, 131.3, 130.5, 129.6, 128.7, 127.5, 127.3, 126.4, 124.8, 118.9, 117.7, 114.5, 112.8, 71.2; HRMS: M⁺H m/z=414.1612.

### Synthesis of 6-pyridin-4-yl-4′-(quinolin-2-ylmethoxy)-biphenyl-2-carbonitrile

### Example 383

### 3-Bromo-2-methoxy-benzonitrile

### Error! Objects cannot be created from editing field codes

To a solution of a mixture of 3-bromo-2-hydroxybenzonitrile and 3,5-dibromo-2-hydroxybenzonitrile (1.05 g) in DMF (10 mL) were added iodomethane (2.68 g) and K₂CO₃ (1.56 g) at room temperature and the reaction mixture was stirred for 24 h at the same temperature. The reaction mixture was then diluted with water (100 mL) and extracted with EtOAc (3×30 mL). The combined organic phases were washed with 1 N aqueous NaOH solution (15 mL), water (15 mL) and brine (15 mL), and dried over MgSO₄. Concentration and purification by silica gel chromatography eluting with 1-5% EtOAc/heptane provided 3-bromo-2-methoxy-benzonitrile (0.51 g) as a white solid. ¹H NMR (300 MHz, CDCl₃/TMS) δ 7.79 (dd, J=8.0, 1.4 Hz, 1H), 7.56 (dd, J=7.7, 1.4 Hz, 1H), 7.08 (t, J=7.8 Hz, 1H), 4.07 (s, 3H); ¹³C NMR (75 MHz, CDCl₃/TMS) δ 159.0, 138.1, 132.7, 125.0, 117.5, 115.3, 107.7, 62.0.

### 2-Methoxy-3-pyridin-4-yl-benzonitrile

### Error! Objects cannot be created from editing field codes

To a solution of 3-bromo-2-methoxy-benzonitrile (470 mg), pyridine-4-boronic acid (409 mg) in dioxane (15 mL) was added 2M aqueous Na₂CO₃ solution (3.3 mL) and the mixture was purged with argon. Pd(PPh₃)₄ (128 mg) was added and the mixture was purged again with argon. The reaction mixture was heated to reflux for 17 h. The mixture was cooled to room temperature and the solvent was removed under reduced pressure. The residue was suspended in EtOAc and filtered through a silica gel plug. Evaporation and purification by silica gel chromatography eluting with 0-40% EtOAc/heptane provided 2-methoxy-3-pyridin-4-yl-benzonitrile (330 mg) as a yellow solid. ¹H NMR (300 MHz, CDCl₃/TMS) δ 8.71 (d, J=5.1 Hz, 2H), 7.67 (d, J=7.2 Hz, 1H), 7.61 (dd, J=7.5, 1.2 Hz, 1H), 7.49 (d, J=5.7 Hz, 2H), 7.32 (t, J=7.8 Hz, 1H), 3.76 (s, 3H); ¹³C NMR (75 MHz, CDCl₃/TMS) δ 159.6, 149.9, 144.0, 135.0, 134.0, 132.8, 124.5, 123.4, 116.0, 107.2, 62.0.

2-Hydroxy-3-pyridin-4-yl-benzonitrile

### Error! Objects cannot be created from editing field codes

A stirred mixture of 2-methoxy-3-pyridin-4-yl-benzonitrile (326 mg), thiophenol (222 mg) and K₂CO₃ (22 mg) in dry NMP (1.5 mL) was heated to 190° C. for 0.5 h. The cooled reaction mixture was diluted with water (15 mL), made alkaline with 1 N aqueous NaOH solution, and extracted with diethyl ether (2×7 mL). The aqueous solution was neutralized with 2 N HCl. The resulting yellow precipitate was filtered, washed with EtOAc, and dried over high vacuum to afford the title compound 2-hydroxy-3-pyridin-4-yl-benzonitrile (260 mg) as a yellow solid. ¹H NMR (300 MHz, CDCl₃/CD₃OD/TMS) δ 8.59 (d, J=6.0 Hz, 2H), 7.64-7.55 (m, 4H), 7.11 (t, J=7.7 Hz, 1H); ¹³C NMR (75 MHz, CDCl₃/CD₃OD/TMS) δ 157.2, 149.2, 146.7, 135.7, 134.5, 128.8, 125.1, 121.4, 117.0, 102.8.

### Trifluoromethanesulfonic acid 2-cyano-6-pyridin-4-yl-phenyl ester

### Error! Objects cannot be created from editing field codes

To a solution of 2-hydroxy-3-pyridin-4-yl-benzonitrile (260 mg) in pyridine (7 mL) was added trifluoromethanesulfonic anhydride (561 mg) and DMAP (16 mg) and the mixture was stirred for 24 h under argon at room temperature. The solvent was removed under reduced pressure and the residue was dissolved in CH₂Cl₂ (50 mL) and washed with cold saturated aqueous NaHCO₃ solution (2×20 mL), and dried over MgSO₄. Evaporation and purification by silica gel chromatography eluting with 0-1% MeOH/CH₂Cl₂ provided trifluoromethanesulfonic acid 2-cyano-6-pyridin-4-yl-phenyl ester (330 mg) as a light yellow wax. ¹H NMR (300 MHz, CDCl₃/TMS) δ 8.77 (d, J=4.8 Hz, 2H), 7.88 (d, J=7.8 Hz, 1H), 7.80 (dd, J=7.8, 1.2 Hz, 1H), 7.69 (t, J=7.7 Hz, 1H), 7.44 (d, J=5.1 Hz, 2H); ¹³C NMR (75 MHz, CDCl₃/TMS) δ 150.4, 146.4, 142.2, 136.3, 135.1, 134.8, 129.6, 123.8, 118.1 (J=318 Hz) 113.8, 109.2.

### 6-Pyridin-4-yl-4′-(quinolin-2-ylmethoxy)-biphenyl-2-carbonitrile

### Example 383

### Error! Objects cannot be created from editing field codes

To a solution of trifluoromethanesulfonic acid 2-cyano-6-pyridin-4-yl-phenyl ester (320 mg), and 2-(4-(4,4,5,5-tetramethyl (1,3,2)dioxaborolan-2-yl)-phenoxymethyl)-quinoline (388 mg) in dioxane (15 mL) was added 2M Na₂CO₃ aqueous solution (1.5 mL) and the mixture was purged with argon. Pd(PPh₃)₄ (58 mg) was added and the mixture was purged again with argon. The reaction mixture was heated to reflux for 17 h. The mixture was then cooled to room temperature and the solvent was removed under reduced pressure. The residue was passed through a silica gel plug. Evaporation and purification by chromatography eluting with 0-4% MeOH/CH₂Cl₂ provided 6-pyridin-4-yl-4′-(quinolin-2-ylmethoxy)-biphenyl-2-carbonitrile (350 mg) as a white wax. ¹H NMR (300 MHz, CDCl₃/TMS) δ 8.45 (dd, J=4.2, 1.6 Hz, 2H), 8.21 (d, J=8.7 Hz, 1H), 8.08 (d, J=8.1 Hz, 1H), 7.84 (d, J=8.1 Hz, 1H), 7.81 (dd, J=7.5, 1.5 Hz, 1H), 7.74 (dt, J=6.9, 1.2 Hz, 1H), 7.66 (d, J=8.4 Hz, 1H), 7.60 (dt, J=8.1, 1.3 Hz, 1H), 7.54 (d, J=7.8 Hz, 2H), 7.12-7.06 (m, 2H), 7.01-6.93 (m, 4H), 5.36 (s, 2H); ¹³C NMR (75 MHz, CDCl₃/TMS) δ 158.4, 157.1, 149.3, 147.4, 147.3, 143.5, 139.5, 136.8, 133.8, 133.2, 131.2, 129.6, 128.74, 128.68, 127.8, 127.5, 127.4, 126.4, 124.1, 118.9, 118.0, 114.7, 114.1, 71.2; HRMS: M⁺H m/z=414.1606.

### Synthesis of 2-(2′-nitro-6′-pyridin-4-yl-biphenyl-4-yloxymethyl)-quinoline

### Example 384

### 2-Bromo-3-nitrophenol

### Error! Objects cannot be created from editing field codes

BBr₃ (1.0 M in CH₂Cl₂, 88 mL) was added dropwise over 1 h, to a stirred solution of 2-bromo-3-nitroanisole in CH₂Cl₂ (35 mL) under argon at −70° C. The resulting deep burgundy-colored reaction mixture was allowed to warm to room temperature slowly (over 2 h) and stirred at room temperature for 23 h. The reaction mixture was poured onto 350 g crushed ice and extracted with EtOAc (300 mL). The organic phase was separated, washed with brine (75 mL), and dried over MgSO₄. Concentration and purification by silica gel chromatography eluting with 5-70% EtOAc/heptane gave 2-bromo-3-nitrophenol (5.36 g) as a yellow solid. ¹H NMR (300 MHz, CDCl₃/TMS) δ 7.48 (d, J=8.1 Hz, 1H), 7.37 (t, J=8.1 Hz, 1H), 7.27 (d, J=8.4 Hz, 1H), 6.13 (br s, 1H); ¹³C NMR (75 MHz, CDCl₃/TMS) δ 153.7, 128.7, 119.8, 117.5, 102.9.

### 4′-Benzyloxy-6-nitro-biphenyl-2-ol

### Error! Objects cannot be created from editing field codes

To a solution of 2-bromo-3-nitrophenol (5.36 g) and 4-benzyloxyphenyl boronic acid (6.73 g) in dioxane (220 mL) was added 2 M aqueous Na₂CO₃ solution (55.4 mL) and the mixture was purged with argon. Pd(PPh₃)₄ (1.42 g) was added and the mixture was purged again with argon. The reaction mixture was heated to reflux for 24 h. The mixture was cooled to room temperature and the organic solvent was removed under reduced pressure. The residue was diluted with water (150 mL), neutralized with 2 N HCl, filtered through a Celite® plug, and washed with EtOAc. The filtrate was extracted with EtOAc (3×100 mL). The combined organic phases were washed with brine (50 mL) and dried over MgSO₄. Concentration and purification by silica gel chromatography eluting with 5-40% EtOAc/heptane provided 4′-benzyloxy-6-nitro-biphenyl-2-ol (6.35 g) as yellow solid. ¹H NMR (300 MHz, CDCl₃/TMS) δ 7.52-7.30 (m, 7H), 7.27-7.15 (m, 3H), 7.09 (d, J=7.8 Hz, 2H), 5.73 (s, 1H), 5.09 (s, 2H); ¹³C NMR (75 MHz, CDCl₃/TMS) δ 159.1, 154.1, 149.9, 136.3, 130.4, 128.7, 128.4, 127.9, 127.3, 122.7, 121.8, 119.4, 115.7, 115.5, 70.0.

### 4′-(Benzyloxy)-6-nitrobiphenyl-2-yl trifluoromethanesulfonate

### Error! Objects cannot be created from editing field codes

A solution of 4′-benzyloxy-6-nitro-biphenyl-2-ol (6.37 g) in dry pyridine (120 mL) was treated with trifluoromethanesulfonic anhydride at 0° C. under argon. The resulting mixture was stirred at 0° C. for 0.5 h, then allowed to warm to room temperature and stirred for 18 h. The solvent was removed under reduced pressure, and the residue was dissolved in CH₂Cl₂ (500 mL), washed with cold saturated NaHCO₃ aqueous solution (2×150 mL), and dried over MgSO₄. Filtration and concentration gave 4′-(benzyloxy)-6-nitrobiphenyl-2-yltrifluoromethanesulfonate (9.00 g) as a yellow solid, which was used for the next step without further purification. ¹H NMR (300 MHz, CDCl₃/TMS) δ 7.83 (dd, J=7.2, 1.8 Hz, 1H), 7.63-7.52 (m, 2H), 7.45-7.28 (m, 5H), 7.22 (d, J=8.7 Hz, 2H), 7.06 (d, J=8.7 Hz, 2H), 5.10 (s, 2H); ¹³C NMR (75 MHz, CDCl₃/TMS) δ 159.4, 151.0, 147.2, 136.2, 130.3, 129.0, 128.4, 127.9, 127.4, 125.3, 123.2, 121.4, 118.0 (J=318 Hz), 114.9, 69.9.

### 4-(4′-Benzyloxy-6-nitro-biphenyl-2-yl)-pyridine

### Error! Objects cannot be created from editing field codes

To a solution of 4′-(benzyloxy)-6-nitrobiphenyl-2-yl trifluoromethanesulfonate (4.77 g) and pyridine-4-boronic acid (1.94 g) in dioxane (150 mL) was added 2M aqueous Na₂CO₃ solution (15.8 mL), and the mixture was purged with argon. Pd(PPh₃)₄ (0.61 g) was added and the mixture was purged again with argon. The reaction mixture was heated to reflux for 21 h. The reaction mixture was cooled to room temperature and the solvent was removed under reduced pressure. The residue was partitioned between EtOAc (150 mL) and water (150 mL), and neutralized with 2N aqueous HCl solution. The resulting mixture was passed through a Celite® plug. The organic phase was separated from the aqueous phase, and the latter was extracted with EtOAc (2×50 mL). The combined organic phases were washed with brine (50 mL) and dried over MgSO₄. Concentration and purification by silica gel chromatography eluting with 10-100% EtOAc/heptane provided 4-(4′-benzyloxy-6-nitro-biphenyl-2-yl)-pyridine (3.10 g) as a yellow solid. ¹H NMR (300 MHz, CDCl₃/TMS) δ 8.45 (dd, J=4.5, 1.2 Hz, 2H), 7.79 (dd, J=6.6, 2.7 Hz, 1H), 7.60-7.50 (m, 2H), 7.50-7.20 (m, 5H), 6.96 (dd, J=6.3, 1.5 Hz, 4H), 6.85 (d, J=8.7 Hz, 2H), 5.00 (s, 2H); ¹³C NMR (75 MHz, CDCl₃/TMS) δ 158.4, 151.0, 149.2, 147.2, 140.7, 136.2, 133.4, 132.8, 130.3, 128.4, 128.1, 127.9, 127.4, 126.2, 124.1, 123.1, 114.6, 69.8.

2′-Nitro-6′ pyridin-4-yl-biphenyl-4-ol

### Error! Objects cannot be created from editing field codes

To a solution of 4-(4′-benzyloxy-6-nitro-biphenyl-2-yl)-pyridine (0.74 g) in CH₂Cl₂ (10 mL) was added trifluoroacetic acid (10 mL). The resulting solution was stirred and heated to reflux for 2 h under argon. The solvent was removed under reduced pressure. The residue was partitioned between water (25 mL) and EtOAc (25 mL), and neutralized with a saturated aqueous NaHCO₃ solution. The organic phase was separated from the aqueous phase, and the aqueous phase was extracted with EtOAc (2×25 mL). The combined organic layers were washed with brine and dried over MgSO₄. Concentration and purification by silica gel chromatography eluting with 5-100% EtOAc/heptane afforded 2′-nitro-6′ pyridin-4-yl-biphenyl-4-ol (0.26 g) as a yellow solid. ¹H NMR (300 MHz, CD₃OD/CDCl₃/TMS) δ 8.38 (b s, 2H), 7.82 (d, J=6.9 Hz, 1H), 7.68-7.56 (m, 2H), 7.22-7.02 (m, 2H), 6.87 (d, J=8.4 Hz, 2H), 6.68 (d, J=8.4 Hz, 2H); ¹³C NMR (75 MHz, CD₃OD/CDCl₃/TMS) δ 157.9, 152.1, 149.6, 148.9, 141.3, 134.4, 133.5, 131.3, 129.0, 128.7, 125.8, 123.9, 115.8.

### 2-(2′-Nitro-6′-pyridin-4-yl-biphenyl-4-yloxymethyl)-quinoline

### Example 384

### Error! Objects cannot be created from editing field codes

To a stirred suspension of 2′-nitro-6′ pyridin-4-yl-biphenyl-4-ol (260 mg) in acetonitrile (20 mL) was added K₂CO₃ (615 mg) and the mixture was stirred for 15 min at room temperature. To this suspension, 2-chloromethylquinoline mono-hydrochloride (200 mg) was added at room temperature and the mixture was heated to reflux for 18 h under an argon atmosphere. The reaction mixture was cooled to ambient temperature and the inorganic salts were filtered and washed with acetonitrile. The filtrate was concentrated and the residue was purified via chromatography eluting with 10-100% EtOAc/heptane to provide 2-(2′-nitro-6′-pyridin-4-yl-biphenyl-4-yloxymethyl)-quinoline (240 mg) as a yellow solid. ¹H NMR (300 MHz, CDCl₃/TMS) δ 8.41 (d, J=6.0 Hz, 2H), 8.16 (d, J=8.7 Hz, 1H), 8.05 (d, J=8.1 Hz, 1H), 7.80 (d, J=8.4 Hz, 1H), 7.75 (dd, J=6.6, 2.5 Hz, 1H), 7.70 (dt, J=7.6, 1.2 Hz, 1H), 7.59 (d, J=8.7 Hz, 1H), 7.56-7.44 (m, 3H), 6.98-6.82 (m, 6H), 5.30 (s, 2H); ¹³C NMR (75 MHz, CDCl₃/TMS) δ 158.0, 157.0, 150.9, 149.1, 147.2, 147.1, 140.7, 136.7, 133.3, 132.7, 130.4, 129.5, 128.6, 128.0, 127.4, 127.3, 126.5, 126.3, 124.0, 123.0, 118.8, 114.6, 71.0; HRMS: M⁺H m/z=434.1498.

### Synthesis of 6-Pyridin-4-yl-4′ quinolin-2-ylmethoxy)-biphenyl-2-ylamine

### Example 1881

### 6-Pyridin-4-yl-4′-(quinolin-2-ylmethoxy)-biphenyl-2-ylamine

### Example 1881

### Error! Objects cannot be created from editing field codes

To a solution of 2-(2′-nitro-6′-pyridin-4-yl-biphenyl-4-yloxymethyl)-quinoline (190 mg) in EtOAc (10 mL) and water (0.2 mL) was added SnCl₂ (500 mg) in one portion. The reaction mixture was stirred at room temperature for 18 h. 1N aqueous NaOH solution (20 mL) and EtOAc (10 mL) were added to quench the reaction. The organic layer was separated from the aqueous layer, and the latter was extracted with CHCl₃ (3×10 mL). The combined organic phases were dried over MgSO₄. Filtration, concentration and purification via chromatography eluting with 30-100% EtOAc/heptane provided 6-pyridin-4-yl-4′-(quinolin-2-ylmethoxy)-biphenyl-2-ylamine (150 mg) as light yellow solid. ¹H NMR (300 MHz, CDCl₃/TMS) δ 8.35 (d, J=6.0 Hz, 2H), 8.20 (d, J=8.7 Hz, 1H), 8.08 (d, J=8.4 Hz, 1H), 7.84 (d, J=7.8 Hz, 1H), 7.74 (dt, J=7.7, 1.3 Hz, 1H), 7.65 (d, J=8.4 Hz, 1H), 7.55 (dt, J=8.0, 0.9 Hz, 1H), 7.22 (t, J=7.8 Hz, 1H), 7.07-7.00 (m, 2H), 7.00-6.90 (m, 4H), 6.85-6.75 (m, 2H), 5.35 (s, 2H), 3.58 (b s, 2H); ¹³C NMR (75 MHz, CDCl₃/TMS) δ 157.4, 149.9, 148.5, 147.3, 144.6, 139.3, 136.8, 131.7, 129.6, 129.1, 128.7, 128.2, 127.5, 127.4, 126.4, 125.1, 124.4, 119.4, 118.9, 115.2, 115.1, 71.1; HRMS: M⁺H m/z=404.1759.

### Synthesis of 2-((2′-(Pyridin-4-yl)biphenyl-4-yloxy)methyl)quinoline

### Example 380

### 4-(2-(benzyloxy)phenyl)pyridine

### Error! Objects cannot be created from editing field codes

A mixture of benzyl 2-bromophenyl ether (0.12 g), 4-pyridine-boronic acid (84 mg), triphenylphosphine (24 g), cesium carbonate (0.60 g) in DMF (3 mL) was degassed four times before Pd(dppf)Cl₂ (33 mg) was added. The mixture was then degassed four times and heated at 110° C. for 24 h. The solvent was evaporated and the residue was filtered and washed with dichloromethane/MeOH (1:1). The crude material was purified via medium pressure flash chromatography eluting with 5% methanol in dichloromethane to yield 4-(2-(benzyloxy)phenyl)pyridine as an oil (80 mg). ¹H NMR (300 MHz, CDCl₃/TMS), δ 8.61 (d, J=6.0 Hz, 2H), 7.51 (d, J=5.7 Hz, 2H), 7.38-7.32 (m, 7H), 7.08 (m, 2H), 5.11 (s, 2H); ¹³C NMR (75 MHz, CDCl₃/TMS) δ 155.34, 149.97, 149.16, 146.05, 136.40, 130.33, 129.86, 128.30, 128.01, 127.62, 126.65, 124.19, 121.25, 112.99, 70.33.

### 2-(pyridin-4-yl)phenol

### Error! Objects cannot be created from editing field codes

4-(2-Benzyloxy-phenyl)-pyridine (3.27 g) and 10% palladium on carbon (0.75 g) in 50 mL of ethanol was hydrogenated at 30 psi for 18 h. The mixture was filtered, washed with methanol, and purified by silica gel flash chromatography eluting with methanol/dichloromethane (20/1) to give 2-(pyridin-4-yl)phenol as a white solid (2.11 g). mp 218-220° C. ¹H NMR (300 MHz, CD₃OD/TMS) δ 8.49 (m, 2H), 7.67 (dd, J=6.3, 1.5 Hz, 2H), 7.35 (dd, J=7.2, 1.5 Hz, 1H), 7.24 (m, 1H), 6.95-6.91 (m, 2H), 4.94 (s, 1H); ¹³C NMR (75 MHz, CD₃OD/TMS) δ 155.89, 149.26, 131.23, 131.05, 125.89, 125.56, 120.95, 117.08.

### 2-(Pyridin-4-yl)phenyl trifluoromethanesulfonate

### Error! Objects cannot be created from editing field codes

A solution of 2-(pyridin-4-yl)phenol (0.39 g) in dry pyridine (7 mL) was treated with trifluoromethanesulfonic anhydride (0.71 g) at 0° C. under argon. The resulting mixture was stirred at 0° C. for 30 min, then at room temperature overnight. The solvent was removed under vacuum, the residue was dissolved in dichloromethane, washed with cold sodium bicarbonate solution, and dried over Na₂SO₄. The crude mixture was used directly in the next step without any purification. ¹H NMR (300 MHz, CDCl₃/TMS) δ 8.72 (d, J=4.2 Hz, 2H), 7.51 (m, 3H), 7.46-7.40 (m, 3H). ¹³C NMR (75 MHz, CDCl₃/TMS) δ 150.22, 146.55, 143.63, 132.94, 131.68, 130.64, 129.07, 124.15, 122.62, 118.50 (q, J=318.4 Hz). ¹⁹F NMR (282 MHz, CDCl₃) δ −74.52.

### 2-((2′-(Pyridin-4-yl)biphenyl-4-yloxy)methyl)quinoline

### Example 380

### Error! Objects cannot be created from editing field codes

A mixture of 2-(pyridin-4-yl)phenyl trifluoromethanesulfonate (0.185 g), 4-(quinolin-2-ylmethoxy)phenylboronic acid (0.187 g) and cesium carbonate (0.597 g) in DMF (4 mL) was degassed four times before Pd(dppf)Cl₂ (22 mg) was added. The mixture was degassed four more times, then heated to 110° C. for 21 h. The mixture was filtered and the solid was washed with dichloromethane/methanol (1:1). The filtrate was concentrated and purified on silica gel column eluting with 50% ethyl acetate in heptane to give 2-((2′-(pyridin-4-yl)biphenyl-4-yloxy)methyl)quinoline as a waxy solid (142 mg). HRMS (DIP-CI-MS): Calcd for C₂₇H₂₁N₂O [M+H]⁺, 389.1611, found, 389.1621; ¹H NMR (300 MHz, CDCl₃/TMS) δ 8.44 (d, J=5.4 Hz, 2H), 8.17 (d, J=8.4 Hz, 1H), 8.08 (d, J=8.4 Hz, 1H), 7.81 (d, J=8.1 Hz, 1H), 7.72 (dd, J=8.1, 7.2 Hz, 1H), 7.65 (d, J=8.7 Hz, 1H), 7.53, (dd, J=7.8, 7.2 Hz, 1H), 7.42-7.38 (m, 4H), 7.06-7.01, (m, 4H), 6.90 (d, J=8.4 Hz, 1H), 5.35 (s, 2H). ¹³C NMR (75 MHz, CDCl₃/TMS) δ 157.8, 157.7, 149.8, 149.5, 147.7, 140.3, 137.8, 137.2, 133.6, 131.2, 131.0, 130.3, 130.0, 129.1, 128.9, 127.9, 127.8, 127.7, 126.7, 124.9, 119.4, 114.8, 71.6.

### Synthesis of Example 1863

### Biphenyl-2-yl trifluoromethanesulfonate

### Error! Objects cannot be created from editing field codes

A solution of 2-phenylphenol (1.0 g) in dry pyridine (10 mL) was treated with trifluoromethanesulfonic anhydride (1.82 g) at 0° C. under argon. The resulting mixture was stirred for 30 min at 0° C., then at room temperature overnight. The solvent was removed, the residue was diluted with methylene chloride, washed with cold sodium bicarbonate solution, and dried over Na₂SO₄. The crude mixture was used directly in the next step without any purification. ¹H NMR (300 MHz, CDCl₃/TMS) δ 7.46-7.45 (m, 6H), 7.41-7.39 (m, 3H). ¹³C NMR (75 MHz, CDCl₃/TMS) δ 146.57, 135.36, 131.78, 130.73, 129.16, 128.78, 128.32, 128.29, 128.10, 121.89, 118.16 (q, J=318.4 Hz). ¹⁹F NMR (282 MHz, CDCl₃) δ −74.80.

### Example 1863

### Error! Objects cannot be created from editing field codes

A mixture of biphenyl-2-yl trifluoromethanesulfonate (0.2 g), 2-((4-(4,4,5,5-tetramethyl-1,3,2-dioxaborolan-2-yl)phenoxy)methyl)quinoline (0.263 g) and cesium carbonate (0.65 g) in DMF (5 mL) was degassed four times before Pd(dppf)Cl₂ (24 mg) was added. The mixture was degassed four more times, then heated to 110° C. for 28 h. The mixture was filtered and the solid was washed with dichloromethane/methanol (1:1). The filtrate was concentrated and purified on a silica gel column eluting with 20% ethyl acetate in heptane to give 200 mg of a white solid, mp 90-92° C. HRMS (DIP-CI-MS): Calcd for C₂₈H₂₂NO [M+H]⁺, 388.1701, found, 388.1669; calcd for C₂₈H₂₁NO [M]⁺, 387.1623, found, 387.1595; ¹H NMR (300 MHz, CDCl₃/TMS), δ 8.16 (d, J=8.7 Hz, 1H), 8.07 (d, J=7.8 Hz, 1H), 7.81 (d, J=7.5 Hz, 1H), 7.72 (dd, J=7.2, 7.8 Hz, 1H), 7.65, (d, J=8.4 Hz, 1H), 7.53 (dd, J=7.5, 6.6 Hz, 1H), 7.38 (m, 4H), 7.18-7.14 (m, 5H), 7.05 (d, J=7.8 Hz, 2H), 6.87 (d, J=8.4 Hz, 1H), 5.33 (s, 2H). ¹³C NMR (75 MHz, CDCl₃/TMS) δ 158.1, 157.3, 147.7, 141.8, 140.9, 140.2, 137.1, 134.7, 131.2, 130.8, 130.7, 130.1, 129.9, 129.2, 128.1, 127.9, 127.8, 127.7, 127.4, 126.7, 126.6, 119.4, 114.6, 71.6.

### Synthesis of Example 330

### 2-(2-Iodophenoxy)tetrahydro-2H-pyran

### Error! Objects cannot be created from editing field codes

2-Iodophenol (4.31 g) and pyridinium p-toluenesulfonate (49 mg) was stirred in 80 mL of dry dichloromethane and 3,4-dihydro-2H-pyran (1.97 g) was added dropwise at room temperature. The mixture was stirred at room temperature overnight. The solvent was removed and the residue was purified by silica gel flash chromatography eluting with 20% ethyl acetate in heptane to give 2-(2-iodophenoxy)tetrahydro-2H-pyran as a of a colorless oil (5.53 g). ¹H NMR (300 MHz, CDCl₃/TMS) δ 7.75 (d, J=8.1 Hz, 1H), 7.26 (m, 1H), 7.07 (d, J=8.1 Hz, 1H), 6.72 (m, 1H), 5.54 (s, 1H), 3.87 (m 1H), 3.59 (m, 1H), 2.15 (m, 1H), 1.98 (m, 1H), 1.88 (m, 1H), 1.72-1.66 (m, 3H); ¹³C NMR (75 MHz, CDCl₃/TMS) δ 155.23, 139.02, 129.12, 123.04, 114.93, 96.27, 87.27, 61.58, 30.13, 25.18, 18.25.

### 2-((2′-(Tetrahydro-2H-pyran-2-yloxy)biphenyl-4-yloxy)methyl)quinoline

### Error! Objects cannot be created from editing field codes

A mixture of 2-(2-iodophenoxy)-tetrahydropyran (3.96 g), 2-[4-(4,4,5,5-tetramethyl-[1,3,2]dioxaborolan-2-yl)-phenoxymethyl]-quinoline (2.6 g), cesium carbonate (8.95 g) in 70 mL of DMF was degassed four times before Pd(dppf)Cl₂ (340 mg) was added. The mixture was degassed four more times, then heated to 90° C. for 25 h. The mixture was filtered and washed with dichloromethane/methanol (1:1). The filtrate was concentrated and purified by silica gel flash chromatography eluting with 20% ethyl acetate in heptane to give 2-((2′-(tetrahydro-2H-pyran-2-yloxy)biphenyl-4-yloxy)methyl)quinoline as a colorless oil (3.73 g). ¹H NMR (300 MHz, CDCl₃/TMS) δ 8.19 (d, J=8.7 Hz, 1H), 8.09 (d, J=8.7 Hz, 1H), 7.82 (d, J=8.4 Hz, 1H), 7.76-7.69 (m, 2H), 7.57-7.49 (m, 3H), 7.31 (d, J=7.2 Hz, 1H), 7.28-7.19 (m, 2H), 7.08-7.01 (m, 3H), 5.43 (s, 2H), 5.39 (s, 1H), 3.81-3.74 (m, 1H), 3.56-3.52 (m, 1H), 1.79-1.51 (m, 6H); ¹³C NMR (75 MHz, CDCl₃/TMS) δ 158.19, 157.54, 153.95, 147.75, 137.17, 131.82, 131.33, 130.98, 130.76, 129.98, 129.14, 128.36, 127.92, 127.79, 126.71, 122.14, 119.39, 116.06, 114.47, 96.88, 71.61, 62.09, 30.64, 25.60, 18.88.

### 4′-(Quinolin-2-ylmethoxy)biphenyl-2-ol

### Error! Objects cannot be created from editing field codes

2-[2′-(Tetrahydropyran-2-yloxy)-biphenyl-4-yloxymethyl]-quinoline (3.73 g) in methanol was treated with pyridinium p-toluenesulfonate (22 mg) at 50° C. for 6 h. The solvent was removed and the residue was purified by silica gel flash chromatography eluting with 50% ethyl acetate in heptane to give 4′-(quinolin-2-ylmethoxy)biphenyl-2-ol as a yellow solid (2.67 g). ¹H NMR (300 MHz, CD₃OD/CDCl₃/TMS) δ 8.26 (d, J=8.7 Hz, 1H), 8.08 (d, J=7.8 Hz, 1H), 7.6 (d, J=8.1 Hz, 1H), 7.79-7.71 (m, 2H), 7.59 (d, J=7.2 Hz, 1H), 7.52 (d, J=8.7 Hz, 2H), 7.24 (d, J=7.8 Hz, 1H), 7.15 (m, 1H), 7.08 (d, J=8.7 Hz, 2H), 6.92 (d, J=7.5 Hz, 2H), 5.39 (s, 2H), 4.29 (s, 1H); ¹³C NMR (75 MHz, CD₃OD/CDCl₃/TMS) δ 157.98, 157.33, 153.65, 147.08, 137.74, 131.78, 131.48, 130.53, 130.21, 128.23, 128.06, 127.87, 127.78, 126.82, 120.05, 119.42, 115.85, 114.68, 70.85.

### 4′-(Quinolin-2-ylmethoxy)biphenyl-2-yl trifluoromethanesulfonate

### Error! Objects cannot be created from editing field codes

4′-(Quinolin-2-ylmethoxy)-biphenyl-2-ol (1.08 g) in pyridine (15 mL) was treated with trifluoromethanesulfonic anhydride (1.12 g) at 0° C. under argon. The resulting mixture stirred for 30 min at 0° C., then room temperature overnight. The solvent was removed, the residue was diluted with methylene chloride, washed with cold sodium bicarbonate solution, and dried over Na₂SO₄. The crude mixture was purified by silica gel flash chromatography eluting with 0.5% methanol in dichloromethane to give 4′-(quinolin-2-ylmethoxy)biphenyl-2-yltrifluoromethanesulfonate as an off-white solid (0.90 g). ¹H NMR (300 MHz, CDCl₃/TMS) δ 8.16 (d, J=8.4 Hz, 1H), 8.09 (d, J=8.4 Hz, 1H), 7.79 (d, J=8.4 Hz, 1H), 7.74-7.65 (m, 2H), 7.52 (dd, J=7.2, 7.5 Hz, 1H), 7.39-7.34 (m, 6H), 7.10 (d, J=8.4 Hz, 1H), 5.41 (s, 2H); ¹³C NMR (75 MHz, CDCl₃/TMS) δ 158.68, 157.74, 147.75, 147.04, 137.21, 135.28, 132.07, 130.89, 130.01, 129.15, 128.79, 128.71, 127.93, 127.82, 126.76, 122.28, 119.35, 118.59 (q, J=317.8 Hz), 115.20, 71.61. ¹⁹F NMR (282 MHz, CDCl₃) δ −74.49.

### Example 330

### Error! Objects cannot be created from editing field codes

A mixture of trifluoromethanesulfonic acid 4′-(quinolin-2-ylmethoxy)-biphenyl-2-yl ester (0.168 g), 4-methoxybenzeneboronic acid (84 mg), and cesium carbonate (0.36 g) in DMF (5 mL) was degassed four times before Pd(dppf)Cl₂ (14 mg) was added. The mixture was degassed four more times, then heated to 110° C. for 24 h. The mixture was filtered and washed with dichloromethane/methanol (1:1). The filtrate was concentrated and purified by silica gel flash chromatography eluting with 20% ethyl acetate in heptane to give the desired product as a semi-solid (51 mg). HRMS (TOF-MS): Calcd for C₂₉H₂₄NO₂ [M+H]⁺: 418.1802, found, 418.1815; ¹H NMR (300 MHz, CDCl₃/TMS) δ 8.16 (d, J=8.4 Hz, 1H), 8.08 (d, J=8.4 Hz, 1H), 7.81 (d, J=7.8 Hz, 1H), 7.72 (dd, J=6.9, 8.4 Hz, 1H), 7.66 (d, J=8.4 Hz, 1H), 7.53 (dd, J=7.5, 7.2 Hz, 1H), 7.36 (m, 4H), 7.05 (m, 5H), 6.88 (d, J=8.4 Hz, 2H), 6.75 (d, J=8.4 Hz, 2H), 5.34 (s, 2H), 3.75 (s, 3H); ¹³C NMR (75 MHz, CDCl₃/TMS) δ 158.40, 158.09, 147.72, 140.27, 140.13, 137.16, 134.88, 134.20, 132.04, 131.22, 131.09, 130.73, 130.11, 129.99, 129.14, 128.07, 127.92, 127.40, 127.34, 126.72, 119.41, 114.60, 113.62, 71.54, 55.48.

### Example [[EP42700]]

### Error! Objects cannot be created from editing field codes

A mixture of trifluoromethanesulfonic acid 4′-(quinolin-2-ylmethoxy)-biphenyl-2-yl ester (0.17 g), 3-methoxybenzeneboronic acid (84 mg), and cesium carbonate (0.36 g) in DMF (5 mL) was degassed four times before Pd(dppf)Cl₂ (14 mg) was added. The mixture was degassed four more times, then heated to 110° C. for 24 h. The mixture was filtered and washed with dichloromethane/methanol (1:1). The filtrate was concentrated and purified by silica gel flash chromatography eluting with 20% ethyl acetate in heptane to give the desired product as a semi-solid (120 mg). HRMS (DIP-CI-MS): Calcd for C₂₉H₂₄NO₂ [M+H]⁺: 418.1801, found 418.1802; ¹H NMR (300 MHz, CDCl₃/TMS) δ 8.13 (d, J=8.4 Hz, 1H), 8.07 (d, J=8.4 Hz, 1H), 7.78 (d, J=8.1 Hz, 1H), 7.70 (m, 1H), 7.62 (d, J=8.4 Hz, 1H), 7.51 (m, 1H), 7.37 (m, 4H), 7.13-7.05 (m, 3H), 6.88 (d, J=8.4 Hz, 2H), 6.74 (m, 2H), 6.66 (m, 1H), 5.33 (s, 2H), 3.58 (s, 3H); ¹³C NMR (75 MHz, CDCl₃/TMS) δ 159.23, 158.08, 157.37, 147.73, 143.18, 140.55, 140.25, 137.16, 134.74, 131.17, 130.67, 130.69, 130.00, 129.15, 129.11, 128.08, 127.93, 127.77, 127.42, 126.73, 122.58, 119.38, 115.48, 114.64, 112.75, 71.56, 55.39.

### Example 75 [[43800]]

### 2-((2′-(Pyridin-3-yl)biphenyl-4-yloxy)methyl)quinoline

### Error! Objects cannot be created from editing field codes

A mixture of trifluoromethanesulfonic acid 4′-(quinolin-2-ylmethoxy)-biphenyl-2-yl ester (0.15 g), 3-pyridineboronic acid (60 mg), and cesium carbonate (0.32 g) in 1,4-dioxane (5 mL) was degassed four times before Pd(dppf)Cl₂ (12 mg) was added. The mixture was degassed four more times, then heated to 110° C. for 24 h. The mixture was filtered and washed with dichloromethane/methanol (1:1). The filtrate was concentrated and purified by silica gel flash chromatography eluting with 5% methanol in dichloromethane to give 2-((2′-(pyridin-3-yl)biphenyl-4-yloxy)methyl)quinoline as a light yellow oil (99 mg). HRMS (TOF-MS): Calcd for C₂₇H₂₁N₂O [M+H]⁺: 389.1648, found, 389.1669; ¹H NMR (300 MHz, CDCl₃/TMS) δ 8.45 (s, 1H), 8.42 (d, J=4.5 Hz, 1H), 8.16 (d, J=8.7 Hz, 1H), 8.07 (d, J=8.7 Hz, 1H), 7.80 (d, J=8.1 Hz, 1H), 7.71 (dd, J=8.1, 7.2 Hz, 1H), 7.64, (d, J=8.4 Hz, 1H), 7.52 (dd, J=8.1, 7.2 Hz, 1H), 7.41-7.36 (m, 5H), 7.09 (dd, J=4.8, 7.5 Hz, 1H), 7.02 (d, J=8.7 Hz, 1H), 6.89 (d, J=8.7 Hz, 1H), 5.35 (s, 2H); ¹³C NMR (75 MHz, CDCl₃/TMS) δ 157.89, 157.62, 150.48, 147.76, 140.62, 17.48, 137.27, 137.18, 136.89, 135.06, 133.80, 131.31, 130.92, 130.66, 129.97, 129.14, 128.52, 127.91, 127.70, 126.72, 122.93, 119.38, 114.86, 71.57.

### Synthesis of 2-((2′-(2-methylpyridin-4-yl)biphenyl-4-yloxy)methyl)quinoline

### Example 1859

### 2-((2′-(2-methylpyridin-4-yl)biphenyl-4-yloxy)methyl)quinoline

### Example 1859

### Error! Objects cannot be created from editing field codes

A mixture of trifluoromethanesulfonic acid 4′-(quinolin-2-ylmethoxy)-biphenyl-2-yl ester (0.21 g), 2-picoline-4-boronic acid (94 mg), and 2 M Na₂CO₃ solution (0.93 mL) in 1,4-dioxane (5 mL) was degassed four times before Pd(dppf)Cl₂ (17 mg) was added. The mixture was degassed four more times, then heated to 110° C. for 18 h. The mixture was filtered and washed with dichloromethane/methanol (1:1). The filtrate was concentrated and purified by silica gel flash chromatography eluting with 2% isopropanol in dichloromethane to give 2-((2′-(2-methylpyridin-4-yl)biphenyl-4-yloxy)methyl)quinoline as an oil (90 mg). HRMS (ESI-TOF): Calcd for C₂₈H₂₃N₂O [M+H]⁺: 403.1805; found: 403.1803. ¹H NMR (300 MHz, CDCl₃/TMS) δ 8.29 (d, J=5.1 Hz, 1H), 8.17 (d, J=8.1 Hz, 1H), 8.08 (d, J=8.1 Hz, 1H), 7.82 (d, J=8.1 Hz, 1H), 7.72 (m, 1H), 7.65 (d, J=8.4 Hz, 1H), 7.54 (m, 1H), 7.41-7.38 (m, 4H), 7.04 (d, J=8.4 Hz, 2H), 6.97 (s, 1H), 6.90 (d, J=8.7 Hz, 2H), 6.81 (d, J=4.5 Hz, 1H), 5.36 (s, 2H), 2.46 (s, 3H); ¹³C NMR (75 MHz, CDCl₃/TMS) δ 158.13, 157.90, 157.67, 150.07, 148.69, 147.72, 140.29, 138.02, 137.16, 133.77, 131.14, 130.92, 130.30, 129.99, 129.14, 128.75, 127.91, 127.77, 127.60, 126.74, 124.38, 122.19, 119.32, 114.80, 71.56, 24.77.

### Synthesis of 2-((4′-Chloro-2′-(pyridin-4-yl)biphenyl-4-yloxy)methyl)quinoline

### Example 1876

### 2-(2-Bromo-4-chlorophenoxy)tetrahydro-2H-pyran

### Error! Objects cannot be created from editing field codes

A mixture of 2-bromo-4-chlorophenol (5.0 g) and pyridinium p-toluenesulfonate (60 mg) was stirred in 80 mL of dry dichloromethane and 3,4-dihydro-2H-pyran (1.97 g) was added dropwise at room temperature. The mixture was stirred at room temperature for 24 h. The solvent was removed and the residue was purified by silica gel flash chromatography eluting with 20% ethyl acetate in heptane to give 2-(2-bromo-4-chlorophenoxy)tetrahydro-2H-pyran (5.58 g) as a colorless oil. ¹H NMR (300 MHz, CDCl₃/TMS) δ 7.53 (d, J=2.1 Hz, 1H), 7.19 (m, 1H), 7.08 (d, J=9.0 Hz, 1H), 5.46 (m, 1H), 3.84 (m, 1H), 3.60 (m, 1H), 2.09-1.65 (m, 6H); ¹³C NMR (75 MHz, CDCl₃/TMS) δ 151.97, 132.42, 128.02, 126.66, 116.99, 113.31, 96.77, 61.02, 30.02, 25.08, 18.16.

### 4-(5-Chloro-2-(tetrahydro-2H-pyran-2-yloxy)phenyl)pyridine

### Error! Objects cannot be created from editing field codes

A mixture of 2-(2-bromo-4-chlorophenoxy)-tetrahydropyran (2.0 g), 4-pyridineboronic acid (1.01 g), and cesium carbonate (6.71 g) in 1,4-dioxane (40 mL) was degassed four times before Pd(PPh₃)₄ (0.40 g) was added. The mixture was degassed four more times, then heated to 110° C. for 24 h. The mixture was filtered and washed with dichloromethane/methanol (1:1). The filtrate was concentrated and purified by silica gel flash chromatography eluting with 50% ethyl acetate in heptane to give 4-(5-chloro-2-(tetrahydro-2H-pyran-2-yloxy)phenyl)pyridine (1.23 g) as a clear oil. ¹H NMR (300 MHz, CDCl₃/TMS) δ 8.64 (d, J=6.0 Hz, 2H), 7.46 (m, 2H), 7.32-7.28 (m, 2H), 7.19 (d, J=8.4 Hz, 1H), 5.41 (s, 1H), 3.72 (m, 1H), 3.58 (m, 1H), 1.79-1.56 (m, 6H); ¹³C NMR (75 MHz, CDCl₃/TMS) δ 152.67, 149.67, 145.29, 130.10, 129.83, 127.07, 124.34, 117.18, 97.13, 62.19, 30.41, 25.35, 18.78.

### 4-Chloro-2-(pyridin-4-yl)phenol

### Error! Objects cannot be created from editing field codes

A solution of 4-[5-chloro-2-(tetrahydropyran-2-yloxy)-phenyl]-pyridine (1.23 g) in methanol (50 mL) was treated with pyridinium p-toluenesulfonate (11 mg) at 50° C. for 48 h. The solvent was removed and the residue was washed with dichloromethane to give 4-chloro-2-(pyridin-4-yl)phenol (0.40 g) as a light yellow solid. ¹H NMR (300 MHz, CD₃OD/CDCl₃/TMS) δ 8.54 (d, J=4.2 Hz, 2H), 7.62 (d, J=6.0 Hz, 2H), 7.30 (d, J=2.4 Hz, 1H), 7.20 (dd, J=2.4, 8.4 Hz, 1H), 6.91 (d, J=8.7 Hz, 1H), 4.40 (s, 1H); ¹³C NMR (75 MHz, CD₃OD/CDCl₃/TMS) δ 153.47, 148.70, 146.70, 129.96, 129.75, 126.56, 124.77, 124.56, 117.68.

### 4-Chloro-2-(pyridin-4-yl)phenyl trifluoromethanesulfonate

### Error! Objects cannot be created from editing field codes

A solution of 4-chloro-2-pyridin-4-yl-phenol (0.48 g) in dry pyridine (10 mL) was treated with trifluoromethanesulfonic anhydride (0.79 g) at 0° C. under argon. The resulting mixture was stirred for 30 min at 0° C., then room temperature overnight. The solvent was removed, the residue was diluted with methylene chloride, washed with cold sodium bicarbonate solution, and dried over Na₂SO₄. The crude mixture (0.80 g) was used directly in the next step without any purification. ¹H NMR (300 MHz, CDCl₃/TMS) δ 8.73 (s, 2H), 7.48 (m, 2H), 7.39 (m, 3H); ¹³C NMR (75 MHz, CDCl₃/TMS) δ 150.35, 144.83, 142.34, 134.75, 134.48, 131.42, 130.44, 123.94, 123.86, 118.43 (q, J=317.7 Hz); ¹⁹F NMR (282 MHz, CDCl₃) δ −74.15.

2-((4′-Chloro-2′-(pyridin-4-yl)biphenyl-4-yloxy)methyl)quinoline

### Example 1876

### Error! Objects cannot be created from editing field codes

A mixture of 4-chloro-2-(pyridin-4-yl)phenyl trifluoromethanesulfonate (0.33 g), 2-[4-(4,4,5,5-tetramethyl-[1,3,2]dioxaborolan-2-yl)-phenoxymethyl]-quinoline (0.388 g), and 2M Na₂CO₃ solution (1.5 mL) in 1,4-dioxane (10 mL) was degassed four times before Pd(PPh₃)₄ (56 mg) was added. The mixture was degassed four more times and then heated to reflux for 24 h. The mixture was filtered and washed with dichloromethane/methanol (1:1). The filtrate was concentrated and purified by silica gel flash chromatography eluting with 2.5% methanol in dichloromethane to give 2-((4′-chloro-2′-(pyridin-4-yl)biphenyl-4-yloxy)methyl)quinoline (0.38 g) as a white foam. HRMS (ESI-TOF-MS): Calcd for C₂₇H₂₀ClN₂O [M+H]⁺: 423.1259, found 423.1259. ¹H NMR (300 MHz, CDCl₃/TMS) δ 8.45 (s, 2H), 8.18 (d, J=8.7 Hz, 1H), 8.07 (d, J=8.4 Hz, 1H), 7.82 (d, J=7.8 Hz, 1H), 7.73 (dd, J=7.2, 7.2 Hz, 1H), 7.64 (d, J=8.4 Hz, 1H), 7.54 (dd, J=7.2, 7.2 Hz, 1H), 7.42-7.32 (m, 3H), 7.02-6.97 (m, 4H), 6.90 (d, J=8.4 Hz, 2H), 5.35 (s, 2H). ¹³C NMR (75 MHz, CDCl₃/TMS) δ 157.95, 157.70, 149.70, 148.46, 147.71, 139.24, 138.81, 137.18, 133.48, 132.41, 132.24, 131.05, 130.11, 130.01, 129.14, 128.85, 127.90, 127.78, 126.77, 124.65, 119.33, 114.99, 71.6.

### Synthesis of 2-((5′-Chloro-2′-(pyridin-4-yl)biphenyl-4-yloxy)methyl)quinoline

### Example 405

### 2-((5′-chloro-2′-(tetrahydro-2H-pyran-2-yloxy)biphenyl-4-yloxy)methyl)quinoline

### Error! Objects cannot be created from editing field codes

A mixture of 2-(2-bromo-4-chlorophenoxy)-tetrahydropyran (1.98 g), 2-[4-(4,4,5,5-tetramethyl-[1,3,2]dioxaborolan-2-yl)-phenoxymethyl]-quinoline (2.45 g), and 2M Na₂CO₃ solution (10.2 mL) in 1,4-dioxane (60 mL) was degassed four times before Pd(PPh₃)₄ (0.40 g) was added. The mixture was degassed four more times, then heated to reflux for 24 h. The mixture was filtered and washed with dichloromethane/methanol (1:1). The filtrate was concentrated and purified by silica gel flash chromatography eluting with 50% ethyl acetate in heptane to give 2-((5′-chloro-2′-(tetrahydro-2H-pyran-2-yloxy)biphenyl-4-yloxy)methyl)quinoline (2.58 g) as a semi-solid. ¹H NMR (300 MHz, CDCl₃/TMS) δ 8.08 (dd, J=8.4, 3.9 Hz, 2H), 7.23-7.61 (m, 3H), 7.45 (m, 3H), 7.26 (d, J=2.1 Hz, 1H), 7.16-7.10 (m, 2H), 7.05 (d, J=9.0 Hz, 2H), 5.37 (s, 2H), 5.28 (s, 1H), 3.69 (m, 1H), 3.49 (m, 1H), 1.75-1.45 (m, 6H); ¹³C NMR (75 MHz, CDCl₃/TMS) δ 157.92, 152.55, 147.56, 137.28, 132.89, 130.89, 130.47, 130.31, 130.04, 128.91, 127.90, 127.76, 126.92, 126.76, 119.38, 117.41, 114.62, 97.09, 71.38, 62.06, 30.49, 25.48, 18.79.

### 5-Chloro-4′-(quinolin-2-ylmethoxy)biphenyl-2-ol

### Error! Objects cannot be created from editing field codes

A solution of 2-[5′-chloro-2′-(tetrahydropyran-2-yloxy)-biphenyl-4-yloxymethyl]-quinoline (2.58 g) in methanol (50 mL) was treated with pyridinium p-toluenesulfonate (11 mg) at 50° C. for 16 h. The solvent was removed and the residue was washed with dichloromethane to give 5-chloro-4′-(quinolin-2-ylmethoxy)biphenyl-2-ol

(2.31 g) as an off-white solid was used directly in the next step ¹H NMR (300 MHz, CDCl₃/TMS) δ 8.44 (d, J=9.0 Hz, 1H), 8.13 (d, J=9.0 Hz, 1H), 7.97 (d, J=7.2 Hz, 1H), 7.83 (m, 2H), 7.57-7.50 (m, 3H), 7.20 (s, 2H), 7.09 (m, 3H), 5.46 (s, 2H).

### 5-Chloro-4′-(quinolin-2-ylmethoxy)biphenyl-2-yl trifluoromethanesulfonate

### Error! Objects cannot be created from editing field codes

A solution of 5-chloro-4′-(quinolin-2-ylmethoxy)-biphenyl-2-ol (2.31 g) in dry pyridine (20 mL) was treated with trifluoromethanesulfonic anhydride (1.96 g) at 0° C. under argon. The resulting mixture stirred for 30 min at 0° C., then room temperature overnight. The solvent was removed and the residue was diluted with methylene chloride, washed with cold sodium bicarbonate solution, and dried over Na₂SO₄. The crude mixture (2.07 g) was used directly in the next step without any purification. ¹H NMR (300 MHz, CDCl₃/TMS) δ 8.16 (d, J=8.4 Hz, 1H), 8.09 (d, J=8.7 Hz, 1H), 7.79 (d, J=7.8 Hz, 1H), 7.15 (m, 1H), 7.65 (d, J=8.7 Hz, 1H), 7.52 (m, 1H), 7.40-7.34 (m, 3H), 7.29-7.23 (m, 2H), 7.10 (d, J=8.7 Hz, 2H), 5.41 (s, 2H); ¹³C NMR (75 MHz, CDCl₃/TMS) δ 159.05, 157.57, 147.72, 145.33, 137.24, 136.91, 134.28, 131.77, 130.80, 130.04, 129.15, 128.59, 127.92, 127.79, 126.79, 123.61, 119.31, 118.37 (q, J=328.5 Hz), 115.35, 71.61. ¹⁹F NMR (282 MHz, CDCl₃) δ −74.32.

### 2-((5′-Chloro-2′-(pyridin-4-yl)biphenyl-4-yloxy)methyl)quinoline

### Example 405

### Error! Objects cannot be created from editing field codes

A mixture of 5-chloro-4′-(quinolin-2-ylmethoxy)biphenyl-2-yl trifluoromethanesulfonate (0.36 g), 4-pyridineboronic acid (107 mg), and 2 M Na₂CO₃ solution (1.09 mL) in 1,4-dioxane (10 mL) was degassed four times before Pd(PPh₃)₄ (42 mg) was added. The mixture was degassed four more times, then heated to reflux for 24 h. The mixture was filtered and washed with dichloromethane/methanol (1:1). The filtrate was concentrated and purified by silica gel flash chromatography eluting with 50% ethyl acetate in heptane to give 2-((5′-chloro-2′-(pyridin-4-yl)biphenyl-4-yloxy)methyl)quinoline (0.2 g) as a white foam. HRMS (ESI-TOF-MS): Calcd for C₂₇H₂₀ClN₂O [M+H]⁺: 423.1259, found 423.1264. ¹H NMR (300 MHz, CDCl₃/TMS) δ 8.43 (d, J=4.5 Hz, 2H), 8.15 (d, J=8.7 Hz, 1H), 8.07 (d, J=8.4 Hz, 1H), 7.79 (d, J=8.4 Hz, 1H), 7.71 (dd, J=7.2, 7.5 Hz, 1H), 7.62 (d, J=8.1 Hz, 1H), 7.52 (dd, J=6.9, 7.5 Hz, 1H), 7.38-7.34 (m, 2H), 7.27 (d, J=8.1 Hz, 1H), 7.00-6.98, (m, 4H), 6.89 (d, J=8.7 Hz, 1H), 5.33 (s, 2H). ¹³C NMR (75 MHz, CDCl₃/TMS) δ 158.09, 157.63, 149.66, 148.61, 147.69, 141.93, 137.16, 136.18, 134.73, 132.29, 131.57, 131.03, 130.83, 129.99, 129.14, 127.89, 127.76, 127.68, 126.76, 124.71, 119.32, 115.00, 71.58.

### Synthesis of 6-(pyridin-4-yl)-4′-(quinolin-2-ylmethoxy)biphenyl-3-carbonitrile

### Example 406

### 3-Bromo-4-(tetrahydro-2H-pyran-2-yloxy)benzonitrile

### Error! Objects cannot be created from editing field codes

A solution of 2-bromo-4-cyanophenol (5.0 g) and pyridinium p-toluenesulfonate (63 mg) was stirred in 80 mL of dry dichloromethane and 3,4-dihydro-2H-pyran (2.55 g) was added dropwise at room temperature. The mixture was stirred at room temperature for 24 h. The solvent was removed and the residue was purified by silica gel flash chromatography eluting with 50% ethyl acetate in heptane to give 3-bromo-4-(tetrahydro-2H-pyran-2-yloxy)benzonitrile (4.90 g) as a white solid. ¹H NMR (300 MHz, CDCl₃/TMS) δ 7.83 (d, J=1.8 Hz, 1H), 7.54 (dd, J=8.4, 1.8 Hz, 1H), 7.21 (d, J=8.7 Hz, 1H), 5.62 (s, 1H), 3.77 (m, 1H), 3.63 (m, 1H), 2.15-1.66 (m, 6H); ¹³C NMR (75 MHz, CDCl₃/TMS) δ 157.18, 136.87, 132.96, 117.99, 116.12, 113.40, 106.02, 97.00, 62.19, 30.19, 25.29, 18.31.

### 4′-(Quinolin-2-ylmethoxy)-6-(tetrahydro-2H-pyran-2-yloxy)biphenyl-3-carbonitrile

### Error! Objects cannot be created from editing field codes

A mixture of 3-bromo-4-(tetrahydropyran-2-yloxy)-benzonitrile (1.0 g), 2-[4-(4,4,5,5-tetramethyl-[1,3,2]dioxaborolan-2-yl)-phenoxymethyl]-quinoline (1.40 g), and cesium carbonate (3.46 g) in 1,4-dioxane (30 mL) was degassed four times before Pd(PPh₃)₄ (0.21 g) was added. The mixture was degassed four more times, then heated to 110° C. for 24 h. The mixture was filtered and washed with dichloromethane/methanol (1:1). The filtrate was concentrated and purified by silica gel flash chromatography eluting with 50% ethyl acetate in heptane to give 4′-(quinolin-2-ylmethoxy)-6-(tetrahydro-2H-pyran-2-yloxy)biphenyl-3-carbonitrile (1.26 g) as a white foam. ¹H NMR (300 MHz, CDCl₃/TMS) δ 8.17 (d, J=8.1 Hz, 1H), 8.08 (d, J=8.4 Hz, 1H), 7.80 (d, J=7.8 Hz, 1H), 7.75-7.67 (m, 2H), 7.56-7.51 (m, 3H), 7.43 (d, J=8.4 Hz, 2H), 7.24 (d, J=8.4 Hz, 1H), 7.09 (d, J=8.7 Hz, 2H), 5.49 (s, 1H), 5.41 (s, 2H), 3.73-3.57 (m, 2H), 1.76-1.54 (m, 6H); ¹³C NMR (75 MHz, CDCl₃/TMS) δ 158.23, 157.83, 157.32, 147.72, 137.23, 134.39, 132.59, 132.15, 130.83, 130.03, 129.48, 129.11, 127.93, 127.79, 126.78, 119.36, 115.78, 114.77, 105.15, 96.65, 71.64, 62.20, 30.27, 25.31, 18.53.

### 6-Hydroxy-4′-(quinolin-2-ylmethoxy)biphenyl-3-carbonitrile

### Error! Objects cannot be created from editing field codes

A solution of 4′-(quinolin-2-ylmethoxy)-6-(tetrahydropyran-2-yloxy)-biphenyl-3-carbonitrile (1.26 g) in methanol (30 mL) was treated with pyridinium p-toluenesulfonate (7.3 mg) at 50° C. for 20 h. The solvent was removed and the residue was washed with dichloromethane to give 6-hydroxy-4′-(quinolin-2-ylmethoxy)biphenyl-3-carbonitrile (0.54 g) as a white solid. ¹H NMR (300 MHz, DMSO-d₆/TMS) δ 10.89 (s, 1H), 8.43 (d, J=8.1 Hz, 1H), 8.03 (m, 2H), 7.80 (m, 1H), 7.72-7.66 (m, 2H), 7.63-7.52 (m, 4H), 7.13-7.06 (m, 3H), 5.43 (s, 2H); ¹³C NMR (75 MHz, DMSO-d₆/TMS) δ 159.18, 158.23, 158.15, 147.59, 137.72, 134.72, 133.10, 131.29, 131.05, 130.55, 129.77, 129.19, 128.63, 128.01, 127.86, 127.26, 120.19, 117.56, 115.19, 102.29, 71.59.

### 5-Cyano-4′-(quinolin-2-ylmethoxy)biphenyl-2-yl trifluoromethanesulfonate

### Error! Objects cannot be created from editing field codes

A solution of 6-hydroxy-4′-(quinolin-2-ylmethoxy)-biphenyl-3-carbonitrile (0.54 g) in dry pyridine (20 mL) was treated with trifluoromethanesulfonic anhydride (0.52 g) at 0° C. under argon. The resulting mixture stirred for 30 min at 0° C., then at room temperature overnight. The solvent was removed, the residue was dissolved in methylene chloride, washed with cold sodium bicarbonate solution, and dried over Na₂SO₄. The crude mixture was purified by silica gel flash chromatography eluting with 2% methanol in dichloromethane to give 5-cyano-4′-(quinolin-2-ylmethoxy)biphenyl-2-yl trifluoromethanesulfonate (0.44 g) as a yellow foam. ¹HNMR (300 MHz, CDCl₃/TMS) δ 8.19 (d, J=8.4 Hz, 1H), 8.09 (d, J=8.7 Hz, 1H), 7.82 (d, J=8.1 Hz, 1H), 7.73 (m, 2H), 7.67-7.64 (m, 2H), 7.54 (d, J=7.5, 7.5 Hz, 1H), 7.46 (d, J=8.4 Hz, 1H), 7.37 (d, J=8.7 Hz, 2H), 7.14 (d, J=8.7 Hz, 2H), 5.43 (s, 2H); ¹³C NMR (75 MHz, CDCl₃/TMS) δ 159.38, 157.38, 149.37, 147.72, 137.27, 136.88, 135.80, 132.34, 130.83, 130.07, 129.14, 127.92, 127.79, 126.84, 126.33, 123.62, 119.31, 118.44 (q, J=318.3 Hz), 117.41, 115.57, 113.15, 71.65. ¹⁹F NMR (282 MHz, CDCl₃) δ −74.23.

### 6-(Pyridin-4-yl)-4′-(quinolin-2-ylmethoxy)biphenyl-3-carbonitrile

### Example 406

### Error! Objects cannot be created from editing field codes

A mixture of 5-cyano-4′-(quinolin-2-ylmethoxy)biphenyl-2-yl trifluoromethanesulfonate (0.24 g), 4-pyridineboronic acid (73 mg), and 2 M Na₂CO₃ solution (0.74 mL) in 1,4-dioxane (10 mL) was degassed four times before Pd(PPh₃)₄ (28 mg) was added. The mixture was degassed four more times, then heated to reflux for 24 h. The mixture was filtered and washed with dichloromethane/methanol (1:1). The filtrate was concentrated and purified by silica gel flash chromatography eluting with 50% ethyl acetate in heptane to give 6-(pyridin-4-yl)-4′-(quinolin-2-ylmethoxy)biphenyl-3-carbonitrile (0.151 g) as a white foam. HRMS (ESI-TOF-MS): Calcd for C₂₈H₂₀N₃O [M+H]⁺: 414.1601, found 414.1600. ¹H NMR (300 MHz, CDCl₃/TMS) δ 8.49 (br, 2H), 8.18 (d, J=8.4 Hz, 1H), 8.07 (d, J=8.4 Hz, 1H), 7.82 (d, J=7.8 Hz, 1H), 7.75-7.62 (m, 4H), 7.55 (d, J=8.1 Hz, 1H), 7.46 (d, J=8.4 Hz, 1H), 7.03-6.91 (m, 6H), 5.35 (s, 2H). ¹³C NMR (75 MHz, CDCl₃/TMS) δ 158.38, 157.46, 149.84, 147.89, 147.66, 142.15, 141.54, 137.24, 134.36, 131.26, 131.11, 131.00, 130.94, 130.04, 129.09, 127.91, 127.76, 126.82, 124.38, 119.32, 118.54, 115.22, 112.84, 71.58.

### Synthesis of 2-(pyridin-4-yl)-4′-(u uinolin-2-ylmethoxy)biphenyl-4-carbonitrile

### Example 1885

### 3-(Pyridin-4-yl)-4-(tetrahydro-2H-pyran-2-yloxy)benzonitrile

### Error! Objects cannot be created from editing field codes

A mixture of 3-bromo-4-(tetrahydropyran-2-yloxy)-benzonitrile (1.50 g), 4-pyridine boronic acid (0.78 g), and cesium carbonate (5.20 g) in 1,4-dioxane (50 mL) was degassed four times before Pd(PPh₃)₄ (0.31 g) was added. The mixture was degassed four more times and then heated to reflux for 24 h. The mixture was filtered and washed with dichloromethane/methanol (1:1). The filtrate was concentrated and purified by silica gel flash chromatography eluting with 50% ethyl acetate in heptane to give 3-(pyridin-4-yl)-4-(tetrahydro-2H-pyran-2-yloxy)benzonitrile (0.64 g) as a white solid. ¹H NMR (300 MHz, CDCl₃/CD₃OD/TMS) δ 8.67 (br, 2H), 7.66 (br, 2H), 7.51 (br, 2H), 7.39 (d, J=6.6 Hz, 1H), 5.62 (br, 1H), 3.73-3.68 (m, 2H), 1.82-1.59 (m, 6H); ¹³C NMR (75 MHz, CDCl₃/CD₃OD/TMS) δ 157.26, 149.41, 144.61, 134.31, 134.18, 129.27, 124.30, 118.69, 115.93, 105.24, 96.82, 62.27, 30.01, 25.05, 18.46.

### 4-Hydroxy-3-(pyridin-4-yl)benzonitrile

### Error! Objects cannot be created from editing field codes

A solution of 3-pyridin-4-yl-4-(tetrahydropyran-2-yloxy)-benzonitrile (0.64 g) in methanol (30 mL) was treated with pyridinium p-toluenesulfonate (10 mg) at 50° C. for 48 h. The solvent was removed to give 0.61 g yellow solid, which was used directly in the next step without any further purification. ¹H NMR (300 MHz, CD₃OD/CDCl₃/TMS) δ 8.57 (br, 2H), 7.69-7.64 (m, 3H), 7.59 (dd, J=8.4, 1.8 Hz, 1H), 7.07 (d, J=8.4 Hz, 1H), 4.78 (br, 1H); ¹³C NMR (75 MHz, CD₃OD/CDCl₃/TMS) δ 159.19, 148.76, 147.78, 134.55, 134.31, 126.52, 124.53, 119.04, 117.22, 102.86.

### 4-Cyano-2-(pyridin-4-yl)phenyl trifluoromethanesulfonate

### Error! Objects cannot be created from editing field codes

A solution of 4-hydroxy-3-pyridin-4-ylbenzonitrile (0.61 g) in dry pyridine (10 mL) was treated with trifluoromethanesulfonic anhydride (0.76 g) at 0° C. under argon. The resulting mixture was stirred for 30 min at 0° C., then at room temperature overnight. The solvent was removed, the residue was diluted with methylene chloride, washed with cold sodium bicarbonate solution, and dried over Na₂SO₄. The crude mixture was purified by silica gel flash chromatography eluting with 30% ethyl acetate in heptane to give 4-cyano-2-(pyridin-4-yl)phenyl trifluoromethanesulfonate (0.38 g) as a yellow foam. ¹H NMR (300 MHz, CDCl₃/TMS) δ 8.78 (d, J=5.4 Hz, 2H), 7.87-7.84 (m, 2H), 7.61 (d, J=8.4 Hz, 1H), 7.41 (d, J=5.7 Hz, 2H); ¹³C NMR (75 MHz, CDCl₃/TMS) δ 150.55, 148.97, 141.42, 135.47, 134.52, 134.28, 123.93, 123.82, 118.37 (q, J=318.4 Hz), 116.89, 113.63; ¹⁹F NMR (282 MHz, CDCl₃) δ −74.24.

### 2-(Pyridin-4-yl)-4′-(quinolin-2-ylmethoxy)biphenyl-4-carbonitrile

### Example 1885

### Error! Objects cannot be created from editing field codes

A mixture of 4-cyano-2-(pyridin-4-yl)phenyl trifluoromethanesulfonate (0.38 g), 2-[4-(4,4,5,5-tetramethyl-[1,3,2]dioxaborolan-2-yl)-phenoxymethyl]-quinoline (0.51 g), and 2 M Na₂CO₃ solution (1.75 mL) in 1,4-dioxane (20 mL) was degassed four times before Pd(PPh₃)₄ (68 mg) was added. The mixture was degassed four more times, then heated to reflux for 24 h. The mixture was filtered and washed with dichloromethane/methanol (1:1). The filtrate was concentrated and purified by silica gel flash chromatography eluting with 50% ethyl acetate in heptane to give 2-(pyridin-4-yl)-4′-(quinolin-2-ylmethoxy)biphenyl-4-carbonitrile (0.45 g) as a light yellow solid, mp 190-193° C. HRMS (ESI-TOF-MS): Calcd for C₂₈H₂₀N₃O [M+H]⁺: 414.1601, found 414.1609. ¹H NMR (300 MHz, CDCl₃/TMS) δ 8.49 (d, J=4.8 Hz, 2H), 8.18 (d, J=8.4 Hz, 1H), 8.07 (d, J=8.4 Hz, 1H), 7.82 (d, J=8.4 Hz, 1H), 7.75-7.61 (m, 4H), 7.56-7.49 (m, 2H), 7.03-6.99 (m, 4H), 6.93 (d, J=8.7 Hz, 2H), 5.34 (s, 2H). ¹³C NMR (75 MHz, CDCl₃/TMS) δ 158.56, 157.42, 149.95, 147.69, 147.56, 144.91, 138.86, 137.19, 133.84, 132.16, 131.71, 131.00, 130.03, 129.14, 128.59, 127.89, 127.77, 126.82, 124.48, 119.31, 118.55, 115.19, 111.58, 71.64.

### Synthesis of 2-((2′-Chloro-6′-(pyridin-4-yl)biphenyl-4-yloxy)methybquinoline

### Example 382

### 2-Chloro-6-iodophenol

### Error! Objects cannot be created from editing field codes

To a solution of 2-iodophenol (5.0 g) in toluene (200 mL) was added diisopropylamine (32 μL) and sulfuryl chloride (3.07 g) dropwise at 70° C. After the addition, the mixture was stirred for another hour at 70° C., before it was quenched with 1 N HCl solution. The organic layer was separated, the aqueous layer was extracted with dichloromethane (3×50 mL), and dried over Na₂SO₄. The product was purified by silica gel flash chromatography eluting with 20% ethyl acetate in heptane to give 2-chloro-6-iodophenol (4.84 g) as an off-white solid. ¹H NMR (300 MHz, CDCl₃/TMS) δ 7.60 (dd, J=8.1, 1.2 Hz, 1H), 7.30 (dd, J=8.1, 1.5 Hz, 1H), 6.62 (dd, J=8.1, 7.8 Hz, 1H), 5.96 (br, 1H); ¹³C NMR (75 MHz, CDCl₃/TMS) δ 151.01, 137.94, 129.85, 123.03, 119.44, 83.81.

### 2-(2-Chloro-6-iodophenoxy)tetrahydro-2H-pyran

### Error! Objects cannot be created from editing field codes

A solution of 2-chloro-6-iodo-phenol (4.46 g) and pyridinium p-toluenesulfonate (47 mg) was stirred in 80 mL of dry dichloromethane and 3,4-dihydro-2H-pyran (1.89 g) was added dropwise at room temperature. The mixture was stirred at room temperature for 24 h. The solvent was removed and the residue was purified by silica gel flash chromatography eluting with 20% ethyl acetate in heptane to give 2-(2-chloro-6-iodophenoxy)tetrahydro-2H-pyran (1.78 g) as a white solid. ¹H NMR (300 MHz, CDCl₃/TMS) δ 7.69 (dd, J=8.1, 1.5 Hz, 1H), 7.34 (dd, J=7.8, 1.8 Hz, 1H), 7.64 (dd, J=8.1, 7.8 Hz, 1H), 5.44 (m, 1H), 4.35 (m, 1H), 3.61 (m, 1H), 2.21-1.89 (m, 6H). ¹³C NMR (75 MHz, CDCl₃/TMS) δ 153.92, 138.65, 131.26, 127.95, 126.35, 103.02, 93.34, 64.14, 30.89, 25.42, 19.30.

### 2-Chloro-6-(pyridin-4-yl)phenol

### Error! Objects cannot be created from editing field codes

A mixture of 2-(2-chloro-6-iodo-phenoxy)-tetrahydro-pyran (0.73 g), 4-pyridineboronic acid (0.32 g), and 2M Na₂CO₃ solution (3.24 mL) in 1,4-dioxane (40 mL) was degassed four times before Pd(PPh₃)₄ (125 mg) was added. The mixture was degassed four more times, then heated to reflux for 24 h. The mixture was filtered and washed with dichloromethane/methanol (1:1). The filtrate was concentrated and purified by silica gel flash chromatography eluting with 50% ethyl acetate in heptane to give 2-chloro-6-pyridin-4-yl-phenol (0.30 g) as a white solid and 4-[3-chloro-2-(tetrahydropyran-2-yloxy)-phenyl]-pyridine (0.15 g) as a light yellow oil. 4-[3-Chloro-2-(tetrahydropyran-2-yloxy)-phenyl]-pyridine was directly hydrolyzed with TFA to the phenol derivative.

A solution of 4-[3-chloro-2-(tetrahydropyran-2-yloxy)-phenyl]-pyridine (0.15 g) in methanol (30 mL) was treated with trifluoroacetic acid (0.177 g) at room temperature for 24 h. The solvent was removed, the residue was diluted with dichloromethane, washed with sodium bicarbonate solution, and dried over Na₂SO₄. The crude mixture was purified by silica gel flash chromatography eluting with 5% methanol in dichloromethane to give 2-chloro-6-pyridin-4-yl-phenol (70 mg) as a white solid. ¹H NMR (300 MHz, CDCl₃/CD₃OD/TMS) δ 8.58 (br, 2H), 7.55 (d, J=8.7 Hz, 2H), 7.40 (d, J=7.8 Hz, 1H), 7.25 (d, J=7.8 Hz, 1H), 7.96 (dd, J=7.8, 7.8 Hz, 1H), 2.95 (br, 1H); ¹³C NMR (75 MHz, CDCl₃/CD₃OD/TMS) δ 149.25, 149.14, 146.27, 130.00, 129.15, 127.18, 124.44, 121.69, 121.30.

### 2-Chloro-6-(pyridin-4-yl)phenyl trifluoromethanesulfonate

### Error! Objects cannot be created from editing field codes

A solution of 2-chloro-6-pyridin-4-yl-phenol (0.34 g) in dry pyridine (10 mL) was treated with trifluoromethanesulfonic anhydride (0.56 g) at 0° C. under argon. The resulting mixture was stirred for 30 min at 0° C., then at room temperature overnight. The solvent was removed, the residue was dissolved in methylene chloride, washed with cold sodium bicarbonate solution, and dried over Na₂SO₄. The crude mixture was purified by silica gel flash chromatography eluting with 50% ethyl acetate in heptane to give 2-chloro-6-(pyridin-4-yl)phenyl trifluoromethanesulfonate (0.47 g) as a white solid. ¹H NMR (300 MHz, CDCl₃/TMS) δ 8.73 (d, J=4.5 Hz, 2H), 7.60 (dd, J=8.1, 1.5 Hz, 1H), 7.46-7.35 (m, 4H); ¹³C NMR (75 MHz, CDCl₃/TMS) δ 150.37, 143.40, 142.99, 135.40, 131.67, 130.12, 129.46, 129.13, 124.02, 118.17 (q, J=318.3 Hz). ¹⁹F NMR (282 MHz, CDCl₃) δ −74.09.

### 2-((2′-Chloro-6′-(pyridin-4-yl)biphenyl-4-yloxy)methyl)quinoline

### Example 382

### Error! Objects cannot be created from editing field codes

A mixture of 2-chloro-6-(pyridin-4-yl)phenyl trifluoromethanesulfonate (0.22 g), 2-[4-(4,4,5,5-tetramethyl-[1,3,2]dioxaborolan-2-yl)-phenoxymethyl]-quinoline (0.28 g), and 2 M Na₂CO₃ solution (0.98 mL) in 1,4-dioxane (20 mL) was degassed four times before Pd(PPh₃)₄ (37 mg) was added. The mixture was degassed four more times, then heated to reflux for 24 h. The mixture was filtered and washed with dichloromethane/methanol (1:1). The filtrate was concentrated and purified by silica gel flash chromatography eluting with 50% ethyl acetate in heptane to give 242′-chloro-6′-(pyridin-4-yl)biphenyl-4-yloxy)methyl)quinoline (0.19 g) as a white solid. HRMS (ESI-TOF-MS): Calcd for C₂₇H₂₀ClN₂O [M+H]⁺: 423.1259, found 423.1255. ¹H NMR (300 MHz, CDCl₃/TMS) δ 8.39 (d, J=4.2 Hz, 2H), 8.18 (d, J=8.7 Hz, 1H), 8.08 (d, J=8.7 Hz, 1H), 7.82 (d, J=8.4 Hz, 1H), 7.72 (m, 1H), 7.64 (d, J=8.7 Hz, 1H), 7.56-7.51 (m, 2H), 7.34 (m, 1H), 7.28-7.26 (m, 1H), 7.00 (d, J=8.7 Hz, 2H), 6.95-6.90 (m, 4H), 5.34 (s, 2H). ¹³C NMR (75 MHz, CDCl₃/TMS) δ 157.92, 157.76, 149.36, 149.02, 147.71, 141.02, 138.87, 137.16, 134.91, 131.99, 130.17, 130.01, 129.98, 129.143 128.73, 128.37, 127.92, 127.78, 126.72, 124.65, 119.35, 114.59, 71.49.

### Synthesis of 2-((3′-chloro-2′-(pyridin-4-yl)biphenyl-4-yloxy)methybquinoline

### Example 1872

### 2-((3′-Chloro-2′-(tetrahydro-2H-pyran-2-yloxy)biphenyl-4-yloxy)methyl)quinoline

### Error! Objects cannot be created from editing field codes

A mixture of 2-(2-chloro-6-iodo-phenoxy)-tetrahydropyran (0.97 g), 2-[4-(4,4,5,5-tetramethyl-[1,3,2]dioxaborolan-2-yl)-phenoxymethyl]-quinoline (1.24 g), and 2 M Na₂CO₃ solution (4.3 mL) in 1,4-dioxane (80 mL) was degassed four times before Pd(PPh₃)₄ (165 mg) was added. The mixture was degassed four more times, then heated to reflux for 24 h. The mixture was filtered and washed with dichloromethane/methanol (1:1). The filtrate was concentrated and purified by silica gel flash chromatography eluting with 20% ethyl acetate in heptane to give 2-((3′-chloro-2′-(tetrahydro-2H-pyran-2-yloxy)biphenyl-4-yloxy)methyl)quinoline (0.32 g) as a white solid. ¹H NMR (300 MHz, CDCl₃/TMS) δ 8.14 (d, J=8.1 Hz, 1H), 8.09 (d, J=8.4 Hz, 1H), 7.79 (d, J=8.1 Hz, 1H), 7.72-7.63 (m, 2H), 7.52 (dd, J=8.1, 6.9 Hz, 1H), 7.41 (d, J=8.7 Hz, 2H), 7.31 (dd, J=7.8, 1.5 Hz, 1H), 7.14 (m, 1H), 7.08-6.99 (m, 3H), 5.41 (s, 2H), 5.03 (br, 1H), 3.45 (m, 1H), 3.18 (m, 1H), 1.76-1.31 (m, 6H). ¹³C NMR (75 MHz, CDCl₃/TMS) δ 161.09, 157.93, 151.05, 147.74, 137.12, 136.81, 131.77, 130.93, 130.01, 129.79, 129.50, 129.15, 128.51, 127.89, 127.77, 126.77, 124.66, 119.34, 114.94, 101.21, 71.82, 62.30, 30.22, 25.42, 18.46.

### 3-Chloro-4′-(quinolin-2-ylmethoxy)biphenyl-2-ol

### Error! Objects cannot be created from editing field codes

A solution of 2-[3′-chloro-2′-(tetrahydropyran-2-yloxy)-biphenyl-4-yloxymethyl]-quinoline (0.32 g) in methanol (20 mL) was treated with pyridinium p-toluenesulfonate (4 mg) at 50° C. for 24 h. The solvent was removed and the residue was purified by silica gel flash chromatography eluting with 50% ethyl acetate in heptane to give 3-chloro-4′-(quinolin-2-ylmethoxy)biphenyl-2-ol (0.21 g) as a white solid. ¹H NMR (300 MHz, CDCl₃/TMS) δ 8.23 47 (d, J=8.1 Hz, 1H), 8.09 (d, J=8.7 Hz, 1H), 7.84 (d, J=7.5 Hz, 1H), 7.75-7.69 (m, 2H), 7.56 (m, 1H), 7.48 (d, J=7.2 Hz, 1H), 7.28 (d, J=7.5 Hz, 1H), 7.17 (d, J=7.2 Hz, 1H), 7.09 (d, J=6.9 Hz, 2H), 6.89 (m, 1H), 5.42 (s, 2H); ¹³C NMR (75 MHz, CDCl₃/TMS) δ 158.04, 157.94, 148.74, 147.45, 137.55, 130.63, 130.40, 130.18, 129.61, 129.37, 128.71, 128.21, 127.92, 127.81, 126.85, 121.08, 119.38, 115.05, 71.29.

### 3-Chloro-4′-(quinolin-2-ylmethoxy)biphenyl-2-yl trifluoromethanesulfonate

### Error! Objects cannot be created from editing field codes

A solution of 3-chloro-4′-(quinolin-2-ylmethoxy)-biphenyl-2-ol (0.28 g) in dry pyridine (10 mL) was treated with trifluoromethanesulfonic anhydride (0.56 g) at 0° C. under argon. The resulting mixture was stirred for 30 min at 0° C., then room temperature overnight. The solvent was removed, the residue was diluted with methylene chloride, washed with cold sodium bicarbonate solution, and dried over Na₂SO₄. The crude mixture was purified by silica gel flash chromatography eluting with 50% ethyl acetate in heptane to give 3-chloro-4′-(quinolin-2-ylmethoxy)biphenyl-2-yl trifluoromethanesulfonate (0.32 g) as a white solid. ¹H NMR (300 MHz, CDCl₃/TMS) δ 8.19 (d, J=8.4 Hz, 1H), 8.09 (d, J=8.4 Hz, 1H), 7.83 (d, J=8.4 Hz, 1H), 7.74 (m, 1H), 7.67 (d, J=8.4 Hz, 1H), 7.55 (dd, J=7.5, 7.2 Hz, 1H), 7.44 (m, 1H), 7.36 (d, J=9.0 Hz, 2H), 7.30 (m, 2H), 7.10 (d, J=8.4 Hz, 2H), 5.43 (s, 2H). ¹³C NMR (75 MHz, CDCl₃/TMS) δ 158.96, 157.62, 147.75, 143.40, 137.71, 137.21, 130.88, 130.56, 130.05, 129.87, 12914, 128.99, 128.60, 128.47, 127.93, 127.81, 126.81, 119.33, 118.26 (q, J=308.77 Hz), 115.34, 71.64. ¹⁹F NMR (282 MHz, CDCl₃) δ −74.34.

### 2-((3′-Chloro-2′-(pyridin-4-yl)biphenyl-4-yloxy)methyl)quinoline

### Example 1872

### Error! Objects cannot be created from editing field codes

A mixture of 3-chloro-4′-(quinolin-2-ylmethoxy)biphenyl-2-yl trifluoromethanesulfonate (0.16 g), 4-pyridineboronic acid (48 mg), and 2 M Na₂CO₃ (0.49 mL) in 1,4-dioxane (10 mL) was degassed four times before Pd(PPh₃)₄ (19 mg) was added. The mixture was degassed four more times, then heated to reflux for 24 h. The mixture was filtered and washed with dichloromethane/methanol (1:1). The filtrate was concentrated and purified by silica gel flash chromatography eluting with 30% ethyl acetate in heptane to give 2-((3′-chloro-2′-(pyridin-4-yl)biphenyl-4-yloxy)methyl)quinoline (0.15 g) as an off-white foam. HRMS (ESI-TOF-MS): Calcd for C₂₇H₂₀ClN₂O [M+H]⁺: 423.1259, found 423.1257. ¹H NMR (300 MHz, CDCl₃/TMS) δ 8.48 (d, J=4.2 Hz, 2H), 8.15 (d, J=8.4 Hz, 1H), 8.06 (d, J=8.7 Hz, 1H), 7.79 (d, J=8.4 Hz, 1H), 7.71 (m, 1H), 7.60 (d, J=8.7 Hz, 1H), 7.52 (m, 1H), 7.45 (m, 1H), 7.36-7.2 (m, 2H), 7.03 (d, J=5.4 Hz, 2H), 6.92 (d, J=8.7 Hz, 2H), 6.82 (d, J=8.7 Hz, 2H), 5.30 (s, 2H). ¹³C NMR (75 MHz, CDCl₃/TMS) δ 157.73, 149.44, 147.71, 146.62, 142.91, 137.16, 136.69, 133.35, 132.99, 130.94, 129.99, 129.40, 129.14, 129.09, 128.74, 127.91, 127.77, 126.74, 125.99, 119.31, 114.62 71.53.

### Synthesis of 2-((2′-(1,3-Dioxan-2-yl)-6′-(pyridin-4-yl)biphenyl-4-yloxy)methyl)quinoline

### Example 1857

### 3-Bromo-2-hydroxybenzaldehyde

### Error! Objects cannot be created from editing field codes

A dry 2-L three-neck flask equipped with a reflux condenser and rubber septum was charged with MgCl₂ (34.23 g) and solid powdered paraformaldehyde (16.4 g). Dry THF (500 mL) was added, followed by dropwise addition of Et₃N (36.4 g). The mixture was stirred for 15 min, before 2-bromophenol (27.0 g) was added dropwise. The mixture became of opaque, light pink color. The mixture was heated to 75° C. and kept at this temperature for 4 h. It was cooled to room temperature, methyl tert-butyl ether (500 mL) was added and the mixture was transferred to a 2-L reparatory funnel. The mixture was washed with 1 N HCl (4×300 mL) and water (4×400 mL), and dried over Na₂SO₄. The crude mixture (29.80 g) was crystallized from heptane to give 3-bromo-2-hydroxybenzaldehyde (27.0 g) as light yellow crystals. ¹H NMR (300 MHz, CDCl₃/TMS) δ 11.62 (s, 1H), 9.86 (s, 1H), 7.78 (d, J=8.1 Hz, 1H), 7.56 (dd, J=7.5, 1.2 Hz, 1H), 6.96 (dd, J=7.8, 7.5 Hz, 1H); ¹³C NMR (75 MHz, CDCl₃/TMS) δ 196.16, 158.19, 140.17, 133.16, 121.50, 121.04, 111.40.

### 2-Hydroxy-3-(pyridin-4-yl)benzaldehyde

### Error! Objects cannot be created from editing field codes

A mixture of 3-bromo-2-hydroxybenzaldehyde (2.01 g), 4-pyridineboronic acid (1.48 g), and 2 M Na₂CO₃ solution (20 mL) in toluene (400 mL) and ethanol (80 mL) was degassed four times before Pd(PPh₃)₄ (0.58 g) was added. The mixture was degassed four more times, then heated to reflux for 24 h. The mixture was filtered and washed with dichloromethane/methanol (1:1). The filtrate was concentrated and purified by silica gel flash chromatography eluting with 10% acetone in dichloromethane to give 2-hydroxy-3-(pyridin-4-yl)benzaldehyde (0.70 g) as a yellow solid. ¹H NMR (300 MHz, CDCl₃/TMS) δ 11.67 (br, 1H), 9.96 (s, 1H), 8.68 (d, J=8.1 Hz, 1H), 7.65 (d, J=7.8 Hz, 1H), 7.55 (m, 1H), 7.16 (dd, J=7.5, 7.8 Hz, 1H); ¹³C NMR (75 MHz, CDCl₃/TMS) δ 196.83, 159.12, 149.96, 144.22, 137.55, 134.77, 127.58, 124.07, 121.23, 120.40.

### 2-(1,3-Dioxan-2-yl)-6-(pyridin-4-yl)phenol

### Error! Objects cannot be created from editing field codes

A solution of 2-hydroxy-3-pyridin-4-ylbenzaldehyde (0.30 g), 1,3-propanediol (0.14 g) and p-toluenesulfonic acid monohydrate (10 mg) in toluene (15 mL) was refluxed for 24 h on a Dean-stark apparatus. The solvent was removed and the residue was purified by silica gel flash chromatography eluting with 60% ethyl acetate in heptane to give 2-(1,3-dioxan-2-yl)-6-(pyridin-4-yl)phenol (0.22 g) as a white solid. ¹H NMR (300 MHz, CDCl₃/TMS) δ 8.60 (d, J=5.4 Hz, 2H), 8.39 (br, 1H), 7.51 (d, J=6.0 Hz, 2H), 7.31 (d, J=7.5 Hz, 1H), 7.25 (d, J=7.8 Hz, 1H), 6.96 (dd, J=7.8, 7.5 Hz, 1H), 5.70 (s, 1H), 4.31 (dd, J=11.1, 4.5 Hz, 2H), 4.02 (m, 2H), 2.25 (m, 1H), 1.52 (d, J=13.8 Hz, 1H); ¹³C NMR (75 MHz, CDCl₃/TMS) δ 152.75, 149.56, 146.27, 131.39, 128.85, 127.16, 124.47, 123.30, 120.23, 103.26, 67.86, 26.01.

### 2-(1,3-Dioxan-2-yl)-6-(pyridin-4-yl)phenyl trifluoromethanesulfonate

### Error! Objects cannot be created from editing field codes

A solution of 2-[1,3]dioxan-2-yl-6-pyridin-4-yl-phenol (0.22 g) in dry pyridine (10 mL) was treated with trifluoromethanesulfonic anhydride (0.289 g) at 0° C. under argon. The resulting mixture was stirred for 30 min at 0° C., then at room temperature overnight. The solvent was removed, the residue was diluted with methylene chloride, washed with cold sodium bicarbonate solution, and dried over Na₂SO₄. The crude brown solid (0.33 g) was used directly in the next step with any purification. ¹H NMR (300 MHz, CDCl₃/TMS) δ 8.69 (br, 2H), 7.89 (d, J=7.8 Hz, 1H), 7.51 (dd, J=7.8, 7.8 Hz, 1H), 7.40 (d, J=7.5 Hz, 1H), 7.35 (d, J=3.6 Hz, 2H), 5.87 (s, 1H), 4.28 (dd, J=11.4, 4.8 Hz, 2H), 4.02 (dd, J=12.0, 11.1 Hz, 2H), 2.26 (m, 1H), 1.48 (d, J=13.5 Hz, 1H); ¹³C NMR (75 MHz, CDCl₃/TMS) δ 148.68, 148.48, 142.68, 141.44, 134.69, 132.25, 132.13, 131.04, 127.97, 127.67, 122.94, 116.75 (q, J=317.7 Hz), 95.58, 66.40, 24.46. ¹⁹F NMR (282 MHz, CDCl₃) δ −74.75.

### 2-((2′-(1,3-Dioxan-2-yl)-6′-(pyridin-4-yl)biphenyl-4-yloxy)methyl)quinoline

### Example 1857

### Error! Objects cannot be created from editing field codes

A mixture of 2-(1,3-dioxan-2-yl)-6-(pyridin-4-yl)phenyl trifluoromethanesulfonate (0.36 g), 2-[4-(4,4,5,5-tetramethyl-[1,3,2]dioxaborolan-2-yl)-phenoxymethyl]-quinoline (0.37 g), and 2M Na₂CO₃ solution (1.3 mL) in 1,4-dioxane (10 mL) was degassed four times before Pd(dppf)Cl₂ (32 mg) was added. The mixture was degassed four more times, then heated to reflux for 24 h. The mixture was filtered and washed with dichloromethane/methanol (1:1). The filtrate was concentrated and purified by silica gel flash chromatography eluting with 60% ethyl acetate in heptane to give 2-((2′-(1,3-dioxan-2-yl)-6′-(pyridin-4-yl)biphenyl-4-yloxy)methyl)quinoline (0.40 g) as a white foam. HRMS (ESI-MS): Calcd for C₃₁H₂₆N₂O₃ [M+H]⁺: 475.2016, found 475.2039. ¹H NMR (300 MHz, CDCl₃/TMS) δ 8.36 (m, 2H), 8.23 (d, J=8.1 Hz, 1H), 8.09 (d, J=8.1 Hz, 1H), 7.86 (d, J=8.1 Hz, 2H), 7.75 (m, 1H), 7.68 (d, J=8.4 Hz, 1H), 7.57 (m, 1H), 7.50 (m, 1H), 7.36 (d, J=6.9 Hz, 1H), 7.02 (d, J=8.7 Hz, 2H), 6.96 (d, J=5.1 Hz, 2H), 6.90 (d, J=8.7 Hz, 2H), 5.37 (s, 2H), 5.16 (s, 1H), 4.15 (dd, J=11.7, 4.5 Hz, 2H), 3.68 (t, J=11.4 Hz, 2H), 2.20 (m, 1H), 1.33 (d, J=13.2 Hz, 1H). ¹³C NMR (75 MHz, CDCl₃/TMS) δ 157.29, 149.99, 148.27, 147.28, 138.42, 138.20, 137.53, 136.78, 131.75, 129.86, 129.63, 128.70, 127.81, 127.52, 127.38, 126.54, 126.38, 124.69, 119.03, 113.91, 99.55 71.14, 67.17, 25.56.

### Synthesis of 6-(pyridin-4-yl)-4′-(quinolin-2-ylmethoxy)biphenyl-2-carbaldehyde

### Example 1854

### 6-(Pyridin-4-yl)-4′-(quinolin-2-ylmethoxy)biphenyl-2-carbaldehyde

### Example 1854

### Error! Objects cannot be created from editing field codes

A solution of 2-(6′-[1,3]dioxan-2-yl-2′-pyridin-4-yl-biphenyl-4-yloxymethyl)-quinoline (0.39 g) in acetone/water (10 mL/2 mL) was treated with p-toluenesulfonic acid monohydrate (0.39 g) at 30° C. for 18 h. The solvent was removed and the residue was dissolved in dichloromethane. The organic layer was washed with sodium bicarbonate solution and dried over Na₂SO₄. 6-(Pyridin-4-yl)-4′-(quinolin-2-ylmethoxy)biphenyl-2-carbaldehyde (0.267 g) was obtained after removal of the solvent. HRMS (DIP-CI-MS): Calcd for C₂₈H₂₀N₂O₂ [M+H]⁺: 417.1603, found 417.1581. ¹H NMR (300 MHz, CDCl₃/TMS) δ 9.83 (s, 1H), 8.43 (m, 2H), 8.21 (d, J=8.4 Hz, 1H), 8.07 (m, 2H), 7.84 (d, J=7.8 Hz, 1H), 7.74 (dd, J=7.2, 8.1, 1H), 7.64 (d, J=8.4 Hz, 1H), 7.57 (m, 3H), 6.96 (m, 6H), 5.37 (s, 2H). ¹³C NMR (75 MHz, CDCl₃/TMS) δ 191.97, 157.99, 157.03, 148.99, 148.09, 147.29, 143.24, 139.56, 136.83, 134.76, 134.56, 132.16, 129.64, 128.72, 127.79, 127.52, 127.41, 126.40, 124.39, 118.88, 114.43, 71.19.

### Synthesis of 2-((2′-Methoxy-6′-(pyridin-4-yl)biphenyl-4-yloxy)methyl)quinoline

### Example 385

### 4′-(Benzyloxy)-2-methoxy-6-nitrobiphenyl

### Error! Objects cannot be created from editing field codes

2-Bromo-3-nitroanisole (2.50 g), 4-benzyloxyphenyl boronic acid (2.94 g), and 2 M Na₂CO₃ solution (16.2 mL) in 150 ml dioxane was degassed four times before Pd(dppf)Cl₂ (0.39 g) was added. The mixture was degassed four more times, then heated to reflux for 24 h. The mixture was cooled down to room temperature and the solvent was removed. The residue was washed with dichloromethane, and the filtrate was concentrated and purified by silica gel flash chromatography eluting with 50% ethyl acetate in heptane to give 4′-(benzyloxy)-2-methoxy-6-nitrobiphenyl (3.4 g) as a yellow solid. ¹H NMR (300 MHz, CDCl₃/TMS) δ 7.47-7.33 (m, 7H), 7.20 (d, J=8.7 Hz, 2H), 7.13 (d, J=7.8 Hz, 1H), 7.02 (d, J=8.7 Hz, 2H), 5.05 (s, 2H), 3.75 (s, 3H); ¹³C NMR (75 MHz, CDCl₃/TMS) δ 158.83, 157.84, 151.48, 137.05, 130.63, 128.82, 128.24, 127.82, 124.97, 124.80, 115.56, 114.88, 114.44, 70.29, 56.74.

### 4′-(Benzyloxy)-6-methoxybiphenyl-2-amine

### Error! Objects cannot be created from editing field codes

4′-Benzyloxy-2-methoxy-6-nitro-biphenyl (3.92 g) in 150 mL of ethyl acetate and water (4 mL) was treated with SnCl₂ (4.28 g) and stirred for 24 h at room temperature. A 1 N NaOH solution (200 mL) was added and the mixture extracted with ethyl acetate (4×50 mL). The organic layer was dried over Na₂SO₄. The organic layer was concentrated and purified by silica gel flash chromatography eluting with 30% ethyl acetate in heptane to give 4′-(benzyloxy)-6-methoxybiphenyl-2-amine (3.21 g) as a yellow solid. ¹H NMR (300 MHz, CDCl₃/TMS) δ 7.47-7.31 (m, 6H), 7.27-7.19 (m, 2H), 7.13-7.03 (m, 3H), 6.42 (dd, J=8.1, 9.0 Hz, 1H), 5.08 (s, 2H), 3.69 (s, 3H); ¹³C NMR (75 MHz, CDCl₃/TMS) δ 158.13, 157.93, 145.35, 137.27, 131.89, 130.64, 128.84, 128.22, 127.87, 127.79, 115.35, 114.89, 108.84, 101.45, 70.28, 56.02.

### 4′-(Benzyloxy)-2-iodo-6-methoxybiphenyl

### Error! Objects cannot be created from editing field codes

To a solution of p-TsOH.H₂O (1.87 g) in acetonitrile (15 mL) was added 4′-(benzyloxy)-6-methoxybiphenyl-2-amine (1.0 g). The resulting suspension was cooled to 10-15° C., and a solution of NaNO₂ (0.45 g) and KI (5.44 g) in water (2 mL) was added gradually. The mixture was stirred for 2 h at RT, then water (20 mL) and NaHCO₃ solution (5 mL) were added. The mixture was extracted with ethyl acetate (4×50 mL) and the organic layer was dried over Na₂SO₄. The organic layer was concentrated and purified by silica gel flash chromatography eluting with 30% ethyl acetate in heptane to give 4′-(benzyloxy)-2-iodo-6-methoxybiphenyl (0.86 g) as a yellow oil. ¹H NMR (300 MHz, CDCl₃/TMS) δ 7.55 (d, J=7.8 Hz, 1H), 7.47 (d, J=6.9 Hz, 2H), 7.43-7.34 (m, 3H), 7.14 (d, J=8.1 Hz, 2H), 7.05 (d, J=8.1 Hz, 2H), 6.99 (d, J=8.1 Hz, 1H), 6.92 (d, J=8.4 Hz, 1H), 5.09 (s, 2H), 3.69 (s, 3H); ¹³C NMR (75 MHz, CDCl₃/TMS) δ 158.36, 157.47, 137.22, 135.54, 133.84, 131.37, 131.31, 129.94, 128.79, 128.19, 127.87, 114.44, 110.97, 102.53, 70.26, 56.30.

### 4-(4′-(Benzyloxy)-6-methoxybiphenyl-2-yl)pyridine

### Error! Objects cannot be created from editing field codes

4′-Benzyloxy-6-iodo-2-methoxy-biphenyl (0.86 g), 4-pyridineboronic acid (0.30 g), and 2 M aqueous Na₂CO₃ solution (3.1 mL) in 50 mL dioxane was degassed four times before Pd(PPh₃)₄ (120 mg) was added. The mixture was degassed four more times, then heated to reflux for 24 h. The mixture was cooled down to room temperature and the solvent was removed. The residue was washed with dichloromethane, and the filtrate was concentrated and purified by silica gel flash chromatography eluting with 30% ethyl acetate in heptane to give 4-(4′-(benzyloxy)-6-methoxybiphenyl-2-yl)pyridine (0.66 g) as a thick colorless oil. ¹H NMR (300 MHz, CDCl₃/TMS) δ 8.37 (d, J=5.1 Hz, 1H), 7.41-7.28 (m, 3H), 7.03-6.96 (m, 3H), 6.83 (d, J=9.0 Hz, 1H), 4.99 (s, 2H), 3.76 (s, 3H); ¹³C NMR (75 MHz, CDCl₃/TMS) δ 157.86, 157.41, 149.87, 149.25, 140.21, 137.16, 132.46, 129.42, 128.76, 128.69, 128.55, 128.18, 127.81, 125.00, 122.38, 114.44, 111.35, 70.22, 56.27.

### 2′-Methoxy-6′-(pyridin-4-yl)biphenyl-4-ol

### Error! Objects cannot be created from editing field codes

4-(4′-Benzyloxy-6-methoxy-biphenyl-2-yl)-pyridine (0.64 g) in 20 mL methanol was treated with 10% Pd/C (100 mg) under 50 psi hydrogen atmosphere for 17 h. The mixture was filtered and washed with methanol. The filtrate was concentrated to give 2′-methoxy-6′-(pyridin-4-yl)biphenyl-4-ol (0.38 g) as a white solid. ¹H NMR (300 MHz, CD₃OD/TMS) δ 8.28 (d, J=5.1 Hz, 2H), 7.39 (dd, J=8.4, 7.5 Hz, 1H), 7.14-7.09 (m, 3H), 6.83 (d, J=9.0 Hz, 1H), 6.84 (d, J=9.0 Hz, 2H), 6.62 (d, J=8.7 Hz, 2H), 3.75 (s, 3H); ¹³C NMR (75 MHz, CD₃OD/TMS) δ 158.54, 157.48, 152.42, 148.93, 140.61, 133.21, 131.90, 129.37, 127.99, 126.39, 122.82, 115.46, 112.49, 56.22.

### 2-((2′-Methoxy-6′-(pyridin-4-yl)biphenyl-4-yloxy)methyl)quinoline

### Example 385

### Error! Objects cannot be created from editing field codes

2′-Methoxy-6′-pyridin-4-yl-biphenyl-4-ol (0.32 g) in DMF (10 mL) was treated with 2-chloromethylquinoline hydrochloride (0.27 g) and potassium carbonate (0.399 g). The mixture was stirred at 40° C. for 6 h. The mixture was filtered and washed with dichloromethane/methanol (1:1). The concentrated crude mixture was purified by silica gel flash chromatography eluting with 5% methanol in dichloromethane to give 2-((2′-methoxy-6′-(pyridin-4-yl)biphenyl-4-yloxy)methyl)quinoline (0.36 g) as a yellow wax. HRMS (TOF-MS): Calcd for C₂₈H₂₂N₂O₂ [M+H]⁺: 419.1754, found 419.1756; ¹H NMR (300 MHz, CDCl₃/TMS) δ 8.37 (d, J=4.8 Hz, 2H), 8.17 (d, J=8.4 Hz, 1H), 8.07 (d, J=8.1 Hz, 1H), 7.81 (d, J=8.1 Hz, 1H), 7.71 (dd, J=6.9, 7.5, 1H), 7.64 (d, J=8.4 Hz, 1H), 7.52 (dd, J=7.5, 7.2 Hz, 1H), 7.38 (dd, J=7.8, 8.1 Hz, 1H), 7.03-6.99 (m, 6H), 6.89 (d, J=8.7 Hz, 2H), 5.33 (s, 2H), 3.76 (s, 3H); ¹³C NMR (75 MHz, CDCl₃/TMS) δ 157.96, 157.51, 157.36, 150.54, 148.50, 147.66, 139.90, 137.16, 132.52, 129.96, 129.28, 129.08, 128.78, 128.72, 127.94, 127.78, 126.70, 125.16, 122.29, 119.38, 114.51, 111.46, 71.45, 56.24.

### Synthesis of 2-(2′-Nitro-6′-pyridin-4-yl-biphenyl-4-yloxymethyl)-quinoline

### Example 384

### 2-Bromo-3-nitrophenol

### Error! Objects cannot be created from editing field codes

BBr₃ (1.0M in CH₂Cl₂, 88 mL, 88 mmol) was added dropwise over 1 h to a stirred solution of 2-bromo-3-nitroanisole in CH₂Cl₂ (35 mL) under argon at −70° C. The resulting deep burgundy-colored reaction mixture was allowed to warm up to RT slowly (over 2 h) and stirred at RT for 23 h. The reaction mixture was poured onto 350 g crushed ice and extracted with EtOAc (300 mL). The organic phase was separated, washed with brine (75 mL), and dried over MgSO₄. Concentration and purification by chromatography (5-70% EtOAc/heptane) gave the title compound 2-bromo-3-nitrophenol (5.36 g, 98%) as a yellow solid. ¹H NMR (300 MHz, CDCl₃/TMS) δ 7.48 (d, J=8.1 Hz, 1H), 7.37 (t, J=8.1 Hz, 1H), 7.27 (d, J=8.4 Hz, 1H), 6.13 (br s, 1H); ¹³C NMR (75 MHz, CDCl₃/TMS) δ 153.7, 128.7, 119.8, 117.5, 102.9.

### 4′-Benzyloxy-6-nitro-biphenyl-2-ol

### Error! Objects cannot be created from editing field codes

To a solution of 2-bromo-3-nitrophenol (5.36 g, 24.6 mmol) and 4-benzyloxyphenylboronic acid (6.73 g, 29.5 mmol) in dioxane was added 2M aqueous Na₂CO₃ solution (55.4 mL) and the mixture was purged with argon. Pd(PPh₃)₄ (1.42 g, 1.23 mmol) was added and the mixture was purged again with argon. The reaction mixture was heated to reflux for 24 h. The mixture was cooled to RT and the organic solvent was removed under reduced pressure. The residue was diluted with water (150 mL), neutralized with 2N HCl, filtered through a Celite® plug washing with EtOAc, and extracted with EtOAc (3×100 mL). The combined organic phases were washed with brine (50 mL) and dried over MgSO₄. Concentration and purification by chromatography (5-40% EtOAc/heptane) gave the title compound 4′-benzyloxy-6-nitro-biphenyl-2-ol (6.35 g, 80%) as a yellow solid. ¹H NMR (300 MHz, CDCl₃/TMS) δ 7.52-7.30 (m, 7H), 7.27-7.15 (m, 3H), 7.09 (d, J=7.8 Hz, 2H), 5.73 (s, 1H), 5.09 (s, 2H); ¹³C NMR (75 MHz, CDCl₃/TMS) δ 159.1, 154.1, 149.9, 136.3, 130.4, 128.7, 128.4, 127.9, 127.3, 122.7, 121.8, 119.4, 115.7, 115.5, 70.0.

### 4′-(Benzyloxy)-6-nitrobiphenyl-2-yl trifluoromethanesulfonate

### Error! Objects cannot be created from editing field codes

A solution of 4′-benzyloxy-6-nitro-biphenyl-2-ol (6.37 g, 19.8 mmol) in dry pyridine (120 mL) was treated with trifluoromethanesulfonic anhydride at 0° C. under argon. The resulting mixture stirred at 0° C. for 0.5 h, then allowed to warm up to RT and stirred for 18 h. The solvent was removed under reduced pressure, the residue was dissolved in CH₂Cl₂ (500 mL), washed with cold saturated NaHCO₃ aqueous solution (2×150 mL), and dried over MgSO₄. Filtration and concentration gave the title compound 4′-(benzyloxy)-6-nitrobiphenyl-2-yl trifluoromethanesulfonate (9.00 g, 100%) as a yellow solid, which was used for the next step without further purification. ¹H NMR (300 MHz, CDCl₃/TMS) δ 7.83 (dd, J=7.2, 1.8 Hz, 1H), 7.63-7.52 (m, 2H), 7.45-7.28 (m, 5H), 7.22 (d, J=8.7 Hz, 2H), 7.06 (d, J=8.7 Hz, 2H), 5.10 (s, 2H); ¹³C NMR (75 MHz, CDCl₃/TMS) δ 159.4, 151.0, 147.2, 136.2, 130.3, 129.0, 128.4, 127.9, 127.4, 125.3, 123.2, 121.4, 118.0 (J=318 Hz), 114.9, 69.9.

### 4-(4′-Benzyloxy-6-nitro-biphenyl-2-yl)-pyridine

### Error! Objects cannot be created from editing field codes

To a solution of 4′-(benzyloxy)-6-nitrobiphenyl-2-yl trifluoromethanesulfonate (4.77 g, 10.5 mmol) and 4-benzyloxyphenylboronic acid (1.94 g, 15.8 mmol) in dioxane (150 mL) was added 2M aqueous Na₂CO₃ solution (15.8 mL) and the mixture was purged with argon. Pd(PPh₃)₄ (0.61 g, 0.53 mmol) was added and the mixture was purged again with argon. The reaction mixture was heated to reflux for 21 h. The mixture was cooled to RT and the solvent was removed under reduced pressure. The residue was partitioned between EtOAc (150 mL) and water (150 mL) and neutralized with 2N aqueous HCl solution. The resulting mixture was passed through a Celite® plug. The organic phase was separated from the aqueous phase and the latter was extracted with EtOAc (2×50 mL). The combined organic phases were washed with brine (50 mL) and dried over MgSO₄. Concentration and purification by chromatography eluting with 10-100% EtOAc/heptane provided 4′-benzyloxy-6-nitro-biphenyl-2-ol (0.38 g, 11%) and the title compound 4-(4′-benzyloxy-6-nitro-biphenyl-2-yl)-pyridine (3.10 g, 77%) as a yellow solids. ¹H NMR (300 MHz, CDCl₃/TMS) δ 8.45 (dd, J=4.5, 1.2 Hz, 2H), 7.79 (dd, J=6.6, 2.7 Hz, 1H), 7.60-7.50 (m, 2H), 7.50-7.20 (m, 5H), 6.96 (dd, J=6.3, 1.5 Hz, 4H), 6.85 (d, J=8.7 Hz, 2H), 5.00 (s, 2H); ¹³C NMR (75 MHz, CDCl₃/TMS) δ 158.4, 151.0, 149.2, 147.2, 140.7, 136.2, 133.4, 132.8, 130.3, 128.4, 128.1, 127.9, 127.4, 126.2, 124.1, 123.1, 114.6, 69.8.

### 2′-Nitro-6′ pyridin-4-yl-biphenyl-4-ol

### Error! Objects cannot be created from editing field codes

To a solution of 4-(4′-benzyloxy-6-nitro-biphenyl-2-yl)-pyridine (0.74 g, 1.94 mmol) in CH₂Cl₂ (10 mL) was added trifluoroacetic acid (10 mL). The resulting solution was stirred and heated to reflux for 2 h under argon. The solvent was removed under reduced pressure, the residue was partitioned between water (25 mL) and EtOAc (25 mL), and neutralized with saturated NaHCO₃. The organic phase was separated from the aqueous phase and the latter was extracted with EtOAc (2×25 mL). The combined organic layers were washed with brine and dried over MgSO₄. Concentration and purification by chromatography (5-100% EtOAc/heptane) afforded the title compound 2′-nitro-6′ pyridin-4-yl-biphenyl-4-ol (0.26 g, 46%) as a yellow solid. ¹H NMR (300 MHz, CD₃OD/CDCl₃/TMS) δ 8.38 (br s, 2H), 7.82 (d, J=6.9 Hz, 1H), 7.68-7.56 (m, 2H), 7.22-7.02 (m, 2H), 6.87 (d, J=8.4 Hz, 2H), 6.68 (d, J=8.4 Hz, 2H); ¹³C NMR (75 MHz, CD₃OD/CDCl₃/TMS) δ 157.9, 152.1, 149.6, 148.9, 141.3, 134.4, 133.5, 131.3, 129.0, 128.7, 125.8, 123.9, 115.8.

### 2-(2′-Nitro-6′-pyridin-4-yl-biphenyl-4-yloxymethyl)-quinoline

### Example 384

### Error! Objects cannot be created from editing field codes

To a stirred suspension of 2′-nitro-6′ pyridin-4-yl-biphenyl-4-ol (260 mg, 0.89 mmol) was added K₂CO₃ (615 mg, 4.45 mmol) and the mixture was stirred for 15 min at RT. To this suspension 2-chloromethylquinoline monohydrochloride (200 mg, 0.93 mmol) was added at RT and the mixture heated to reflux for 18 h under argon atmosphere. The reaction mixture was cooled to ambient temperature and the inorganic salts were filtered off and washed with acetonitrile. The filtrate was concentrated and the residue was purified via chromatography (10-100% EtOAc/heptane) to provide the title compound 2-(2′-nitro-6′-pyridin-4-yl-biphenyl-4-yloxymethyl)-quinoline (240 mg, 62%) as a yellow solid. Mass spectrometry (ESI): calcd for C₂₇H₂₀N₃O₃ (MH⁺): 434.1499; found: 434.1498; HPLC 96.8% (Rt=13.01 min); ¹H NMR (300 MHz, CDCl₃/TMS) δ 8.41 (d, J=6.0 Hz, 2H), 8.16 (d, J=8.7 Hz, 1H), 8.05 (d, J=8.1 Hz, 1H), 7.80 (d, J=8.4 Hz, 1H), 7.75 (dd, J=6.6, 2.5 Hz, 1H), 7.70 (dt, J=7.6, 1.2 Hz, 1H), 7.59 (d, J=8.7 Hz, 1H), 7.56-7.44 (m, 3H), 6.98-6.82 (m, 6H), 5.30 (s, 2H); ¹³C NMR (75 MHz, CDCl₃/TMS) δ 158.0, 157.0, 150.9, 149.1, 147.2, 147.1, 140.7, 136.7, 133.3, 132.7, 130.4, 129.5, 128.6, 128.0, 127.4, 127.3, 126.5, 126.3, 124.0, 123.0, 118.8, 114.6, 71.0.

### Synthesis of 6-pyridin-4-yl-4′-(quinolin-2-ylmethoxy)-biphenyl-2-ylamine

### Example 1881

### 6-Pyridin-4-yl-4′-(quinolin-2-ylmethoxy)-biphenyl-2-ylamine

### Example 1881

### Error! Objects cannot be created from editing field codes

To a solution of 2-(2′-nitro-6′-pyridin-4-yl-biphenyl-4-yloxymethyl)-quinoline (190 mg, 0.44 mmol) in EtOAc (10 mL) and water (0.2 mL) was added SnCl₂ (500 mg, 2.63 mmol) in one portion. The reaction mixture was stirred at RT for 18 h. 1N aqueous NaOH solution (20 mL) and EtOAc (10 mL) were added to quench the reaction. The organic layer was separated from the aqueous layer and the latter was extracted with CHCl₃ (3×10 mL). The combined organic phases were dried over MgSO₄. Filtration, concentration and purification via chromatography (30-100% EtOAc/heptane) provided the title compound 6-pyridin-4-yl-4′-(quinolin-2-ylmethoxy)-biphenyl-2-ylamine (150 mg, 85%) as a light yellow solid. Mass spectrometry (ESI): calcd for C₂₇H₂₂N₃O (MH⁺): 404.1757; found: 404.1759; HPLC 95.5% (Rt=10.88 min); ¹H NMR (300 MHz, CDCl₃/TMS) δ 8.35 (d, J=6.0 Hz, 2H), 8.20 (d, J=8.7 Hz, 1H), 8.08 (d, J=8.4 Hz, 1 H), 7.84 (d, J=7.8 Hz, 1H), 7.74 (dt, J=7.7, 1.3 Hz, 1H), 7.65 (d, J=8.4 Hz, 1H), 7.55 (dt, J=8.0, 0.9 Hz, 1H), 7.22 (t, J=7.8 Hz, 1H), 7.07-7.00 (m, 2H), 7.00-6.90 (m, 4H), 6.85-6.75 (m, 2H), 5.35 (s, 2H), 3.58 (br s, 2H); ¹³C NMR (75 MHz, CDCl₃/TMS) δ 157.4, 149.9, 148.5, 147.3, 144.6, 139.3, 136.8, 131.7, 129.6, 129.1, 128.7, 128.2, 127.5, 127.4, 126.4, 125.1, 124.4, 119.4, 118.9, 115.2, 115.1, 71.1.

### Synthesis of 2-(6′-methanesulfonyl-2′-pyridin-4-yl-biphenyl-4-yloxymethyl)-quinoline

### Example 392

### 4′-Benzyloxy-6-pyridin-4-yl-biphenyl-2-ylamine

### Error! Objects cannot be created from editing field codes

To a solution of 4-(4′-benzyloxy-6-nitro-biphenyl-2-yl)-pyridine (2.78 g, 7.27 mmol) in EtOAc (100 mL) and water (2.9 mL) was added SnCl₂ (8.27 g, 43.62 mmol) in one portion. The reaction mixture was heated to 40° C. and stirred for 5 h. The mixture was cooled to RT and diluted with EtOAc (100 mL) and quenched with 1N aqueous NaOH solution (200 mL). The organic phase was separated from the aqueous phase and the latter was extracted with CHCl₃ (4×100 mL). The combined organic phases were dried over MgSO₄. Filtration and concentration provided the title compound 4′-benzyloxy-6-pyridin-4-yl-biphenyl-2-ylamine (2.43 g, 95%) as a yellow solid. ¹H NMR (300 MHz, CDCl₃/TMS) δ 8.36 (d, J=5.1 Hz, 2H), 7.48-7.26 (m, 4H), 7.22 (t, J=7.8 Hz, 2H), 7.04 (d, J=9.0 Hz, 2H), 6.98 (dd, J=4.2, 1.5 Hz, 2H), 6.89 (d, J=9.0 Hz, 2H), 6.81 (t, J=7.8 Hz, 2H), 5.03 (s, 2H), 3.69 (br s, 2H); ¹³C NMR (75 MHz, CDCl₃/TMS) δ 157.7, 149.8, 148.6, 144.6, 139.3, 136.5, 131.5, 128.8, 128.3, 128.1, 127.8, 127.3, 125.2, 124.4, 119.4, 115.1, 115.0, 69.8.

### 4-(4′-Benzyloxy-6-iodo-biphenyl-2-yl)-pyridine

### Error! Objects cannot be created from editing field codes

4′-Benzyloxy-6-pyridin-4-yl-biphenyl-2-ylamine (2.21 g, 6.27 mmol) was dissolved in a minimum of glacial acetic acid (12 mL) and diluted with acetonitrile (30 mL). This solution was cooled to 10-15° C. and to this solution were added dropwise a solution of NaNO₂ (0.87 g, 12.54 mmol) and KI (10.41 g, 62.7 mmol) in minimum water (9 mL). The reaction mixture was stirred for 0.5 h at 10-15° C., then allowed to warm up to RT and stirred for 5 h. To the reaction mixture was added water (100 mL), the pH value was adjusted to 9-10, the mixture was treated with saturated Na₂SO₃, and extracted with EtOAc (3×70 mL). The combined organic phases were washed with brine (30 mL) and dried over MgSO₄. Concentration and purification by chromatography (0.5-3.0% MeOH/CH₂Cl₂) provided the title compound 4-(4′-benzyloxy-6-iodo-biphenyl-2-yl)-pyridine (2.38 g, 82%) as an off-white solid. ¹H NMR (300 MHz, CDCl₃/TMS) δ 8.40 (d, J=5.7 Hz, 2H), 8.03 (d, J=7.5 Hz, 1H), 7.51-7.20 (m, 6H), 7.12 (t, J=7.8 Hz, 1H), 7.00-6.90 (m, 4H), 6.87 (d, J=9.0 Hz, 2H), 5.02 (s, 2H); ¹³C NMR (75 MHz, CDCl₃/TMS) δ 157.8, 149.0, 148.8, 144.0, 139.7, 139.2, 136.4, 135.0, 131.2, 129.2, 128.8, 128.2, 127.7, 127.3, 124.0, 113.9, 102.4, 69.7

### 4-(4′-Benzyloxy-6-methanesulfonyl-biphenyl-2-yl)-pyridine

### Error! Objects cannot be created from editing field codes

A mixture of 4-(4′-benzyloxy-6-iodo-biphenyl-2-yl)-pyridine (303 mg, 0.65 mmol), sodium methanesulfinate (107 mg, 1.05 mmol), copper (I) iodide (187 mg, 0.98 mmol), and DMF (2 mL) was flushed with nitrogen, then heated to 110° C. for 7 h under nitrogen. After cooling, water (10 mL) and EtOAc (20 mL) were added with stirring and the insoluble materials were removed by filtration. The organic phase was separated, washed with brine (5 mL), and dried over MgSO₄. Removal of the solvent under reduced pressure left a yellow wax (0.44 g). Chromatography (0-2% MeOH/CH₂Cl₂) provided the title compound 4-(4′-benzyloxy-6-methanesulfonyl-biphenyl-2-yl)-pyridine (100 mg, 37%) as alight yellow wax. ¹H NMR (300 MHz, CDCl₃/TMS) δ 8.50 (br s, 2H), 8.35 (dd, J=6.6, 3.0 Hz, 1H), 7.68-7.60 (m, 2H), 7.43-7.28 (m, 5H), 7.14 (d, J=8.4 Hz, 2H), 6.98 (br s, 2H), 6.86 (d, J=8.7 Hz, 2H), 5.02 (s, 2H), 2.57 (s, 3H); ¹³C NMR (75 MHz, CDCl₃/TMS) δ 158.4, 149.1, 148.0, 141.5, 140.8, 138.8, 136.1, 134.2, 132.5, 128.4, 128.3, 127.9, 127.8, 127.3, 126.9, 124.3, 113.9, 69.8, 43.2.

### 6′-Methanesulfonyl-2′-pyridin-4-yl-biphenyl-4-ol

### Error! Objects cannot be created from editing field codes

4-(4′-Benzyloxy-6-methanesulfonyl-biphenyl-2-yl)-pyridine (100 mg, 0.24 mmol) was dissolved in CH₂Cl₂ (5 mL) and diluted with MeOH (15 mL). To this solution was added 10% Pd/C (100 mg), and the mixture was placed on a Parr hydrogenation apparatus for 16 h (20 psi H₂ pressure). The catalyst was filtered off and washed with a mixture of MeOH and CH₂Cl₂. Concentration and purification by chromatography (0-5% MeOH/CH₂Cl₂) provided title compound 6′-methanesulfonyl-2′-pyridin-4-yl-biphenyl-4-ol (70 mg, 90%) as a white wax. ¹H NMR (300 MHz, CD₃OD/CDCl₃/TMS) δ 8.34 (br s, 2H), 8.31 (t, J=7.8 Hz, 1H), 7.70 (d, J=5.1 Hz, 2H), 7.11 (br s, 2H), 7.06 (d, J=8.1 Hz, 2H), 6.72 (d, J=8.4 Hz, 2H), 2.64 (s, 3H); ¹³C NMR (75 MHz, CD₃OD/CDCl₃/TMS) δ 157.8, 149.8, 148.6, 142.2, 141.3, 140.0, 135.0, 133.2, 128.8, 128.5, 126.0, 125.5, 115.0, 43.5.

### 2-(6′-Methanesulfonyl-2′-pyridin-4-yl-biphenyl-4-yloxymethyl)-quinoline

### Example 392

### Error! Objects cannot be created from editing field codes

To a stirred solution of 6′-methanesulfonyl-2′-pyridin-4-yl-biphenyl-4-ol (70 mg, 0.22 mmol) in warm acetonitrile (15 mL) was added K₂CO₃ (152 mg, 1.10 mmol) and 2-chloromethyl-quinoline hydrochloride (51 mg, 0.24 mmol). The reaction mixture was heated to reflux and stirred under argon for 24 h. The mixture was cooled to RT and the inorganic salts were filtered and washed with EtOAc. Concentration and purification by chromatography (0-100% EtOAc/heptane) provided title compound 2-(6′-methanesulfonyl-2′-pyridin-4-yl-biphenyl-4-yloxymethyl)-quinoline (70 mg, 70%) as a light yellow wax. Mass spectrometry (DIP-CI): calcd for C₂₈H₂₃N₂O₃S (MH⁺): 467.1429; found: 467.1403; HPLC 95.3% (Rt=7.42 min); ¹H NMR (300 MHz, CDCl₃/TMS) δ 8.42 (br s, 1H), 8.34 (dd, J=6.3, 3.0 Hz, 1H), 8.21 (d, J=8.4 Hz, 1H), 8.07 (d, J=8.4 Hz, 1H), 7.85 (d, J=8.4 Hz, 1H), 7.74 (dt, J=7.7, 1.5 Hz, 1H), 7.68-7.59 (m, 3H), 7.56 (t, J=7.5 Hz, 1H), 7.15 (d, J=8.7 Hz, 2H), 7.10-6.78 (m, 5H), 5.34 (s, 2H), 2.57 (s, 3H); ¹³C NMR (75 MHz, CDCl₃/TMS) δ 158.2, 156.9, 149.0, 147.9, 147.3, 141.7, 140.8, 138.8, 136.8, 134.3, 132.7, 129.6, 128.7, 128.4, 127.9, 127.5, 127.4, 126.4, 124.3, 118.9, 114.0, 71.1, 43.3.

### Tables

Additional compounds of the disclosure are embodied in with distinct examples listed in the table below taken from Formula (I):

| Ex. # | X | Y | Z | R₁ | R₂ |
|---|---|---|---|---|---|
| 1 | 4-pyridinyl | CH₂O | 2-benzimidazolyl | H | H |
| 2 | 4-pyridinyl | CH₂O | 2-benzoxazolyl | H | H |
| 3 | 4-pyridinyl | CH₂O | 2-benzthiazolyl | H | H |
| 4 | 4-pyridinyl | CH₂O | 2-pyridinyl | H | H |
| 5 | 4-pyridinyl | CH₂O | 2-quinazolinyl | H | H |
| 6 | 4-pyridinyl | CH₂O | 2-quinolinyl | H | H |
| 7 | 4-pyridinyl | CH₂O | 2-quinolinyl | 3-F | H |
| 8 | 4-pyridinyl | CH₂O | 2-quinolinyl | 3-Cl | H |
| 9 | 4-pyridinyl | CH₂O | 2-quinolinyl | 3-CN | H |
| 10 | 4-pyridinyl | CH₂O | 2-quinolinyl | 3-NO₂ | H |
| 11 | 4-pyridinyl | CH₂O | 2-quinolinyl | 3-OMe | H |
| 12 | 4-pyridinyl | CH₂O | 2-quinolinyl | 3-Me | H |
| 13 | 4-pyridinyl | CH₂O | 2-quinolinyl | 3-Et | H |
| 14 | 4-pyridinyl | CH₂O | 2-quinolinyl | 3-ⁱPr | H |
| 15 | 4-pyridinyl | CH₂O | 2-quinolinyl | 3-^{t}Bu | H |
| 16 | 4-pyridinyl | CH₂O | 2-quinolinyl | 3-CF₃ | H |
| 17 | 4-pyridinyl | CH₂O | 2-quinolinyl | 3-SO₂Me | H |
| 18 | 4-pyridinyl | CH₂O | 2-quinolinyl | 3-SO₂Et | H |
| 19 | 4-pyridinyl | CH₂O | 2-quinolinyl | 3-SO₂ⁱPr | H |
| 20 | 4-pyridinyl | CH₂O | 2-quinolinyl | 3-OCF₃ | H |
| 21 | 4-pyridinyl | CH₂O | 2-quinolinyl | 3-OCH₂CF₃ | H |
| 22 | 4-pyridinyl | CH₂O | 2-quinolinyl | 3-NHMe | H |
| 23 | 4-pyridinyl | CH₂O | 2-quinolinyl | 3-NMe₂ | H |
| 24 | 4-pyridinyl | CH₂O | 2-quinolinyl | 3-cyclopropyl | H |
| 25 | 4-pyridinyl | CH₂O | 2-quinolinyl | 3-OEt | H |
| 26 | 4-pyridinyl | CH₂O | 2-quinolinyl | 3-OⁱPr | H |
| 27 | 4-pyridinyl | CH₂O | 2-quinolinyl | 3-CH₂-cyclopropyl | H |
| 28 | 4-pyridinyl | CH₂O | 2-quinolinyl | 3-SMe | H |
| 29 | 4-pyridinyl | CH₂O | 2-quinolinyl | 3-SEt | H |
| 30 | 4-pyridinyl | CH₂O | 2-quinolinyl | 3-SⁱPr | H |
| 31 | 4-pyridinyl | CH₂O | 2-quinolinyl | 4-F | H |
| 32 | 4-pyridinyl | CH₂O | 2-quinolinyl | 4-Cl | H |
| 33 | 4-pyridinyl | CH₂O | 2-quinolinyl | 4-CN | H |
| 34 | 4-pyridinyl | CH₂O | 2-quinolinyl | 4-NO₂ | H |
| 35 | 4-pyridinyl | CH₂O | 2-quinolinyl | 4-OMe | H |
| 36 | 4-pyridinyl | CH₂O | 2-quinolinyl | 4-Me | H |
| 37 | 4-pyridinyl | CH₂O | 2-quinolinyl | 4-Et | H |
| 38 | 4-pyridinyl | CH₂O | 2-quinolinyl | 4-ⁱPr | H |
| 39 | 4-pyridinyl | CH₂O | 2-quinolinyl | 4-^{t}Bu | H |
| 40 | 4-pyridinyl | CH₂O | 2-quinolinyl | 4-CF₃ | H |
| 41 | 4-pyridinyl | CH₂O | 2-quinolinyl | 4-SO₂Me | H |
| 42 | 4-pyridinyl | CH₂O | 2-quinolinyl | 4-SO₂Et | H |
| 43 | 4-pyridinyl | CH₂O | 2-quinolinyl | 4-SO₂ⁱPr | H |
| 44 | 4-pyridinyl | CH₂O | 2-quinolinyl | 4-OCF₃ | H |
| 45 | 4-pyridinyl | CH₂O | 2-quinolinyl | 4-OCH₂CF₃ | H |
| 46 | 4-pyridinyl | CH₂O | 2-quinolinyl | 4-NHMe | H |
| 47 | 4-pyridinyl | CH₂O | 2-quinolinyl | 4-NMe₂ | H |
| 48 | 4-pyridinyl | CH₂O | 2-quinolinyl | 4-cyclopropyl | H |
| 49 | 4-pyridinyl | CH₂O | 2-quinolinyl | 4-OEt | H |
| 50 | 4-pyridinyl | CH₂O | 2-quinolinyl | 4-OⁱPr | H |
| 51 | 4-pyridinyl | CH₂O | 2-quinolinyl | 4-CH₂-cyclopropyl | H |
| 52 | 4-pyridinyl | CH₂O | 2-quinolinyl | 4-SMe | H |
| 53 | 4-pyridinyl | CH₂O | 2-quinolinyl | 4-SEt | H |
| 54 | 4-pyridinyl | CH₂O | 2-quinolinyl | 4-SⁱPr | H |
| 55 | ⁱPr | CH₂O | 2-quinolinyl | H | H |
| 56 | Me | CH₂O | 2-quinolinyl | H | H |
| 57 | morpholinyl | CH₂O | 2-quinolinyl | H | H |
| 58 | N-piperazino | CH₂O | 2-quinolinyl | H | H |
| 59 | piperazino | CH₂O | 2-quinolinyl | H | H |
| 60 | piperidino | CH₂O | 2-quinolinyl | H | H |
| 61 | 4-pyridinyl | CH₂O | 2-quinoxalinyl | H | H |
| 62 | 4-pyridinyl | CH₂O | 5,6,7,8-tetrahydro-2-quinolyl | H | H |
| 63 | 3-pyridinyl | OCH₂ | 2-benzimidazolyl | H | H |
| 64 | 4-pyridinyl | OCH₂ | 2-benzimidazolyl | H | H |
| 65 | morpholinyl | OCH₂ | 2-benzimidazolyl | H | H |
| 66 | 3-pyridinyl | OCH₂ | 2-benzoxazolyl | H | H |
| 67 | 4-pyridinyl | OCH₂ | 2-benzoxazolyl | H | H |
| 68 | morpholinyl | OCH₂ | 2-benzoxazolyl | H | H |
| 69 | 3-pyridinyl | OCH₂ | 2-benzthiazolyl | H | H |
| 70 | 4-pyridinyl | OCH₂ | 2-benzthiazolyl | H | H |
| 71 | morpholinyl | OCH₂ | 2-benzthiazolyl | H | H |
| 72 | 3-pyridinyl | OCH₂ | 2-pyridinyl | H | H |
| 73 | 4-pyridinyl | OCH₂ | 2-pyridinyl | H | H |
| 74 | morpholinyl | OCH₂ | 2-pyridinyl | H | H |
| 75 | 3-pyridinyl | OCH₂ | 2-quinazolinyl | H | H |
| 76 | 4-pyridinyl | OCH₂ | 2-quinazolinyl | H | H |
| 77 | morpholinyl | OCH₂ | 2-quinazolinyl | H | H |
| 78 | 2-hydroxy-4-pyridinyl | OCH₂ | 2-quinolinyl | H | H |
| 79 | 2-hydroxy-4-pyridinyl | OCH₂ | 2-quinolinyl | 3-F | H |
| 80 | 2-hydroxy-4-pyridinyl | OCH₂ | 2-quinolinyl | 3-Cl | H |
| 81 | 2-hydroxy-4-pyridinyl | OCH₂ | 2-quinolinyl | 3-CN | H |
| 82 | 2-hydroxy-4-pyridinyl | OCH₂ | 2-quinolinyl | 3-NO₂ | H |
| 83 | 2-hydroxy-4-pyridinyl | OCH₂ | 2-quinolinyl | 3-OMe | H |
| 84 | 2-hydroxy-4-pyridinyl | OCH₂ | 2-quinolinyl | 3-Me | H |
| 85 | 2-hydroxy-4-pyridinyl | OCH₂ | 2-quinolinyl | 3-Et | H |
| 86 | 2-hydroxy-4-pyridinyl | OCH₂ | 2-quinolinyl | 3-ⁱPr | H |
| 87 | 2-hydroxy-4-pyridinyl | OCH₂ | 2-quinolinyl | 3-^{t}Bu | H |
| 88 | 2-hydroxy-4-pyridinyl | OCH₂ | 2-quinolinyl | 3-CF₃ | H |
| 89 | 2-hydroxy-4-pyridinyl | OCH₂ | 2-quinolinyl | 3-SO₂Me | H |
| 90 | 2-hydroxy-4-pyridinyl | OCH₂ | 2-quinolinyl | 3-SO₂Et | H |
| 91 | 2-hydroxy-4-pyridinyl | OCH₂ | 2-quinolinyl | 3-SO₂ⁱPr | H |
| 92 | 2-hydroxy-4-pyridinyl | OCH₂ | 2-quinolinyl | 3-OCF₃ | H |
| 93 | 2-hydroxy-4-pyridinyl | OCH₂ | 2-quinolinyl | 3-OCH₂CF₃ | H |
| 94 | 2-hydroxy-4-pyridinyl | OCH₂ | 2-quinolinyl | 3-NHMe | H |
| 95 | 2-hydroxy-4-pyridinyl | OCH₂ | 2-quinolinyl | 3-NMe₂ | H |
| 96 | 2-hydroxy-4-pyridinyl | OCH₂ | 2-quinolinyl | 3-cyclopropyl | H |
| 97 | 2-hydroxy-4-pyridinyl | OCH₂ | 2-quinolinyl | 3-OEt | H |
| 98 | 2-hydroxy-4-pyridinyl | OCH₂ | 2-quinolinyl | 3-OⁱPr | H |
| 99 | 2-hydroxy-4-pyridinyl | OCH₂ | 2-quinolinyl | 3-CH₂-cyclopropyl | H |
| 100 | 2-hydroxy-4-pyridinyl | OCH₂ | 2-quinolinyl | 3-SMe | H |
| 101 | 2-hydroxy-4-pyridinyl | OCH₂ | 2-quinolinyl | 3-SEt | H |
| 102 | 2-hydroxy-4-pyridinyl | OCH₂ | 2-quinolinyl | 3-SⁱPr | H |
| 103 | 2-hydroxy-5-pyridinyl | OCH₂ | 2-quinolinyl | H | H |
| 104 | 2-hydroxy-5-pyridinyl | OCH₂ | 2-quinolinyl | 3-F | H |
| 105 | 2-hydroxy-5-pyridinyl | OCH₂ | 2-quinolinyl | 3-Cl | H |
| 106 | 2-hydroxy-5-pyridinyl | OCH₂ | 2-quinolinyl | 3-CN | H |
| 107 | 2-hydroxy-5-pyridinyl | OCH₂ | 2-quinolinyl | 3-NO₂ | H |
| 108 | 2-hydroxy-5-pyridinyl | OCH₂ | 2-quinolinyl | 3-OMe | H |
| 109 | 2-hydroxy-5-pyridinyl | OCH₂ | 2-quinolinyl | 3-Me | H |
| 110 | 2-hydroxy-5-pyridinyl | OCH₂ | 2-quinolinyl | 3-Et | H |
| 111 | 2-hydroxy-5-pyridinyl | OCH₂ | 2-quinolinyl | 3-ⁱPr | H |
| 112 | 2-hydroxy-5-pyridinyl | OCH₂ | 2-quinolinyl | 3-^{t}Bu | H |
| 113 | 2-hydroxy-5-pyridinyl | OCH₂ | 2-quinolinyl | 3-CF₃ | H |
| 114 | 2-hydroxy-5-pyridinyl | OCH₂ | 2-quinolinyl | 3-SO₂Me | H |
| 115 | 2-hydroxy-5-pyridinyl | OCH₂ | 2-quinolinyl | 3-SO₂Et | H |
| 116 | 2-hydroxy-5-pyridinyl | OCH₂ | 2-quinolinyl | 3-SO₂ⁱPr | H |
| 117 | 2-hydroxy-5-pyridinyl | OCH₂ | 2-quinolinyl | 3-OCF₃ | H |
| 118 | 2-hydroxy-5-pyridinyl | OCH₂ | 2-quinolinyl | 3-OCH₂CF₃ | H |
| 119 | 2-hydroxy-5-pyridinyl | OCH₂ | 2-quinolinyl | 3-NHMe | H |
| 120 | 2-hydroxy-5-pyridinyl | OCH₂ | 2-quinolinyl | 3-NMe₂ | H |
| 121 | 2-hydroxy-5-pyridinyl | OCH₂ | 2-quinolinyl | 3-cyclopropyl | H |
| 122 | 2-hydroxy-5-pyridinyl | OCH₂ | 2-quinolinyl | 3-OEt | H |
| 123 | 2-hydroxy-5-pyridinyl | OCH₂ | 2-quinolinyl | 3-OⁱPr | H |
| 124 | 2-hydroxy-5-pyridinyl | OCH₂ | 2-quinolinyl | 3-CH₂-cyclopropyl | H |
| 125 | 2-hydroxy-5-pyridinyl | OCH₂ | 2-quinolinyl | 3-SMe | H |
| 126 | 2-hydroxy-5-pyridinyl | OCH₂ | 2-quinolinyl | 3-SEt | H |
| 127 | 2-hydroxy-5-pyridinyl | OCH₂ | 2-quinolinyl | 3-SⁱPr | H |
| 128 | 2-methoxy-4-pyridinyl | OCH₂ | 2-quinolinyl | H | H |
| 129 | 2-methoxy-4-pyridinyl | OCH₂ | 2-quinolinyl | 3-F | H |
| 130 | 2-methoxy-4-pyridinyl | OCH₂ | 2-quinolinyl | 3-Cl | H |
| 131 | 2-methoxy-4-pyridinyl | OCH₂ | 2-quinolinyl | 3-CN | H |
| 132 | 2-methoxy-4-pyridinyl | OCH₂ | 2-quinolinyl | 3-NO₂ | H |
| 133 | 2-methoxy-4-pyridinyl | OCH₂ | 2-quinolinyl | 3-OMe | H |
| 134 | 2-methoxy-4-pyridinyl | OCH₂ | 2-quinolinyl | 3-Me | H |
| 135 | 2-methoxy-4-pyridinyl | OCH₂ | 2-quinolinyl | 3-Et | H |
| 136 | 2-methoxy-4-pyridinyl | OCH₂ | 2-quinolinyl | 3-ⁱPr | H |
| 137 | 2-methoxy-4-pyridinyl | OCH₂ | 2-quinolinyl | 3-^{t}Bu | H |
| 138 | 2-methoxy-4-pyridinyl | OCH₂ | 2-quinolinyl | 3-CF₃ | H |
| 139 | 2-methoxy-4-pyridinyl | OCH₂ | 2-quinolinyl | 3-SO₂Me | H |
| 140 | 2-methoxy-4-pyridinyl | OCH₂ | 2-quinolinyl | 3-SO₂Et | H |
| 141 | 2-methoxy-4-pyridinyl | OCH₂ | 2-quinolinyl | 3-SO₂ⁱPr | H |
| 142 | 2-methoxy-4-pyridinyl | OCH₂ | 2-quinolinyl | 3-OCF₃ | H |
| 143 | 2-methoxy-4-pyridinyl | OCH₂ | 2-quinolinyl | 3-OCH₂CF₃ | H |
| 144 | 2-methoxy-4-pyridinyl | OCH₂ | 2-quinolinyl | 3-NHMe | H |
| 145 | 2-methoxy-4-pyridinyl | OCH₂ | 2-quinolinyl | 3-NMe₂ | H |
| 146 | 2-methoxy-4-pyridinyl | OCH₂ | 2-quinolinyl | 3-cyclopropyl | H |
| 147 | 2-methoxy-4-pyridinyl | OCH₂ | 2-quinolinyl | 3-OEt | H |
| 148 | 2-methoxy-4-pyridinyl | OCH₂ | 2-quinolinyl | 3-OⁱPr | H |
| 149 | 2-methoxy-4-pyridinyl | OCH₂ | 2-quinolinyl | 3-CH₂-cyclopropyl | H |
| 150 | 2-methoxy-4-pyridinyl | OCH₂ | 2-quinolinyl | 3-SMe | H |
| 151 | 2-methoxy-4-pyridinyl | OCH₂ | 2-quinolinyl | 3-SEt | H |
| 152 | 2-methoxy-4-pyridinyl | OCH₂ | 2-quinolinyl | 3-SⁱPr | H |
| 153 | 2-methoxy-5-pyridinyl | OCH₂ | 2-quinolinyl | H | H |
| 154 | 2-methoxy-5-pyridinyl | OCH₂ | 2-quinolinyl | 3-F | H |
| 155 | 2-methoxy-5-pyridinyl | OCH₂ | 2-quinolinyl | 3-Cl | H |
| 156 | 2-methoxy-5-pyridinyl | OCH₂ | 2-quinolinyl | 3-CN | H |
| 157 | 2-methoxy-5-pyridinyl | OCH₂ | 2-quinolinyl | 3-NO₂ | H |
| 158 | 2-methoxy-5-pyridinyl | OCH₂ | 2-quinolinyl | 3-OMe | H |
| 159 | 2-methoxy-5-pyridinyl | OCH₂ | 2-quinolinyl | 3-Me | H |
| 160 | 2-methoxy-5-pyridinyl | OCH₂ | 2-quinolinyl | 3-Et | H |
| 161 | 2-methoxy-5-pyridinyl | OCH₂ | 2-quinolinyl | 3-ⁱPr | H |
| 162 | 2-methoxy-5-pyridinyl | OCH₂ | 2-quinolinyl | 3-^{t}Bu | H |
| 163 | 2-methoxy-5-pyridinyl | OCH₂ | 2-quinolinyl | 3-CF₃ | H |
| 164 | 2-methoxy-5-pyridinyl | OCH₂ | 2-quinolinyl | 3-SO₂Me | H |
| 165 | 2-methoxy-5-pyridinyl | OCH₂ | 2-quinolinyl | 3-SO₂Et | H |
| 166 | 2-methoxy-5-pyridinyl | OCH₂ | 2-quinolinyl | 3-SO₂ⁱPr | H |
| 167 | 2-methoxy-5-pyridinyl | OCH₂ | 2-quinolinyl | 3-OCF₃ | H |
| 168 | 2-methoxy-5-pyridinyl | OCH₂ | 2-quinolinyl | 3-OCH₂CF₃ | H |
| 169 | 2-methoxy-5-pyridinyl | OCH₂ | 2-quinolinyl | 3-NHMe | H |
| 170 | 2-methoxy-5-pyridinyl | OCH₂ | 2-quinolinyl | 3-NMe₂ | H |
| 171 | 2-methoxy-5-pyridinyl | OCH₂ | 2-quinolinyl | 3-cyclopropyl | H |
| 172 | 2-methoxy-5-pyridinyl | OCH₂ | 2-quinolinyl | 3-OEt | H |
| 173 | 2-methoxy-5-pyridinyl | OCH₂ | 2-quinolinyl | 3-OⁱPr | H |
| 174 | 2-methoxy-5-pyridinyl | OCH₂ | 2-quinolinyl | 3-CH₂-cyclopropyl | H |
| 175 | 2-methoxy-5-pyridinyl | OCH₂ | 2-quinolinyl | 3-SMe | H |
| 176 | 2-methoxy-5-pyridinyl | OCH₂ | 2-quinolinyl | 3-SEt | H |
| 177 | 2-methoxy-5-pyridinyl | OCH₂ | 2-quinolinyl | 3-SⁱPr | H |
| 178 | 3,4-dimethoxyphenyl | OCH₂ | 2-quinolinyl | H | H |
| 180 | 4-chloro-phenyl | OCH₂ | 2-quinolinyl | H | H |
| 181 | 4-chloro-phenyl | OCH₂ | 2-quinolinyl | 3-F | H |
| 182 | 4-chloro-phenyl | OCH₂ | 2-quinolinyl | 3-Cl | H |
| 183 | 4-chloro-phenyl | OCH₂ | 2-quinolinyl | 3-CN | H |
| 184 | 4-chloro-phenyl | OCH₂ | 2-quinolinyl | 3-NO₂ | H |
| 185 | 4-chloro-phenyl | OCH₂ | 2-quinolinyl | 3-OMe | H |
| 186 | 4-chloro-phenyl | OCH₂ | 2-quinolinyl | 3-Me | H |
| 187 | 4-chloro-phenyl | OCH₂ | 2-quinolinyl | 3-Et | H |
| 188 | 4-chloro-phenyl | OCH₂ | 2-quinolinyl | 3-ⁱPr | H |
| 189 | 4-chloro-phenyl | OCH₂ | 2-quinolinyl | 3-^{t}Bu | H |
| 190 | 4-chloro-phenyl | OCH₂ | 2-quinolinyl | 3-CF₃ | H |
| 191 | 4-chloro-phenyl | OCH₂ | 2-quinolinyl | 3-SO₂Me | H |
| 192 | 4-chloro-phenyl | OCH₂ | 2-quinolinyl | 3-SO₂Et | H |
| 193 | 4-chloro-phenyl | OCH₂ | 2-quinolinyl | 3-SO₂ⁱPr | H |
| 194 | 4-chloro-phenyl | OCH₂ | 2-quinolinyl | 3-OCF₃ | H |
| 195 | 4-chloro-phenyl | OCH₂ | 2-quinolinyl | 3-OCH₂CF₃ | H |
| 196 | 4-chloro-phenyl | OCH₂ | 2-quinolinyl | 3-NHMe | H |
| 197 | 4-chloro-phenyl | OCH₂ | 2-quinolinyl | 3-NMe₂ | H |
| 198 | 4-chloro-phenyl | OCH₂ | 2-quinolinyl | 3-cyclopropyl | H |
| 199 | 4-chloro-phenyl | OCH₂ | 2-quinolinyl | 3-OEt | H |
| 200 | 4-chloro-phenyl | OCH₂ | 2-quinolinyl | 3-OⁱPr | H |
| 201 | 4-chloro-phenyl | OCH₂ | 2-quinolinyl | 3-CH₂-cyclopropyl | H |
| 202 | 4-chloro-phenyl | OCH₂ | 2-quinolinyl | 3-SMe | H |
| 203 | 4-chloro-phenyl | OCH₂ | 2-quinolinyl | 3-SEt | H |
| 204 | 4-chloro-phenyl | OCH₂ | 2-quinolinyl | 3-SⁱPr | H |
| 205 | 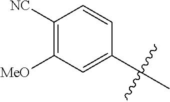 | OCH₂ | 2-quinolinyl | H | H |
| 206 | 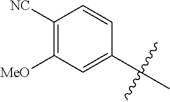 | OCH₂ | 2-quinolinyl | 3-F | H |
| 207 | 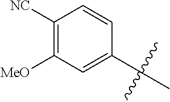 | OCH₂ | 2-quinolinyl | 3-Cl | H |
| 208 | 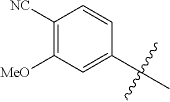 | OCH₂ | 2-quinolinyl | 3-CN | H |
| 209 | 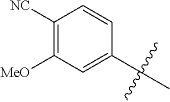 | OCH₂ | 2-quinolinyl | 3-NO₂ | H |
| 210 | 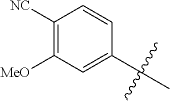 | OCH₂ | 2-quinolinyl | 3-OMe | H |
| 211 | 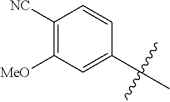 | OCH₂ | 2-quinolinyl | 3-Me | H |
| 212 | 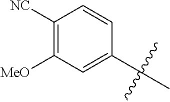 | OCH₂ | 2-quinolinyl | 3-Et | H |
| 213 | 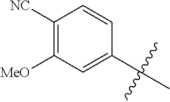 | OCH₂ | 2-quinolinyl | 3-ⁱPr | H |
| 214 | 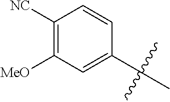 | OCH₂ | 2-quinolinyl | 3-^{t}Bu | H |
| 215 | 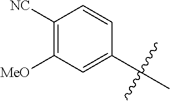 | OCH₂ | 2-quinolinyl | 3-CF₃ | H |
| 216 | 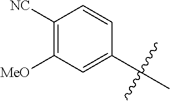 | OCH₂ | 2-quinolinyl | 3-SO₂Me | H |
| 217 | 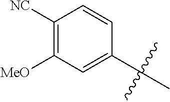 | OCH₂ | 2-quinolinyl | 3-SO₂Et | H |
| 218 | 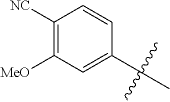 | OCH₂ | 2-quinolinyl | 3-SO₂ⁱPr | H |
| 219 | 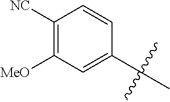 | OCH₂ | 2-quinolinyl | 3-OCF₃ | H |
| 220 | 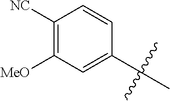 | OCH₂ | 2-quinolinyl | 3-OCH₂CF₃ | H |
| 221 | 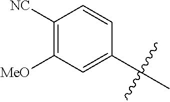 | OCH₂ | 2-quinolinyl | 3-NHMe | H |
| 222 | 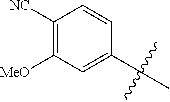 | OCH₂ | 2-quinolinyl | 3-NMe₂ | H |
| 223 | 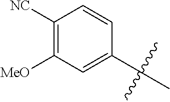 | OCH₂ | 2-quinolinyl | 3-cyclopropyl | H |
| 224 | 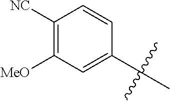 | OCH₂ | 2-quinolinyl | 3-OEt | H |
| 225 | 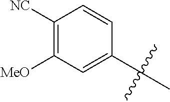 | OCH₂ | 2-quinolinyl | 3-OⁱPr | H |
| 226 | 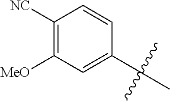 | OCH₂ | 2-quinolinyl | 3-CH₂-cyclopropyl | H |
| 227 | 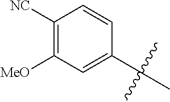 | OCH₂ | 2-quinolinyl | 3-SMe | H |
| 228 | 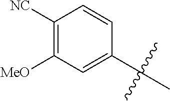 | OCH₂ | 2-quinolinyl | 3-SEt | H |
| 229 | 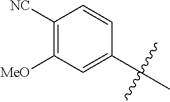 | OCH₂ | 2-quinolinyl | 3-SⁱPr | H |
| 231 | 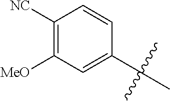 | OCH₂ | 2-quinolinyl | 4-F | H |
| 232 | 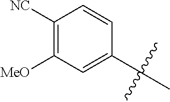 | OCH₂ | 2-quinolinyl | 4-Cl | H |
| 233 | 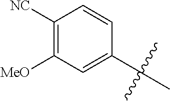 | OCH₂ | 2-quinolinyl | 4-CN | H |
| 234 | 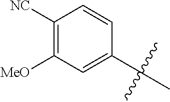 | OCH₂ | 2-quinolinyl | 4-NO₂ | H |
| 235 | 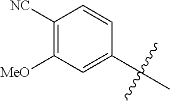 | OCH₂ | 2-quinolinyl | 4-OMe | H |
| 236 | 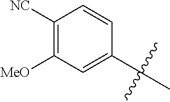 | OCH₂ | 2-quinolinyl | 4-Me | H |
| 237 | 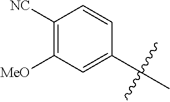 | OCH₂ | 2-quinolinyl | 4-Et | H |
| 238 | 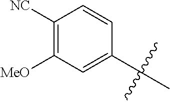 | OCH₂ | 2-quinolinyl | 4-iPr | H |
| 239 | 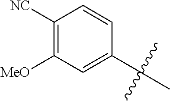 | OCH₂ | 2-quinolinyl | 4-tBu | H |
| 240 | 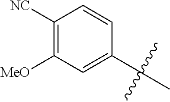 | OCH₂ | 2-quinolinyl | 4-CF3 | H |
| 241 | 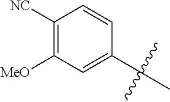 | OCH₂ | 2-quinolinyl | 4-SO2Me | H |
| 242 | 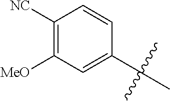 | OCH₂ | 2-quinolinyl | 4-SO2Et | H |
| 243 | 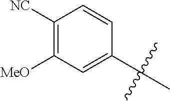 | OCH₂ | 2-quinolinyl | 4-SO2iPr | H |
| 244 | 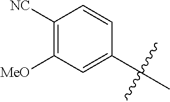 | OCH₂ | 2-quinolinyl | 4-OCF3 | H |
| 245 | 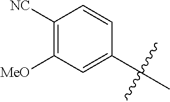 | OCH₂ | 2-quinolinyl | 4-OCH2CF3 | H |
| 246 | 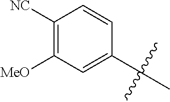 | OCH₂ | 2-quinolinyl | 4-NHMe | H |
| 247 | 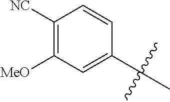 | OCH₂ | 2-quinolinyl | 4-NMe2 | H |
| 248 | 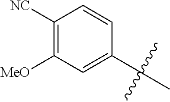 | OCH₂ | 2-quinolinyl | 4-cyclopropyl | H |
| 249 | 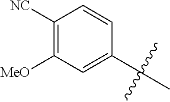 | OCH₂ | 2-quinolinyl | 4-OEt | H |
| 250 | 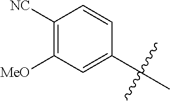 | OCH₂ | 2-quinolinyl | 4-OiPr | H |
| 251 | 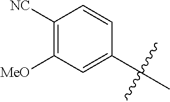 | OCH₂ | 2-quinolinyl | 4-CH2-cyclopropyl | H |
| 252 | 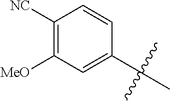 | OCH₂ | 2-quinolinyl | 4-SMe | H |
| 253 | 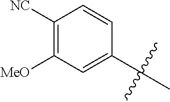 | OCH₂ | 2-quinolinyl | 4-SEt | H |
| 254 | 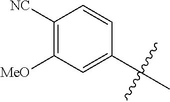 | OCH₂ | 2-quinolinyl | 4-SiPr | H |
| 255 | 4-cyano-phenyl | OCH₂ | 2-quinolinyl | H | H |
| 256 | 4-cyano-phenyl | OCH₂ | 2-quinolinyl | 3-F | H |
| 257 | 4-cyano-phenyl | OCH₂ | 2-quinolinyl | 3-Cl | H |
| 258 | 4-cyano-phenyl | OCH₂ | 2-quinolinyl | 3-CN | H |
| 259 | 4-cyano-phenyl | OCH₂ | 2-quinolinyl | 3-NO₂ | H |
| 260 | 4-cyano-phenyl | OCH₂ | 2-quinolinyl | 3-OMe | H |
| 261 | 4-cyano-phenyl | OCH₂ | 2-quinolinyl | 3-Me | H |
| 262 | 4-cyano-phenyl | OCH₂ | 2-quinolinyl | 3-Et | H |
| 263 | 4-cyano-phenyl | OCH₂ | 2-quinolinyl | 3-ⁱPr | H |
| 264 | 4-cyano-phenyl | OCH₂ | 2-quinolinyl | 3-^{t}Bu | H |
| 265 | 4-cyano-phenyl | OCH₂ | 2-quinolinyl | 3-CF₃ | H |
| 266 | 4-cyano-phenyl | OCH₂ | 2-quinolinyl | 3-SO₂Me | H |
| 267 | 4-cyano-phenyl | OCH₂ | 2-quinolinyl | 3-SO₂Et | H |
| 268 | 4-cyano-phenyl | OCH₂ | 2-quinolinyl | 3-SO₂ⁱPr | H |
| 269 | 4-cyano-phenyl | OCH₂ | 2-quinolinyl | 3-OCF₃ | H |
| 270 | 4-cyano-phenyl | OCH₂ | 2-quinolinyl | 3-OCH₂CF₃ | H |
| 271 | 4-cyano-phenyl | OCH₂ | 2-quinolinyl | 3-NHMe | H |
| 272 | 4-cyano-phenyl | OCH₂ | 2-quinolinyl | 3-NMe₂ | H |
| 273 | 4-cyano-phenyl | OCH₂ | 2-quinolinyl | 3-cyclopropyl | H |
| 274 | 4-cyano-phenyl | OCH₂ | 2-quinolinyl | 3-OEt | H |
| 275 | 4-cyano-phenyl | OCH₂ | 2-quinolinyl | 3-OⁱPr | H |
| 276 | 4-cyano-phenyl | OCH₂ | 2-quinolinyl | 3-CH₂-cyclopropyl | H |
| 277 | 4-cyano-phenyl | OCH₂ | 2-quinolinyl | 3-SMe | H |
| 278 | 4-cyano-phenyl | OCH₂ | 2-quinolinyl | 3-SEt | H |
| 279 | 4-cyano-phenyl | OCH₂ | 2-quinolinyl | 3-SⁱPr | H |
| 281 | 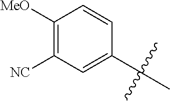 | OCH₂ | 2-quinolinyl | H | H |
| 282 | 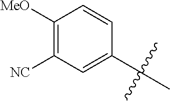 | OCH₂ | 2-quinolinyl | 3-F | H |
| 283 | 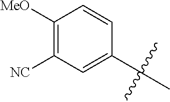 | OCH₂ | 2-quinolinyl | 3-Cl | H |
| 284 | 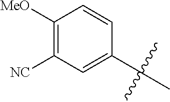 | OCH₂ | 2-quinolinyl | 3-CN | H |
| 285 | 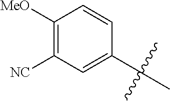 | OCH₂ | 2-quinolinyl | 3-NO₂ | H |
| 286 | 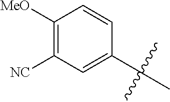 | OCH₂ | 2-quinolinyl | 3-OMe | H |
| 287 | 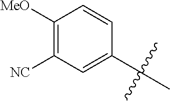 | OCH₂ | 2-quinolinyl | 3-Me | H |
| 288 | 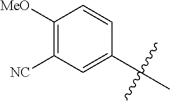 | OCH₂ | 2-quinolinyl | 3-Et | H |
| 289 | 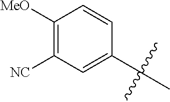 | OCH₂ | 2-quinolinyl | 3-ⁱPr | H |
| 290 | 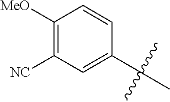 | OCH₂ | 2-quinolinyl | 3-^{t}Bu | H |
| 291 | 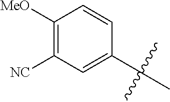 | OCH₂ | 2-quinolinyl | 3-CF₃ | H |
| 292 | 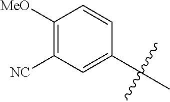 | OCH₂ | 2-quinolinyl | 3-SO₂Me | H |
| 293 | 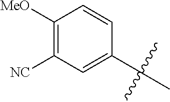 | OCH₂ | 2-quinolinyl | 3-SO₂Et | H |
| 294 | 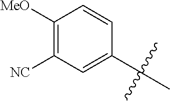 | OCH₂ | 2-quinolinyl | 3-SO₂ⁱPr | H |
| 295 | 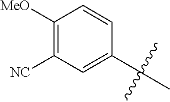 | OCH₂ | 2-quinolinyl | 3-OCF₃ | H |
| 296 | 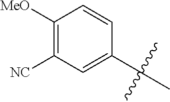 | OCH₂ | 2-quinolinyl | 3-OCH₂CF₃ | H |
| 297 | 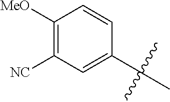 | OCH₂ | 2-quinolinyl | 3-NHMe | H |
| 298 | 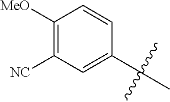 | OCH₂ | 2-quinolinyl | 3-NMe₂ | H |
| 299 | 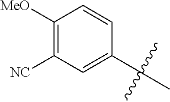 | OCH₂ | 2-quinolinyl | 3-cyclopropyl | H |
| 300 | 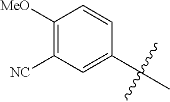 | OCH₂ | 2-quinolinyl | 3-OEt | H |
| 301 | 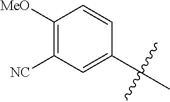 | OCH₂ | 2-quinolinyl | 3-OⁱPr | H |
| 302 | 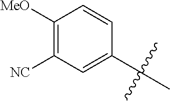 | OCH₂ | 2-quinolinyl | 3-CH₂-cyclopropyl | H |
| 303 | 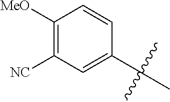 | OCH₂ | 2-quinolinyl | 3-SMe | H |
| 304 | 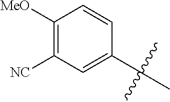 | OCH₂ | 2-quinolinyl | 3-SEt | H |
| 305 | 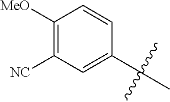 | OCH₂ | 2-quinolinyl | 3-SⁱPr | H |
| 306 | 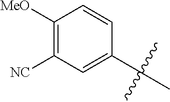 | OCH₂ | 2-quinolinyl | 4-F | H |
| 307 | 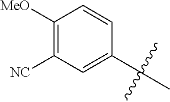 | OCH₂ | 2-quinolinyl | 4-Cl | H |
| 308 | 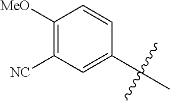 | OCH₂ | 2-quinolinyl | 4-CN | H |
| 309 | 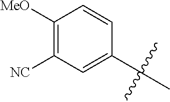 | OCH₂ | 2-quinolinyl | 4-NO₂ | H |
| 310 | 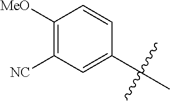 | OCH₂ | 2-quinolinyl | 4-OMe | H |
| 311 | 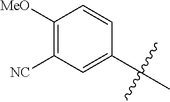 | OCH₂ | 2-quinolinyl | 4-Me | H |
| 312 | 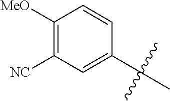 | OCH₂ | 2-quinolinyl | 4-Et | H |
| 313 | 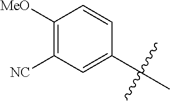 | OCH₂ | 2-quinolinyl | 4-iPr | H |
| 314 | 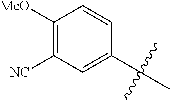 | OCH₂ | 2-quinolinyl | 4-tBu | H |
| 315 | 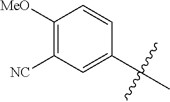 | OCH₂ | 2-quinolinyl | 4-CF3 | H |
| 316 | 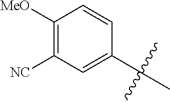 | OCH₂ | 2-quinolinyl | 4-SO2Me | H |
| 317 | 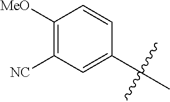 | OCH₂ | 2-quinolinyl | 4-SO2Et | H |
| 318 | 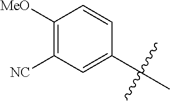 | OCH₂ | 2-quinolinyl | 4-SO2iPr | H |
| 319 | 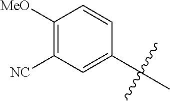 | OCH₂ | 2-quinolinyl | 4-OCF3 | H |
| 320 | 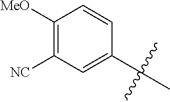 | OCH₂ | 2-quinolinyl | 4-OCH2CF3 | H |
| 321 | 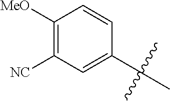 | OCH₂ | 2-quinolinyl | 4-NHMe | H |
| 322 | 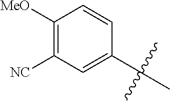 | OCH₂ | 2-quinolinyl | 4-NMe2 | H |
| 323 | 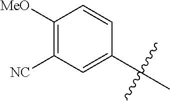 | OCH₂ | 2-quinolinyl | 4-cyclopropyl | H |
| 324 | 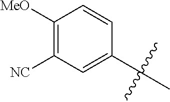 | OCH₂ | 2-quinolinyl | 4-OEt | H |
| 325 | 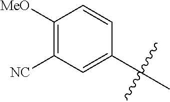 | OCH₂ | 2-quinolinyl | 4-OiPr | H |
| 326 | 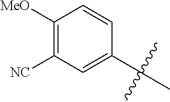 | OCH₂ | 2-quinolinyl | 4-CH2-cyclopropyl | H |
| 327 | 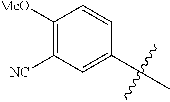 | OCH₂ | 2-quinolinyl | 4-SMe | H |
| 328 | 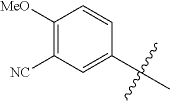 | OCH₂ | 2-quinolinyl | 4-SEt | H |
| 329 | 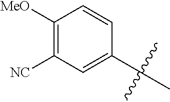 | OCH₂ | 2-quinolinyl | 4-SiPr | H |
| 330 | 4-methoxy-phenyl | OCH₂ | 2-quinolinyl | H | H |
| 331 | 4-methoxy-phenyl | OCH₂ | 2-quinolinyl | 3-F | H |
| 332 | 4-methoxy-phenyl | OCH₂ | 2-quinolinyl | 3-Cl | H |
| 333 | 4-methoxy-phenyl | OCH₂ | 2-quinolinyl | 3-CN | H |
| 334 | 4-methoxy-phenyl | OCH₂ | 2-quinolinyl | 3-NO₂ | H |
| 335 | 4-methoxy-phenyl | OCH₂ | 2-quinolinyl | 3-OMe | H |
| 336 | 4-methoxy-phenyl | OCH₂ | 2-quinolinyl | 3-Me | H |
| 337 | 4-methoxy-phenyl | OCH₂ | 2-quinolinyl | 3-Et | H |
| 338 | 4-methoxy-phenyl | OCH₂ | 2-quinolinyl | 3-ⁱPr | H |
| 339 | 4-methoxy-phenyl | OCH₂ | 2-quinolinyl | 3-^{t}Bu | H |
| 340 | 4-methoxy-phenyl | OCH₂ | 2-quinolinyl | 3-CF₃ | H |
| 341 | 4-methoxy-phenyl | OCH₂ | 2-quinolinyl | 3-SO₂Me | H |
| 342 | 4-methoxy-phenyl | OCH₂ | 2-quinolinyl | 3-SO₂Et | H |
| 343 | 4-methoxy-phenyl | OCH₂ | 2-quinolinyl | 3-SO₂ⁱPr | H |
| 344 | 4-methoxy-phenyl | OCH₂ | 2-quinolinyl | 3-OCF₃ | H |
| 345 | 4-methoxy-phenyl | OCH₂ | 2-quinolinyl | 3-OCH₂CF₃ | H |
| 346 | 4-methoxy-phenyl | OCH₂ | 2-quinolinyl | 3-NHMe | H |
| 347 | 4-methoxy-phenyl | OCH₂ | 2-quinolinyl | 3-NMe₂ | H |
| 348 | 4-methoxy-phenyl | OCH₂ | 2-quinolinyl | 3-cyclopropyl | H |
| 349 | 4-methoxy-phenyl | OCH₂ | 2-quinolinyl | 3-OEt | H |
| 350 | 4-methoxy-phenyl | OCH₂ | 2-quinolinyl | 3-OⁱPr | H |
| 351 | 4-methoxy-phenyl | OCH₂ | 2-quinolinyl | 3-CH₂-cyclopropyl | H |
| 352 | 4-methoxy-phenyl | OCH₂ | 2-quinolinyl | 3-SMe | H |
| 353 | 4-methoxy-phenyl | OCH₂ | 2-quinolinyl | 3-SEt | H |
| 354 | 4-methoxy-phenyl | OCH₂ | 2-quinolinyl | 3-SⁱPr | H |
| 356 | 4-methoxy-phenyl | OCH₂ | 2-quinolinyl | 4-F | H |
| 357 | 4-methoxy-phenyl | OCH₂ | 2-quinolinyl | 4-Cl | H |
| 358 | 4-methoxy-phenyl | OCH₂ | 2-quinolinyl | 4-CN | H |
| 359 | 4-methoxy-phenyl | OCH₂ | 2-quinolinyl | 4-NO2 | H |
| 360 | 4-methoxy-phenyl | OCH₂ | 2-quinolinyl | 4-OMe | H |
| 361 | 4-methoxy-phenyl | OCH₂ | 2-quinolinyl | 4-Me | H |
| 362 | 4-methoxy-phenyl | OCH₂ | 2-quinolinyl | 4-Et | H |
| 363 | 4-methoxy-phenyl | OCH₂ | 2-quinolinyl | 4-iPr | H |
| 364 | 4-methoxy-phenyl | OCH₂ | 2-quinolinyl | 4-tBu | H |
| 365 | 4-methoxy-phenyl | OCH₂ | 2-quinolinyl | 4-CF3 | H |
| 366 | 4-methoxy-phenyl | OCH₂ | 2-quinolinyl | 4-SO2Me | H |
| 367 | 4-methoxy-phenyl | OCH₂ | 2-quinolinyl | 4-SO2Et | H |
| 368 | 4-methoxy-phenyl | OCH₂ | 2-quinolinyl | 4-SO2iPr | H |
| 369 | 4-methoxy-phenyl | OCH₂ | 2-quinolinyl | 4-OCF3 | H |
| 370 | 4-methoxy-phenyl | OCH₂ | 2-quinolinyl | 4-OCH2CF3 | H |
| 371 | 4-methoxy-phenyl | OCH₂ | 2-quinolinyl | 4-NHMe | H |
| 372 | 4-methoxy-phenyl | OCH₂ | 2-quinolinyl | 4-NMe2 | H |
| 373 | 4-methoxy-phenyl | OCH₂ | 2-quinolinyl | 4-cyclopropyl | H |
| 374 | 4-methoxy-phenyl | OCH₂ | 2-quinolinyl | 4-OEt | H |
| 375 | 4-methoxy-phenyl | OCH₂ | 2-quinolinyl | 4-OiPr | H |
| 376 | 4-methoxy-phenyl | OCH₂ | 2-quinolinyl | 4-CH2-cyclopropyl | H |
| 377 | 4-methoxy-phenyl | OCH₂ | 2-quinolinyl | 4-SMe | H |
| 378 | 4-methoxy-phenyl | OCH₂ | 2-quinolinyl | 4-SEt | H |
| 379 | 4-methoxy-phenyl | OCH₂ | 2-quinolinyl | 4-SiPr | H |
| 380 | 4-pyridinyl | OCH₂ | 2-quinolinyl | H | H |
| 381 | 4-pyridinyl | OCH₂ | 2-quinolinyl | 3-F | H |
| 382 | 4-pyridinyl | OCH₂ | 2-quinolinyl | 3-Cl | H |
| 383 | 4-pyridinyl | OCH₂ | 2-quinolinyl | 3-CN | H |
| 384 | 4-pyridinyl | OCH₂ | 2-quinolinyl | 3-NO₂ | H |
| 385 | 4-pyridinyl | OCH₂ | 2-quinolinyl | 3-OMe | H |
| 386 | 4-pyridinyl | OCH₂ | 2-quinolinyl | 3-OEt | H |
| 387 | 4-pyridinyl | OCH₂ | 2-quinolinyl | 3-Me | H |
| 388 | 4-pyridinyl | OCH₂ | 2-quinolinyl | 3-Et | H |
| 389 | 4-pyridinyl | OCH₂ | 2-quinolinyl | 3-ⁱPr | H |
| 390 | 4-pyridinyl | OCH₂ | 2-quinolinyl | 3-^{t}Bu | H |
| 391 | 4-pyridinyl | OCH₂ | 2-quinolinyl | 3-CF₃ | H |
| 392 | 4-pyridinyl | OCH₂ | 2-quinolinyl | 3-SO₂Me | H |
| 393 | 4-pyridinyl | OCH₂ | 2-quinolinyl | 3-SO₂Et | H |
| 394 | 4-pyridinyl | OCH₂ | 2-quinolinyl | 3-SO₂ⁱPr | H |
| 395 | 4-pyridinyl | OCH₂ | 2-quinolinyl | 3-OCF₃ | H |
| 396 | 4-pyridinyl | OCH₂ | 2-quinolinyl | 3-OCH₂CF₃ | H |
| 397 | 4-pyridinyl | OCH₂ | 2-quinolinyl | 3-NHMe | H |
| 398 | 4-pyridinyl | OCH₂ | 2-quinolinyl | 3-NMe₂ | H |
| 399 | 4-pyridinyl | OCH₂ | 2-quinolinyl | 3-OⁱPr | H |
| 400 | 4-pyridinyl | OCH₂ | 2-quinolinyl | 3-CH₂-cyclopropyl | H |
| 401 | 4-pyridinyl | OCH₂ | 2-quinolinyl | 3-SMe | H |
| 402 | 4-pyridinyl | OCH₂ | 2-quinolinyl | 3-SEt | H |
| 403 | 4-pyridinyl | OCH₂ | 2-quinolinyl | 3-SⁱPr | H |
| 404 | 4-pyridinyl | OCH₂ | 2-quinolinyl | 4-F | H |
| 405 | 4-pyridinyl | OCH₂ | 2-quinolinyl | 4-Cl | H |
| 406 | 4-pyridinyl | OCH₂ | 2-quinolinyl | 4-CN | H |
| 407 | 4-pyridinyl | OCH₂ | 2-quinolinyl | 4-OMe | H |
| 408 | 4-pyridinyl | OCH₂ | 2-quinolinyl | 4-Me | H |
| 409 | 4-pyridinyl | OCH₂ | 2-quinolinyl | 4-Et | H |
| 410 | 4-pyridinyl | OCH₂ | 2-quinolinyl | 4-ⁱPr | H |
| 411 | 4-pyridinyl | OCH₂ | 2-quinolinyl | 4-^{t}Bu | H |
| 412 | 4-pyridinyl | OCH₂ | 2-quinolinyl | 4-CF₃ | H |
| 413 | 4-pyridinyl | OCH₂ | 2-quinolinyl | 4-SO₂Me | H |
| 414 | 4-pyridinyl | OCH₂ | 2-quinolinyl | 4-SO₂Et | H |
| 415 | 4-pyridinyl | OCH₂ | 2-quinolinyl | 4-SO₂ⁱPr | H |
| 416 | 4-pyridinyl | OCH₂ | 2-quinolinyl | 4-OCF₃ | H |
| 417 | 4-pyridinyl | OCH₂ | 2-quinolinyl | 4-OCH₂CF₃ | H |
| 418 | 4-pyridinyl | OCH₂ | 2-quinolinyl | 4-NHMe | H |
| 419 | 4-pyridinyl | OCH₂ | 2-quinolinyl | 4-NMe₂ | H |
| 420 | 4-pyridinyl | OCH₂ | 2-quinolinyl | 4-cyclopropyl | H |
| 421 | 4-pyridinyl | OCH₂ | 2-quinolinyl | 4-OEt | H |
| 422 | 4-pyridinyl | OCH₂ | 2-quinolinyl | 4-OⁱPr | H |
| 423 | 4-pyridinyl | OCH₂ | 2-quinolinyl | 4-CH₂-cyclopropyl | H |
| 424 | 4-pyridinyl | OCH₂ | 2-quinolinyl | 4-SMe | H |
| 425 | 4-pyridinyl | OCH₂ | 2-quinolinyl | 4-SEt | H |
| 426 | 4-pyridinyl | OCH₂ | 2-quinolinyl | 4-SⁱPr | H |
| 427 | 4-pyridinyl | OCH₂ | 2-quinolinyl | 3-F | 4-F |
| 428 | 4-pyridinyl | OCH₂ | 2-quinolinyl | 3-F | 4-OMe |
| 429 | 4-pyridinyl | OCH₂ | 2-quinolinyl | 3-F | 4-Cl |
| 430 | 4-pyridinyl | OCH₂ | 2-quinolinyl | 3-Cl | 4-OMe |
| 431 | 4-pyridinyl | OCH₂ | 2-quinolinyl | 3-Cl | 4-CN |
| 432 | 4-pyridinyl | OCH₂ | 2-quinolinyl | 3-OMe | 4-F |
| 433 | 4-pyridinyl | OCH₂ | 2-quinolinyl | 3-CN | 4-OMe |
| 434 | 4-pyridinyl | OCH₂ | 2-quinolinyl | 3-CF₃ | 4-CN |
| 435 | 4-pyridinyl | OCH₂ | 2-quinolinyl | 3-NMe₂ | 4-F |
| 436 | 4-pyridinyl | OCH₂ | 2-quinolinyl | 3-F | 4-NMe₂ |
| 437 | 4-pyridinyl | OCH₂ | 2-quinolinyl | 3-O-cyclopropyl | 4-CN |
| 438 | 4-pyridinyl | OCH₂ | 2-quinolinyl | 3-Cl | 4-Cl |
| 439 | 4-pyridinyl | OCH₂ | 2-quinolinyl | 3-cyclopropyl | H |
| 442 | 4-pyridinyl | OCH₂ | 2-quinolinyl | 4-NO₂ | H |
| 443 | ⁱPr | OCH₂ | 2-quinolinyl | H | H |
| 444 | Me | OCH₂ | 2-quinolinyl | H | H |
| 445 | morpholinyl | OCH₂ | 2-quinolinyl | H | H |
| 446 | morpholinyl | OCH₂ | 2-quinolinyl | 3-F | H |
| 447 | morpholinyl | OCH₂ | 2-quinolinyl | 3-Cl | H |
| 448 | morpholinyl | OCH₂ | 2-quinolinyl | 3-CN | H |
| 449 | morpholinyl | OCH₂ | 2-quinolinyl | 3-NO₂ | H |
| 450 | morpholinyl | OCH₂ | 2-quinolinyl | 3-OMe | H |
| 451 | morpholinyl | OCH₂ | 2-quinolinyl | 3-OEt | H |
| 452 | morpholinyl | OCH₂ | 2-quinolinyl | 3-Me | H |
| 453 | morpholinyl | OCH₂ | 2-quinolinyl | 3-Et | H |
| 454 | morpholinyl | OCH₂ | 2-quinolinyl | 3-ⁱPr | H |
| 455 | morpholinyl | OCH₂ | 2-quinolinyl | 3-^{t}Bu | H |
| 456 | morpholinyl | OCH₂ | 2-quinolinyl | 3-CF₃ | H |
| 457 | morpholinyl | OCH₂ | 2-quinolinyl | 3-SO₂Me | H |
| 458 | morpholinyl | OCH₂ | 2-quinolinyl | 3-SO₂Et | H |
| 459 | morpholinyl | OCH₂ | 2-quinolinyl | 3-SO₂ⁱPr | H |
| 460 | morpholinyl | OCH₂ | 2-quinolinyl | 3-OCF₃ | H |
| 461 | morpholinyl | OCH₂ | 2-quinolinyl | 3-OCH₂CF₃ | H |
| 462 | morpholinyl | OCH₂ | 2-quinolinyl | 3-NHMe | H |
| 463 | morpholinyl | OCH₂ | 2-quinolinyl | 3-NMe₂ | H |
| 464 | morpholinyl | OCH₂ | 2-quinolinyl | 3-OⁱPr | H |
| 465 | morpholinyl | OCH₂ | 2-quinolinyl | 3-CH₂-cyclopropyl | H |
| 466 | morpholinyl | OCH₂ | 2-quinolinyl | 3-SMe | H |
| 467 | morpholinyl | OCH₂ | 2-quinolinyl | 3-SEt | H |
| 468 | morpholinyl | OCH₂ | 2-quinolinyl | 3-SⁱPr | H |
| 469 | morpholinyl | OCH₂ | 2-quinolinyl | 4-F | H |
| 470 | morpholinyl | OCH₂ | 2-quinolinyl | 4-Cl | H |
| 471 | morpholinyl | OCH₂ | 2-quinolinyl | 4-CN | H |
| 472 | morpholinyl | OCH₂ | 2-quinolinyl | 4-OMe | H |
| 473 | morpholinyl | OCH₂ | 2-quinolinyl | 4-Me | H |
| 474 | morpholinyl | OCH₂ | 2-quinolinyl | 4-Et | H |
| 475 | morpholinyl | OCH₂ | 2-quinolinyl | 4-ⁱPr | H |
| 476 | morpholinyl | OCH₂ | 2-quinolinyl | 4-^{t}Bu | H |
| 477 | morpholinyl | OCH₂ | 2-quinolinyl | 4-CF₃ | H |
| 478 | morpholinyl | OCH₂ | 2-quinolinyl | 4-SO₂Me | H |
| 479 | morpholinyl | OCH₂ | 2-quinolinyl | 4-SO₂Et | H |
| 480 | morpholinyl | OCH₂ | 2-quinolinyl | 4-SO₂ⁱPr | H |
| 481 | morpholinyl | OCH₂ | 2-quinolinyl | 4-OCF₃ | H |
| 482 | morpholinyl | OCH₂ | 2-quinolinyl | 4-OCH₂CF₃ | H |
| 483 | morpholinyl | OCH₂ | 2-quinolinyl | 4-NHMe | H |
| 484 | morpholinyl | OCH₂ | 2-quinolinyl | 4-NMe₂ | H |
| 485 | morpholinyl | OCH₂ | 2-quinolinyl | 4-cyclopropyl | H |
| 486 | morpholinyl | OCH₂ | 2-quinolinyl | 4-OEt | H |
| 487 | morpholinyl | OCH₂ | 2-quinolinyl | 4-OⁱPr | H |
| 488 | morpholinyl | OCH₂ | 2-quinolinyl | 4-CH₂-cyclopropyl | H |
| 489 | morpholinyl | OCH₂ | 2-quinolinyl | 4-SMe | H |
| 490 | morpholinyl | OCH₂ | 2-quinolinyl | 4-SEt | H |
| 491 | morpholinyl | OCH₂ | 2-quinolinyl | 4-SⁱPr | H |
| 492 | N-piperazinyl | OCH₂ | 2-quinolinyl | H | H |
| 493 | piperazinyl | OCH₂ | 2-quinolinyl | H | H |
| 494 | piperidinyl | OCH₂ | 2-quinolinyl | H | H |
| 495 | 3-pyridinyl | OCH₂ | 2-quinoxalinyl | H | H |
| 496 | 4-pyridinyl | OCH₂ | 2-quinoxalinyl | H | H |
| 497 | morpholinyl | OCH₂ | 2-quinoxalinyl | H | H |
| 498 | 3-pyridinyl | OCH₂ | 5,6,7,8-tetrahydro-2-quinolyl | H | H |
| 499 | 4-pyridinyl | OCH₂ | 5,6,7,8-tetrahydro-2-quinolyl | H | H |
| 500 | morpholinyl | OCH₂ | 5,6,7,8-tetrahydro-2-quinolyl | H | H |
| 501 | 4-pyridinyl | OCH₂ | 5-methylpyridin-2-yl | H | H |
| 502 | 4-pyridinyl | OCH₂ | 5-methylpyridin-2-yl | 3-F | H |
| 503 | 4-pyridinyl | OCH₂ | 5-methylpyridin-2-yl | 3-Cl | H |
| 504 | 4-pyridinyl | OCH₂ | 5-methylpyridin-2-yl | 3-CN | H |
| 505 | 4-pyridinyl | OCH₂ | 5-methylpyridin-2-yl | 3-NO₂ | H |
| 506 | 4-pyridinyl | OCH₂ | 5-methylpyridin-2-yl | 3-OMe | H |
| 507 | 4-pyridinyl | OCH₂ | 5-methylpyridin-2-yl | 3-OEt | H |
| 508 | 4-pyridinyl | OCH₂ | 5-methylpyridin-2-yl | 3-Me | H |
| 509 | 4-pyridinyl | OCH₂ | 5-methylpyridin-2-yl | 3-Et | H |
| 510 | 4-pyridinyl | OCH₂ | 5-methylpyridin-2-yl | 3-ⁱPr | H |
| 511 | 4-pyridinyl | OCH₂ | 5-methylpyridin-2-yl | 3-^{t}Bu | H |
| 512 | 4-pyridinyl | OCH₂ | 5-methylpyridin-2-yl | 3-CF₃ | H |
| 513 | 4-pyridinyl | OCH₂ | 5-methylpyridin-2-yl | 3-SO₂Me | H |
| 514 | 4-pyridinyl | OCH₂ | 5-methylpyridin-2-yl | 3-SO₂Et | H |
| 515 | 4-pyridinyl | OCH₂ | 5-methylpyridin-2-yl | 3-SO₂ⁱPr | H |
| 516 | 4-pyridinyl | OCH₂ | 5-methylpyridin-2-yl | 3-OCF₃ | H |
| 517 | 4-pyridinyl | OCH₂ | 5-methylpyridin-2-yl | 3-OCH₂CF₃ | H |
| 518 | 4-pyridinyl | OCH₂ | 5-methylpyridin-2-yl | 3-NHMe | H |
| 519 | 4-pyridinyl | OCH₂ | 5-methylpyridin-2-yl | 3-NMe₂ | H |
| 520 | 4-pyridinyl | OCH₂ | 5-methylpyridin-2-yl | 3-OⁱPr | H |
| 521 | 4-pyridinyl | OCH₂ | 5-methylpyridin-2-yl | 3-CH₂-cyclopropyl | H |
| 522 | 4-pyridinyl | OCH₂ | 5-methylpyridin-2-yl | 3-SMe | H |
| 523 | 4-pyridinyl | OCH₂ | 5-methylpyridin-2-yl | 3-SEt | H |
| 524 | 4-pyridinyl | OCH₂ | 5-methylpyridin-2-yl | 3-SⁱPr | H |
| 525 | 4-pyridinyl | OCH₂ | 5-methylpyridin-2-yl | 4-F | H |
| 526 | 4-pyridinyl | OCH₂ | 5-methylpyridin-2-yl | 4-Cl | H |
| 527 | 4-pyridinyl | OCH₂ | 5-methylpyridin-2-yl | 4-CN | H |
| 528 | 4-pyridinyl | OCH₂ | 5-methylpyridin-2-yl | 4-OMe | H |
| 529 | 4-pyridinyl | OCH₂ | 5-methylpyridin-2-yl | 4-Me | H |
| 530 | 4-pyridinyl | OCH₂ | 5-methylpyridin-2-yl | 4-Et | H |
| 531 | 4-pyridinyl | OCH₂ | 5-methylpyridin-2-yl | 4-ⁱPr | H |
| 532 | 4-pyridinyl | OCH₂ | 5-methylpyridin-2-yl | 4-^{t}Bu | H |
| 533 | 4-pyridinyl | OCH₂ | 5-methylpyridin-2-yl | 4-CF₃ | H |
| 534 | 4-pyridinyl | OCH₂ | 5-methylpyridin-2-yl | 4-SO₂Me | H |
| 535 | 4-pyridinyl | OCH₂ | 5-methylpyridin-2-yl | 4-SO₂Et | H |
| 536 | 4-pyridinyl | OCH₂ | 5-methylpyridin-2-yl | 4-SO₂ⁱPr | H |
| 537 | 4-pyridinyl | OCH₂ | 5-methylpyridin-2-yl | 4-OCF₃ | H |
| 538 | 4-pyridinyl | OCH₂ | 5-methylpyridin-2-yl | 4-OCH₂CF₃ | H |
| 539 | 4-pyridinyl | OCH₂ | 5-methylpyridin-2-yl | 4-NHMe | H |
| 540 | 4-pyridinyl | OCH₂ | 5-methylpyridin-2-yl | 4-NMe₂ | H |
| 541 | 4-pyridinyl | OCH₂ | 5-methylpyridin-2-yl | 4-cyclopropyl | H |
| 542 | 4-pyridinyl | OCH₂ | 5-methylpyridin-2-yl | 4-OEt | H |
| 543 | 4-pyridinyl | OCH₂ | 5-methylpyridin-2-yl | 4-OⁱPr | H |
| 544 | 4-pyridinyl | OCH₂ | 5-methylpyridin-2-yl | 4-CH₂-cyclopropyl | H |
| 545 | 4-pyridinyl | OCH₂ | 5-methylpyridin-2-yl | 4-SMe | H |
| 546 | 4-pyridinyl | OCH₂ | 5-methylpyridin-2-yl | 4-SEt | H |
| 547 | 4-pyridinyl | OCH₂ | 5-methylpyridin-2-yl | 4-SⁱPr | H |
| 548 | 4-pyridinyl | OCH₂ | 5-methylpyridin-2-yl | 3-F | 4-F |
| 549 | 4-pyridinyl | OCH₂ | 5-methylpyridin-2-yl | 3-F | 4-OMe |
| 550 | 4-pyridinyl | OCH₂ | 5-methylpyridin-2-yl | 3-F | 4-Cl |
| 551 | 4-pyridinyl | OCH₂ | 5-methylpyridin-2-yl | 3-Cl | 4-OMe |
| 552 | 4-pyridinyl | OCH₂ | 5-methylpyridin-2-yl | 3-Cl | 4-CN |
| 553 | 4-pyridinyl | OCH₂ | 5-methylpyridin-2-yl | 3-OMe | 4-F |
| 554 | 4-pyridinyl | OCH₂ | 5-methylpyridin-2-yl | 3-CN | 4-OMe |
| 555 | 4-pyridinyl | OCH₂ | 5-methylpyridin-2-yl | 3-CF₃ | 4-CN |
| 556 | 4-pyridinyl | OCH₂ | 5-methylpyridin-2-yl | 3-NMe₂ | 4-F |
| 557 | 4-pyridinyl | OCH₂ | 5-methylpyridin-2-yl | 3-F | 4-NMe₂ |
| 558 | 4-pyridinyl | OCH₂ | 5-methylpyridin-2-yl | 3-O-cyclopropyl | 4-CN |
| 559 | 4-pyridinyl | OCH₂ | 5-methylpyridin-2-yl | 3-Cl | 4-Cl |
| 560 | 4-pyridinyl | OCH₂ | 6-fluoroquinolin-2-yl | H | H |
| 561 | 4-pyridinyl | OCH₂ | 6-fluoroquinolin-2-yl | 3-F | H |
| 562 | 4-pyridinyl | OCH₂ | 6-fluoroquinolin-2-yl | 3-Cl | H |
| 563 | 4-pyridinyl | OCH₂ | 6-fluoroquinolin-2-yl | 3-CN | H |
| 564 | 4-pyridinyl | OCH₂ | 6-fluoroquinolin-2-yl | 3-NO₂ | H |
| 565 | 4-pyridinyl | OCH₂ | 6-fluoroquinolin-2-yl | 3-OMe | H |
| 566 | 4-pyridinyl | OCH₂ | 6-fluoroquinolin-2-yl | 3-OEt | H |
| 567 | 4-pyridinyl | OCH₂ | 6-fluoroquinolin-2-yl | 3-Me | H |
| 568 | 4-pyridinyl | OCH₂ | 6-fluoroquinolin-2-yl | 3-Et | H |
| 569 | 4-pyridinyl | OCH₂ | 6-fluoroquinolin-2-yl | 3-ⁱPr | H |
| 570 | 4-pyridinyl | OCH₂ | 6-fluoroquinolin-2-yl | 3-^{t}Bu | H |
| 571 | 4-pyridinyl | OCH₂ | 6-fluoroquinolin-2-yl | 3-CF₃ | H |
| 572 | 4-pyridinyl | OCH₂ | 6-fluoroquinolin-2-yl | 3-SO₂Me | H |
| 573 | 4-pyridinyl | OCH₂ | 6-fluoroquinolin-2-yl | 3-SO₂Et | H |
| 574 | 4-pyridinyl | OCH₂ | 6-fluoroquinolin-2-yl | 3-SO₂ⁱPr | H |
| 575 | 4-pyridinyl | OCH₂ | 6-fluoroquinolin-2-yl | 3-OCF₃ | H |
| 576 | 4-pyridinyl | OCH₂ | 6-fluoroquinolin-2-yl | 3-OCH₂CF₃ | H |
| 577 | 4-pyridinyl | OCH₂ | 6-fluoroquinolin-2-yl | 3-NHMe | H |
| 578 | 4-pyridinyl | OCH₂ | 6-fluoroquinolin-2-yl | 3-NMe₂ | H |
| 579 | 4-pyridinyl | OCH₂ | 6-fluoroquinolin-2-yl | 3-OⁱPr | H |
| 580 | 4-pyridinyl | OCH₂ | 6-fluoroquinolin-2-yl | 3-CH₂-cyclopropyl | H |
| 581 | 4-pyridinyl | OCH₂ | 6-fluoroquinolin-2-yl | 3-SMe | H |
| 582 | 4-pyridinyl | OCH₂ | 6-fluoroquinolin-2-yl | 3-SEt | H |
| 583 | 4-pyridinyl | OCH₂ | 6-fluoroquinolin-2-yl | 3-SⁱPr | H |
| 584 | 4-pyridinyl | OCH₂ | 6-fluoroquinolin-2-yl | 4-F | H |
| 585 | 4-pyridinyl | OCH₂ | 6-fluoroquinolin-2-yl | 4-Cl | H |
| 586 | 4-pyridinyl | OCH₂ | 6-fluoroquinolin-2-yl | 4-CN | H |
| 587 | 4-pyridinyl | OCH₂ | 6-fluoroquinolin-2-yl | 4-OMe | H |
| 588 | 4-pyridinyl | OCH₂ | 6-fluoroquinolin-2-yl | 4-Me | H |
| 589 | 4-pyridinyl | OCH₂ | 6-fluoroquinolin-2-yl | 4-Et | H |
| 590 | 4-pyridinyl | OCH₂ | 6-fluoroquinolin-2-yl | 4-ⁱPr | H |
| 591 | 4-pyridinyl | OCH₂ | 6-fluoroquinolin-2-yl | 4-^{t}Bu | H |
| 592 | 4-pyridinyl | OCH₂ | 6-fluoroquinolin-2-yl | 4-CF₃ | H |
| 593 | 4-pyridinyl | OCH₂ | 6-fluoroquinolin-2-yl | 4-SO₂Me | H |
| 594 | 4-pyridinyl | OCH₂ | 6-fluoroquinolin-2-yl | 4-SO₂Et | H |
| 595 | 4-pyridinyl | OCH₂ | 6-fluoroquinolin-2-yl | 4-SO₂ⁱPr | H |
| 596 | 4-pyridinyl | OCH₂ | 6-fluoroquinolin-2-yl | 4-OCF₃ | H |
| 597 | 4-pyridinyl | OCH₂ | 6-fluoroquinolin-2-yl | 4-OCH₂CF₃ | H |
| 598 | 4-pyridinyl | OCH₂ | 6-fluoroquinolin-2-yl | 4-NHMe | H |
| 599 | 4-pyridinyl | OCH₂ | 6-fluoroquinolin-2-yl | 4-NMe₂ | H |
| 600 | 4-pyridinyl | OCH₂ | 6-fluoroquinolin-2-yl | 4-cyclopropyl | H |
| 601 | 4-pyridinyl | OCH₂ | 6-fluoroquinolin-2-yl | 4-OEt | H |
| 602 | 4-pyridinyl | OCH₂ | 6-fluoroquinolin-2-yl | 4-OⁱPr | H |
| 603 | 4-pyridinyl | OCH₂ | 6-fluoroquinolin-2-yl | 4-CH₂-cyclopropyl | H |
| 604 | 4-pyridinyl | OCH₂ | 6-fluoroquinolin-2-yl | 4-SMe | H |
| 605 | 4-pyridinyl | OCH₂ | 6-fluoroquinolin-2-yl | 4-SEt | H |
| 606 | 4-pyridinyl | OCH₂ | 6-fluoroquinolin-2-yl | 4-SⁱPr | H |
| 607 | 4-pyridinyl | OCH₂ | 6-fluoroquinolin-2-yl | 3-F | 4-F |
| 608 | 4-pyridinyl | OCH₂ | 6-fluoroquinolin-2-yl | 3-F | 4-OMe |
| 609 | 4-pyridinyl | OCH₂ | 6-fluoroquinolin-2-yl | 3-F | 4-Cl |
| 610 | 4-pyridinyl | OCH₂ | 6-fluoroquinolin-2-yl | 3-Cl | 4-OMe |
| 611 | 4-pyridinyl | OCH₂ | 6-fluoroquinolin-2-yl | 3-Cl | 4-CN |
| 612 | 4-pyridinyl | OCH₂ | 6-fluoroquinolin-2-yl | 3-OMe | 4-F |
| 613 | 4-pyridinyl | OCH₂ | 6-fluoroquinolin-2-yl | 3-CN | 4-OMe |
| 614 | 4-pyridinyl | OCH₂ | 6-fluoroquinolin-2-yl | 3-CF₃ | 4-CN |
| 615 | 4-pyridinyl | OCH₂ | 6-fluoroquinolin-2-yl | 3-NMe₂ | 4-F |
| 616 | 4-pyridinyl | OCH₂ | 6-fluoroquinolin-2-yl | 3-F | 4-NMe₂ |
| 617 | 4-pyridinyl | OCH₂ | 6-fluoroquinolin-2-yl | 3-O-cyclopropyl | 4-CN |
| 618 | 4-pyridinyl | OCH₂ | 6-fluoroquinolin-2-yl | 3-Cl | 4-Cl |
| 619 | 4-pyridinyl | OCH₂ | imidazo[1,2-a]pyridin-2-yl | H | H |
| 620 | 4-pyridinyl | OCH₂ | imidazo[1,2-a]pyridin-2-yl | 3-F | H |
| 621 | 4-pyridinyl | OCH₂ | imidazo[1,2-a]pyridin-2-yl | 3-Cl | H |
| 622 | 4-pyridinyl | OCH₂ | imidazo[1,2-a]pyridin-2-yl | 3-CN | H |
| 623 | 4-pyridinyl | OCH₂ | imidazo[1,2-a]pyridin-2-yl | 3-NO₂ | H |
| 624 | 4-pyridinyl | OCH₂ | imidazo[1,2-a]pyridin-2-yl | 3-OMe | H |
| 625 | 4-pyridinyl | OCH₂ | imidazo[1,2-a]pyridin-2-yl | 3-OEt | H |
| 626 | 4-pyridinyl | OCH₂ | imidazo[1,2-a]pyridin-2-yl | 3-Me | H |
| 627 | 4-pyridinyl | OCH₂ | imidazo[1,2-a]pyridin-2-yl | 3-Et | H |
| 628 | 4-pyridinyl | OCH₂ | imidazo[1,2-a]pyridin-2-yl | 3-ⁱPr | H |
| 629 | 4-pyridinyl | OCH₂ | imidazo[1,2-a]pyridin-2-yl | 3-^{t}Bu | H |
| 630 | 4-pyridinyl | OCH₂ | imidazo[1,2-a]pyridin-2-yl | 3-CF₃ | H |
| 631 | 4-pyridinyl | OCH₂ | imidazo[1,2-a]pyridin-2-yl | 3-SO₂Me | H |
| 632 | 4-pyridinyl | OCH₂ | imidazo[1,2-a]pyridin-2-yl | 3-SO₂Et | H |
| 633 | 4-pyridinyl | OCH₂ | imidazo[1,2-a]pyridin-2-yl | 3-SO₂ⁱPr | H |
| 634 | 4-pyridinyl | OCH₂ | imidazo[1,2-a]pyridin-2-yl | 3-OCF₃ | H |
| 635 | 4-pyridinyl | OCH₂ | imidazo[1,2-a]pyridin-2-yl | 3-OCH₂CF₃ | H |
| 636 | 4-pyridinyl | OCH₂ | imidazo[1,2-a]pyridin-2-yl | 3-NHMe | H |
| 637 | 4-pyridinyl | OCH₂ | imidazo[1,2-a]pyridin-2-yl | 3-NMe₂ | H |
| 638 | 4-pyridinyl | OCH₂ | imidazo[1,2-a]pyridin-2-yl | 3-OⁱPr | H |
| 639 | 4-pyridinyl | OCH₂ | imidazo[1,2-a]pyridin-2-yl | 3-CH₂-cyclopropyl | H |
| 640 | 4-pyridinyl | OCH₂ | imidazo[1,2-a]pyridin-2-yl | 3-SMe | H |
| 641 | 4-pyridinyl | OCH₂ | imidazo[1,2-a]pyridin-2-yl | 3-SEt | H |
| 642 | 4-pyridinyl | OCH₂ | imidazo[1,2-a]pyridin-2-yl | 3-SⁱPr | H |
| 643 | 4-pyridinyl | OCH₂ | imidazo[1,2-a]pyridin-2-yl | 4-F | H |
| 644 | 4-pyridinyl | OCH₂ | imidazo[1,2-a]pyridin-2-yl | 4-Cl | H |
| 645 | 4-pyridinyl | OCH₂ | imidazo[1,2-a]pyridin-2-yl | 4-CN | H |
| 646 | 4-pyridinyl | OCH₂ | imidazo[1,2-a]pyridin-2-yl | 4-OMe | H |
| 647 | 4-pyridinyl | OCH₂ | imidazo[1,2-a]pyridin-2-yl | 4-Me | H |
| 648 | 4-pyridinyl | OCH₂ | imidazo[1,2-a]pyridin-2-yl | 4-Et | H |
| 649 | 4-pyridinyl | OCH₂ | imidazo[1,2-a]pyridin-2-yl | 4-ⁱPr | H |
| 650 | 4-pyridinyl | OCH₂ | imidazo[1,2-a]pyridin-2-yl | 4-^{t}Bu | H |
| 651 | 4-pyridinyl | OCH₂ | imidazo[1,2-a]pyridin-2-yl | 4-CF₃ | H |
| 652 | 4-pyridinyl | OCH₂ | imidazo[1,2-a]pyridin-2-yl | 4-SO₂Me | H |
| 653 | 4-pyridinyl | OCH₂ | imidazo[1,2-a]pyridin-2-yl | 4-SO₂Et | H |
| 654 | 4-pyridinyl | OCH₂ | imidazo[1,2-a]pyridin-2-yl | 4-SO₂ⁱPr | H |
| 655 | 4-pyridinyl | OCH₂ | imidazo[1,2-a]pyridin-2-yl | 4-OCF₃ | H |
| 656 | 4-pyridinyl | OCH₂ | imidazo[1,2-a]pyridin-2-yl | 4-OCH₂CF₃ | H |
| 657 | 4-pyridinyl | OCH₂ | imidazo[1,2-a]pyridin-2-yl | 4-NHMe | H |
| 658 | 4-pyridinyl | OCH₂ | imidazo[1,2-a]pyridin-2-yl | 4-NMe₂ | H |
| 659 | 4-pyridinyl | OCH₂ | imidazo[1,2-a]pyridin-2-yl | 4-cyclopropyl | H |
| 660 | 4-pyridinyl | OCH₂ | imidazo[1,2-a]pyridin-2-yl | 4-OEt | H |
| 661 | 4-pyridinyl | OCH₂ | imidazo[1,2-a]pyridin-2-yl | 4-OⁱPr | H |
| 662 | 4-pyridinyl | OCH₂ | imidazo[1,2-a]pyridin-2-yl | 4-CH₂-cyclopropyl | H |
| 663 | 4-pyridinyl | OCH₂ | imidazo[1,2-a]pyridin-2-yl | 4-SMe | H |
| 664 | 4-pyridinyl | OCH₂ | imidazo[1,2-a]pyridin-2-yl | 4-SEt | H |
| 665 | 4-pyridinyl | OCH₂ | imidazo[1,2-a]pyridin-2-yl | 4-SⁱPr | H |
| 1854 | 4-pyridinyl | OCH₂ | 2-quinoline | 3-CHO | H |
| 1855 | 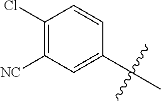 | OCH₂ | 2-quinoline | H | H |
| 1856 | 4-pyridinyl | OCH₂ | 2-quinoline | 5-F | H |
| 1857 | 4-pyridinyl | OCH₂ | 2-quinoline | 3-(1,3-dioxan-2-yl) | H |
| 1858 | 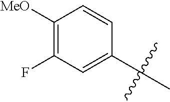 | OCH₂ | 2-quinoline | H | H |
| 1859 | 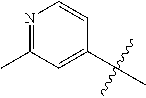 | OCH₂ | 2-quinoline | H | H |
| 1860 | 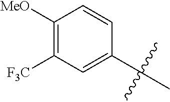 | OCH₂ | 2-quinoline | H | H |
| 1861 | 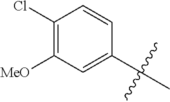 | OCH₂ | 2-quinoline | H | H |
| 1862 | 4-pyridinyl | OCH₂ | 2-quinoline | 3-OMe | 4-OMe |
| 1863 | phenyl | OCH₂ | 2-quinoline | 3-OMe | 4-OMe |
| 1864 | 4-pyridinyl | OCH₂ | 2-quinoline | 3-(C(O)-morpholinyl) | H |
| 1865 | 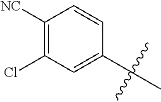 | OCH₂ | 2-quinoline | H | H |
| 1866 | n-propyl | OCH₂ | 2-quinoline | H | H |
| 1867 | 4-pyridinyl | OCH₂ | 2-quinoline | 5-Me | H |
| 1868 | 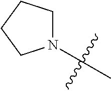 | OCH₂ | 2-quinoline | H | H |
| 1869 | 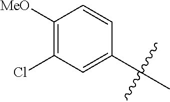 | OCH₂ | 2-quinoline | H | H |
| 1870 | 4-pyridinyl | OCH₂ | 2-quinoline | 6-CN | H |
| 1871 | 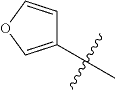 | OCH₂ | 2-quinoline | H | H |
| 1872 | 4-pyridinyl | OCH₂ | 2-quinoline | 6-Cl | H |
| 1873 | morpholinyl | OCH₂ | 2-quinoline | 3-(4-pyridyl) | H |
| 1874 | 4-pyridinyl | OCH₂ | 2-quinoline | 3-CH₂NMe₂ | H |
| 1875 | Et | OCH₂ | 2-quinoline | H | H |
| 1876 | 4-pyridinyl | OCH₂ | 2-quinoline | 5-Cl | H |
| 1877 | cyclohexyl | OCH₂ | 2-quinoline | H | H |
| 1878 | 4-pyridinyl | OCH₂ | 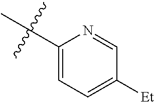 | H | H |
| 1879 | OⁱPr | OCH₂ | 2-quinoline | H | H |
| 1880 | 4-pyridinyl | OCH₂ | 2-quinoline | 3-Me | 4-Me |
| 1881 | 4-pyridinyl | OCH₂ | 2-quinoline | 3-NH₂ | H |
| 1882 | 4-pyridinyl | OCH₂ | 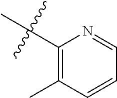 | H | H |
| 1883 | OMe | OCH₂ | 2-quinoline | H | H |
| 1884 | 4-pyridinyl | OCH₂ | 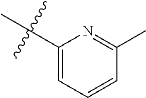 | H | H |
| 1885 | 4-pyridinyl | OCH₂ | 2-quinoline | 5-CN | H |
| 1886 | 4-pyridinyl | OCH₂ | 2-quinoline | 6-Me | H |
| 1887 | 4-pyridinyl | OCH₂ | 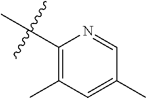 | H | H |
| 1888 | 4-pyridinyl | OCH₂ | 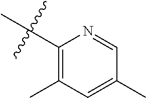 | 3-F | H |
| 1889 | 4-pyridinyl | OCH₂ | 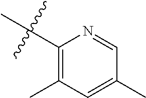 | 3-Cl | H |
| 1890 | 4-pyridinyl | OCH₂ | 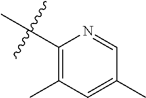 | 3-CN | H |
| 1891 | 4-pyridinyl | OCH₂ | 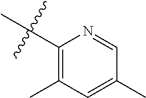 | 3-NO2 | H |
| 1892 | 4-pyridinyl | OCH₂ | 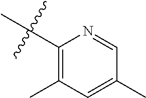 | 3-OMe | H |
| 1893 | 4-pyridinyl | OCH₂ | 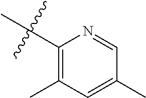 | 3-OEt | H |
| 1894 | 4-pyridinyl | OCH₂ | 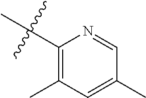 | 3-Me | H |
| 1895 | 4-pyridinyl | OCH₂ | 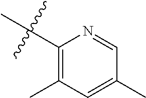 | 3-Et | H |
| 1896 | 4-pyridinyl | OCH₂ | 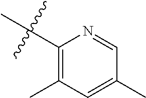 | 3-iPr | H |
| 1897 | 4-pyridinyl | OCH₂ | 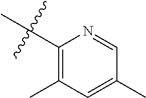 | 3-tBu | H |
| 1898 | 4-pyridinyl | OCH₂ | 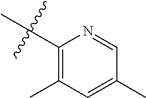 | 3-CF3 | H |
| 1899 | 4-pyridinyl | OCH₂ | 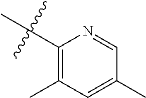 | 3-SO2Me | H |
| 1900 | 4-pyridinyl | OCH₂ | 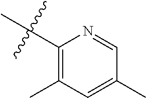 | 3-SO2Et | H |
| 1901 | 4-pyridinyl | OCH₂ | 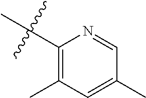 | 3-SO2iPr | H |
| 1902 | 4-pyridinyl | OCH₂ | 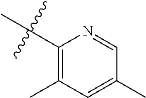 | 3-OCF3 | H |
| 1903 | 4-pyridinyl | OCH₂ | 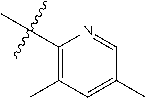 | 3-OCH2CF3 | H |
| 1904 | 4-pyridinyl | OCH₂ | 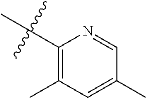 | 3-NHMe | H |
| 1905 | 4-pyridinyl | OCH₂ | 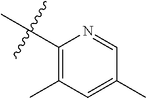 | 3-NMe2 | H |
| 1906 | 4-pyridinyl | OCH₂ | 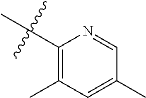 | 3-OiPr | H |
| 1907 | 4-pyridinyl | OCH₂ | 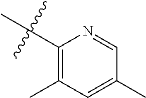 | 3-CH2-cyclopropyl | H |
| 1908 | 4-pyridinyl | OCH₂ | 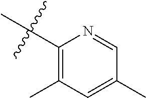 | 3-SMe | H |
| 1909 | 4-pyridinyl | OCH₂ | 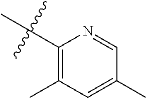 | 3-SEt | H |
| 1910 | 4-pyridinyl | OCH₂ | 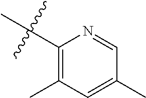 | 3-SiPr | H |
| 1911 | 4-pyridinyl | OCH₂ | 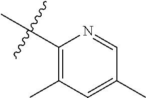 | 4-F | H |
| 1912 | 4-pyridinyl | OCH₂ | 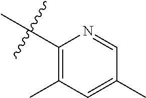 | 4-Cl | H |
| 1913 | 4-pyridinyl | OCH₂ | 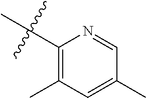 | 4-CN | H |
| 1914 | 4-pyridinyl | OCH₂ | 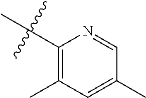 | 4-OMe | H |
| 1915 | 4-pyridinyl | OCH₂ | 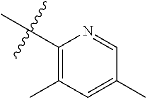 | 4-Me | H |
| 1916 | 4-pyridinyl | OCH₂ | 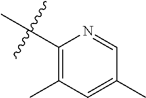 | 4-Et | H |
| 1917 | 4-pyridinyl | OCH₂ | 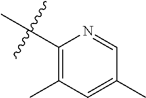 | 4-iPr | H |
| 1918 | 4-pyridinyl | OCH₂ | 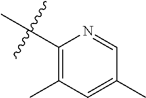 | 4-tBu | H |
| 1919 | 4-pyridinyl | OCH₂ | 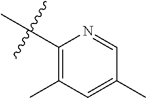 | 4-CF3 | H |
| 1920 | 4-pyridinyl | OCH₂ | 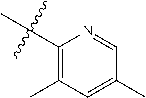 | 4-SO2Me | H |
| 1921 | 4-pyridinyl | OCH₂ | 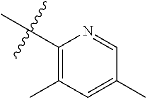 | 4-SO2Et | H |
| 1922 | 4-pyridinyl | OCH₂ | 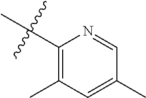 | 4-SO2iPr | H |
| 1923 | 4-pyridinyl | OCH₂ | 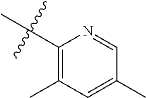 | 4-OCF3 | H |
| 1924 | 4-pyridinyl | OCH₂ | 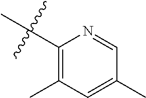 | 4-OCH2CF3 | H |
| 1925 | 4-pyridinyl | OCH₂ | 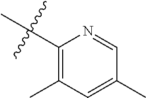 | 4-NHMe | H |
| 1926 | 4-pyridinyl | OCH₂ | 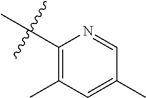 | 4-NMe2 | H |
| 1927 | 4-pyridinyl | OCH₂ | 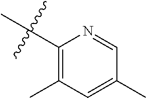 | 4-cyclopropyl | H |
| 1928 | 4-pyridinyl | OCH₂ | 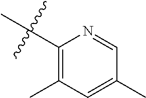 | 4-OEt | H |
| 1929 | 4-pyridinyl | OCH₂ | 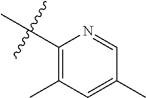 | 4-OiPr | H |
| 1930 | 4-pyridinyl | OCH₂ | 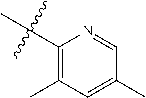 | 4-CH2-cyclopropyl | H |
| 1931 | 4-pyridinyl | OCH₂ | 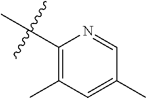 | 4-SMe | H |
| 1932 | 4-pyridinyl | OCH₂ | 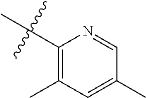 | 4-SEt | H |
| 1933 | 4-pyridinyl | OCH₂ | 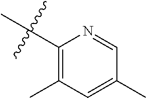 | 4-SiPr | H |
| 1934 | 4-pyridinyl | OCH₂ | 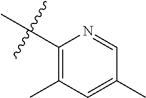 | 3-F | 4-F |
| 1935 | 4-pyridinyl | OCH₂ | 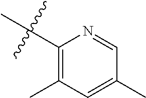 | 3-F | 4-OMe |
| 1936 | 4-pyridinyl | OCH₂ | 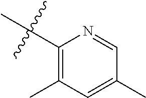 | 3-F | 4-Cl |
| 1937 | 4-pyridinyl | OCH₂ | 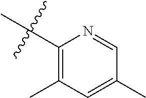 | 3-Cl | 4-OMe |
| 1938 | 4-pyridinyl | OCH₂ | 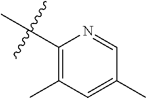 | 3-Cl | 4-CN |
| 1939 | 4-pyridinyl | OCH₂ | 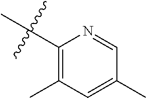 | 3-OMe | 4-F |
| 1940 | 4-pyridinyl | OCH₂ | 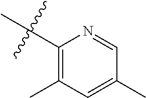 | 3-CN | 4-OMe |
| 1941 | 4-pyridinyl | OCH₂ | 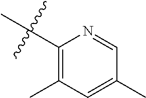 | 3-CF3 | 4-CN |
| 1942 | 4-pyridinyl | OCH₂ | 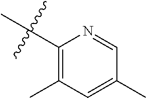 | 3-NMe2 | 4-F |
| 1943 | 4-pyridinyl | OCH₂ | 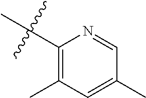 | 3-F | 4-NMe2 |
| 1944 | 4-pyridinyl | OCH₂ | 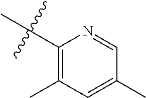 | 3-O-cyclopropyl | 4-CN |
| 1945 | 4-pyridinyl | OCH₂ | 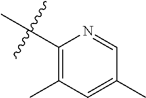 | 3-Cl | 4-Cl |
| 1946 | 4-pyridinyl | OCH₂ | 2-quinolinyl | 6-F | H |
| 1947 | 4-pyridinyl | OCH₂ | 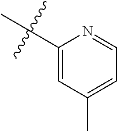 | H | H |

In a further aspect the compounds of the disclosure are embodied in with distinct examples listed in the table below taken from Formula (II):

| Ex PCT | X | Y | Z | R₁ | R₂ |
|---|---|---|---|---|---|
| 666 | 4-pyridinyl | CH₂O | 2-benzimidazolyl | H | H |
| 667 | 4-pyridinyl | CH₂O | 2-benzoxazolyl | H | H |
| 668 | 4-pyridinyl | CH₂O | 2-benzthiazolyl | H | H |
| 669 | 4-pyridinyl | CH₂O | 2-pyridinyl | H | H |
| 670 | 4-pyridinyl | CH₂O | 2-quinazolinyl | H | H |
| 671 | 4-pyridinyl | CH₂O | 2-quinolinyl | H | H |
| 672 | 4-pyridinyl | CH₂O | 2-quinolinyl | 3-F | H |
| 673 | 4-pyridinyl | CH₂O | 2-quinolinyl | 3-Cl | H |
| 674 | 4-pyridinyl | CH₂O | 2-quinolinyl | 3-CN | H |
| 675 | 4-pyridinyl | CH₂O | 2-quinolinyl | 3-NO₂ | H |
| 676 | 4-pyridinyl | CH₂O | 2-quinolinyl | 3-OMe | H |
| 677 | 4-pyridinyl | CH₂O | 2-quinolinyl | 3-Me | H |
| 678 | 4-pyridinyl | CH₂O | 2-quinolinyl | 3-Et | H |
| 679 | 4-pyridinyl | CH₂O | 2-quinolinyl | 3-ⁱPr | H |
| 680 | 4-pyridinyl | CH₂O | 2-quinolinyl | 3-^{t}Bu | H |
| 681 | 4-pyridinyl | CH₂O | 2-quinolinyl | 3-CF₃ | H |
| 682 | 4-pyridinyl | CH₂O | 2-quinolinyl | 3-SO₂Me | H |
| 683 | 4-pyridinyl | CH₂O | 2-quinolinyl | 3-SO₂Et | H |
| 684 | 4-pyridinyl | CH₂O | 2-quinolinyl | 3-SO₂ⁱPr | H |
| 685 | 4-pyridinyl | CH₂O | 2-quinolinyl | 3-OCF₃ | H |
| 686 | 4-pyridinyl | CH₂O | 2-quinolinyl | 3-OCH₂CF₃ | H |
| 687 | 4-pyridinyl | CH₂O | 2-quinolinyl | 3-NHMe | H |
| 688 | 4-pyridinyl | CH₂O | 2-quinolinyl | 3-NMe₂ | H |
| 689 | 4-pyridinyl | CH₂O | 2-quinolinyl | 3-cyclopropyl | H |
| 690 | 4-pyridinyl | CH₂O | 2-quinolinyl | 3-OEt | H |
| 691 | 4-pyridinyl | CH₂O | 2-quinolinyl | 3-OⁱPr | H |
| 692 | 4-pyridinyl | CH₂O | 2-quinolinyl | 3-CH₂-cyclopropyl | H |
| 693 | 4-pyridinyl | CH₂O | 2-quinolinyl | 3-SMe | H |
| 694 | 4-pyridinyl | CH₂O | 2-quinolinyl | 3-SEt | H |
| 695 | 4-pyridinyl | CH₂O | 2-quinolinyl | 3-SⁱPr | H |
| 696 | 4-pyridinyl | CH₂O | 2-quinolinyl | 4-F | H |
| 697 | 4-pyridinyl | CH₂O | 2-quinolinyl | 4-Cl | H |
| 698 | 4-pyridinyl | CH₂O | 2-quinolinyl | 4-CN | H |
| 699 | 4-pyridinyl | CH₂O | 2-quinolinyl | 4-NO₂ | H |
| 700 | 4-pyridinyl | CH₂O | 2-quinolinyl | 4-OMe | H |
| 701 | 4-pyridinyl | CH₂O | 2-quinolinyl | 4-Me | H |
| 702 | 4-pyridinyl | CH₂O | 2-quinolinyl | 4-Et | H |
| 703 | 4-pyridinyl | CH₂O | 2-quinolinyl | 4-ⁱPr | H |
| 704 | 4-pyridinyl | CH₂O | 2-quinolinyl | 4-^{t}Bu | H |
| 705 | 4-pyridinyl | CH₂O | 2-quinolinyl | 4-CF₃ | H |
| 706 | 4-pyridinyl | CH₂O | 2-quinolinyl | 4-SO₂Me | H |
| 707 | 4-pyridinyl | CH₂O | 2-quinolinyl | 4-SO₂Et | H |
| 708 | 4-pyridinyl | CH₂O | 2-quinolinyl | 4-SO₂ⁱPr | |
| 709 | 4-pyridinyl | CH₂O | 2-quinolinyl | 4-OCF₃ | H |
| 710 | 4-pyridinyl | CH₂O | 2-quinolinyl | 4-OCH₂CF₃ | H |
| 711 | 4-pyridinyl | CH₂O | 2-quinolinyl | 4-NHMe | H |
| 712 | 4-pyridinyl | CH₂O | 2-quinolinyl | 4-NMe₂ | H |
| 713 | 4-pyridinyl | CH₂O | 2-quinolinyl | 4-cyclopropyl | |
| 714 | 4-pyridinyl | CH₂O | 2-quinolinyl | 4-OEt | H |
| 715 | 4-pyridinyl | CH₂O | 2-quinolinyl | 4-OⁱPr | H |
| 716 | 4-pyridinyl | CH₂O | 2-quinolinyl | 4-CH₂-cyclopropyl | |
| 717 | 4-pyridinyl | CH₂O | 2-quinolinyl | 4-SMe | H |
| 718 | 4-pyridinyl | CH₂O | 2-quinolinyl | 4-SEt | H |
| 719 | 4-pyridinyl | CH₂O | 2-quinolinyl | 4-SⁱPr | H |
| 720 | ⁱPr | CH₂O | 2-quinolinyl | H | H |
| 721 | Me | CH₂O | 2-quinolinyl | H | H |
| 722 | morpholinyl | CH₂O | 2-quinolinyl | H | H |
| 723 | N-piperazino | CH₂O | 2-quinolinyl | H | H |
| 724 | piperazino | CH₂O | 2-quinolinyl | H | H |
| 725 | piperidino | CH₂O | 2-quinolinyl | H | H |
| 726 | 4-pyridinyl | CH₂O | 2-quinoxaline | H | H |
| 727 | 4-pyridinyl | CH₂O | 5,6,7,8-tetrahydro- | H | H |
| | | | 2-quinolyl | | |
| 728 | 3-pyridinyl | OCH₂ | 2-benzimidazole | H | H |
| 729 | 4-pyridinyl | OCH₂ | 2-benzimidazole | H | H |
| 730 | morpholinyl | OCH₂ | 2-benzimidazole | H | H |
| 731 | 3-pyridinyl | OCH₂ | 2-benzoxazole | H | H |
| 732 | 4-pyridinyl | OCH₂ | 2-benzoxazole | H | H |
| 733 | morpholinyl | OCH₂ | 2-benzoxazole | H | H |
| 734 | 3-pyridinyl | OCH₂ | 2-benzthiazole | H | H |
| 735 | 4-pyridinyl | OCH₂ | 2-benzthiazole | H | H |
| 736 | morpholinyl | OCH₂ | 2-benzthiazole | H | H |
| 737 | 3-pyridinyl | OCH₂ | 2-pyridinyl | H | H |
| 738 | 4-pyridinyl | OCH₂ | 2-pyridinyl | H | H |
| 739 | morpholinyl | OCH₂ | 2-pyridinyl | H | H |
| 740 | 3-pyridinyl | OCH₂ | 2-quinazoline | H | H |
| 741 | 4-pyridinyl | OCH₂ | 2-quinazoline | H | H |
| 742 | morpholinyl | OCH₂ | 2-quinazoline | H | H |
| 743 | 3,4-dimethoxyphenyl | OCH₂ | 2-quinolinyl | H | H |
| 744 | 2-methoxy-4-pyridinyl | OCH₂ | 2-quinolinyl | H | H |
| 746 | 2-methoxy-4-pyridinyl | OCH₂ | 2-quinolinyl | 3-F | H |
| 747 | 2-methoxy-4-pyridinyl | OCH₂ | 2-quinolinyl | 3-Cl | H |
| 748 | 2-methoxy-4-pyridinyl | OCH₂ | 2-quinolinyl | 3-CN | H |
| 749 | 2-methoxy-4-pyridinyl | OCH₂ | 2-quinolinyl | 3-NO₂ | H |
| 750 | 2-methoxy-4-pyridinyl | OCH₂ | 2-quinolinyl | 3-OMe | H |
| 751 | 2-methoxy-4-pyridinyl | OCH₂ | 2-quinolinyl | 3-Me | H |
| 752 | 2-methoxy-4-pyridinyl | OCH₂ | 2-quinolinyl | 3-Et | H |
| 753 | 2-methoxy-4-pyridinyl | OCH₂ | 2-quinolinyl | 3-ⁱPr | H |
| 754 | 2-methoxy-4-pyridinyl | OCH₂ | 2-quinolinyl | 3-^{t}Bu | H |
| 755 | 2-methoxy-4-pyridinyl | OCH₂ | 2-quinolinyl | 3-CF₃ | H |
| 756 | 2-methoxy-4-pyridinyl | OCH₂ | 2-quinolinyl | 3-SO₂Me | H |
| 757 | 2-methoxy-4-pyridinyl | OCH₂ | 2-quinolinyl | 3-SO₂Et | H |
| 758 | 2-methoxy-4-pyridinyl | OCH₂ | 2-quinolinyl | 3-SO₂ⁱPr | H |
| 759 | 2-methoxy-4-pyridinyl | OCH₂ | 2-quinolinyl | 3-OCF₃ | H |
| 760 | 2-methoxy-4-pyridinyl | OCH₂ | 2-quinolinyl | 3-OCH₂CF₃ | H |
| 761 | 2-methoxy-4-pyridinyl | OCH₂ | 2-quinolinyl | 3-NHMe | H |
| 762 | 2-methoxy-4-pyridinyl | OCH₂ | 2-quinolinyl | 3-NMe₂ | H |
| 763 | 2-methoxy-4-pyridinyl | OCH₂ | 2-quinolinyl | 3-cyclopropyl | H |
| 764 | 2-methoxy-4-pyridinyl | OCH₂ | 2-quinolinyl | 3-OEt | H |
| 765 | 2-methoxy-4-pyridinyl | OCH₂ | 2-quinolinyl | 3-OⁱPr | H |
| 766 | 2-methoxy-4-pyridinyl | OCH₂ | 2-quinolinyl | 3-CH₂-cyclopropyl | H |
| 767 | 2-methoxy-4-pyridinyl | OCH₂ | 2-quinolinyl | 3-SMe | H |
| 768 | 2-methoxy-4-pyridinyl | OCH₂ | 2-quinolinyl | 3-SEt | H |
| 769 | 2-methoxy-4-pyridinyl | OCH₂ | 2-quinolinyl | 3-SⁱPr | H |
| 770 | 2-methoxy-4-pyridinyl | OCH₂ | 2-quinolinyl | 4-F | H |
| 771 | 2-methoxy-4-pyridinyl | OCH₂ | 2-quinolinyl | 4-Cl | H |
| 772 | 2-methoxy-4-pyridinyl | OCH₂ | 2-quinolinyl | 4-CN | H |
| 773 | 2-methoxy-4-pyridinyl | OCH₂ | 2-quinolinyl | 4-NO₂ | H |
| 774 | 2-methoxy-4-pyridinyl | OCH₂ | 2-quinolinyl | 4-OMe | H |
| 775 | 2-methoxy-4-pyridinyl | OCH₂ | 2-quinolinyl | 4-Me | H |
| 776 | 2-methoxy-4-pyridinyl | OCH₂ | 2-quinolinyl | 4-Et | H |
| 777 | 2-methoxy-4-pyridinyl | OCH₂ | 2-quinolinyl | 4-ⁱPr | H |
| 778 | 2-methoxy-4-pyridinyl | OCH₂ | 2-quinolinyl | 4-^{t}Bu | H |
| 779 | 2-methoxy-4-pyridinyl | OCH₂ | 2-quinolinyl | 4-CF₃ | H |
| 780 | 2-methoxy-4-pyridinyl | OCH₂ | 2-quinolinyl | 4-SO₂Me | H |
| 781 | 2-methoxy-4-pyridinyl | OCH₂ | 2-quinolinyl | 4-SO₂Et | H |
| 782 | 2-methoxy-4-pyridinyl | OCH₂ | 2-quinolinyl | 4-SO₂ⁱPr | H |
| 783 | 2-methoxy-4-pyridinyl | OCH₂ | 2-quinolinyl | 4-OCF₃ | H |
| 784 | 2-methoxy-4-pyridinyl | OCH₂ | 2-quinolinyl | 4-OCH₂CF₃ | H |
| 785 | 2-methoxy-4-pyridinyl | OCH₂ | 2-quinolinyl | 4-NHMe | H |
| 786 | 2-methoxy-4-pyridinyl | OCH₂ | 2-quinolinyl | 4-NMe₂ | H |
| 787 | 2-methoxy-4-pyridinyl | OCH₂ | 2-quinolinyl | 4-cyclopropyl | H |
| 788 | 2-methoxy-4-pyridinyl | OCH₂ | 2-quinolinyl | 4-OEt | H |
| 789 | 2-methoxy-4-pyridinyl | OCH₂ | 2-quinolinyl | 4-OⁱPr | H |
| 790 | 2-methoxy-4-pyridinyl | OCH₂ | 2-quinolinyl | 4-CH₂-cyclopropyl | H |
| 791 | 2-methoxy-4-pyridinyl | OCH₂ | 2-quinolinyl | 4-SMe | H |
| 792 | 2-methoxy-4-pyridinyl | OCH₂ | 2-quinolinyl | 4-SEt | H |
| 793 | 2-methoxy-4-pyridinyl | OCH₂ | 2-quinolinyl | 4-SⁱPr | H |
| 794 | 2-hydroxy-4-pyridinyl | OCH₂ | 2-quinolinyl | H | H |
| 795 | 2-hydroxy-4-pyridinyl | OCH₂ | 2-quinolinyl | 3-F | H |
| 796 | 2-hydroxy-4-pyridinyl | OCH₂ | 2-quinolinyl | 3-Cl | H |
| 797 | 2-hydroxy-4-pyridinyl | OCH₂ | 2-quinolinyl | 3-CN | H |
| 798 | 2-hydroxy-4-pyridinyl | OCH₂ | 2-quinolinyl | 3-NO₂ | H |
| 799 | 2-hydroxy-4-pyridinyl | OCH₂ | 2-quinolinyl | 3-OMe | H |
| 800 | 2-hydroxy-4-pyridinyl | OCH₂ | 2-quinolinyl | 3-Me | H |
| 801 | 2-hydroxy-4-pyridinyl | OCH₂ | 2-quinolinyl | 3-Et | H |
| 802 | 2-hydroxy-4-pyridinyl | OCH₂ | 2-quinolinyl | 3-ⁱPr | H |
| 803 | 2-hydroxy-4-pyridinyl | OCH₂ | 2-quinolinyl | 3-^{t}Bu | H |
| 804 | 2-hydroxy-4-pyridinyl | OCH₂ | 2-quinolinyl | 3-CF₃ | H |
| 805 | 2-hydroxy-4-pyridinyl | OCH₂ | 2-quinolinyl | 3-SO₂Me | H |
| 806 | 2-hydroxy-4-pyridinyl | OCH₂ | 2-quinolinyl | 3-SO₂Et | H |
| 807 | 2-hydroxy-4-pyridinyl | OCH₂ | 2-quinolinyl | 3-SO₂ⁱPr | H |
| 808 | 2-hydroxy-4-pyridinyl | OCH₂ | 2-quinolinyl | 3-OCF₃ | H |
| 809 | 2-hydroxy-4-pyridinyl | OCH₂ | 2-quinolinyl | 3-OCH₂CF₃ | H |
| 810 | 2-hydroxy-4-pyridinyl | OCH₂ | 2-quinolinyl | 3-NHMe | H |
| 811 | 2-hydroxy-4-pyridinyl | OCH₂ | 2-quinolinyl | 3-NMe₂ | H |
| 812 | 2-hydroxy-4-pyridinyl | OCH₂ | 2-quinolinyl | 3-cyclopropyl | H |
| 813 | 2-hydroxy-4-pyridinyl | OCH₂ | 2-quinolinyl | 3-OEt | H |
| 814 | 2-hydroxy-4-pyridinyl | OCH₂ | 2-quinolinyl | 3-OⁱPr | H |
| 815 | 2-hydroxy-4-pyridinyl | OCH₂ | 2-quinolinyl | 3-CH₂-cyclopropyl | H |
| 816 | 2-hydroxy-4-pyridinyl | OCH₂ | 2-quinolinyl | 3-SMe | H |
| 817 | 2-hydroxy-4-pyridinyl | OCH₂ | 2-quinolinyl | 3-SEt | H |
| 818 | 2-hydroxy-4-pyridinyl | OCH₂ | 2-quinolinyl | 3-SⁱPr | H |
| 819 | 2-hydroxy-4-pyridinyl | OCH₂ | 2-quinolinyl | 4-F | H |
| 820 | 2-hydroxy-4-pyridinyl | OCH₂ | 2-quinolinyl | 4-Cl | H |
| 821 | 2-hydroxy-4-pyridinyl | OCH₂ | 2-quinolinyl | 4-CN | H |
| 822 | 2-hydroxy-4-pyridinyl | OCH₂ | 2-quinolinyl | 4-NO₂ | H |
| 823 | 2-hydroxy-4-pyridinyl | OCH₂ | 2-quinolinyl | 4-OMe | H |
| 824 | 2-hydroxy-4-pyridinyl | OCH₂ | 2-quinolinyl | 4-Me | H |
| 825 | 2-hydroxy-4-pyridinyl | OCH₂ | 2-quinolinyl | 4-Et | H |
| 826 | 2-hydroxy-4-pyridinyl | OCH₂ | 2-quinolinyl | 4-ⁱPr | H |
| 827 | 2-hydroxy-4-pyridinyl | OCH₂ | 2-quinolinyl | 4-^{t}Bu | H |
| 828 | 2-hydroxy-4-pyridinyl | OCH₂ | 2-quinolinyl | 4-CF₃ | H |
| 829 | 2-hydroxy-4-pyridinyl | OCH₂ | 2-quinolinyl | 4-SO₂Me | H |
| 830 | 2-hydroxy-4-pyridinyl | OCH₂ | 2-quinolinyl | 4-SO₂Et | H |
| 831 | 2-hydroxy-4-pyridinyl | OCH₂ | 2-quinolinyl | 4-SO₂ⁱPr | H |
| 832 | 2-hydroxy-4-pyridinyl | OCH₂ | 2-quinolinyl | 4-OCF₃ | H |
| 833 | 2-hydroxy-4-pyridinyl | OCH₂ | 2-quinolinyl | 4-OCH₂CF₃ | H |
| 834 | 2-hydroxy-4-pyridinyl | OCH₂ | 2-quinolinyl | 4-NHMe | H |
| 835 | 2-hydroxy-4-pyridinyl | OCH₂ | 2-quinolinyl | 4-NMe₂ | H |
| 836 | 2-hydroxy-4-pyridinyl | OCH₂ | 2-quinolinyl | 4-cyclopropyl | H |
| 837 | 2-hydroxy-4-pyridinyl | OCH₂ | 2-quinolinyl | 4-OEt | H |
| 838 | 2-hydroxy-4-pyridinyl | OCH₂ | 2-quinolinyl | 4-OⁱPr | H |
| 839 | 2-hydroxy-4-pyridinyl | OCH₂ | 2-quinolinyl | 4-CH₂-cyclopropyl | H |
| 840 | 2-hydroxy-4-pyridinyl | OCH₂ | 2-quinolinyl | 4-SMe | H |
| 841 | 2-hydroxy-4-pyridinyl | OCH₂ | 2-quinolinyl | 4-SEt | H |
| 842 | 2-hydroxy-4-pyridinyl | OCH₂ | 2-quinolinyl | 4-SⁱPr | H |
| 843 | 4-chloro-phenyl | OCH₂ | 2-quinolinyl | H | H |
| 844 | 4-chloro-phenyl | OCH₂ | 2-quinolinyl | 3-F | H |
| 845 | 4-chloro-phenyl | OCH₂ | 2-quinolinyl | 3-Cl | H |
| 846 | 4-chloro-phenyl | OCH₂ | 2-quinolinyl | 3-CN | H |
| 847 | 4-chloro-phenyl | OCH₂ | 2-quinolinyl | 3-NO₂ | H |
| 848 | 4-chloro-phenyl | OCH₂ | 2-quinolinyl | 3-OMe | H |
| 849 | 4-chloro-phenyl | OCH₂ | 2-quinolinyl | 3-Me | H |
| 850 | 4-chloro-phenyl | OCH₂ | 2-quinolinyl | 3-Et | H |
| 851 | 4-chloro-phenyl | OCH₂ | 2-quinolinyl | 3-ⁱPr | H |
| 852 | 4-chloro-phenyl | OCH₂ | 2-quinolinyl | 3-^{t}Bu | H |
| 853 | 4-chloro-phenyl | OCH₂ | 2-quinolinyl | 3-CF₃ | H |
| 854 | 4-chloro-phenyl | OCH₂ | 2-quinolinyl | 3-SO₂Me | H |
| 855 | 4-chloro-phenyl | OCH₂ | 2-quinolinyl | 3-SO₂Et | H |
| 856 | 4-chloro-phenyl | OCH₂ | 2-quinolinyl | 3-SO₂ⁱPr | H |
| 857 | 4-chloro-phenyl | OCH₂ | 2-quinolinyl | 3-OCF₃ | H |
| 858 | 4-chloro-phenyl | OCH₂ | 2-quinolinyl | 3-OCH₂CF₃ | H |
| 859 | 4-chloro-phenyl | OCH₂ | 2-quinolinyl | 3-NHMe | H |
| 860 | 4-chloro-phenyl | OCH₂ | 2-quinolinyl | 3-NMe₂ | H |
| 861 | 4-chloro-phenyl | OCH₂ | 2-quinolinyl | 3-cyclopropyl | H |
| 862 | 4-chloro-phenyl | OCH₂ | 2-quinolinyl | 3-OEt | H |
| 863 | 4-chloro-phenyl | OCH₂ | 2-quinolinyl | 3-OⁱPr | H |
| 864 | 4-chloro-phenyl | OCH₂ | 2-quinolinyl | 3-CH₂-cyclopropyl | H |
| 865 | 4-chloro-phenyl | OCH₂ | 2-quinolinyl | 3-SMe | H |
| 866 | 4-chloro-phenyl | OCH₂ | 2-quinolinyl | 3-SEt | H |
| 867 | 4-chloro-phenyl | OCH₂ | 2-quinolinyl | 3-SⁱPr | H |
| 868 | 4-chloro-phenyl | OCH₂ | 2-quinolinyl | 4-F | H |
| 869 | 4-chloro-phenyl | OCH₂ | 2-quinolinyl | 4-Cl | H |
| 870 | 4-chloro-phenyl | OCH₂ | 2-quinolinyl | 4-CN | H |
| 871 | 4-chloro-phenyl | OCH₂ | 2-quinolinyl | 4-NO₂ | H |
| 872 | 4-chloro-phenyl | OCH₂ | 2-quinolinyl | 4-OMe | H |
| 873 | 4-chloro-phenyl | OCH₂ | 2-quinolinyl | 4-Me | H |
| 874 | 4-chloro-phenyl | OCH₂ | 2-quinolinyl | 4-Et | H |
| 875 | 4-chloro-phenyl | OCH₂ | 2-quinolinyl | 4-ⁱPr | H |
| 876 | 4-chloro-phenyl | OCH₂ | 2-quinolinyl | 4-^{t}Bu | H |
| 877 | 4-chloro-phenyl | OCH₂ | 2-quinolinyl | 4-CF₃ | H |
| 878 | 4-chloro-phenyl | OCH₂ | 2-quinolinyl | 4-SO₂Me | H |
| 879 | 4-chloro-phenyl | OCH₂ | 2-quinolinyl | 4-SO₂Et | H |
| 880 | 4-chloro-phenyl | OCH₂ | 2-quinolinyl | 4-SO₂ⁱPr | H |
| 881 | 4-chloro-phenyl | OCH₂ | 2-quinolinyl | 4-OCF₃ | H |
| 882 | 4-chloro-phenyl | OCH₂ | 2-quinolinyl | 4-OCH₂CF₃ | H |
| 883 | 4-chloro-phenyl | OCH₂ | 2-quinolinyl | 4-NHMe | H |
| 884 | 4-chloro-phenyl | OCH₂ | 2-quinolinyl | 4-NMe₂ | H |
| 885 | 4-chloro-phenyl | OCH₂ | 2-quinolinyl | 4-cyclopropyl | H |
| 886 | 4-chloro-phenyl | OCH₂ | 2-quinolinyl | 4-OEt | H |
| 887 | 4-chloro-phenyl | OCH₂ | 2-quinolinyl | 4-OⁱPr | H |
| 888 | 4-chloro-phenyl | OCH₂ | 2-quinolinyl | 4-CH₂-cyclopropyl | H |
| 889 | 4-chloro-phenyl | OCH₂ | 2-quinolinyl | 4-SMe | H |
| 890 | 4-chloro-phenyl | OCH₂ | 2-quinolinyl | 4-SEt | H |
| 891 | 4-chloro-phenyl | OCH₂ | 2-quinolinyl | 4-SⁱPr | H |
| 892 | 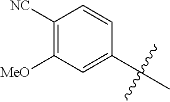 | OCH₂ | 2-quinolinyl | H | H |
| 893 | 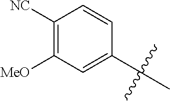 | OCH₂ | 2-quinolinyl | 3-F | H |
| 894 | 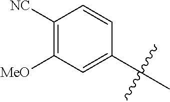 | OCH₂ | 2-quinolinyl | 3-Cl | H |
| 895 | 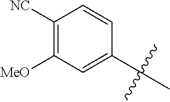 | OCH₂ | 2-quinolinyl | 3-CN | H |
| 896 | 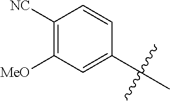 | OCH₂ | 2-quinolinyl | 3-NO₂ | H |
| 897 | 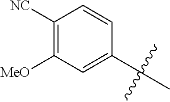 | OCH₂ | 2-quinolinyl | 3-OMe | H |
| 898 | 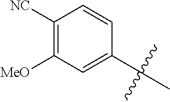 | OCH₂ | 2-quinolinyl | 3-Me | H |
| 899 | 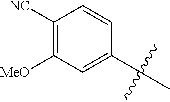 | OCH₂ | 2-quinolinyl | 3-Et | H |
| 900 | 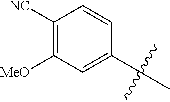 | OCH₂ | 2-quinolinyl | 3-ⁱPr | H |
| 901 | 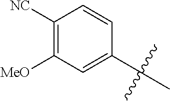 | OCH₂ | 2-quinolinyl | 3-^{t}Bu | H |
| 902 | 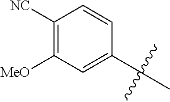 | OCH₂ | 2-quinolinyl | 3-CF₃ | H |
| 903 | 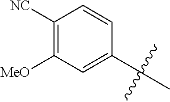 | OCH₂ | 2-quinolinyl | 3-SO₂Me | H |
| 904 | 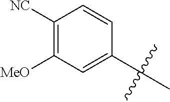 | OCH₂ | 2-quinolinyl | 3-SO₂Et | H |
| 905 | 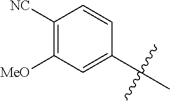 | OCH₂ | 2-quinolinyl | 3-SO₂ⁱPr | H |
| 906 | 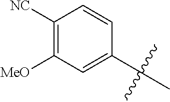 | OCH₂ | 2-quinolinyl | 3-OCF₃ | H |
| 907 | 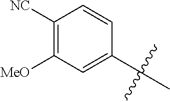 | OCH₂ | 2-quinolinyl | 3-OCH₂CF₃ | H |
| 908 | 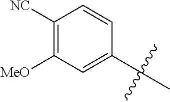 | OCH₂ | 2-quinolinyl | 3-NHMe | H |
| 909 | 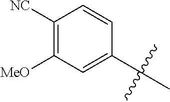 | OCH₂ | 2-quinolinyl | 3-NMe₂ | H |
| 910 | 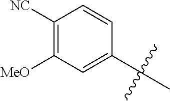 | OCH₂ | 2-quinolinyl | 3-cyclopropyl | H |
| 911 | 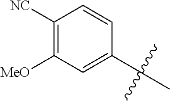 | OCH₂ | 2-quinolinyl | 3-OEt | H |
| 912 | 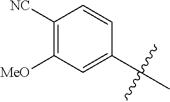 | OCH₂ | 2-quinolinyl | 3-OⁱPr | H |
| 913 | 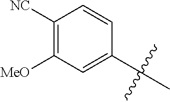 | OCH₂ | 2-quinolinyl | 3-CH₂-cyclopropyl | H |
| 914 | 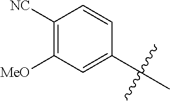 | OCH₂ | 2-quinolinyl | 3-SMe | H |
| 915 | 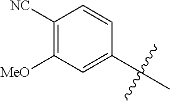 | OCH₂ | 2-quinolinyl | 3-SEt | H |
| 916 | 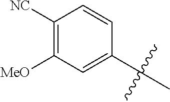 | OCH₂ | 2-quinolinyl | 3-SⁱPr | H |
| 917 | 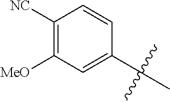 | OCH₂ | 2-quinolinyl | 4-F | H |
| 918 | 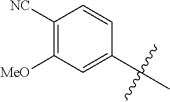 | OCH₂ | 2-quinolinyl | 4-Cl | H |
| 919 | 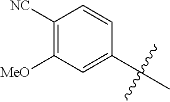 | OCH₂ | 2-quinolinyl | 4-CN | H |
| 920 | 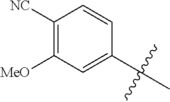 | OCH₂ | 2-quinolinyl | 4-NO₂ | H |
| 921 | 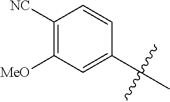 | OCH₂ | 2-quinolinyl | 4-OMe | H |
| 922 | 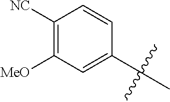 | OCH₂ | 2-quinolinyl | 4-Me | H |
| 923 | 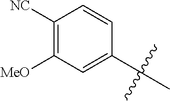 | OCH₂ | 2-quinolinyl | 4-Et | H |
| 924 | 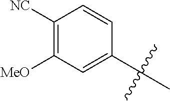 | OCH₂ | 2-quinolinyl | 4-ⁱPr | H |
| 925 | 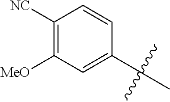 | OCH₂ | 2-quinolinyl | 4-^{t}Bu | H |
| 926 | 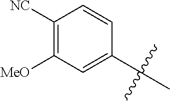 | OCH₂ | 2-quinolinyl | 4-CF₃ | H |
| 927 | 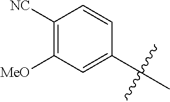 | OCH₂ | 2-quinolinyl | 4-SO₂Me | H |
| 928 | 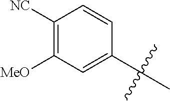 | OCH₂ | 2-quinolinyl | 4-SO₂Et | H |
| 929 | 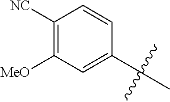 | OCH₂ | 2-quinolinyl | 4-SO₂ⁱPr | H |
| 930 | 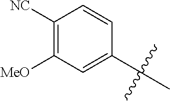 | OCH₂ | 2-quinolinyl | 4-OCF₃ | H |
| 931 | 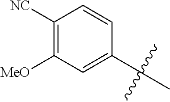 | OCH₂ | 2-quinolinyl | 4-OCH₂CF₃ | H |
| 932 | 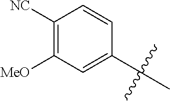 | OCH₂ | 2-quinolinyl | 4-NHMe | H |
| 933 | 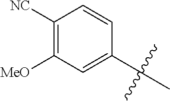 | OCH₂ | 2-quinolinyl | 4-NMe₂ | H |
| 934 | 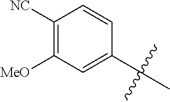 | OCH₂ | 2-quinolinyl | 4-cyclopropyl | H |
| 935 | 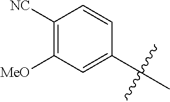 | OCH₂ | 2-quinolinyl | 4-OEt | H |
| 936 | 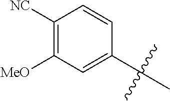 | OCH₂ | 2-quinolinyl | 4-OⁱPr | H |
| 937 | 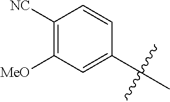 | OCH₂ | 2-quinolinyl | 4-CH₂-cyclopropyl | H |
| 938 | 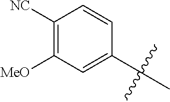 | OCH₂ | 2-quinolinyl | 4-SMe | H |
| 939 | 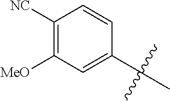 | OCH₂ | 2-quinolinyl | 4-SEt | H |
| 940 | 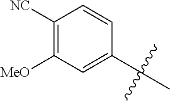 | OCH₂ | 2-quinolinyl | 4-SⁱPr | H |
| 941 | 4-cyano-phenyl | OCH₂ | 2-quinolinyl | H | H |
| 942 | 4-cyano-phenyl | OCH₂ | 2-quinolinyl | 3-F | H |
| 943 | 4-cyano-phenyl | OCH₂ | 2-quinolinyl | 3-Cl | H |
| 944 | 4-cyano-phenyl | OCH₂ | 2-quinolinyl | 3-CN | H |
| 945 | 4-cyano-phenyl | OCH₂ | 2-quinolinyl | 3-NO₂ | H |
| 946 | 4-cyano-phenyl | OCH₂ | 2-quinolinyl | 3-OMe | H |
| 947 | 4-cyano-phenyl | OCH₂ | 2-quinolinyl | 3-Me | H |
| 948 | 4-cyano-phenyl | OCH₂ | 2-quinolinyl | 3-Et | H |
| 949 | 4-cyano-phenyl | OCH₂ | 2-quinolinyl | 3-ⁱPr | H |
| 950 | 4-cyano-phenyl | OCH₂ | 2-quinolinyl | 3-^{t}Bu | H |
| 951 | 4-cyano-phenyl | OCH₂ | 2-quinolinyl | 3-CF₃ | H |
| 952 | 4-cyano-phenyl | OCH₂ | 2-quinolinyl | 3-SO₂Me | H |
| 953 | 4-cyano-phenyl | OCH₂ | 2-quinolinyl | 3-SO₂Et | H |
| 954 | 4-cyano-phenyl | OCH₂ | 2-quinolinyl | 3-SO₂ⁱPr | H |
| 955 | 4-cyano-phenyl | OCH₂ | 2-quinolinyl | 3-OCF₃ | H |
| 956 | 4-cyano-phenyl | OCH₂ | 2-quinolinyl | 3-OCH₂CF₃ | H |
| 957 | 4-cyano-phenyl | OCH₂ | 2-quinolinyl | 3-NHMe | H |
| 958 | 4-cyano-phenyl | OCH₂ | 2-quinolinyl | 3-NMe₂ | H |
| 959 | 4-cyano-phenyl | OCH₂ | 2-quinolinyl | 3-cyclopropyl | H |
| 960 | 4-cyano-phenyl | OCH₂ | 2-quinolinyl | 3-OEt | H |
| 961 | 4-cyano-phenyl | OCH₂ | 2-quinolinyl | 3-OⁱPr | H |
| 962 | 4-cyano-phenyl | OCH₂ | 2-quinolinyl | 3-CH₂-cyclopropyl | H |
| 963 | 4-cyano-phenyl | OCH₂ | 2-quinolinyl | 3-SMe | H |
| 964 | 4-cyano-phenyl | OCH₂ | 2-quinolinyl | 3-SEt | H |
| 965 | 4-cyano-phenyl | OCH₂ | 2-quinolinyl | 3-SⁱPr | H |
| 966 | 4-cyano-phenyl | OCH₂ | 2-quinolinyl | 4-F | H |
| 967 | 4-cyano-phenyl | OCH₂ | 2-quinolinyl | 4-Cl | H |
| 968 | 4-cyano-phenyl | OCH₂ | 2-quinolinyl | 4-CN | H |
| 969 | 4-cyano-phenyl | OCH₂ | 2-quinolinyl | 4-NO₂ | H |
| 970 | 4-cyano-phenyl | OCH₂ | 2-quinolinyl | 4-OMe | H |
| 971 | 4-cyano-phenyl | OCH₂ | 2-quinolinyl | 4-Me | H |
| 972 | 4-cyano-phenyl | OCH₂ | 2-quinolinyl | 4-Et | H |
| 973 | 4-cyano-phenyl | OCH₂ | 2-quinolinyl | 4-ⁱPr | H |
| 974 | 4-cyano-phenyl | OCH₂ | 2-quinolinyl | 4-^{t}Bu | H |
| 975 | 4-cyano-phenyl | OCH₂ | 2-quinolinyl | 4-CF₃ | H |
| 976 | 4-cyano-phenyl | OCH₂ | 2-quinolinyl | 4-SO₂Me | H |
| 977 | 4-cyano-phenyl | OCH₂ | 2-quinolinyl | 4-SO₂Et | H |
| 978 | 4-cyano-phenyl | OCH₂ | 2-quinolinyl | 4-SO₂ⁱPr | H |
| 979 | 4-cyano-phenyl | OCH₂ | 2-quinolinyl | 4-OCF₃ | H |
| 980 | 4-cyano-phenyl | OCH₂ | 2-quinolinyl | 4-OCH₂CF₃ | H |
| 981 | 4-cyano-phenyl | OCH₂ | 2-quinolinyl | 4-NHMe | H |
| 982 | 4-cyano-phenyl | OCH₂ | 2-quinolinyl | 4-NMe₂ | H |
| 983 | 4-cyano-phenyl | OCH₂ | 2-quinolinyl | 4-cyclopropyl | H |
| 984 | 4-cyano-phenyl | OCH₂ | 2-quinolinyl | 4-OEt | H |
| 985 | 4-cyano-phenyl | OCH₂ | 2-quinolinyl | 4-OⁱPr | H |
| 986 | 4-cyano-phenyl | OCH₂ | 2-quinolinyl | 4-CH₂-cyclopropyl | H |
| 987 | 4-cyano-phenyl | OCH₂ | 2-quinolinyl | 4-SMe | H |
| 988 | 4-cyano-phenyl | OCH₂ | 2-quinolinyl | 4-SEt | H |
| 989 | 4-cyano-phenyl | OCH₂ | 2-quinolinyl | 4-SⁱPr | H |
| 991 | 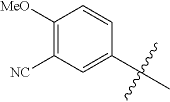 | OCH₂ | 2-quinolinyl | H | H |
| 992 | 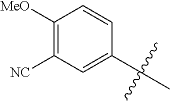 | OCH₂ | 2-quinolinyl | 3-F | H |
| 993 | 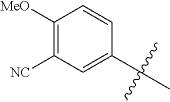 | OCH₂ | 2-quinolinyl | 3-Cl | H |
| 994 | 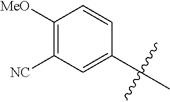 | OCH₂ | 2-quinolinyl | 3-CN | H |
| 995 | 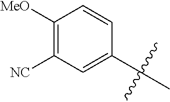 | OCH₂ | 2-quinolinyl | 3-NO₂ | H |
| 996 | 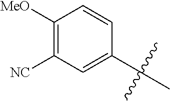 | OCH₂ | 2-quinolinyl | 3-OMe | H |
| 997 | 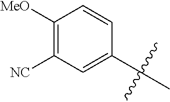 | OCH₂ | 2-quinolinyl | 3-Me | H |
| 998 | 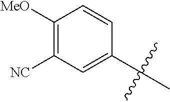 | OCH₂ | 2-quinolinyl | 3-Et | H |
| 999 | 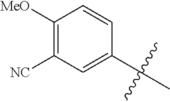 | OCH₂ | 2-quinolinyl | 3-ⁱPr | H |
| 1000 | 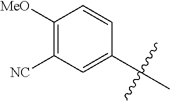 | OCH₂ | 2-quinolinyl | 3-^{t}Bu | H |
| 1001 | 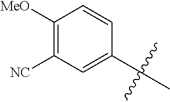 | OCH₂ | 2-quinolinyl | 3-CF₃ | H |
| 1002 | 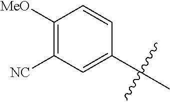 | OCH₂ | 2-quinolinyl | 3-SO₂Me | H |
| 1003 | 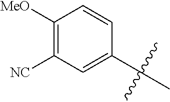 | OCH₂ | 2-quinolinyl | 3-SO₂Et | H |
| 1004 | 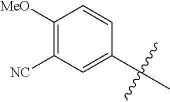 | OCH₂ | 2-quinolinyl | 3-SO₂ⁱPr | H |
| 1005 | 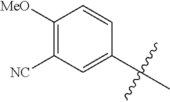 | OCH₂ | 2-quinolinyl | 3-OCF₃ | H |
| 1006 | 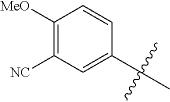 | OCH₂ | 2-quinolinyl | 3-OCH₂CF₃ | H |
| 1007 | 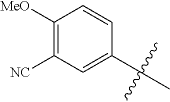 | OCH₂ | 2-quinolinyl | 3-NHMe | H |
| 1008 | 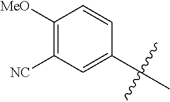 | OCH₂ | 2-quinolinyl | 3-NMe₂ | H |
| 1009 | 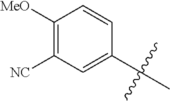 | OCH₂ | 2-quinolinyl | 3-cyclopropyl | H |
| 1010 | 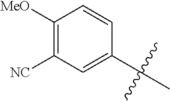 | OCH₂ | 2-quinolinyl | 3-OEt | H |
| 1011 | 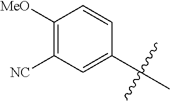 | OCH₂ | 2-quinolinyl | 3-OⁱPr | H |
| 1012 | 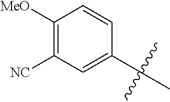 | OCH₂ | 2-quinolinyl | 3-CH₂-cyclopropyl | H |
| 1013 | 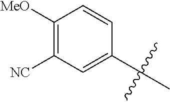 | OCH₂ | 2-quinolinyl | 3-SMe | H |
| 1014 | 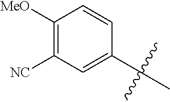 | OCH₂ | 2-quinolinyl | 3-SEt | H |
| 1015 | 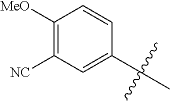 | OCH₂ | 2-quinolinyl | 3-SⁱPr | H |
| 1016 | 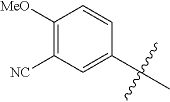 | OCH₂ | 2-quinolinyl | 4-F | H |
| 1017 | 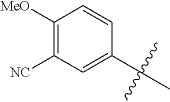 | OCH₂ | 2-quinolinyl | 4-Cl | H |
| 1018 | 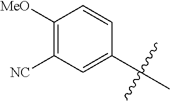 | OCH₂ | 2-quinolinyl | 4-CN | H |
| 1019 | 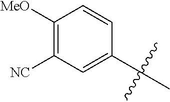 | OCH₂ | 2-quinolinyl | 4-NO₂ | H |
| 1020 | 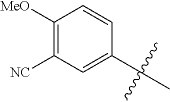 | OCH₂ | 2-quinolinyl | 4-OMe | H |
| 1021 | 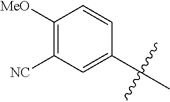 | OCH₂ | 2-quinolinyl | 4-Me | H |
| 1022 | 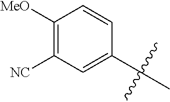 | OCH₂ | 2-quinolinyl | 4-Et | H |
| 1023 | 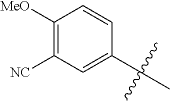 | OCH₂ | 2-quinolinyl | 4-ⁱPr | H |
| 1024 | 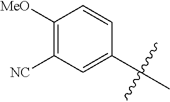 | OCH₂ | 2-quinolinyl | 4-^{t}Bu | H |
| 1025 | 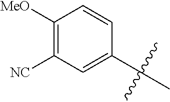 | OCH₂ | 2-quinolinyl | 4-CF₃ | H |
| 1026 | 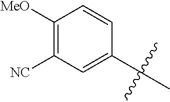 | OCH₂ | 2-quinolinyl | 4-SO₂Me | H |
| 1027 | 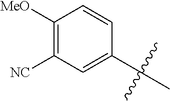 | OCH₂ | 2-quinolinyl | 4-SO₂Et | H |
| 1028 | 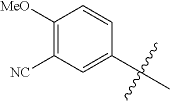 | OCH₂ | 2-quinolinyl | 4-SO₂ⁱPr | H |
| 1029 | 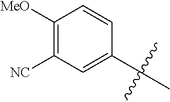 | OCH₂ | 2-quinolinyl | 4-OCF₃ | H |
| 1030 | 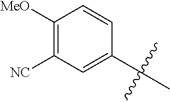 | OCH₂ | 2-quinolinyl | 4-OCH₂CF₃ | H |
| 1031 | 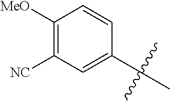 | OCH₂ | 2-quinolinyl | 4-NHMe | H |
| 1032 | 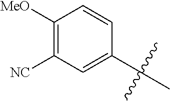 | OCH₂ | 2-quinolinyl | 4-NMe₂ | H |
| 1033 | 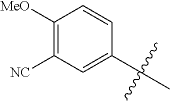 | OCH₂ | 2-quinolinyl | 4-cyclopropyl | H |
| 1034 | 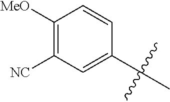 | OCH₂ | 2-quinolinyl | 4-OEt | H |
| 1035 | 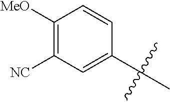 | OCH₂ | 2-quinolinyl | 4-OⁱPr | H |
| 1036 | 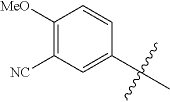 | OCH₂ | 2-quinolinyl | 4-CH₂-cyclopropyl | H |
| 1037 | 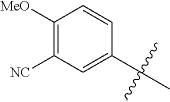 | OCH₂ | 2-quinolinyl | 4-SMe | H |
| 1038 | 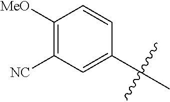 | OCH₂ | 2-quinolinyl | 4-SEt | H |
| 1039 | 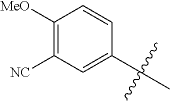 | OCH₂ | 2-quinolinyl | 4-SⁱPr | H |
| 1040 | 4-methoxy-phenyl | OCH₂ | 2-quinolinyl | H | H |
| 1041 | 4-methoxy-phenyl | OCH₂ | 2-quinolinyl | 3-F | H |
| 1042 | 4-methoxy-phenyl | OCH₂ | 2-quinolinyl | 3-Cl | H |
| 1043 | 4-methoxy-phenyl | OCH₂ | 2-quinolinyl | 3-CN | H |
| 1044 | 4-methoxy-phenyl | OCH₂ | 2-quinolinyl | 3-NO₂ | H |
| 1045 | 4-methoxy-phenyl | OCH₂ | 2-quinolinyl | 3-OMe | H |
| 1046 | 4-methoxy-phenyl | OCH₂ | 2-quinolinyl | 3-Me | H |
| 1047 | 4-methoxy-phenyl | OCH₂ | 2-quinolinyl | 3-Et | H |
| 1048 | 4-methoxy-phenyl | OCH₂ | 2-quinolinyl | 3-ⁱPr | H |
| 1049 | 4-methoxy-phenyl | OCH₂ | 2-quinolinyl | 3-^{t}Bu | H |
| 1050 | 4-methoxy-phenyl | OCH₂ | 2-quinolinyl | 3-CF₃ | H |
| 1051 | 4-methoxy-phenyl | OCH₂ | 2-quinolinyl | 3-SO₂Me | H |
| 1052 | 4-methoxy-phenyl | OCH₂ | 2-quinolinyl | 3-SO₂Et | H |
| 1053 | 4-methoxy-phenyl | OCH₂ | 2-quinolinyl | 3-SO₂ⁱPr | H |
| 1054 | 4-methoxy-phenyl | OCH₂ | 2-quinolinyl | 3-OCF₃ | H |
| 1055 | 4-methoxy-phenyl | OCH₂ | 2-quinolinyl | 3-OCH₂CF₃ | H |
| 1056 | 4-methoxy-phenyl | OCH₂ | 2-quinolinyl | 3-NHMe | H |
| 1057 | 4-methoxy-phenyl | OCH₂ | 2-quinolinyl | 3-NMe₂ | H |
| 1058 | 4-methoxy-phenyl | OCH₂ | 2-quinolinyl | 3-cyclopropyl | H |
| 1059 | 4-methoxy-phenyl | OCH₂ | 2-quinolinyl | 3-OEt | H |
| 1060 | 4-methoxy-phenyl | OCH₂ | 2-quinolinyl | 3-OⁱPr | H |
| 1061 | 4-methoxy-phenyl | OCH₂ | 2-quinolinyl | 3-CH₂-cyclopropyl | H |
| 1062 | 4-methoxy-phenyl | OCH₂ | 2-quinolinyl | 3-SMe | H |
| 1063 | 4-methoxy-phenyl | OCH₂ | 2-quinolinyl | 3-SEt | H |
| 1064 | 4-methoxy-phenyl | OCH₂ | 2-quinolinyl | 3-SⁱPr | H |
| 1065 | 4-methoxy-phenyl | OCH₂ | 2-quinolinyl | 4-F | H |
| 1066 | 4-methoxy-phenyl | OCH₂ | 2-quinolinyl | 4-Cl | H |
| 1067 | 4-methoxy-phenyl | OCH₂ | 2-quinolinyl | 4-CN | H |
| 1068 | 4-methoxy-phenyl | OCH₂ | 2-quinolinyl | 4-NO₂ | H |
| 1069 | 4-methoxy-phenyl | OCH₂ | 2-quinolinyl | 4-OMe | H |
| 1070 | 4-methoxy-phenyl | OCH₂ | 2-quinolinyl | 4-Me | H |
| 1071 | 4-methoxy-phenyl | OCH₂ | 2-quinolinyl | 4-Et | H |
| 1072 | 4-methoxy-phenyl | OCH₂ | 2-quinolinyl | 4-ⁱPr | H |
| 1073 | 4-methoxy-phenyl | OCH₂ | 2-quinolinyl | 4-^{t}Bu | H |
| 1074 | 4-methoxy-phenyl | OCH₂ | 2-quinolinyl | 4-CF₃ | H |
| 1075 | 4-methoxy-phenyl | OCH₂ | 2-quinolinyl | 4-SO₂Me | H |
| 1076 | 4-methoxy-phenyl | OCH₂ | 2-quinolinyl | 4-SO₂Et | H |
| 1077 | 4-methoxy-phenyl | OCH₂ | 2-quinolinyl | 4-SO₂ⁱPr | H |
| 1078 | 4-methoxy-phenyl | OCH₂ | 2-quinolinyl | 4-OCF₃ | H |
| 1079 | 4-methoxy-phenyl | OCH₂ | 2-quinolinyl | 4-OCH₂CF₃ | H |
| 1080 | 4-methoxy-phenyl | OCH₂ | 2-quinolinyl | 4-NHMe | H |
| 1081 | 4-methoxy-phenyl | OCH₂ | 2-quinolinyl | 4-NMe₂ | H |
| 1082 | 4-methoxy-phenyl | OCH₂ | 2-quinolinyl | 4-cyclopropyl | H |
| 1083 | 4-methoxy-phenyl | OCH₂ | 2-quinolinyl | 4-OEt | H |
| 1084 | 4-methoxy-phenyl | OCH₂ | 2-quinolinyl | 4-OⁱPr | H |
| 1085 | 4-methoxy-phenyl | OCH₂ | 2-quinolinyl | 4-CH₂-cyclopropyl | H |
| 1086 | 4-methoxy-phenyl | OCH₂ | 2-quinolinyl | 4-SMe | H |
| 1087 | 4-methoxy-phenyl | OCH₂ | 2-quinolinyl | 4-SEt | H |
| 1088 | 4-methoxy-phenyl | OCH₂ | 2-quinolinyl | 4-SⁱPr | H |
| 1089 | 4-pyridinyl | OCH₂ | 2-quinolinyl | H | H |
| 1090 | 4-pyridinyl | OCH₂ | 2-quinolinyl | F | H |
| 1091 | 4-pyridinyl | OCH₂ | 2-quinolinyl | 3-Cl | H |
| 1092 | 4-pyridinyl | OCH₂ | 2-quinolinyl | 3-CN | H |
| 1093 | 4-pyridinyl | OCH₂ | 2-quinolinyl | 3-NO₂ | H |
| 1094 | 4-pyridinyl | OCH₂ | 2-quinolinyl | 3-OMe | H |
| 1095 | 4-pyridinyl | OCH₂ | 2-quinolinyl | 3-Me | H |
| 1096 | 4-pyridinyl | OCH₂ | 2-quinolinyl | 3-Et | H |
| 1097 | 4-pyridinyl | OCH₂ | 2-quinolinyl | 3-ⁱPr | H |
| 1098 | 4-pyridinyl | OCH₂ | 2-quinolinyl | 3-^{t}Bu | H |
| 1099 | 4-pyridinyl | OCH₂ | 2-quinolinyl | 3-CF₃ | H |
| 1100 | 4-pyridinyl | OCH₂ | 2-quinolinyl | 3-SO₂Me | H |
| 1101 | 4-pyridinyl | OCH₂ | 2-quinolinyl | 3-SO₂Et | H |
| 1102 | 4-pyridinyl | OCH₂ | 2-quinolinyl | 3-SO₂ⁱPr | H |
| 1103 | 4-pyridinyl | OCH₂ | 2-quinolinyl | 3-OCF₃ | H |
| 1104 | 4-pyridinyl | OCH₂ | 2-quinolinyl | 3-OCH₂CF₃ | H |
| 1105 | 4-pyridinyl | OCH₂ | 2-quinolinyl | 3-NHMe | H |
| 1106 | 4-pyridinyl | OCH₂ | 2-quinolinyl | 3-NMe₂ | H |
| 1107 | 4-pyridinyl | OCH₂ | 2-quinolinyl | 3-OⁱPr | H |
| 1108 | 4-pyridinyl | OCH₂ | 2-quinolinyl | 3-CH₂-cyclopropyl | H |
| 1109 | 4-pyridinyl | OCH₂ | 2-quinolinyl | 3-SMe | H |
| 1110 | 4-pyridinyl | OCH₂ | 2-quinolinyl | 3-SEt | H |
| 1111 | 4-pyridinyl | OCH₂ | 2-quinolinyl | 3-SⁱPr | H |
| 1112 | 4-pyridinyl | OCH₂ | 2-quinolinyl | 4-F | H |
| 1113 | 4-pyridinyl | OCH₂ | 2-quinolinyl | 4-Cl | H |
| 1114 | 4-pyridinyl | OCH₂ | 2-quinolinyl | 4-OMe | H |
| 1115 | 4-pyridinyl | OCH₂ | 2-quinolinyl | 4-Me | H |
| 1116 | 4-pyridinyl | OCH₂ | 2-quinolinyl | 4-Et | H |
| 1117 | 4-pyridinyl | OCH₂ | 2-quinolinyl | 4-ⁱPr | H |
| 1118 | 4-pyridinyl | OCH₂ | 2-quinolinyl | 4-^{t}Bu | H |
| 1119 | 4-pyridinyl | OCH₂ | 2-quinolinyl | 4-CF₃ | H |
| 1120 | 4-pyridinyl | OCH₂ | 2-quinolinyl | 4-SO₂Me | H |
| 1121 | 4-pyridinyl | OCH₂ | 2-quinolinyl | 4-SO₂Et | H |
| 1122 | 4-pyridinyl | OCH₂ | 2-quinolinyl | 4-SO₂ⁱPr | H |
| 1123 | 4-pyridinyl | OCH₂ | 2-quinolinyl | 4-OCF₃ | H |
| 1124 | 4-pyridinyl | OCH₂ | 2-quinolinyl | 4-OCH₂CF₃ | H |
| 1125 | 4-pyridinyl | OCH₂ | 2-quinolinyl | 4-NHMe | H |
| 1126 | 4-pyridinyl | OCH₂ | 2-quinolinyl | 4-NMe₂ | H |
| 1127 | 4-pyridinyl | OCH₂ | 2-quinolinyl | 4-cyclopropyl | H |
| 1128 | 4-pyridinyl | OCH₂ | 2-quinolinyl | 4-OEt | H |
| 1129 | 4-pyridinyl | OCH₂ | 2-quinolinyl | 4-OⁱPr | H |
| 1130 | 4-pyridinyl | OCH₂ | 2-quinolinyl | 4-CH₂-cyclopropyl | H |
| 1131 | 4-pyridinyl | OCH₂ | 2-quinolinyl | 4-SMe | H |
| 1132 | 4-pyridinyl | OCH₂ | 2-quinolinyl | 4-SEt | H |
| 1133 | 4-pyridinyl | OCH₂ | 2-quinolinyl | 4-SⁱPr | H |
| 1134 | 4-pyridinyl | OCH₂ | 2-quinolinyl | 3-F | 4-F |
| 1135 | 4-pyridinyl | OCH₂ | 2-quinolinyl | 3-F | 4-OMe |
| 1136 | 4-pyridinyl | OCH₂ | 2-quinolinyl | 3-F | 4-Cl |
| 1137 | 4-pyridinyl | OCH₂ | 2-quinolinyl | 3-Cl | 4-OMe |
| 1138 | 4-pyridinyl | OCH₂ | 2-quinolinyl | 3-Cl | 4-CN |
| 1139 | 4-pyridinyl | OCH₂ | 2-quinolinyl | 3-OMe | 4-F |
| 1140 | 4-pyridinyl | OCH₂ | 2-quinolinyl | 3-CN | 4-OMe |
| 1141 | 4-pyridinyl | OCH₂ | 2-quinolinyl | 3-CF₃ | 4-CN |
| 1142 | 4-pyridinyl | OCH₂ | 2-quinolinyl | 3-NMe₂ | 4-F |
| 1143 | 4-pyridinyl | OCH₂ | 2-quinolinyl | 3-F | 4-NMe₂ |
| 1144 | 4-pyridinyl | OCH₂ | 2-quinolinyl | 3-O-cyclopropyl | 4-CN |
| 1145 | 4-pyridinyl | OCH₂ | 2-quinolinyl | 3-Cl | 4-Cl |
| 1146 | 4-pyridinyl | OCH₂ | 2-quinolinyl | 3-cyclopropyl | H |
| 1147 | 4-pyridinyl | OCH₂ | 2-quinolinyl | 3-OEt | H |
| 1148 | 4-pyridinyl | OCH₂ | 2-quinolinyl | 4-CN | H |
| 1149 | 4-pyridinyl | OCH₂ | 2-quinolinyl | 4-NO₂ | H |
| 1150 | 2-methoxy-5-pyridinyl | OCH₂ | 2-quinolinyl | H | H |
| 1151 | 2-methoxy-5-pyridinyl | OCH₂ | 2-quinolinyl | 3-F | H |
| 1152 | 5-(2-methoxy-pyridinyl) | OCH₂ | 2-quinolinyl | 3-Cl | H |
| 1153 | 2-methoxy-5-pyridinyl | OCH₂ | 2-quinolinyl | 3-CN | H |
| 1154 | 5-(2-methoxy-pyridinyl) | OCH₂ | 2-quinolinyl | 3-NO₂ | H |
| 1155 | 2-methoxy-5-pyridinyl | OCH₂ | 2-quinolinyl | 3-OMe | H |
| 1156 | 5-(2-methoxy-pyridinyl) | OCH₂ | 2-quinolinyl | 3-Me | H |
| 1157 | 2-methoxy-5-pyridinyl | OCH₂ | 2-quinolinyl | 3-Et | H |
| 1158 | 5-(2-methoxy-pyridinyl) | OCH₂ | 2-quinolinyl | 3-ⁱPr | H |
| 1159 | 2-methoxy-5-pyridinyl | OCH₂ | 2-quinolinyl | 3-^{t}Bu | H |
| 1160 | 5-(2-methoxy-pyridinyl) | OCH₂ | 2-quinolinyl | 3-CF₃ | H |
| 1161 | 2-methoxy-5-pyridinyl | OCH₂ | 2-quinolinyl | 3-SO₂Me | H |
| 1162 | 5-(2-methoxy-pyridinyl) | OCH₂ | 2-quinolinyl | 3-SO₂Et | H |
| 1163 | 2-methoxy-5-pyridinyl | OCH₂ | 2-quinolinyl | 3-SO₂ⁱPr | H |
| 1164 | 5-(2-methoxy-pyridinyl) | OCH₂ | 2-quinolinyl | 3-OCF₃ | H |
| 1165 | 2-methoxy-5-pyridinyl | OCH₂ | 2-quinolinyl | 3-OCH₂CF₃ | H |
| 1166 | 5-(2-methoxy-pyridinyl) | OCH₂ | 2-quinolinyl | 3-NHMe | H |
| 1167 | 2-methoxy-5-pyridinyl | OCH₂ | 2-quinolinyl | 3-NMe₂ | H |
| 1168 | 5-(2-methoxy-pyridinyl) | OCH₂ | 2-quinolinyl | 3-cyclopropyl | H |
| 1169 | 2-methoxy-5-pyridinyl | OCH₂ | 2-quinolinyl | 3-OEt | H |
| 1170 | 5-(2-methoxy-pyridinyl) | OCH₂ | 2-quinolinyl | 3-OⁱPr | H |
| 1171 | 2-methoxy-5-pyridinyl | OCH₂ | 2-quinolinyl | 3-CH₂-cyclopropyl | H |
| 1172 | 5-(2-methoxy-pyridinyl) | OCH₂ | 2-quinolinyl | 3-SMe | H |
| 1173 | 2-methoxy-5-pyridinyl | OCH₂ | 2-quinolinyl | 3-SEt | H |
| 1174 | 5-(2-methoxy-pyridinyl) | OCH₂ | 2-quinolinyl | 3-SⁱPr | H |
| 1175 | 2-methoxy-5-pyridinyl | OCH₂ | 2-quinolinyl | 4-F | H |
| 1176 | 2-methoxy-5-pyridinyl | OCH₂ | 2-quinolinyl | 4-Cl | H |
| 1177 | 2-methoxy-5-pyridinyl | OCH₂ | 2-quinolinyl | 4-CN | H |
| 1178 | 2-methoxy-5-pyridinyl | OCH₂ | 2-quinolinyl | 4-NO₂ | H |
| 1179 | 2-methoxy-5-pyridinyl | OCH₂ | 2-quinolinyl | 4-OMe | H |
| 1180 | 2-methoxy-5-pyridinyl | OCH₂ | 2-quinolinyl | 4-Me | H |
| 1181 | 2-methoxy-5-pyridinyl | OCH₂ | 2-quinolinyl | 4-Et | H |
| 1182 | 2-methoxy-5-pyridinyl | OCH₂ | 2-quinolinyl | 4-ⁱPr | H |
| 1183 | 2-methoxy-5-pyridinyl | OCH₂ | 2-quinolinyl | 4-^{t}Bu | H |
| 1184 | 2-methoxy-5-pyridinyl | OCH₂ | 2-quinolinyl | 4-CF₃ | H |
| 1185 | 2-methoxy-5-pyridinyl | OCH₂ | 2-quinolinyl | 4-SO₂Me | H |
| 1186 | 2-methoxy-5-pyridinyl | OCH₂ | 2-quinolinyl | 4-SO₂Et | H |
| 1187 | 2-methoxy-5-pyridinyl | OCH₂ | 2-quinolinyl | 4-SO₂ⁱPr | H |
| 1188 | 2-methoxy-5-pyridinyl | OCH₂ | 2-quinolinyl | 4-OCF₃ | H |
| 1189 | 2-methoxy-5-pyridinyl | OCH₂ | 2-quinolinyl | 4-OCH₂CF₃ | H |
| 1190 | 2-methoxy-5-pyridinyl | OCH₂ | 2-quinolinyl | 4-NHMe | H |
| 1191 | 2-methoxy-5-pyridinyl | OCH₂ | 2-quinolinyl | 4-NMe₂ | H |
| 1192 | 2-methoxy-5-pyridinyl | OCH₂ | 2-quinolinyl | 4-cyclopropyl | H |
| 1193 | 2-methoxy-5-pyridinyl | OCH₂ | 2-quinolinyl | 4-OEt | H |
| 1194 | 2-methoxy-5-pyridinyl | OCH₂ | 2-quinolinyl | 4-OⁱPr | H |
| 1195 | 2-methoxy-5-pyridinyl | OCH₂ | 2-quinolinyl | 4-CH₂-cyclopropyl | H |
| 1196 | 2-methoxy-5-pyridinyl | OCH₂ | 2-quinolinyl | 4-SMe | H |
| 1197 | 2-methoxy-5-pyridinyl | OCH₂ | 2-quinolinyl | 4-SEt | H |
| 1198 | 2-methoxy-5-pyridinyl | OCH₂ | 2-quinolinyl | 4-SⁱPr | H |
| 1199 | 2-hydroxy-5-pyridinyl | OCH₂ | 2-quinolinyl | H | H |
| 1200 | 2-hydroxy-5-pyridinyl | OCH₂ | 2-quinolinyl | 3-F | H |
| 1201 | 2-hydroxy-5-pyridinyl | OCH₂ | 2-quinolinyl | 3-Cl | H |
| 1202 | 2-hydroxy-5-pyridinyl | OCH₂ | 2-quinolinyl | 3-CN | H |
| 1203 | 2-hydroxy-5-pyridinyl | OCH₂ | 2-quinolinyl | 3-NO₂ | H |
| 1204 | 2-hydroxy-5-pyridinyl | OCH₂ | 2-quinolinyl | 3-OMe | H |
| 1205 | 2-hydroxy-5-pyridinyl | OCH₂ | 2-quinolinyl | 3-Me | H |
| 1206 | 2-hydroxy-5-pyridinyl | OCH₂ | 2-quinolinyl | 3-Et | H |
| 1207 | 2-hydroxy-5-pyridinyl | OCH₂ | 2-quinolinyl | 3-ⁱPr | H |
| 1208 | 2-hydroxy-5-pyridinyl | OCH₂ | 2-quinolinyl | 3-^{t}Bu | H |
| 1209 | 2-hydroxy-5-pyridinyl | OCH₂ | 2-quinolinyl | 3-CF₃ | H |
| 1210 | 2-hydroxy-5-pyridinyl | OCH₂ | 2-quinolinyl | 3-SO₂Me | H |
| 1211 | 2-hydroxy-5-pyridinyl | OCH₂ | 2-quinolinyl | 3-SO₂Et | H |
| 1212 | 2-hydroxy-5-pyridinyl | OCH₂ | 2-quinolinyl | 3-SO₂ⁱPr | H |
| 1213 | 2-hydroxy-5-pyridinyl | OCH₂ | 2-quinolinyl | 3-OCF₃ | H |
| 1214 | 2-hydroxy-5-pyridinyl | OCH₂ | 2-quinolinyl | 3-OCH₂CF₃ | H |
| 1215 | 2-hydroxy-5-pyridinyl | OCH₂ | 2-quinolinyl | 3-NHMe | H |
| 1216 | 2-hydroxy-5-pyridinyl | OCH₂ | 2-quinolinyl | 3-NMe₂ | H |
| 1217 | 2-hydroxy-5-pyridinyl | OCH₂ | 2-quinolinyl | 3-cyclopropyl | H |
| 1218 | 2-hydroxy-5-pyridinyl | OCH₂ | 2-quinolinyl | 3-OEt | H |
| 1219 | 2-hydroxy-5-pyridinyl | OCH₂ | 2-quinolinyl | 3-OⁱPr | H |
| 1220 | 2-hydroxy-5-pyridinyl | OCH₂ | 2-quinolinyl | 3-CH₂-cyclopropyl | H |
| 1221 | 2-hydroxy-5-pyridinyl | OCH₂ | 2-quinolinyl | 3-SMe | H |
| 1222 | 2-hydroxy-5-pyridinyl | OCH₂ | 2-quinolinyl | 3-SEt | H |
| 1223 | 2-hydroxy-5-pyridinyl | OCH₂ | 2-quinolinyl | 3-SⁱPr | H |
| 1224 | 2-hydroxy-5-pyridinyl | OCH₂ | 2-quinolinyl | 4-F | H |
| 1225 | 2-hydroxy-5-pyridinyl | OCH₂ | 2-quinolinyl | 4-Cl | H |
| 1226 | 2-hydroxy-5-pyridinyl | OCH₂ | 2-quinolinyl | 4-CN | H |
| 1227 | 2-hydroxy-5-pyridinyl | OCH₂ | 2-quinolinyl | 4-NO₂ | H |
| 1228 | 2-hydroxy-5-pyridinyl | OCH₂ | 2-quinolinyl | 4-OMe | H |
| 1229 | 2-hydroxy-5-pyridinyl | OCH₂ | 2-quinolinyl | 4-Me | H |
| 1230 | 2-hydroxy-5-pyridinyl | OCH₂ | 2-quinolinyl | 4-Et | H |
| 1231 | 2-hydroxy-5-pyridinyl | OCH₂ | 2-quinolinyl | 4-ⁱPr | H |
| 1232 | 2-hydroxy-5-pyridinyl | OCH₂ | 2-quinolinyl | 4-^{t}Bu | H |
| 1233 | 2-hydroxy-5-pyridinyl | OCH₂ | 2-quinolinyl | 4-CF₃ | H |
| 1234 | 2-hydroxy-5-pyridinyl | OCH₂ | 2-quinolinyl | 4-SO₂Me | H |
| 1235 | 2-hydroxy-5-pyridinyl | OCH₂ | 2-quinolinyl | 4-SO₂Et | H |
| 1236 | 2-hydroxy-5-pyridinyl | OCH₂ | 2-quinolinyl | 4-SO₂ⁱPr | H |
| 1237 | 2-hydroxy-5-pyridinyl | OCH₂ | 2-quinolinyl | 4-OCF₃ | H |
| 1238 | 2-hydroxy-5-pyridinyl | OCH₂ | 2-quinolinyl | 4-OCH₂CF₃ | H |
| 1239 | 2-hydroxy-5-pyridinyl | OCH₂ | 2-quinolinyl | 4-NHMe | H |
| 1240 | 2-hydroxy-5-pyridinyl | OCH₂ | 2-quinolinyl | 4-NMe₂ | H |
| 1241 | 2-hydroxy-5-pyridinyl | OCH₂ | 2-quinolinyl | 4-cyclopropyl | H |
| 1242 | 2-hydroxy-5-pyridinyl | OCH₂ | 2-quinolinyl | 4-OEt | H |
| 1243 | 2-hydroxy-5-pyridinyl | OCH₂ | 2-quinolinyl | 4-OⁱPr | H |
| 1244 | 2-hydroxy-5-pyridinyl | OCH₂ | 2-quinolinyl | 4-CH₂-cyclopropyl | H |
| 1245 | 2-hydroxy-5-pyridinyl | OCH₂ | 2-quinolinyl | 4-SMe | H |
| 1246 | 2-hydroxy-5-pyridinyl | OCH₂ | 2-quinolinyl | 4-SEt | H |
| 1247 | 2-hydroxy-5-pyridinyl | OCH₂ | 2-quinolinyl | 4-SⁱPr | H |
| 1248 | ⁱPr | OCH₂ | 2-quinolinyl | H | H |
| 1249 | Me | OCH₂ | 2-quinolinyl | H | H |
| 1250 | morpholinyl | OCH₂ | 2-quinolinyl | H | H |
| 1251 | N-piperazinyl | OCH₂ | 2-quinolinyl | H | H |
| 1252 | piperazinyl | OCH₂ | 2-quinolinyl | H | H |
| 1253 | piperidinyl | OCH₂ | 2-quinolinyl | H | H |
| 1254 | 3-pyridinyl | OCH₂ | 2-quinoxaline | H | H |
| 1255 | 4-pyridinyl | OCH₂ | 2-quinoxaline | H | H |
| 1256 | morpholinyl | OCH₂ | 2-quinoxaline | H | H |
| 1257 | 3-pyridinyl | OCH₂ | 5,6,7,8-tetrahydro- | H | H |
| | | | 2-quinolyl | | |
| 1258 | 4-pyridinyl | OCH₂ | 5,6,7,8-tetrahydro- | H | H |
| | | | 2-quinolyl | | |
| 1259 | morpholinyl | OCH₂ | 5,6,7,8-tetrahydro- | H | H |
| | | | 2-quinolyl | | |

In a further aspect the compounds of the disclosure are embodied in with distinct examples listed in the table below taken from Formula (III):

| Ex PCT | X | Y | Z | R₁ | R₂ |
|---|---|---|---|---|---|
| 1260 | 4-pyridinyl | CH₂O | 2-benzimidazolyl | H | H |
| 1261 | 4-pyridinyl | CH₂O | 2-benzoxazolyl | H | H |
| 1262 | 4-pyridinyl | CH₂O | 2-benzthiazolyl | H | H |
| 1263 | 4-pyridinyl | CH₂O | 2-pyridinyl | H | H |
| 1264 | 4-pyridinyl | CH₂O | 2-quinazolinyl | H | H |
| 1265 | 4-pyridinyl | CH₂O | 2-quinolinyl | H | H |
| 1266 | 4-pyridinyl | CH₂O | 2-quinolinyl | 3-F | H |
| 1267 | 4-pyridinyl | CH₂O | 2-quinolinyl | 3-Cl | H |
| 1268 | 4-pyridinyl | CH₂O | 2-quinolinyl | 3-CN | H |
| 1269 | 4-pyridinyl | CH₂O | 2-quinolinyl | 3-NO₂ | H |
| 1270 | 4-pyridinyl | CH₂O | 2-quinolinyl | 3-OMe | H |
| 1271 | 4-pyridinyl | CH₂O | 2-quinolinyl | 3-Me | H |
| 1272 | 4-pyridinyl | CH₂O | 2-quinolinyl | 3-Et | H |
| 1273 | 4-pyridinyl | CH₂O | 2-quinolinyl | 3-ⁱPr | H |
| 1274 | 4-pyridinyl | CH₂O | 2-quinolinyl | 3-^{t}Bu | H |
| 1275 | 4-pyridinyl | CH₂O | 2-quinolinyl | 3-CF₃ | H |
| 1276 | 4-pyridinyl | CH₂O | 2-quinolinyl | 3-SO₂Me | H |
| 1277 | 4-pyridinyl | CH₂O | 2-quinolinyl | 3-SO₂Et | H |
| 1278 | 4-pyridinyl | CH₂O | 2-quinolinyl | 3-SO₂ⁱPr | H |
| 1279 | 4-pyridinyl | CH₂O | 2-quinolinyl | 3-OCF₃ | H |
| 1280 | 4-pyridinyl | CH₂O | 2-quinolinyl | 3-OCH₂CF₃ | H |
| 1281 | 4-pyridinyl | CH₂O | 2-quinolinyl | 3-NHMe | H |
| 1282 | 4-pyridinyl | CH₂O | 2-quinolinyl | 3-NMe₂ | H |
| 1283 | 4-pyridinyl | CH₂O | 2-quinolinyl | 3- | H |
| | | | | cyclopropyl | |
| 1284 | 4-pyridinyl | CH₂O | 2-quinolinyl | 3-OEt | H |
| 1285 | 4-pyridinyl | CH₂O | 2-quinolinyl | 3-OⁱPr | H |
| 1286 | 4-pyridinyl | CH₂O | 2-quinolinyl | 3-CH₂- | H |
| | | | | cyclopropyl | |
| 1287 | 4-pyridinyl | CH₂O | 2-quinolinyl | 3-SMe | H |
| 1288 | 4-pyridinyl | CH₂O | 2-quinolinyl | 3-SEt | H |
| 1289 | 4-pyridinyl | CH₂O | 2-quinolinyl | 3-SⁱPr | H |
| 1290 | 4-pyridinyl | CH₂O | 2-quinolinyl | 4-F | H |
| 1291 | 4-pyridinyl | CH₂O | 2-quinolinyl | 4-Cl | H |
| 1292 | 4-pyridinyl | CH₂O | 2-quinolinyl | 4-CN | H |
| 1293 | 4-pyridinyl | CH₂O | 2-quinolinyl | 4-NO₂ | H |
| 1294 | 4-pyridinyl | CH₂O | 2-quinolinyl | 4-OMe | H |
| 1295 | 4-pyridinyl | CH₂O | 2-quinolinyl | 4-Me | H |
| 1296 | 4-pyridinyl | CH₂O | 2-quinolinyl | 4-Et | H |
| 1297 | 4-pyridinyl | CH₂O | 2-quinolinyl | 4-ⁱPr | H |
| 1298 | 4-pyridinyl | CH₂O | 2-quinolinyl | 4-^{t}Bu | H |
| 1299 | 4-pyridinyl | CH₂O | 2-quinolinyl | 4-CF₃ | H |
| 1300 | 4-pyridinyl | CH₂O | 2-quinolinyl | 4-SO₂Me | H |
| 1301 | 4-pyridinyl | CH₂O | 2-quinolinyl | 4-SO₂Et | H |
| 1302 | 4-pyridinyl | CH₂O | 2-quinolinyl | 4-SO₂ⁱPr | H |
| 1303 | 4-pyridinyl | CH₂O | 2-quinolinyl | 4-OCF₃ | H |
| 1304 | 4-pyridinyl | CH₂O | 2-quinolinyl | 4-OCH₂CF₃ | H |
| 1305 | 4-pyridinyl | CH₂O | 2-quinolinyl | 4-NHMe | H |
| 1306 | 4-pyridinyl | CH₂O | 2-quinolinyl | 4-NMe₂ | H |
| 1307 | 4-pyridinyl | CH₂O | 2-quinolinyl | 4- | H |
| | | | | cyclopropyl | |
| 1308 | 4-pyridinyl | CH₂O | 2-quinolinyl | 4-OEt | H |
| 1309 | 4-pyridinyl | CH₂O | 2-quinolinyl | 4-OⁱPr | H |
| 1310 | 4-pyridinyl | CH₂O | 2-quinolinyl | 4-CH₂- | H |
| | | | | cyclopropyl | |
| 1311 | 4-pyridinyl | CH₂O | 2-quinolinyl | 4-SMe | H |
| 1312 | 4-pyridinyl | CH₂O | 2-quinolinyl | 4-SEt | H |
| 1313 | 4-pyridinyl | CH₂O | 2-quinolinyl | 4-SⁱPr | H |
| 1314 | ⁱPr | CH₂O | 2-quinolinyl | H | H |
| 1315 | Me | CH₂O | 2-quinolinyl | H | H |
| 1316 | morpholinyl | CH₂O | 2-quinolinyl | H | H |
| 1317 | N-piperazinyl | CH₂O | 2-quinolinyl | H | H |
| 1318 | piperazinyl | CH₂O | 2-quinolinyl | H | H |
| 1319 | piperidinyl | CH₂O | 2-quinolinyl | H | H |
| 1320 | 4-pyridinyl | CH₂O | 2-quinoxalinyl | H | H |
| 1321 | 4-pyridinyl | CH₂O | 5,6,7,8-tetrahydro-2- | H | H |
| | | | quinolyl | | |
| 1322 | 3-pyridinyl | OCH₂ | 2-benzimidazolyl | H | H |
| 1323 | 4-pyridinyl | OCH₂ | 2-benzimidazolyl | H | H |
| 1324 | morpholinyl | OCH₂ | 2-benzimidazolyl | H | H |
| 1325 | 3-pyridinyl | OCH₂ | 2-benzoxazolyl | H | H |
| 1326 | 4-pyridinyl | OCH₂ | 2-benzoxazolyl | H | H |
| 1327 | morpholinyl | OCH₂ | 2-benzoxazolyl | H | H |
| 1328 | 3-pyridinyl | OCH₂ | 2-benzthiazolyl | H | H |
| 1329 | 4-pyridinyl | OCH₂ | 2-benzthiazolyl | H | H |
| 1330 | morpholinyl | OCH₂ | 2-benzthiazolyl | H | H |
| 1331 | 3-pyridinyl | OCH₂ | 2-pyridinyl | H | H |
| 1332 | 4-pyridinyl | OCH₂ | 2-pyridinyl | H | H |
| 1333 | morpholinyl | OCH₂ | 2-pyridinyl | H | H |
| 1334 | 3-pyridinyl | OCH₂ | 2-quinazoline | H | H |
| 1335 | 4-pyridinyl | OCH₂ | 2-quinazoline | H | H |
| 1336 | morpholinyl | OCH₂ | 2-quinazolinyl | H | H |
| 1337 | 3,4- | OCH₂ | 2-quinolinyl | H | H |
| | dimethoxyphenyl | | | | |
| 1339 | 2-methoxy-4- | OCH₂ | 2-quinolinyl | H | H |
| | pyridinyl | | | | |
| 1340 | 2-methoxy-4- | OCH₂ | 2-quinolinyl | 3-F | H |
| | pyridinyl) | | | | |
| 1341 | 2-methoxy-4- | OCH₂ | 2-quinolinyl | 3-Cl | H |
| | pyridinyl | | | | |
| 1342 | 2-methoxy-4- | OCH₂ | 2-quinolinyl | 3-CN | H |
| | pyridinyl) | | | | |
| 1343 | 2-methoxy-4- | OCH₂ | 2-quinolinyl | 3-NO₂ | H |
| | pyridinyl | | | | |
| 1344 | 2-methoxy-4- | OCH₂ | 2-quinolinyl | 3-OMe | H |
| | pyridinyl) | | | | |
| 1345 | 2-methoxy-4- | OCH₂ | 2-quinolinyl | 3-Me | H |
| | pyridinyl | | | | |
| 1346 | 2-methoxy-4- | OCH₂ | 2-quinolinyl | 3-Et | H |
| | pyridinyl) | | | | |
| 1347 | 2-methoxy-4- | OCH₂ | 2-quinolinyl | 3-ⁱPr | H |
| | pyridinyl | | | | |
| 1348 | 2-methoxy-4- | OCH₂ | 2-quinolinyl | 3-^{t}Bu | H |
| | pyridinyl) | | | | |
| 1349 | 2-methoxy-4- | OCH₂ | 2-quinolinyl | 3-CF₃ | H |
| | pyridinyl | | | | |
| 1350 | 2-methoxy-4- | OCH₂ | 2-quinolinyl | 3-SO₂Me | H |
| | pyridinyl) | | | | |
| 1351 | 2-methoxy-4- | OCH₂ | 2-quinolinyl | 3-SO₂Et | H |
| | pyridinyl | | | | |
| 1352 | 2-methoxy-4- | OCH₂ | 2-quinolinyl | 3-SO₂ⁱPr | H |
| | pyridinyl) | | | | |
| 1353 | 2-methoxy-4- | OCH₂ | 2-quinolinyl | 3-OCF₃ | H |
| | pyridinyl | | | | |
| 1354 | 2-methoxy-4- | OCH₂ | 2-quinolinyl | 3-OCH₂CF₃ | H |
| | pyridinyl) | | | | |
| 1355 | 2-methoxy-4- | OCH₂ | 2-quinolinyl | 3-NHMe | H |
| | pyridinyl | | | | |
| 1356 | 2-methoxy-4- | OCH₂ | 2-quinolinyl | 3-NMe₂ | H |
| | pyridinyl) | | | | |
| 1357 | 2-methoxy-4- | OCH₂ | 2-quinolinyl | 3- | H |
| | pyridinyl | | | cyclopropyl | |
| 1358 | 2-methoxy-4- | OCH₂ | 2-quinolinyl | 3-OEt | H |
| | pyridinyl) | | | | |
| 1359 | 2-methoxy-4- | OCH₂ | 2-quinolinyl | 3-OⁱPr | H |
| | pyridinyl | | | | |
| 1360 | 2-methoxy-4- | OCH₂ | 2-quinolinyl | 3-CH₂- | H |
| | pyridinyl) | | | cyclopropyl | |
| 1361 | 2-methoxy-4- | OCH₂ | 2-quinolinyl | 3-SMe | H |
| | pyridinyl | | | | |
| 1362 | 2-methoxy-4- | OCH₂ | 2-quinolinyl | 3-SEt | H |
| | pyridinyl) | | | | |
| 1363 | 2-methoxy-4- | OCH₂ | 2-quinolinyl | 3-SⁱPr | H |
| | pyridinyl | | | | |
| 1364 | 2-methoxy-4- | OCH₂ | 2-quinolinyl | 4-F | H |
| | pyridinyl) | | | | |
| 1365 | 2-methoxy-4- | OCH₂ | 2-quinolinyl | 4-Cl | H |
| | pyridinyl | | | | |
| 1366 | 2-methoxy-4- | OCH₂ | 2-quinolinyl | 4-CN | H |
| | pyridinyl) | | | | |
| 1367 | 2-methoxy-4- | OCH₂ | 2-quinolinyl | 4-NO2 | H |
| | pyridinyl | | | | |
| 1368 | 2-methoxy-4- | OCH₂ | 2-quinolinyl | 4-OMe | H |
| | pyridinyl) | | | | |
| 1369 | 2-methoxy-4- | OCH₂ | 2-quinolinyl | 4-Me | H |
| | pyridinyl | | | | |
| 1370 | 2-methoxy-4- | OCH₂ | 2-quinolinyl | 4-Et | H |
| | pyridinyl) | | | | |
| 1371 | 2-methoxy-4- | OCH₂ | 2-quinolinyl | 4-iPr | H |
| | pyridinyl | | | | |
| 1372 | 2-methoxy-4- | OCH₂ | 2-quinolinyl | 4-tBu | H |
| | pyridinyl) | | | | |
| 1373 | 2-methoxy-4- | OCH₂ | 2-quinolinyl | 4-CF3 | H |
| | pyridinyl | | | | |
| 1374 | 2-methoxy-4- | OCH₂ | 2-quinolinyl | 4-SO2Me | H |
| | pyridinyl) | | | | |
| 1375 | 2-methoxy-4- | OCH₂ | 2-quinolinyl | 4-SO2Et | H |
| | pyridinyl | | | | |
| 1376 | 2-methoxy-4- | OCH₂ | 2-quinolinyl | 4-SO2iPr | H |
| | pyridinyl) | | | | |
| 1377 | 2-methoxy-4- | OCH₂ | 2-quinolinyl | 4-OCF3 | H |
| | pyridinyl | | | | |
| 1378 | 2-methoxy-4- | OCH₂ | 2-quinolinyl | 4-OCH2CF3 | H |
| | pyridinyl) | | | | |
| 1379 | 2-methoxy-4- | OCH₂ | 2-quinolinyl | 4-NHMe | H |
| | pyridinyl | | | | |
| 1380 | 2-methoxy-4- | OCH₂ | 2-quinolinyl | 4-NMe2 | H |
| | pyridinyl) | | | | |
| 1381 | 2-methoxy-4- | OCH₂ | 2-quinolinyl | 4- | H |
| | pyridinyl | | | cyclopropyl | |
| 1382 | 2-methoxy-4- | OCH₂ | 2-quinolinyl | 4-OEt | H |
| | pyridinyl) | | | | |
| 1383 | 2-methoxy-4- | OCH₂ | 2-quinolinyl | 4-OiPr | H |
| | pyridinyl | | | | |
| 1384 | 2-methoxy-4- | OCH₂ | 2-quinolinyl | 4-CH2- | H |
| | pyridinyl) | | | cyclopropyl | |
| 1385 | 2-methoxy-4- | OCH₂ | 2-quinolinyl | 4-SMe | H |
| | pyridinyl | | | | |
| 1386 | 2-methoxy-4- | OCH₂ | 2-quinolinyl | 4-SEt | H |
| | pyridinyl) | | | | |
| 1387 | 2-methoxy-4- | OCH₂ | 2-quinolinyl | 4-SiPr | H |
| | pyridinyl | | | | |
| 1388 | 2-hydroxy-4- | OCH₂ | 2-quinolinyl | H | H |
| | pyridinyl | | | | |
| 1389 | 2-hydroxy-4- | OCH₂ | 2-quinolinyl | 3-F | H |
| | pyridinyl | | | | |
| 1390 | 2-hydroxy-4- | OCH₂ | 2-quinolinyl | 3-Cl | H |
| | pyridinyl | | | | |
| 1391 | 2-hydroxy-4- | OCH₂ | 2-quinolinyl | 3-CN | H |
| | pyridinyl | | | | |
| 1392 | 2-hydroxy-4- | OCH₂ | 2-quinolinyl | 3-NO₂ | H |
| | pyridinyl | | | | |
| 1393 | 2-hydroxy-4- | OCH₂ | 2-quinolinyl | 3-OMe | H |
| | pyridinyl | | | | |
| 1394 | 2-hydroxy-4- | OCH₂ | 2-quinolinyl | 3-Me | H |
| | pyridinyl | | | | |
| 1395 | 2-hydroxy-4- | OCH₂ | 2-quinolinyl | 3-Et | H |
| | pyridinyl | | | | |
| 1396 | 2-hydroxy-4- | OCH₂ | 2-quinolinyl | 3-ⁱPr | H |
| | pyridinyl | | | | |
| 1397 | 2-hydroxy-4- | OCH₂ | 2-quinolinyl | 3-^{t}Bu | H |
| | pyridinyl | | | | |
| 1398 | 2-hydroxy-4- | OCH₂ | 2-quinolinyl | 3-CF₃ | H |
| | pyridinyl | | | | |
| 1399 | 2-hydroxy-4- | OCH₂ | 2-quinolinyl | 3-SO₂Me | H |
| | pyridinyl | | | | |
| 1400 | 2-hydroxy-4- | OCH₂ | 2-quinolinyl | 3-SO₂Et | H |
| | pyridinyl | | | | |
| 1401 | 2-hydroxy-4- | OCH₂ | 2-quinolinyl | 3-SO₂ⁱPr | H |
| | pyridinyl | | | | |
| 1402 | 2-hydroxy-4- | OCH₂ | 2-quinolinyl | 3-OCF₃ | H |
| | pyridinyl | | | | |
| 1403 | 2-hydroxy-4- | OCH₂ | 2-quinolinyl | 3-OCH₂CF₃ | H |
| | pyridinyl | | | | |
| 1404 | 2-hydroxy-4- | OCH₂ | 2-quinolinyl | 3-NHMe | H |
| | pyridinyl | | | | |
| 1405 | 2-hydroxy-4- | OCH₂ | 2-quinolinyl | 3-NMe₂ | H |
| | pyridinyl | | | | |
| 1406 | 2-hydroxy-4- | OCH₂ | 2-quinolinyl | 3- | H |
| | pyridinyl | | | cyclopropyl | |
| 1407 | 2-hydroxy-4- | OCH₂ | 2-quinolinyl | 3-OEt | H |
| | pyridinyl | | | | |
| 1408 | 2-hydroxy-4- | OCH₂ | 2-quinolinyl | 3-OⁱPr | H |
| | pyridinyl | | | | |
| 1409 | 2-hydroxy-4- | OCH₂ | 2-quinolinyl | 3-CH₂- | H |
| | pyridinyl | | | cyclopropyl | |
| 1410 | 2-hydroxy-4- | OCH₂ | 2-quinolinyl | 3-SMe | H |
| | pyridinyl | | | | |
| 1411 | 2-hydroxy-4- | OCH₂ | 2-quinolinyl | 3-SEt | H |
| | pyridinyl | | | | |
| 1412 | 2-hydroxy-4- | OCH₂ | 2-quinolinyl | 3-SⁱPr | H |
| | pyridinyl | | | | |
| 1413 | 2-hydroxy-4- | OCH₂ | 2-quinolinyl | 4-F | H |
| | pyridinyl | | | | |
| 1414 | 2-hydroxy-4- | OCH₂ | 2-quinolinyl | 4-Cl | H |
| | pyridinyl | | | | |
| 1415 | 2-hydroxy-4- | OCH₂ | 2-quinolinyl | 4-CN | H |
| | pyridinyl | | | | |
| 1416 | 2-hydroxy-4- | OCH₂ | 2-quinolinyl | 4-NO2 | H |
| | pyridinyl | | | | |
| 1417 | 2-hydroxy-4- | OCH₂ | 2-quinolinyl | 4-OMe | H |
| | pyridinyl | | | | |
| 1418 | 2-hydroxy-4- | OCH₂ | 2-quinolinyl | 4-Me | H |
| | pyridinyl | | | | |
| 1419 | 2-hydroxy-4- | OCH₂ | 2-quinolinyl | 4-Et | H |
| | pyridinyl | | | | |
| 1420 | 2-hydroxy-4- | OCH₂ | 2-quinolinyl | 4-iPr | H |
| | pyridinyl | | | | |
| 1421 | 2-hydroxy-4- | OCH₂ | 2-quinolinyl | 4-tBu | H |
| | pyridinyl | | | | |
| 1422 | 2-hydroxy-4- | OCH₂ | 2-quinolinyl | 4-CF3 | H |
| | pyridinyl | | | | |
| 1423 | 2-hydroxy-4- | OCH₂ | 2-quinolinyl | 4-SO2Me | H |
| | pyridinyl | | | | |
| 1424 | 2-hydroxy-4- | OCH₂ | 2-quinolinyl | 4-SO2Et | H |
| | pyridinyl | | | | |
| 1425 | 2-hydroxy-4- | OCH₂ | 2-quinolinyl | 4-SO2iPr | H |
| | pyridinyl | | | | |
| 1426 | 2-hydroxy-4- | OCH₂ | 2-quinolinyl | 4-OCF3 | H |
| | pyridinyl | | | | |
| 1427 | 2-hydroxy-4- | OCH₂ | 2-quinolinyl | 4-OCH2CF3 | H |
| | pyridinyl | | | | |
| 1428 | 2-hydroxy-4- | OCH₂ | 2-quinolinyl | 4-NHMe | H |
| | pyridinyl | | | | |
| 1429 | 2-hydroxy-4- | OCH₂ | 2-quinolinyl | 4-NMe2 | H |
| | pyridinyl | | | | |
| 1430 | 2-hydroxy-4- | OCH₂ | 2-quinolinyl | 4- | H |
| | pyridinyl | | | cyclopropyl | |
| 1431 | 2-hydroxy-4- | OCH₂ | 2-quinolinyl | 4-OEt | H |
| | pyridinyl | | | | |
| 1432 | 2-hydroxy-4- | OCH₂ | 2-quinolinyl | 4-OiPr | H |
| | pyridinyl | | | | |
| 1433 | 2-hydroxy-4- | OCH₂ | 2-quinolinyl | 4-CH2- | H |
| | pyridinyl | | | cyclopropyl | |
| 1434 | 2-hydroxy-4- | OCH₂ | 2-quinolinyl | 4-SMe | H |
| | pyridinyl | | | | |
| 1435 | 2-hydroxy-4- | OCH₂ | 2-quinolinyl | 4-SEt | H |
| | pyridinyl | | | | |
| 1436 | 2-hydroxy-4- | OCH₂ | 2-quinolinyl | 4-SiPr | H |
| | pyridinyl | | | | |
| 1437 | 4-chloro-phenyl | OCH₂ | 2-quinolinyl | H | H |
| 1438 | 4-chloro-phenyl | OCH₂ | 2-quinolinyl | 3-F | H |
| 1439 | 4-chloro-phenyl | OCH₂ | 2-quinolinyl | 3-Cl | H |
| 1440 | 4-chloro-phenyl | OCH₂ | 2-quinolinyl | 3-CN | H |
| 1441 | 4-chloro-phenyl | OCH₂ | 2-quinolinyl | 3-NO₂ | H |
| 1442 | 4-chloro-phenyl | OCH₂ | 2-quinolinyl | 3-OMe | H |
| 1443 | 4-chloro-phenyl | OCH₂ | 2-quinolinyl | 3-Me | H |
| 1444 | 4-chloro-phenyl | OCH₂ | 2-quinolinyl | 3-Et | H |
| 1445 | 4-chloro-phenyl | OCH₂ | 2-quinolinyl | 3-ⁱPr | H |
| 1446 | 4-chloro-phenyl | OCH₂ | 2-quinolinyl | 3-^{t}Bu | H |
| 1447 | 4-chloro-phenyl | OCH₂ | 2-quinolinyl | 3-CF₃ | H |
| 1448 | 4-chloro-phenyl | OCH₂ | 2-quinolinyl | 3-SO₂Me | H |
| 1449 | 4-chloro-phenyl | OCH₂ | 2-quinolinyl | 3-SO₂Et | H |
| 1450 | 4-chloro-phenyl | OCH₂ | 2-quinolinyl | 3-SO₂ⁱPr | H |
| 1451 | 4-chloro-phenyl | OCH₂ | 2-quinolinyl | 3-OCF₃ | H |
| 1452 | 4-chloro-phenyl | OCH₂ | 2-quinolinyl | 3-OCH₂CF₃ | H |
| 1453 | 4-chloro-phenyl | OCH₂ | 2-quinolinyl | 3-NHMe | H |
| 1454 | 4-chloro-phenyl | OCH₂ | 2-quinolinyl | 3-NMe₂ | H |
| 1455 | 4-chloro-phenyl | OCH₂ | 2-quinolinyl | 3- | H |
| | | | | cyclopropyl | H |
| 1456 | 4-chloro-phenyl | OCH₂ | 2-quinolinyl | 3-OEt | H |
| 1457 | 4-chloro-phenyl | OCH₂ | 2-quinolinyl | 3-OⁱPr | H |
| 1458 | 4-chloro-phenyl | OCH₂ | 2-quinolinyl | 3-CH₂- | H |
| | | | | cyclopropyl | |
| 1459 | 4-chloro-phenyl | OCH₂ | 2-quinolinyl | 3-SMe | H |
| 1460 | 4-chloro-phenyl | OCH₂ | 2-quinolinyl | 3-SEt | H |
| 1461 | 4-chloro-phenyl | OCH₂ | 2-quinolinyl | 3-SⁱPr | H |
| 1462 | 4-chloro-phenyl | OCH₂ | 2-quinolinyl | 4-F | H |
| 1463 | 4-chloro-phenyl | OCH₂ | 2-quinolinyl | 4-Cl | H |
| 1464 | 4-chloro-phenyl | OCH₂ | 2-quinolinyl | 4-CN | H |
| 1465 | 4-chloro-phenyl | OCH₂ | 2-quinolinyl | 4-NO2 | H |
| 1466 | 4-chloro-phenyl | OCH₂ | 2-quinolinyl | 4-OMe | H |
| 1467 | 4-chloro-phenyl | OCH₂ | 2-quinolinyl | 4-Me | H |
| 1468 | 4-chloro-phenyl | OCH₂ | 2-quinolinyl | 4-Et | H |
| 1469 | 4-chloro-phenyl | OCH₂ | 2-quinolinyl | 4-iPr | H |
| 1470 | 4-chloro-phenyl | OCH₂ | 2-quinolinyl | 4-tBu | H |
| 1471 | 4-chloro-phenyl | OCH₂ | 2-quinolinyl | 4-CF3 | H |
| 1472 | 4-chloro-phenyl | OCH₂ | 2-quinolinyl | 4-SO2Me | H |
| 1473 | 4-chloro-phenyl | OCH₂ | 2-quinolinyl | 4-SO2Et | H |
| 1474 | 4-chloro-phenyl | OCH₂ | 2-quinolinyl | 4-SO2iPr | H |
| 1475 | 4-chloro-phenyl | OCH₂ | 2-quinolinyl | 4-OCF3 | H |
| 1476 | 4-chloro-phenyl | OCH₂ | 2-quinolinyl | 4-OCH2CF3 | H |
| 1477 | 4-chloro-phenyl | OCH₂ | 2-quinolinyl | 4-NHMe | H |
| 1478 | 4-chloro-phenyl | OCH₂ | 2-quinolinyl | 4-NMe2 | H |
| 1479 | 4-chloro-phenyl | OCH₂ | 2-quinolinyl | 4- | H |
| | | | | cyclopropyl | |
| 1480 | 4-chloro-phenyl | OCH₂ | 2-quinolinyl | 4-OEt | H |
| 1481 | 4-chloro-phenyl | OCH₂ | 2-quinolinyl | 4-OiPr | H |
| 1482 | 4-chloro-phenyl | OCH₂ | 2-quinolinyl | 4-CH2- | H |
| | | | | cyclopropyl | |
| 1483 | 4-chloro-phenyl | OCH₂ | 2-quinolinyl | 4-SMe | H |
| 1484 | 4-chloro-phenyl | OCH₂ | 2-quinolinyl | 4-SEt | H |
| 1485 | 4-chloro-phenyl | OCH₂ | 2-quinolinyl | 4-SiPr | H |
| 1486 | 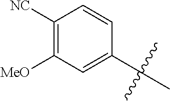 | OCH₂ | 2-quinolinyl | H | H |
| 1487 | 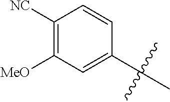 | OCH₂ | 2-quinolinyl | 3-F | H |
| 1488 | 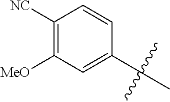 | OCH₂ | 2-quinolinyl | 3-Cl | H |
| 1489 | 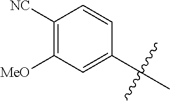 | OCH₂ | 2-quinolinyl | 3-CN | H |
| 1490 | 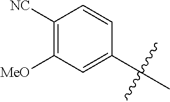 | OCH₂ | 2-quinolinyl | 3-NO₂ | H |
| 1491 | 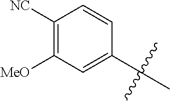 | OCH₂ | 2-quinolinyl | 3-OMe | H |
| 1492 | 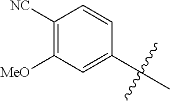 | OCH₂ | 2-quinolinyl | 3-Me | H |
| 1493 | 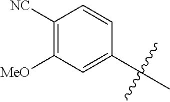 | OCH₂ | 2-quinolinyl | 3-Et | H |
| 1494 | 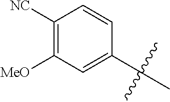 | OCH₂ | 2-quinolinyl | 3-ⁱPr | H |
| 1495 | 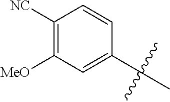 | OCH₂ | 2-quinolinyl | 3-^{t}Bu | H |
| 1496 | 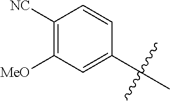 | OCH₂ | 2-quinolinyl | 3-CF₃ | H |
| 1497 | 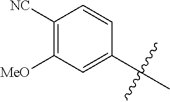 | OCH₂ | 2-quinolinyl | 3-SO₂Me | H |
| 1498 | 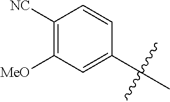 | OCH₂ | 2-quinolinyl | 3-SO₂Et | H |
| 1499 | 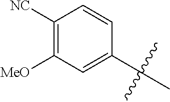 | OCH₂ | 2-quinolinyl | 3-SO₂ⁱPr | H |
| 1500 | 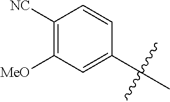 | OCH₂ | 2-quinolinyl | 3-OCF₃ | H |
| 1501 | 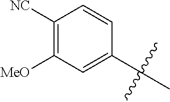 | OCH₂ | 2-quinolinyl | 3-OCH₂CF₃ | H |
| 1502 | 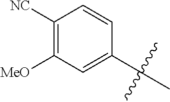 | OCH₂ | 2-quinolinyl | 3-NHMe | H |
| 1503 | 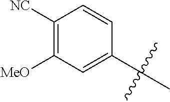 | OCH₂ | 2-quinolinyl | 3-NMe₂ | H |
| 1504 | 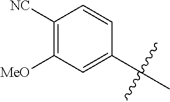 | OCH₂ | 2-quinolinyl | 3- cyclopropyl | H |
| 1505 | 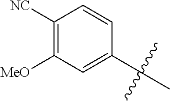 | OCH₂ | 2-quinolinyl | 3-OEt | H |
| 1506 | 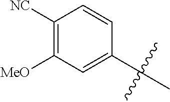 | OCH₂ | 2-quinolinyl | 3-OⁱPr | H |
| 1507 | 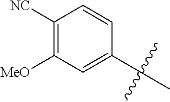 | OCH₂ | 2-quinolinyl | 3-CH₂- cyclopropyl | H |
| 1508 | 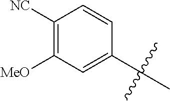 | OCH₂ | 2-quinolinyl | 3-SMe | H |
| 1509 | 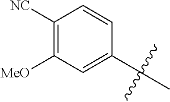 | OCH₂ | 2-quinolinyl | 3-SEt | H |
| 1510 | 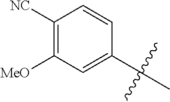 | OCH₂ | 2-quinolinyl | 3-SⁱPr | H |
| 1511 | 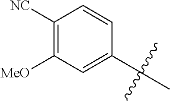 | OCH₂ | 2-quinolinyl | 4-F | H |
| 1512 | 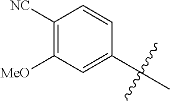 | OCH₂ | 2-quinolinyl | 4-Cl | H |
| 1513 | 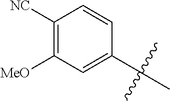 | OCH₂ | 2-quinolinyl | 4-CN | H |
| 1514 | 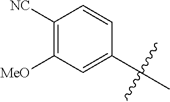 | OCH₂ | 2-quinolinyl | 4-NO2 | H |
| 1515 | 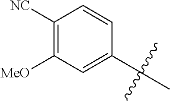 | OCH₂ | 2-quinolinyl | 4-OMe | H |
| 1516 | 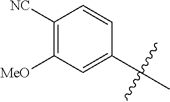 | OCH₂ | 2-quinolinyl | 4-Me | H |
| 1517 | 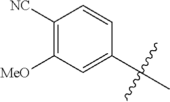 | OCH₂ | 2-quinolinyl | 4-Et | H |
| 1518 | 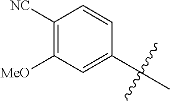 | OCH₂ | 2-quinolinyl | 4-iPr | H |
| 1519 | 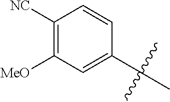 | OCH₂ | 2-quinolinyl | 4-tBu | H |
| 1520 | 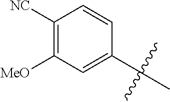 | OCH₂ | 2-quinolinyl | 4-CF3 | H |
| 1521 | 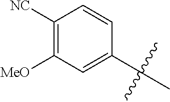 | OCH₂ | 2-quinolinyl | 4-SO2Me | H |
| 1522 | 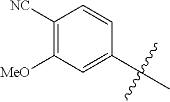 | OCH₂ | 2-quinolinyl | 4-SO2Et | H |
| 1523 | 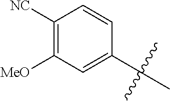 | OCH₂ | 2-quinolinyl | 4-SO2iPr | H |
| 1524 | 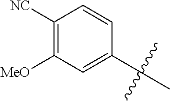 | OCH₂ | 2-quinolinyl | 4-OCF3 | H |
| 1525 | 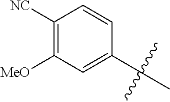 | OCH₂ | 2-quinolinyl | 4-OCH2CF3 | H |
| 1526 | 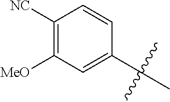 | OCH₂ | 2-quinolinyl | 4-NHMe | H |
| 1527 | 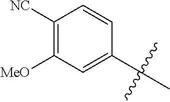 | OCH₂ | 2-quinolinyl | 4-NMe2 | H |
| 1528 | 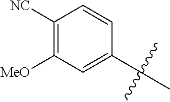 | OCH₂ | 2-quinolinyl | 4- cyclopropyl | H |
| 1529 | 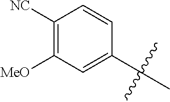 | OCH₂ | 2-quinolinyl | 4-OEt | H |
| 1530 | 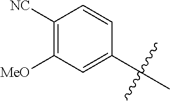 | OCH₂ | 2-quinolinyl | 4-OiPr | H |
| 1531 | 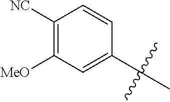 | OCH₂ | 2-quinolinyl | 4-CH2- cyclopropyl | H |
| 1532 | 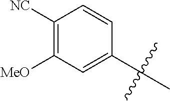 | OCH₂ | 2-quinolinyl | 4-SMe | H |
| 1533 | 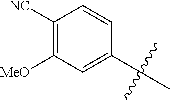 | OCH₂ | 2-quinolinyl | 4-SEt | H |
| 1534 | 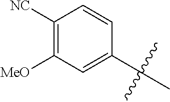 | OCH₂ | 2-quinolinyl | 4-SiPr | H |
| 1535 | 4-cyano-phenyl | OCH₂ | 2-quinolinyl | H | H |
| 1536 | 4-cyano-phenyl | OCH₂ | 2-quinolinyl | 3-F | H |
| 1537 | 4-cyano-phenyl | OCH₂ | 2-quinolinyl | 3-Cl | H |
| 1538 | 4-cyano-phenyl | OCH₂ | 2-quinolinyl | 3-CN | H |
| 1539 | 4-cyano-phenyl | OCH₂ | 2-quinolinyl | 3-NO₂ | H |
| 1540 | 4-cyano-phenyl | OCH₂ | 2-quinolinyl | 3-OMe | H |
| 1541 | 4-cyano-phenyl | OCH₂ | 2-quinolinyl | 3-Me | H |
| 1542 | 4-cyano-phenyl | OCH₂ | 2-quinolinyl | 3-Et | H |
| 1543 | 4-cyano-phenyl | OCH₂ | 2-quinolinyl | 3-ⁱPr | H |
| 1544 | 4-cyano-phenyl | OCH₂ | 2-quinolinyl | 3-^{t}Bu | H |
| 1545 | 4-cyano-phenyl | OCH₂ | 2-quinolinyl | 3-CF₃ | H |
| 1546 | 4-cyano-phenyl | OCH₂ | 2-quinolinyl | 3-SO₂Me | H |
| 1547 | 4-cyano-phenyl | OCH₂ | 2-quinolinyl | 3-SO₂Et | H |
| 1548 | 4-cyano-phenyl | OCH₂ | 2-quinolinyl | 3-SO₂ⁱPr | H |
| 1549 | 4-cyano-phenyl | OCH₂ | 2-quinolinyl | 3-OCF₃ | H |
| 1550 | 4-cyano-phenyl | OCH₂ | 2-quinolinyl | 3-OCH₂CF₃ | H |
| 1551 | 4-cyano-phenyl | OCH₂ | 2-quinolinyl | 3-NHMe | H |
| 1552 | 4-cyano-phenyl | OCH₂ | 2-quinolinyl | 3-NMe₂ | H |
| 1553 | 4-cyano-phenyl | OCH₂ | 2-quinolinyl | 3- | H |
| | | | | cyclopropyl | |
| 1554 | 4-cyano-phenyl | OCH₂ | 2-quinolinyl | 3-OEt | H |
| 1555 | 4-cyano-phenyl | OCH₂ | 2-quinolinyl | 3-OⁱPr | H |
| 1556 | 4-cyano-phenyl | OCH₂ | 2-quinolinyl | 3-CH₂- | H |
| | | | | cyclopropyl | |
| 1557 | 4-cyano-phenyl | OCH₂ | 2-quinolinyl | 3-SMe | H |
| 1558 | 4-cyano-phenyl | OCH₂ | 2-quinolinyl | 3-SEt | H |
| 1559 | 4-cyano-phenyl | OCH₂ | 2-quinolinyl | 3-SⁱPr | H |
| 1560 | 4-cyano-phenyl | OCH₂ | 2-quinolinyl | 4-F | H |
| 1561 | 4-cyano-phenyl | OCH₂ | 2-quinolinyl | 4-Cl | H |
| 1562 | 4-cyano-phenyl | OCH₂ | 2-quinolinyl | 4-CN | H |
| 1563 | 4-cyano-phenyl | OCH₂ | 2-quinolinyl | 4-NO2 | H |
| 1564 | 4-cyano-phenyl | OCH₂ | 2-quinolinyl | 4-OMe | H |
| 1565 | 4-cyano-phenyl | OCH₂ | 2-quinolinyl | 4-Me | H |
| 1566 | 4-cyano-phenyl | OCH₂ | 2-quinolinyl | 4-Et | H |
| 1567 | 4-cyano-phenyl | OCH₂ | 2-quinolinyl | 4-iPr | H |
| 1568 | 4-cyano-phenyl | OCH₂ | 2-quinolinyl | 4-tBu | H |
| 1569 | 4-cyano-phenyl | OCH₂ | 2-quinolinyl | 4-CF3 | H |
| 1570 | 4-cyano-phenyl | OCH₂ | 2-quinolinyl | 4-SO2Me | H |
| 1571 | 4-cyano-phenyl | OCH₂ | 2-quinolinyl | 4-SO2Et | H |
| 1572 | 4-cyano-phenyl | OCH₂ | 2-quinolinyl | 4-SO2iPr | H |
| 1573 | 4-cyano-phenyl | OCH₂ | 2-quinolinyl | 4-OCF3 | H |
| 1574 | 4-cyano-phenyl | OCH₂ | 2-quinolinyl | 4-OCH2CF3 | H |
| 1575 | 4-cyano-phenyl | OCH₂ | 2-quinolinyl | 4-NHMe | H |
| 1576 | 4-cyano-phenyl | OCH₂ | 2-quinolinyl | 4-NMe2 | H |
| 1577 | 4-cyano-phenyl | OCH₂ | 2-quinolinyl | 4- | H |
| | | | | cyclopropyl | |
| 1578 | 4-cyano-phenyl | OCH₂ | 2-quinolinyl | 4-OEt | H |
| 1579 | 4-cyano-phenyl | OCH₂ | 2-quinolinyl | 4-OiPr | H |
| 1580 | 4-cyano-phenyl | OCH₂ | 2-quinolinyl | 4-CH2- | H |
| | | | | cyclopropyl | |
| 1581 | 4-cyano-phenyl | OCH₂ | 2-quinolinyl | 4-SMe | H |
| 1582 | 4-cyano-phenyl | OCH₂ | 2-quinolinyl | 4-SEt | H |
| 1583 | 4-cyano-phenyl | OCH₂ | 2-quinolinyl | 4-SiPr | H |
| 1585 | 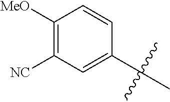 | OCH₂ | 2-quinolinyl | H | H |
| 1586 | 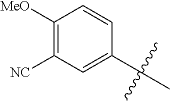 | OCH₂ | 2-quinolinyl | 3-F | H |
| 1587 | 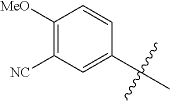 | OCH₂ | 2-quinolinyl | 3-Cl | H |
| 1588 | 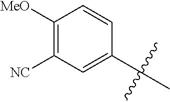 | OCH₂ | 2-quinolinyl | 3-CN | H |
| 1589 | 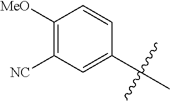 | OCH₂ | 2-quinolinyl | 3-NO₂ | H |
| 1590 | 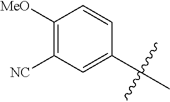 | OCH₂ | 2-quinolinyl | 3-OMe | H |
| 1591 | 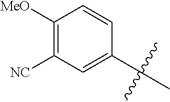 | OCH₂ | 2-quinolinyl | 3-Me | H |
| 1592 | 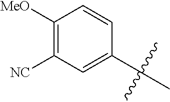 | OCH₂ | 2-quinolinyl | 3-Et | H |
| 1593 | 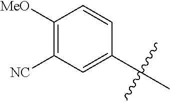 | OCH₂ | 2-quinolinyl | 3-ⁱPr | H |
| 1594 | 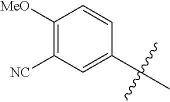 | OCH₂ | 2-quinolinyl | 3-^{t}Bu | H |
| 1595 | 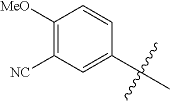 | OCH₂ | 2-quinolinyl | 3-CF₃ | H |
| 1596 | 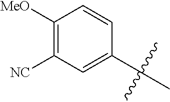 | OCH₂ | 2-quinolinyl | 3-SO₂Me | H |
| 1597 | 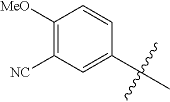 | OCH₂ | 2-quinolinyl | 3-SO₂Et | H |
| 1598 | 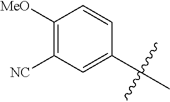 | OCH₂ | 2-quinolinyl | 3-SO₂ⁱPr | H |
| 1599 | 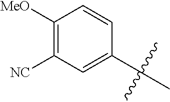 | OCH₂ | 2-quinolinyl | 3-OCF₃ | H |
| 1600 | 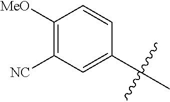 | OCH₂ | 2-quinolinyl | 3-OCH₂CF₃ | H |
| 1601 | 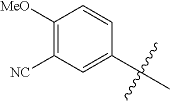 | OCH₂ | 2-quinolinyl | 3-NHMe | H |
| 1602 | 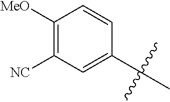 | OCH₂ | 2-quinolinyl | 3-NMe₂ | H |
| 1603 | 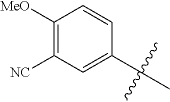 | OCH₂ | 2-quinolinyl | 3- cyclopropyl | H |
| 1604 | 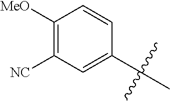 | OCH₂ | 2-quinolinyl | 3-OEt | H |
| 1605 | 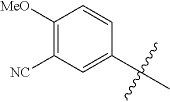 | OCH₂ | 2-quinolinyl | 3-OⁱPr | H |
| 1606 | 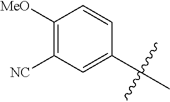 | OCH₂ | 2-quinolinyl | 3-CH₂- cyclopropyl | H |
| 1607 | 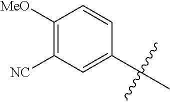 | OCH₂ | 2-quinolinyl | 3-SMe | H |
| 1608 | 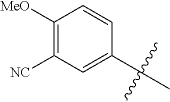 | OCH₂ | 2-quinolinyl | 3-SEt | H |
| 1609 | 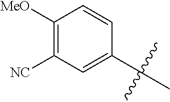 | OCH₂ | 2-quinolinyl | 3-SⁱPr | H |
| 1610 | 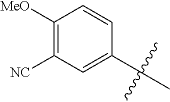 | OCH₂ | 2-quinolinyl | 4-F | H |
| 1611 | 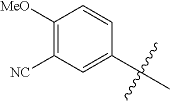 | OCH₂ | 2-quinolinyl | 4-Cl | H |
| 1612 | 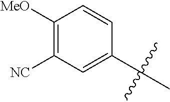 | OCH₂ | 2-quinolinyl | 4-CN | H |
| 1613 | 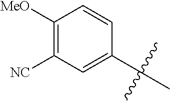 | OCH₂ | 2-quinolinyl | 4-NO2 | H |
| 1614 | 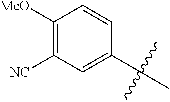 | OCH₂ | 2-quinolinyl | 4-OMe | H |
| 1615 | 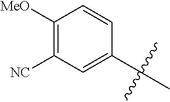 | OCH₂ | 2-quinolinyl | 4-Me | H |
| 1616 | 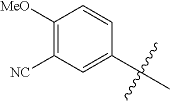 | OCH₂ | 2-quinolinyl | 4-Et | H |
| 1617 | 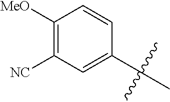 | OCH₂ | 2-quinolinyl | 4-iPr | H |
| 1618 | 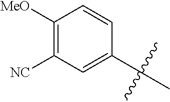 | OCH₂ | 2-quinolinyl | 4-tBu | H |
| 1619 | 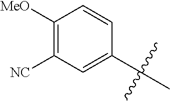 | OCH₂ | 2-quinolinyl | 4-CF3 | H |
| 1620 | 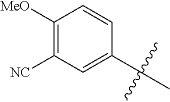 | OCH₂ | 2-quinolinyl | 4-SO2Me | H |
| 1621 | 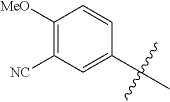 | OCH₂ | 2-quinolinyl | 4-SO2Et | H |
| 1622 | 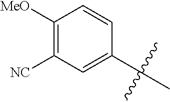 | OCH₂ | 2-quinolinyl | 4-SO2iPr | H |
| 1623 | 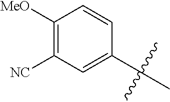 | OCH₂ | 2-quinolinyl | 4-OCF3 | H |
| 1624 | 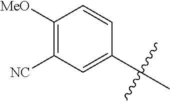 | OCH₂ | 2-quinolinyl | 4-OCH2CF3 | H |
| 1625 | 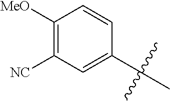 | OCH₂ | 2-quinolinyl | 4-NHMe | H |
| 1626 | 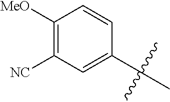 | OCH₂ | 2-quinolinyl | 4-NMe2 | H |
| 1627 | 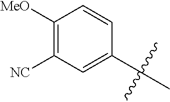 | OCH₂ | 2-quinolinyl | 4- cyclopropyl | H |
| 1628 | 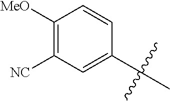 | OCH₂ | 2-quinolinyl | 4-OEt | H |
| 1629 | 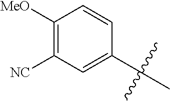 | OCH₂ | 2-quinolinyl | 4-OiPr | H |
| 1630 | 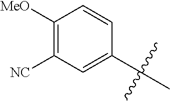 | OCH₂ | 2-quinolinyl | 4-CH2- cyclopropyl | H |
| 1631 | 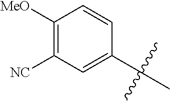 | OCH₂ | 2-quinolinyl | 4-SMe | H |
| 1632 | 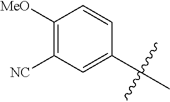 | OCH₂ | 2-quinolinyl | 4-SEt | H |
| 1633 | 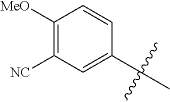 | OCH₂ | 2-quinolinyl | 4-SiPr | H |
| 1634 | 4-methoxy-phenyl | OCH₂ | 2-quinolinyl | H | H |
| 1635 | 4-methoxy-phenyl | OCH₂ | 2-quinolinyl | 3-F | H |
| 1636 | 4-methoxy-phenyl | OCH₂ | 2-quinolinyl | 3-Cl | H |
| 1637 | 4-methoxy-phenyl | OCH₂ | 2-quinolinyl | 3-CN | H |
| 1638 | 4-methoxy-phenyl | OCH₂ | 2-quinolinyl | 3-NO₂ | H |
| 1639 | 4-methoxy-phenyl | OCH₂ | 2-quinolinyl | 3-OMe | H |
| 1640 | 4-methoxy-phenyl | OCH₂ | 2-quinolinyl | 3-Me | H |
| 1641 | 4-methoxy-phenyl | OCH₂ | 2-quinolinyl | 3-Et | H |
| 1642 | 4-methoxy-phenyl | OCH₂ | 2-quinolinyl | 3-ⁱPr | H |
| 1643 | 4-methoxy-phenyl | OCH₂ | 2-quinolinyl | 3-^{t}Bu | H |
| 1644 | 4-methoxy-phenyl | OCH₂ | 2-quinolinyl | 3-CF₃ | H |
| 1645 | 4-methoxy-phenyl | OCH₂ | 2-quinolinyl | 3-SO₂Me | H |
| 1646 | 4-methoxy-phenyl | OCH₂ | 2-quinolinyl | 3-SO₂Et | H |
| 1647 | 4-methoxy-phenyl | OCH₂ | 2-quinolinyl | 3-SO₂ⁱPr | H |
| 1648 | 4-methoxy-phenyl | OCH₂ | 2-quinolinyl | 3-OCF₃ | H |
| 1649 | 4-methoxy-phenyl | OCH₂ | 2-quinolinyl | 3-OCH₂CF₃ | H |
| 1650 | 4-methoxy-phenyl | OCH₂ | 2-quinolinyl | 3-NHMe | H |
| 1651 | 4-methoxy-phenyl | OCH₂ | 2-quinolinyl | 3-NMe₂ | H |
| 1652 | 4-methoxy-phenyl | OCH₂ | 2-quinolinyl | 3- | H |
| | | | | cyclopropyl | |
| 1653 | 4-methoxy-phenyl | OCH₂ | 2-quinolinyl | 3-OEt | H |
| 1654 | 4-methoxy-phenyl | OCH₂ | 2-quinolinyl | 3-OⁱPr | H |
| 1655 | 4-methoxy-phenyl | OCH₂ | 2-quinolinyl | 3-CH₂- | H |
| | | | | cyclopropyl | |
| 1656 | 4-methoxy-phenyl | OCH₂ | 2-quinolinyl | 3-SMe | H |
| 1657 | 4-methoxy-phenyl | OCH₂ | 2-quinolinyl | 3-SEt | H |
| 1658 | 4-methoxy-phenyl | OCH₂ | 2-quinolinyl | 3-SⁱPr | H |
| 1659 | 4-methoxy-phenyl | OCH₂ | 2-quinolinyl | 4-F | H |
| 1660 | 4-methoxy-phenyl | OCH₂ | 2-quinolinyl | 4-Cl | H |
| 1661 | 4-methoxy-phenyl | OCH₂ | 2-quinolinyl | 4-CN | H |
| 1662 | 4-methoxy-phenyl | OCH₂ | 2-quinolinyl | 4-NO2 | H |
| 1663 | 4-methoxy-phenyl | OCH₂ | 2-quinolinyl | 4-OMe | H |
| 1664 | 4-methoxy-phenyl | OCH₂ | 2-quinolinyl | 4-Me | H |
| 1665 | 4-methoxy-phenyl | OCH₂ | 2-quinolinyl | 4-Et | H |
| 1666 | 4-methoxy-phenyl | OCH₂ | 2-quinolinyl | 4-iPr | H |
| 1667 | 4-methoxy-phenyl | OCH₂ | 2-quinolinyl | 4-tBu | H |
| 1668 | 4-methoxy-phenyl | OCH₂ | 2-quinolinyl | 4-CF3 | H |
| 1669 | 4-methoxy-phenyl | OCH₂ | 2-quinolinyl | 4-SO2Me | H |
| 1670 | 4-methoxy-phenyl | OCH₂ | 2-quinolinyl | 4-SO2Et | H |
| 1671 | 4-methoxy-phenyl | OCH₂ | 2-quinolinyl | 4-SO2iPr | H |
| 1672 | 4-methoxy-phenyl | OCH₂ | 2-quinolinyl | 4-OCF3 | H |
| 1673 | 4-methoxy-phenyl | OCH₂ | 2-quinolinyl | 4-OCH2CF3 | H |
| 1674 | 4-methoxy-phenyl | OCH₂ | 2-quinolinyl | 4-NHMe | H |
| 1675 | 4-methoxy-phenyl | OCH₂ | 2-quinolinyl | 4-NMe2 | H |
| 1676 | 4-methoxy-phenyl | OCH₂ | 2-quinolinyl | 4- | H |
| | | | | cyclopropyl | |
| 1677 | 4-methoxy-phenyl | OCH₂ | 2-quinolinyl | 4-OEt | H |
| 1678 | 4-methoxy-phenyl | OCH₂ | 2-quinolinyl | 4-OiPr | H |
| 1679 | 4-methoxy-phenyl | OCH₂ | 2-quinolinyl | 4-CH2- | H |
| | | | | cyclopropyl | |
| 1680 | 4-methoxy-phenyl | OCH₂ | 2-quinolinyl | 4-SMe | H |
| 1681 | 4-methoxy-phenyl | OCH₂ | 2-quinolinyl | 4-SEt | H |
| 1682 | 4-methoxy-phenyl | OCH₂ | 2-quinolinyl | 4-SiPr | H |
| 1683 | 4-pyridinyl | OCH₂ | 2-quinolinyl | H | H |
| 1684 | 4-pyridinyl | OCH₂ | 2-quinolinyl | F | H |
| 1685 | 4-pyridinyl | OCH₂ | 2-quinolinyl | 3-Cl | H |
| 1686 | 4-pyridinyl | OCH₂ | 2-quinolinyl | 3-CN | H |
| 1687 | 4-pyridinyl | OCH₂ | 2-quinolinyl | 3-NO₂ | H |
| 1688 | 4-pyridinyl | OCH₂ | 2-quinolinyl | 3-OMe | H |
| 1689 | 4-pyridinyl | OCH₂ | 2-quinolinyl | 3-Me | H |
| 1690 | 4-pyridinyl | OCH₂ | 2-quinolinyl | 3-Et | H |
| 1691 | 4-pyridinyl | OCH₂ | 2-quinolinyl | 3-ⁱPr | H |
| 1692 | 4-pyridinyl | OCH₂ | 2-quinolinyl | 3-^{t}Bu | H |
| 1693 | 4-pyridinyl | OCH₂ | 2-quinolinyl | 3-CF₃ | H |
| 1694 | 4-pyridinyl | OCH₂ | 2-quinolinyl | 3-SO₂Me | H |
| 1695 | 4-pyridinyl | OCH₂ | 2-quinolinyl | 3-SO₂Et | H |
| 1696 | 4-pyridinyl | OCH₂ | 2-quinolinyl | 3-SO₂ⁱPr | H |
| 1697 | 4-pyridinyl | OCH₂ | 2-quinolinyl | 3-OCF₃ | H |
| 1698 | 4-pyridinyl | OCH₂ | 2-quinolinyl | 3-OCH₂CF₃ | H |
| 1699 | 4-pyridinyl | OCH₂ | 2-quinolinyl | 3-NHMe | H |
| 1700 | 4-pyridinyl | OCH₂ | 2-quinolinyl | 3-NMe₂ | H |
| 1701 | 4-pyridinyl | OCH₂ | 2-quinolinyl | 3-OⁱPr | H |
| 1702 | 4-pyridinyl | OCH₂ | 2-quinolinyl | 3-CH₂- | H |
| | | | | cyclopropyl | |
| 1703 | 4-pyridinyl | OCH₂ | 2-quinolinyl | 3-SMe | H |
| 1704 | 4-pyridinyl | OCH₂ | 2-quinolinyl | 3-SEt | H |
| 1705 | 4-pyridinyl | OCH₂ | 2-quinolinyl | 3-SⁱPr | H |
| 1706 | 4-pyridinyl | OCH₂ | 2-quinolinyl | 4-F | H |
| 1707 | 4-pyridinyl | OCH₂ | 2-quinolinyl | 4-Cl | H |
| 1708 | 4-pyridinyl | OCH₂ | 2-quinolinyl | 4-OMe | H |
| 1709 | 4-pyridinyl | OCH₂ | 2-quinolinyl | 4-Me | H |
| 1710 | 4-pyridinyl | OCH₂ | 2-quinolinyl | 4-Et | H |
| 1711 | 4-pyridinyl | OCH₂ | 2-quinolinyl | 4-ⁱPr | H |
| 1712 | 4-pyridinyl | OCH₂ | 2-quinolinyl | 4-^{t}Bu | H |
| 1713 | 4-pyridinyl | OCH₂ | 2-quinolinyl | 4-CF₃ | H |
| 1714 | 4-pyridinyl | OCH₂ | 2-quinolinyl | 4-SO₂Me | H |
| 1715 | 4-pyridinyl | OCH₂ | 2-quinolinyl | 4-SO₂Et | H |
| 1716 | 4-pyridinyl | OCH₂ | 2-quinolinyl | 4-SO₂ⁱPr | H |
| 1717 | 4-pyridinyl | OCH₂ | 2-quinolinyl | 4-OCF₃ | H |
| 1718 | 4-pyridinyl | OCH₂ | 2-quinolinyl | 4-OCH₂CF₃ | H |
| 1719 | 4-pyridinyl | OCH₂ | 2-quinolinyl | 4-NHMe | H |
| 1720 | 4-pyridinyl | OCH₂ | 2-quinolinyl | 4-NMe₂ | H |
| 1721 | 4-pyridinyl | OCH₂ | 2-quinolinyl | 4- | H |
| | | | | cyclopropyl | |
| 1722 | 4-pyridinyl | OCH₂ | 2-quinolinyl | 4-OEt | H |
| 1723 | 4-pyridinyl | OCH₂ | 2-quinolinyl | 4-OⁱPr | H |
| 1724 | 4-pyridinyl | OCH₂ | 2-quinolinyl | 4-CH₂- | H |
| | | | | cyclopropyl | |
| 1725 | 4-pyridinyl | OCH₂ | 2-quinolinyl | 4-SMe | H |
| 1726 | 4-pyridinyl | OCH₂ | 2-quinolinyl | 4-SEt | H |
| 1727 | 4-pyridinyl | OCH₂ | 2-quinolinyl | 4-SⁱPr | H |
| 1728 | 4-pyridinyl | OCH₂ | 2-quinolinyl | 3-F | 4-F |
| 1729 | 4-pyridinyl | OCH₂ | 2-quinolinyl | 3-F | 4- |
| | | | | | OMe |
| 1730 | 4-pyridinyl | OCH₂ | 2-quinolinyl | 3-F | 4-Cl |
| 1731 | 4-pyridinyl | OCH₂ | 2-quinolinyl | 3-Cl | 4- |
| | | | | | OMe |
| 1732 | 4-pyridinyl | OCH₂ | 2-quinolinyl | 3-Cl | 4-CN |
| 1733 | 4-pyridinyl | OCH₂ | 2-quinolinyl | 3-OMe | 4-F |
| 1734 | 4-pyridinyl | OCH₂ | 2-quinolinyl | 3-CN | 4- |
| | | | | | OMe |
| 1735 | 4-pyridinyl | OCH₂ | 2-quinolinyl | 3-CF₃ | 4-CN |
| 1736 | 4-pyridinyl | OCH₂ | 2-quinolinyl | 3-NMe₂ | 4-F |
| 1737 | 4-pyridinyl | OCH₂ | 2-quinolinyl | 3-F | 4- |
| | | | | | NMe₂ |
| 1738 | 4-pyridinyl | OCH₂ | 2-quinolinyl | 3-O- | 4-CN |
| | | | | cyclopropyl | |
| 1739 | 4-pyridinyl | OCH₂ | 2-quinolinyl | 3-Cl | 4-Cl |
| 1740 | 4-pyridinyl | OCH₂ | 2-quinolinyl | 3- | H |
| | | | | cyclopropyl | |
| 1741 | 4-pyridinyl | OCH₂ | 2-quinolinyl | 3-OEt | H |
| 1742 | 4-pyridinyl | OCH₂ | 2-quinolinyl | 4-CN | H |
| 1743 | 4-pyridinyl | OCH₂ | 2-quinolinyl | 4-NO₂ | H |
| 1744 | 2-methoxy-5- | OCH₂ | 2-quinolinyl | H | H |
| | pyridinyl) | | | | |
| 1745 | 2-methoxy-5- | OCH₂ | 2-quinolinyl | 3-F | H |
| | pyridinyl | | | | |
| 1746 | 2-methoxy-5- | OCH₂ | 2-quinolinyl | 3-Cl | H |
| | pyridinyl | | | | |
| 1747 | 2-methoxy-5- | OCH₂ | 2-quinolinyl | 3-CN | H |
| | pyridinyl | | | | |
| 1748 | 2-methoxy-5- | OCH₂ | 2-quinolinyl | 3-NO₂ | H |
| | pyridinyl) | | | | |
| 1749 | 2-methoxy-5- | OCH₂ | 2-quinolinyl | 3-OMe | H |
| | pyridinyl | | | | |
| 1750 | 2-methoxy-5- | OCH₂ | 2-quinolinyl | 3-Me | H |
| | pyridinyl) | | | | |
| 1751 | 2-methoxy-5- | OCH₂ | 2-quinolinyl | 3-Et | H |
| | pyridinyl | | | | |
| 1752 | 2-methoxy-5- | OCH₂ | 2-quinolinyl | 3-ⁱPr | H |
| | pyridinyl) | | | | |
| 1753 | 2-methoxy-5- | OCH₂ | 2-quinolinyl | 3-^{t}Bu | H |
| | pyridinyl | | | | |
| 1754 | 2-methoxy-5- | OCH₂ | 2-quinolinyl | 3-CF₃ | H |
| | pyridinyl) | | | | |
| 1755 | 2-methoxy-5- | OCH₂ | 2-quinolinyl | 3-SO₂Me | H |
| | pyridinyl | | | | |
| 1756 | 2-methoxy-5- | OCH₂ | 2-quinolinyl | 3-SO₂Et | H |
| | pyridinyl) | | | | |
| 1757 | 2-methoxy-5- | OCH₂ | 2-quinolinyl | 3-SO₂ⁱPr | H |
| | pyridinyl | | | | |
| 1758 | 2-methoxy-5- | OCH₂ | 2-quinolinyl | 3-OCF₃ | H |
| | pyridinyl) | | | | |
| 1759 | 2-methoxy-5- | OCH₂ | 2-quinolinyl | 3-OCH₂CF₃ | H |
| | pyridinyl | | | | |
| 1760 | 2-methoxy-5- | OCH₂ | 2-quinolinyl | 3-NHMe | H |
| | pyridinyl) | | | | |
| 1761 | 2-methoxy-5- | OCH₂ | 2-quinolinyl | 3-NMe₂ | H |
| | pyridinyl | | | | |
| 1762 | 2-methoxy-5- | OCH₂ | 2-quinolinyl | 3- | H |
| | pyridinyl) | | | cyclopropyl | |
| 1763 | 2-methoxy-5- | OCH₂ | 2-quinolinyl | 3-OEt | H |
| | pyridinyl | | | | |
| 1764 | 2-methoxy-5- | OCH₂ | 2-quinolinyl | 3-OⁱPr | H |
| | pyridinyl) | | | | |
| 1765 | 2-methoxy-5- | OCH₂ | 2-quinolinyl | 3-CH₂- | H |
| | pyridinyl | | | cyclopropyl | |
| 1766 | 2-methoxy-5- | OCH₂ | 2-quinolinyl | 3-SMe | H |
| | pyridinyl) | | | | |
| 1767 | 2-methoxy-5- | OCH₂ | 2-quinolinyl | 3-SEt | H |
| | pyridinyl | | | | |
| 1768 | 2-methoxy-5- | OCH₂ | 2-quinolinyl | 3-SⁱPr | H |
| | pyridinyl) | | | | |
| 1769 | 2-methoxy-5- | OCH₂ | 2-quinolinyl | 4-F | H |
| | pyridinyl | | | | |
| 1770 | 2-methoxy-5- | OCH₂ | 2-quinolinyl | 4-Cl | H |
| | pyridinyl) | | | | |
| 1771 | 2-methoxy-5- | OCH₂ | 2-quinolinyl | 4-CN | H |
| | pyridinyl | | | | |
| 1772 | 2-methoxy-5- | OCH₂ | 2-quinolinyl | 4-NO2 | H |
| | pyridinyl) | | | | |
| 1773 | 2-methoxy-5- | OCH₂ | 2-quinolinyl | 4-OMe | H |
| | pyridinyl | | | | |
| 1774 | 2-methoxy-5- | OCH₂ | 2-quinolinyl | 4-Me | H |
| | pyridinyl) | | | | |
| 1775 | 2-methoxy-5- | OCH₂ | 2-quinolinyl | 4-Et | H |
| | pyridinyl | | | | |
| 1776 | 2-methoxy-5- | OCH₂ | 2-quinolinyl | 4-iPr | H |
| | pyridinyl) | | | | |
| 1777 | 2-methoxy-5- | OCH₂ | 2-quinolinyl | 4-tBu | H |
| | pyridinyl | | | | |
| 1778 | 2-methoxy-5- | OCH₂ | 2-quinolinyl | 4-CF3 | H |
| | pyridinyl) | | | | |
| 1779 | 2-methoxy-5- | OCH₂ | 2-quinolinyl | 4-SO2Me | H |
| | pyridinyl | | | | |
| 1780 | 2-methoxy-5- | OCH₂ | 2-quinolinyl | 4-SO2Et | H |
| | pyridinyl) | | | | |
| 1781 | 2-methoxy-5- | OCH₂ | 2-quinolinyl | 4-SO2iPr | H |
| | pyridinyl | | | | |
| 1782 | 2-methoxy-5- | OCH₂ | 2-quinolinyl | 4-OCF3 | H |
| | pyridinyl) | | | | |
| 1783 | 2-methoxy-5- | OCH₂ | 2-quinolinyl | 4-OCH2CF3 | H |
| | pyridinyl | | | | |
| 1784 | 2-methoxy-5- | OCH₂ | 2-quinolinyl | 4-NHMe | H |
| | pyridinyl) | | | | |
| 1785 | 2-methoxy-5- | OCH₂ | 2-quinolinyl | 4-NMe2 | H |
| | pyridinyl | | | | |
| 1786 | 2-methoxy-5- | OCH₂ | 2-quinolinyl | 4- | H |
| | pyridinyl) | | | cyclopropyl | |
| 1787 | 2-methoxy-5- | OCH₂ | 2-quinolinyl | 4-OEt | H |
| | pyridinyl | | | | |
| 1788 | 2-methoxy-5- | OCH₂ | 2-quinolinyl | 4-OiPr | H |
| | pyridinyl) | | | | |
| 1789 | 2-methoxy-5- | OCH₂ | 2-quinolinyl | 4-CH2- | H |
| | pyridinyl | | | cyclopropyl | |
| 1790 | 2-methoxy-5- | OCH₂ | 2-quinolinyl | 4-SMe | H |
| | pyridinyl) | | | | |
| 1791 | 2-methoxy-5- | OCH₂ | 2-quinolinyl | 4-SEt | H |
| | pyridinyl | | | | |
| 1792 | 2-methoxy-5- | OCH₂ | 2-quinolinyl | 4-SiPr | H |
| | pyridinyl) | | | | |
| 1793 | 2-hydroxy-5- | OCH₂ | 2-quinolinyl | H | H |
| | pyridinyl) | | | | |
| 1794 | 2-hydroxy-5- | OCH₂ | 2-quinolinyl | 3-F | H |
| | pyridinyl | | | | |
| 1795 | 2-hydroxy-5- | OCH₂ | 2-quinolinyl | 3-Cl | H |
| | pyridinyl) | | | | |
| 1796 | 2-hydroxy-5- | OCH₂ | 2-quinolinyl | 3-CN | H |
| | pyridinyl | | | | |
| 1797 | 2-hydroxy-5- | OCH₂ | 2-quinolinyl | 3-NO₂ | H |
| | pyridinyl) | | | | |
| 1798 | 2-hydroxy-5- | OCH₂ | 2-quinolinyl | 3-OMe | H |
| | pyridinyl | | | | |
| 1799 | 2-hydroxy-5- | OCH₂ | 2-quinolinyl | 3-Me | H |
| | pyridinyl) | | | | |
| 1800 | 2-hydroxy-5- | OCH₂ | 2-quinolinyl | 3-Et | H |
| | pyridinyl | | | | |
| 1801 | 2-hydroxy-5- | OCH₂ | 2-quinolinyl | 3-ⁱPr | H |
| | pyridinyl) | | | | |
| 1802 | 2-hydroxy-5- | OCH₂ | 2-quinolinyl | 3-^{t}Bu | H |
| | pyridinyl | | | | |
| 1803 | 2-hydroxy-5- | OCH₂ | 2-quinolinyl | 3-CF₃ | H |
| | pyridinyl) | | | | |
| 1804 | 2-hydroxy-5- | OCH₂ | 2-quinolinyl | 3-SO₂Me | H |
| | pyridinyl | | | | |
| 1805 | 2-hydroxy-5- | OCH₂ | 2-quinolinyl | 3-SO₂Et | H |
| | pyridinyl) | | | | |
| 1806 | 2-hydroxy-5- | OCH₂ | 2-quinolinyl | 3-SO₂ⁱPr | H |
| | pyridinyl | | | | |
| 1807 | 2-hydroxy-5- | OCH₂ | 2-quinolinyl | 3-OCF₃ | H |
| | pyridinyl) | | | | |
| 1808 | 2-hydroxy-5- | OCH₂ | 2-quinolinyl | 3-OCH₂CF₃ | H |
| | pyridinyl | | | | |
| 1809 | 2-hydroxy-5- | OCH₂ | 2-quinolinyl | 3-NHMe | H |
| | pyridinyl) | | | | |
| 1810 | 2-hydroxy-5- | OCH₂ | 2-quinolinyl | 3-NMe₂ | H |
| | pyridinyl | | | | |
| 1811 | 2-hydroxy-5- | OCH₂ | 2-quinolinyl | 3- | H |
| | pyridinyl) | | | cyclopropyl | |
| 1812 | 2-hydroxy-5- | OCH₂ | 2-quinolinyl | 3-OEt | H |
| | pyridinyl | | | | |
| 1813 | 2-hydroxy-5- | OCH₂ | 2-quinolinyl | 3-OⁱPr | H |
| | pyridinyl) | | | | |
| 1814 | 2-hydroxy-5- | OCH₂ | 2-quinolinyl | 3-CH₂- | H |
| | pyridinyl | | | cyclopropyl | |
| 1815 | 2-hydroxy-5- | OCH₂ | 2-quinolinyl | 3-SMe | H |
| | pyridinyl) | | | | |
| 1816 | 2-hydroxy-5- | OCH₂ | 2-quinolinyl | 3-SEt | H |
| | pyridinyl | | | | |
| 1817 | 2-hydroxy-5- | OCH₂ | 2-quinolinyl | 3-SⁱPr | H |
| | pyridinyl) | | | | |
| 1818 | 2-hydroxy-5- | OCH₂ | 2-quinolinyl | 4-F | H |
| | pyridinyl | | | | |
| 1819 | 2-hydroxy-5- | OCH₂ | 2-quinolinyl | 4-Cl | H |
| | pyridinyl) | | | | |
| 1820 | 2-hydroxy-5- | OCH₂ | 2-quinolinyl | 4-CN | H |
| | pyridinyl | | | | |
| 1821 | 2-hydroxy-5- | OCH₂ | 2-quinolinyl | 4-NO2 | H |
| | pyridinyl) | | | | |
| 1822 | 2-hydroxy-5- | OCH₂ | 2-quinolinyl | 4-OMe | H |
| | pyridinyl | | | | |
| 1823 | 2-hydroxy-5- | OCH₂ | 2-quinolinyl | 4-Me | H |
| | pyridinyl) | | | | |
| 1824 | 2-hydroxy-5- | OCH₂ | 2-quinolinyl | 4-Et | H |
| | pyridinyl | | | | |
| 1825 | 2-hydroxy-5- | OCH₂ | 2-quinolinyl | 4-iPr | H |
| | pyridinyl) | | | | |
| 1826 | 2-hydroxy-5- | OCH₂ | 2-quinolinyl | 4-tBu | H |
| | pyridinyl | | | | |
| 1827 | 2-hydroxy-5- | OCH₂ | 2-quinolinyl | 4-CF3 | H |
| | pyridinyl) | | | | |
| 1828 | 2-hydroxy-5- | OCH₂ | 2-quinolinyl | 4-SO2Me | H |
| | pyridinyl | | | | |
| 1829 | 2-hydroxy-5- | OCH₂ | 2-quinolinyl | 4-SO2Et | H |
| | pyridinyl) | | | | |
| 1830 | 2-hydroxy-5- | OCH₂ | 2-quinolinyl | 4-SO2iPr | H |
| | pyridinyl | | | | |
| 1831 | 2-hydroxy-5- | OCH₂ | 2-quinolinyl | 4-OCF3 | H |
| | pyridinyl) | | | | |
| 1832 | 2-hydroxy-5- | OCH₂ | 2-quinolinyl | 4-OCH2CF3 | H |
| | pyridinyl | | | | |
| 1833 | 2-hydroxy-5- | OCH₂ | 2-quinolinyl | 4-NHMe | H |
| | pyridinyl) | | | | |
| 1834 | 2-hydroxy-5- | OCH₂ | 2-quinolinyl | 4-NMe2 | H |
| | pyridinyl | | | | |
| 1835 | 2-hydroxy-5- | OCH₂ | 2-quinolinyl | 4- | H |
| | pyridinyl) | | | cyclopropyl | |
| 1836 | 2-hydroxy-5- | OCH₂ | 2-quinolinyl | 4-OEt | H |
| | pyridinyl | | | | |
| 1837 | 2-hydroxy-5- | OCH₂ | 2-quinolinyl | 4-OiPr | H |
| | pyridinyl) | | | | |
| 1838 | 2-hydroxy-5- | OCH₂ | 2-quinolinyl | 4-CH2- | H |
| | pyridinyl | | | cyclopropyl | |
| 1839 | 2-hydroxy-5- | OCH₂ | 2-quinolinyl | 4-SMe | H |
| | pyridinyl) | | | | |
| 1840 | 2-hydroxy-5- | OCH₂ | 2-quinolinyl | 4-SEt | H |
| | pyridinyl | | | | |
| 1841 | 2-hydroxy-5- | OCH₂ | 2-quinolinyl | 4-SiPr | H |
| | pyridinyl) | | | | |
| 1842 | ⁱPr | OCH₂ | 2-quinolinyl | H | H |
| 1843 | Me | OCH₂ | 2-quinolinyl | H | H |
| 1844 | morpholinyl | OCH₂ | 2-quinolinyl | H | H |
| 1845 | N-piperazinyl | OCH₂ | 2-quinolinyl | H | H |
| 1846 | piperazinyl | OCH₂ | 2-quinolinyl | H | H |
| 1847 | piperidinyl | OCH₂ | 2-quinolinyl | H | H |
| 1848 | 3-pyridinyl | OCH₂ | 2-quinoxaline | H | H |
| 1849 | 4-pyridinyl | OCH₂ | 2-quinoxaline | H | H |
| 1850 | morpholinyl | OCH₂ | 2-quinoxalinyl | H | H |
| 1851 | 3-pyridinyl | OCH₂ | 5,6,7,8-tetrahydro- | H | H |
| | | | 2-quinolyl | | |
| 1852 | 4-pyridinyl | OCH₂ | 5,6,7,8-tetrahydro- | H | H |
| | | | 2-quinolyl | | |
| 1853 | morpholinyl | OCH₂ | 5,6,7,8-tetrahydro- | H | H |
| | | | 2-quinolyl | | |

### Dosage and Administration

The present disclosure includes pharmaceutical composition for treating a subject having a neurological disorder comprising a therapeutically effective amount of a compound of Formulas (I), (II) or (III), a derivative or a pharmaceutically acceptable salt thereof, and a pharmaceutically acceptable excipient, carrier or diluent.

The pharmaceutical compositions can be administered in a variety of dosage forms including, but not limited to, a solid dosage form or in a liquid dosage form, an oral dosage form, a parenteral dosage form, an intranasal dosage form, a suppository, a lozenge, a troche, buccal, a controlled release dosage form, a pulsed release dosage form, an immediate release dosage form, an intravenous solution, a suspension or combinations thereof. The dosage can be an oral dosage form that is a controlled release dosage form. The oral dosage form can be a tablet or a caplet. The compounds can be administered, for example, by oral or parenteral routes, including intravenous, intramuscular, intraperitoneal, subcutaneous, transdermal, airway (aerosol), rectal, vaginal and topical (including buccal and sublingual) administration. In one embodiment, the compounds or pharmaceutical compositions comprising the compounds are delivered to a desired site, such as the brain, by continuous injection via a shunt.

In another embodiment, the compound can be administered parenterally, such as intravenous (IV) administration. The formulations for administration will commonly comprise a solution of the compound of Formulas (I), (II) or (III) dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of compound of Formulas (I), (II) or (III) in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In one embodiment, a compound of Formulas (I), (II) or (III) can be administered by introduction into the central nervous system of the subject, e.g., into the cerbrospinal fluid of the subject. The formulations for administration will commonly comprise a solution of the compound of Formulas (I), (II) or (III) dissolved in a pharmaceutically acceptable carrier. In certain aspects, the compound of Formulas (I), (II) or (III) is introduced intrathecally, e.g., into a cerebral ventricle, the lumbar area, or the cisterna magna. In another aspect, the compound of Formulas I is introduced intraocularly, to thereby contact retinal ganglion cells.

The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

In one embodiment, the pharmaceutical composition comprising a compound of Formulas (I), (II) or (III) is administered into a subject intrathecally. As used herein, the term “intrathecal administration” is intended to include delivering a pharmaceutical composition comprising a compound of Formulas (I), (II) or (III) directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of a compound of Formulas (I), (II) or (III) to any of the above mentioned sites can be achieved by direct injection of the pharmaceutical composition comprising the compound of Formulas (I), (II) or (III) or by the use of infusion pumps. For injection, the pharmaceutical compositions can be formulated in liquid solutions, preferably in physiologically compatible buffers such as Hank's solution or Ringer's solution. In addition, the pharmaceutical compositions may be formulated in solid form and re-dissolved or suspended immediately prior to use. Lyophilized forms are also included. The injection can be, for example, in the form of a bolus injection or continuous infusion (e.g., using infusion pumps) of pharmaceutical composition.

In one embodiment, the pharmaceutical composition comprising a compound of Formulas (I), (II) or (III) is administered by lateral cerebro ventricular injection into the brain of a subject. The injection can be made, for example, through a burr hole made in the subject's skull. In another embodiment, the encapsulated therapeutic agent is administered through a surgically inserted shunt into the cerebral ventricle of a subject. For example, the injection can be made into the lateral ventricles, which are larger, even though injection into the third and fourth smaller ventricles can also be made.

In yet another embodiment, the pharmaceutical composition is administered by injection into the cisterna magna, or lumbar area of a subject.

For oral administration, the compounds will generally be provided in unit dosage forms of a tablet, pill, dragee, lozenge or capsule; as a powder or granules; or as an aqueous solution, suspension, liquid, gels, syrup, slurry, etc. suitable for ingestion by the patient. Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutical preparations for oral use can be obtained through combination of a compound of Formulas (I), (II) or (III) with a solid excipient, optionally grinding a resulting mixture, and processing the mixture of granules, after adding suitable additional compounds, if desired, to obtain tablets or dragee cores. Suitable solid excipients in addition to those previously mentioned are carbohydrate or protein fillers that include, but are not limited to, sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For transmucosal administration (e.g., buccal, rectal, nasal, ocular, etc.), penetrants appropriate to the barrier to be permeated are used in the formulation. Such penetrants are generally known in the art.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate. For intramuscular, intraperitoneal, subcutaneous and intravenous use, the compounds will generally be provided in sterile aqueous solutions or suspensions, buffered to an appropriate pH and isotonicity. Suitable aqueous vehicles include Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The suppositories for rectal administration of the drug can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

The compounds can be delivered transdermally, by a topical route, formulated as applicator sticks, solutions, suspensions, emulsions, gels, creams, ointments, pastes, jellies, paints, powders, or aerosols.

The compounds may also be presented as aqueous or liposome formulations. Aqueous suspensions can contain a compound of Formulas (I), (II) or (III) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a compound of Formulas (I), (II) or (III) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

For administration by inhalation, the compounds are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In general a suitable dose will be in the range of 0.01 to 100 mg per kilogram body weight of the recipient per day, preferably in the range of 0.2 to 10 mg per kilogram body weight per day. The desired dose is preferably presented once daily, but may be dosed as two, three, four, five, six or more sub-doses administered at appropriate intervals throughout the day.

The compounds can be administered as the sole active agent, or in combination with other known therapeutics to be beneficial in the treatment of neurological disorders. In any event, the administering physician can provide a method of treatment that is prophylactic or therapeutic by adjusting the amount and timing of drug administration on the basis of observations of one or more symptoms (e.g., motor or cognitive function as measured by standard clinical scales or assessments) of the disorder being treated. Details on techniques for formulation and administration are well described in the scientific and patent literature, see, e.g., the latest edition of Remington's Pharmaceutical Sciences, Maack Publishing Co, Easton Pa. After a pharmaceutical composition has been formulated in an acceptable carrier, it can be placed in an appropriate container and labeled for treatment of an indicated condition. For administration of the compounds of Formulas (I), (II) or (III), such labeling would include, e.g., instructions concerning the amount, frequency and method of administration.

### Biological Examples

### In Vivo Methods

Subjects: Male C57BL/6J mice (Charles River; 20-25 g) were used for all assays except prepulse inhibition (PPI) which used male DBA/2N mice (Charles River, 20-25 g). For all studies, animals were housed five/cage on a 12-h light/dark cycle with food and water available ad libitum.

Conditioned avoidance responding: Testing was performed in commercially available avoidance boxes (Kinder Scientific, Poway CA). The boxes were divided into two compartments separated by an archway. Each side of the chamber has electronic grid flooring that is equipped to administer footshocks and an overhead light. Training consisted of repeated pairings of the light (conditioned stimulus) followed by a shock (unconditioned stimulus). For each trial the light was presented for 5 sec followed by a 0.5 mA shock that would terminate if the mouse crossed to the other chamber or after 10 seconds. The intertrial interval was set to 20 seconds. Each training and test session consisted a four min habituation period followed by 30 trials. The number of avoidances (mouse crossed to other side during presentation of the light), escapes (mouse crossed to the other side during presentation of the shock) and failures (mouse did not cross during the entire trial period) were recorded by a computer. For study inclusion an animal had to reach a criterion of at least 80% avoidances for two consecutive test sessions.

PPI: Mice were individually placed into the test chambers (StartleMonitor, Kinder Scientific, Poway Calif.). The animals were given a five min acclimation period to the test chambers with the background noise level set to 65 decibel (dB) which remained for the entire test session. Following acclimation, four successive trials 120 dB pulse for 40 msec were presented, however these trials were not included in data analysis. The mice were then subjected to five different types of trials in random order: pulse alone (120 dB for 40 msec), no stimulus and three different prepulse+pulse trials with the prepulse set at 67, 69 or 74 dB for 20 msec followed a 100 msec later by a120 dB pulse for 40 msec. Each animal received 12 trials for each condition for a total of 60 trials with an average intertrial interval of 15 sec. Percent PPI was calculated according to the following formula: (1-(startle response to prepulse+pulse)/startle response to pulse alone))×100.

MK-801-induced hyperactivity: After a 30 min acclimatation to the test room mice were individually placed into test cages for a 30 min habituation period. Following habituation to test cages, baseline activity was recorded for 60 min. Mice were then briefly removed and administered test compound and placed immediately back into the test cage. At 5 min prior to test time mice were again briefly removed from test cages and administered MK-801 (0.3 mg/kg, i.p. in 0.9% saline) and then immediately placed back into test cages and activity level recorded 1 hour. Activity level was measured as distance travelled in centimeters (Ethovision tracking software, Noldus Inc. Wageningen, Netherlands).

Catalepsy: Mice were placed on a wire mesh screen set at a 60 degree angle with their heads facing upwards and the latency to move or break stance was recorded. Animals were given three trials per time point with a 30 sec cut-off per trial.

Data analysis: A one-way or two-way ANOVA was used to evaluate overall differences between treatments and a Tukey's post-hoc test or Student's t-test was used to evaluate differences between treatment groups for the one-way ANOVA and a Bonferroni test was used for the two-way ANOVA. The criterion for statistical significance was set to p<0.05.

### In Vitro Methods

hPDE10A1 Enzyme Activity: 50 μl samples of serially diluted Human PDE10A1 enzyme were incubated with 50 μl of [³H]-cAMP for 20 minutes (at 37° C.). Reactions were carried out in Greiner 96 deep well 1 ml master-block. The enzyme was diluted in 20 mM Tris HCl pH7.4 and [³H]-cAMP was diluted in 10 mM MgCl₂, 40 mM Tris.HCl pH 7.4. The reaction was terminated by denaturing the PDE enzyme (at 70° C.) after which [³H]-5′-AMP was converted to [³H]-adenosine by adding 25 μl snake venom nucleotidase and incubating for 10 minutes (at 37° C.). Adenosine, being neutral, was separated from charged cAMP or AMP by the addition of 200 μl Dowex resin. Samples were shaken for 20 minutes then centrifuged for 3 minutes at 2,500 r.p.m. 50 μl of supernatant was removed and added to 200 μl of MicroScint-20 in white plates (Greiner 96-well Optiplate) and shaken for 30 minutes before reading on Perkin Elmer TopCount Scintillation Counter.

hPDE10A1 Enzyme Inhibition: To check inhibition profile 11 μl of serially diluted inhibitor was added to 50 μl of [³H]-cAMP and 50 ul of diluted Human PDE10A1 and assay was carried out as in the enzyme activity assay. Data was analysed using Prism software (GraphPad Inc). Representative compounds of this disclosure are shown in the table below. A compound with the value “A” had an IC₅₀ value less than or equal to 50 nM. A compound with the value “B” had an IC₅₀ value greater than 50 nM:

| | | hPDE10A1 |
|---|---|---|
| Ex | Name | IC₅₀ Band |
| 180 | | B |
| 205 | | A |
| 255 | | A |
| 281 | | A |
| 330 | | B |
| 380 | 2-((2′-(pyridin-4-yl)biphenyl-4-yloxy)methyl)quinoline | A |
| 381 | 2-((2′-fluoro-6′-(pyridin-4-yl)biphenyl-4-yloxy)methyl)quinoline | B |
| 382 | 2-((2′-chloro-6′-(pyridin-4-yl)biphenyl-4-yloxy)methyl)quinoline | A |
| 383 | 6-(pyridin-4-yl)-4′-(quinolin-2-ylmethoxy)biphenyl-2-carbonitrile | A |
| 384 | 2-((2′-nitro-6′-(pyridin-4-yl)biphenyl-4-yloxy)methyl)quinoline | A |
| 385 | 2-((2′-methoxy-6′-(pyridin-4-yl)biphenyl-4-yloxy)methyl)quinoline | A |
| 387 | 2-((2′-methyl-6′-(pyridin-4-yl)biphenyl-4-yloxy)methyl)quinoline | A |
| 392 | 2-((2′-(methylsulfonyl)-6′-(pyridin-4-yl)biphenyl-4- | A |
| | yloxy)methyl)quinoline | |
| 404 | 2-((5′-fluoro-2′-(pyridin-4-yl)biphenyl-4-yloxy)methyl)quinoline | A |
| 405 | 2-((5′-chloro-2′-(pyridin-4-yl)biphenyl-4-yloxy)methyl)quinoline | A |
| 406 | 6-(pyridin-4-yl)-4′-(quinolin-2-ylmethoxy)biphenyl-3-carbonitrile | A |
| 408 | 2-((5′-methyl-2′-(pyridin-4-yl)biphenyl-4-yloxy)methyl)quinoline | A |
| 443 | 2-((2′-isopropylbiphenyl-4-yloxy)methyl)quinoline | B |
| 444 | 2-((2′-methylbiphenyl-4-yloxy)methyl)quinoline | B |
| 445 | 4-(4′-(quinolin-2-ylmethoxy)biphenyl-2-yl)morpholine | B |
| 448 | 6-morpholino-4′-(quinolin-2-ylmethoxy)biphenyl-2-carbonitrile | A |
| 469 | 4-(5-fluoro-4′-(quinolin-2-ylmethoxy)biphenyl-2-yl)morpholine | A |
| 501 | 5-methyl-2-((2′-(pyridin-4-yl)biphenyl-4-yloxy)methyl)pyridine | A |
| 560 | 6-fluoro-2-((2′-(pyridin-4-yl)biphenyl-4-yloxy)methyl)quinoline | A |
| 619 | 2-((2′-(pyridin-4-yl)biphenyl-4-yloxy)methyl)imidazo[1,2-a]pyridine | A |
| 1112 | 1-(5-fluoro-2-(pyridin-4-yl)phenyl)-4-(quinolin-2-ylmethoxy)pyridin- | B |
| | 2(1H)-one | |
| 1706 | 2-((1-(5-fluoro-2-(pyridin-4-yl)phenyl)piperidin-4- | B |
| | yloxy)methyl)quinoline | |
| 1854 | 6-(pyridin-4-yl)-4′-(quinolin-2-ylmethoxy)biphenyl-2-carbaldehyde | A |
| 1855 | | A |
| 1856 | 2-((4′-fluoro-2′-(pyridin-4-yl)biphenyl-4-yloxy)methyl)quinoline | A |
| 1857 | 2-((2′-(1,3-dioxan-2-yl)-6′-(pyridin-4-yl)biphenyl-4- | A |
| | yloxy)methyl)quinoline | |
| 1858 | | A |
| 1859 | 2-((2′-(2-methylpyridin-4-yl)biphenyl-4-yloxy)methyl)quinoline | A |
| 1860 | | A |
| 1861 | | A |
| 1862 | 2-((4′,5′-dimethoxy-2′-(pyridin-4-yl)biphenyl-4-yloxy)methyl)quinoline | B |
| 1863 | | B |
| 1864 | morpholino(6-(pyridin-4-yl)-4′-(quinolin-2-ylmethoxy)biphenyl-2- | B |
| | yl)methanone | |
| 1865 | | B |
| 1866 | 2-((2′-propylbiphenyl-4-yloxy)methyl)quinoline | B |
| 1867 | 2-((4′-methyl-2′-(pyridin-4-yl)biphenyl-4-yloxy)methyl)quinoline | B |
| 1868 | 2-((2′-(pyrrolidin-1-yl)biphenyl-4-yloxy)methyl)quinoline | B |
| 1869 | | B |
| 1870 | 2-(pyridin-4-yl)-4′-(quinolin-2-ylmethoxy)biphenyl-3-carbonitrile | B |
| 1871 | 2-((2′-(furan-3-yl)biphenyl-4-yloxy)methyl)quinoline | B |
| 1872 | 2-((3′-chloro-2′-(pyridin-4-yl)biphenyl-4-yloxy)methyl)quinoline | B |
| 1873 | 4-(6-(pyridin-4-yl)-4′-(quinolin-2-ylmethoxy)biphenyl-2-yl)morpholine | B |
| 1874 | N,N-dimethyl-1-(6-(pyridin-4-yl)-4′-(quinolin-2-ylmethoxy)biphenyl-2- | B |
| | yl)methanamine | |
| 1875 | 2-((2′-ethylbiphenyl-4-yloxy)methyl)quinoline | B |
| 1876 | 2-((4′-chloro-2′-(pyridin-4-yl)biphenyl-4-yloxy)methyl)quinoline | B |
| 1877 | 2-((2′-cyclohexylbiphenyl-4-yloxy)methyl)quinoline | B |
| 1878 | 5-ethyl-2-((2′-(pyridin-4-yl)biphenyl-4-yloxy)methyl)pyridine | B |
| 1879 | 2-((2′-isopropoxybiphenyl-4-yloxy)methyl)quinoline | B |
| 1880 | 2-((4′,5′-dimethyl-2′-(pyridin-4-yl)biphenyl-4-yloxy)methyl)quinoline | B |
| 1881 | 6-(pyridin-4-yl)-4′-(quinolin-2-ylmethoxy)biphenyl-2-amine | B |
| 1882 | 3-methyl-2-((2′-(pyridin-4-yl)biphenyl-4-yloxy)methyl)pyridine | B |
| 1883 | 2-((2′-methoxybiphenyl-4-yloxy)methyl)quinoline | B |
| 1884 | 2-methyl-6-((2′-(pyridin-4-yl)biphenyl-4-yloxy)methyl)pyridine | B |
| 1885 | 2-(pyridin-4-yl)-4′-(quinolin-2-ylmethoxy)biphenyl-4-carbonitrile | B |
| 1886 | 2-((3′-methyl-2′-(pyridin-4-yl)biphenyl-4-yloxy)methyl)quinoline | B |
| 1887 | 3,5-dimethyl-2-((2′-(pyridin-4-yl)biphenyl-4-yloxy)methyl)pyridine | A |
| 1946 | 2-((3′-fluoro-2′-(pyridin-4-yl)biphenyl-4-yloxy)methyl)quinoline | B |
| 1947 | 4-methyl-2-((2′-(pyridin-4-yl)biphenyl-4-yloxy)methyl)pyridine | B |

What is claimed is:

1. A compound of Formulas (I), (II) or (III) or pharmaceutically acceptable salt thereof 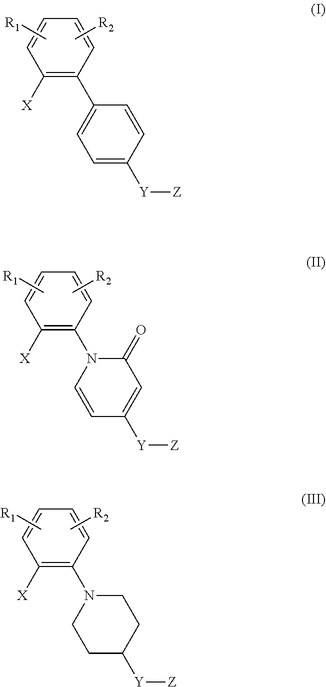
Wherein:
X is selected from C₃-C₈ alkyl, optionally substituted cycloalkyl, optionally substituted cycloalkyloxy, optionally substituted cycloalkylalkyl, optionally substituted cycloalkylalkoxy, optionally substituted heterocycloalkyl, optionally substituted heterocycloalkyloxy, optionally substituted heterocycloalkylalkyl, optionally substituted aryl, optionally substituted arylalkyl, optionally substituted aryloxy, optionally substituted arylalkoxy, optionally substituted heteroaryl, optionally substituted heteroarylalkyl, optionally substituted heteroaryloxy and optionally substituted heteroarylalkoxy;
Y is a bond or a divalent linker group selected from —CH₂—, —O—, —SO₂—, —CH₂O—, —OCH₂— and —CH₂CH₂— with the rightmost radical of the Y group connected to the Z substituent;
Z is optionally substituted heteroaryl;
R₁ is selected from hydrogen, alkyl, CF₃, alkoxy, alkoxyalkyl, optionally substituted cycloalkyl, optionally substituted cycloalkyloxy, optionally substituted cycloalkylalkyl, optionally substituted cycloalkylalkoxy, optionally substituted heterocycloalkyl, optionally substituted heterocycloalkylalkyl, optionally substituted heteroaryl, optionally substituted heteroarylalkyl, halogen, alkylthio, alkylsulfonyl, cyano, amino, alkylamino, dialkylamino, amido, alkylamido, dialkylamido and nitro; and
R₂ is selected from hydrogen, C₁-C₄ alkyl, CF₃, optionally substituted cycloalkyl, halogen, alkoxy, alkylthio, alkylsulfonyl, cyano and nitro.

2. The compound of claim 1 having Formula (I).

3. The compound of claim 1 having Formula (II).

4. The compound of claim 1 having Formula (III).

5. The compound of any of claims 1-4 where X is selected from (C₃-C₈) alkyl, (C₃-C₇)cycloalkyl, (C₃-C₈)cycloalkyloxy, (C₃-C₇)cycloalkyl-(C₁-C₄)alkyl and (C₃-C₇)cycloalkyl-(C₁-C₄)alkoxy

6. The compound of any of claims 1-4 where X is selected from (C₃-C₇) cycloalkyl and (C₃-C₇)cycloalkyl-(C₁-C₄)alkyl

7. The compound of any of claims 1-4 where X is selected from (C₃-C₈) cycloalkyloxy and (C₃-C₇)cycloalkyl-(C₁-C₄)alkoxy

8. The compound of any of claims 1-4 where X is (C₃-C₈) alkyl

9. The compound of any of claims 1-4 where X is heteroaryl

10. The compound of any of claims 1-4 where X is selected from an optionally substituted monocyclic aromatic ring having 5 ring atoms selected from C, O, S and N provided the total number of ring heteroatoms is less than or equal to four and where no more than one of the total number of heteroatoms may be oxygen or sulfur, and a monocyclic aromatic ring having 6 atoms selected from C and N provided that not more than 3 ring atoms are N and where said ring may be optionally and independently substituted with up to two groups selected from (C₁-C₄) alkyl, cycloalkyl, cycloalkyloxy, (C₁-C₄) alkoxy, CF₃, carboxy, alkoxyalkyl, cycloalkylalkoxy, amino, alkylamino, dialkylamino, amido, alkylamido, dialkylamido, thioalkyl, halogen, cyano, and nitro.

11. The compound of any of claims 1-4 where X is an optionally substituted monocyclic aromatic ring having 6 ring atoms selected from C and N provided that not more than 3 ring atoms are N and where said ring may be optionally and independently substituted with up to two groups selected from (C₁-C₄) alkyl, cycloalkyl, cycloalkyloxy, (C₁-C₄) alkoxy, CF₃, carboxy, alkoxyalkyl, cycloalkylalkoxy, amino, alkylamino, dialkylamino, amido, alkylamido, dialkylamido, thioalkyl, halogen, cyano, and nitro.

12. The compound of any of claims 1-4 where X is an optionally substituted monocyclic aromatic ring having 5 ring atoms selected from C, O, S, and N, provided the total number of ring heteroatoms is less than or equal to four and where no more than one of the total number of heteroatoms may be oxygen or sulfur and where said ring may be optionally and independently substituted with up to two groups selected from C₁-C₄ alkyl, cycloalkyl, cycloalkyloxy, C₁-C₄ alkoxy, CF₃, carboxy, alkoxyalkyl, C₁-C₄ cycloalkylalkoxy, amino, alkylamino, dialkylamino, amido, alkylamido, dialkylamido, thioalkyl, halogen, cyano, and nitro.

13. The compound of any of claims 1-4 where X is selected from 2-pyridinyl, 3-pyridinyl or 4-pyridinyl optionally substituted with one group selected from C₁-C₄ alkyl, cyclopropyl, cyclopropyloxy, cyclopropylmethyl, C₁-C₄ alkoxy, CF₃, amino, alkylamino, dialkylamino, thioalkyl, halogen or cyano.

14. The compound of any of claims 1-4 where X is 3-pyridinyl optionally substituted with one group selected from C₁-C₄ alkyl, cyclopropyl, cyclopropyloxy, cyclopropylmethyl, C₁-C₄ alkoxy, CF₃, amino, alkylamino, dialkylamino, thioalkyl, halogen or cyano.

15. The compound of any of claims 1-4 where X is 4-pyridinyl optionally substituted with one group selected from C₁-C₄ alkyl, cyclopropyl, cyclopropyloxy, cyclopropylmethyl, C₁-C₄ alkoxy, CF₃, amino, alkylamino, dialkylamino, thioalkyl, halogen or cyano.

16. The compound of any of claims 1-4 where X is selected from 3-pyridinyl or 4-pyridinyl.

17. The compound of any of claims 1-4 where X is 3-pyridinyl.

18. The compound of any of claims 1-4 where X is 2-methoxy-5-pyridinyl.

19. The compound of any of claims 1-4 where X is X is 4-pyridinyl.

20. The compound of any of claims 1-4 X is 2-methoxy-4-pyridinyl

21. The compound of any of claims 1-4 where X is a heterobicyclic ring system.

22. The compound of any of claims 1-4 where X is a heterobicyclic ring system in which one ring is aromatic.

23. The compound of any of claims 1-4 where X is a heterobicyclic ring system in which both rings are aromatic.

24. The compound of any of claims 1-4 where X is a heterobicyclic ring system containing exactly 9 ring atoms.

25. The compound of any of claims 1-4 where X is a heterobicyclic ring system containing exactly 10 ring atoms.

26. The compound of any of claims 1-4 where X is selected from benzo[d]oxazoyl, benzo[c][1,2,5]oxadiazyl, benzo[c][1,2,5]thiadiazolyl, benzo[d]isoxazolyl, 1H-benzo[d]imidazoyl, benzo[d]thiazoyl, benzo[c]isothiazolyl, benzo[d]isothiazolyl, benzo[c]isoxazolyl, imidazo[1,2-a]pyridinyl and imidazo[1,5-a]pyridinyl

27. The compound of any of claims 1-4 where X is selected from benzo[c][1,2,5]oxadiazyl and benzo[c][1,2,5]thiadiazolyl.

28. The compound of any of claims 1-4 where X is selected from benzo[d]oxazoyl, 1H-benzo[d]imidazoyl and benzo[c]thiazoyl.

29. The compound of any of claims 1-4 where X is benzo[d]oxazoyl.

30. The compound of any of claims 1-4 where X is 1H-benzo[d]imidazoyl.

31. The compound of any of claims 1-4 where X is benzo[d]thiazoyl.

32. The compound of any of claims 1-4 where X is benzo[c][1,2,5]oxadiazoyl.

33. The compound of any of claims 1-4 where X is benzo[c][1,2,5]thiadiazolyl.

34. The compound of any of claims 1-4 where X is benzo[d]isoxazolyl.

35. The compound of any of claims 1-4 where X is benzo[d]isothiazolyl.

36. The compound of any of claims 1-4 where X is benzo[c]isothiazolyl.

37. The compound of any of claims 1-4 where X is benzo[c]isothiazolyl.

38. The compound of any of claims 1-4 where X is benzo[c]isoxazolyl.

39. The compound of any of claims 1-4 where X is imidazo[1,2-a]pyridinyl.

40. The compound of any of claims 1-4 where X is imidazo[1,5-a]pyridinyl.

41. The compound of any of claims 1-4 X is selected from heterocycloalkyl or heterocycloalkyloxy.

42. The compound of any of claims 1-4 where X is heterocycloalkyl consisting of 6 ring atoms.

43. The compound of any of claims 1-4 where X is heterocycloalkyl consisting of 5 ring atoms.

44. The compound of any of claims 1-4 where X is a heterocycloalkyl group selected from Formulas A1-A16 depicted below: 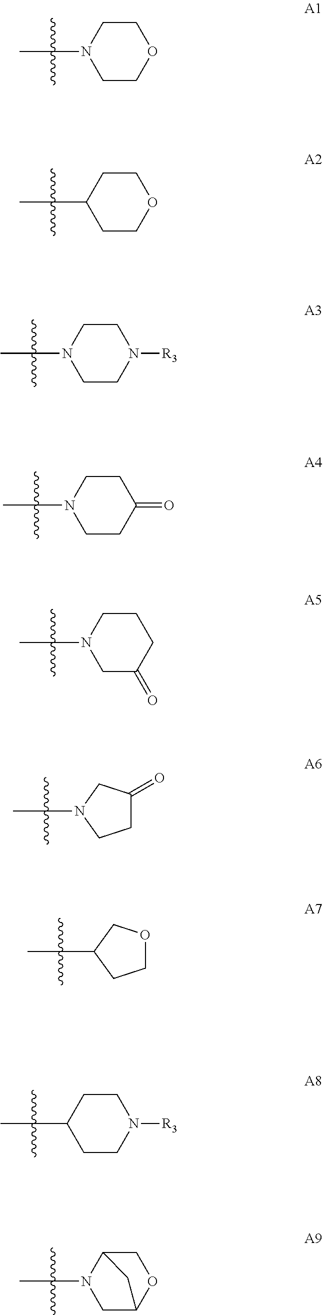 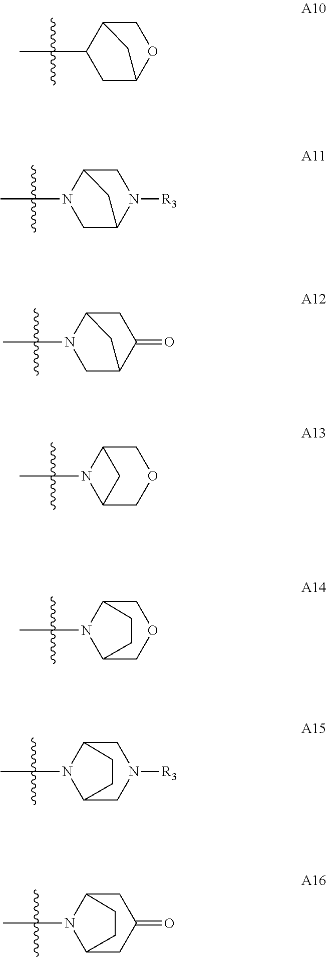
where R₃ is selected from hydrogen, C₁-C₆ alkyl, C₃-C₆ cycloalkyl and C₄-C₈ cycloalkylalkyl.

45. The compound of any of claims 1-4 where X is heterocycloalkyloxy.

46. The compound of any of claims 1-4 where X is aryl.

47. The compound of any of claims 1-4 where X is phenyl.

48. The compound of any of claims 1-4 where X is phenyl optionally substituted with one or more substituents selected from F, Cl, CN, NO₂, CF₃, OCF₃, OCHF₂, CH₂CF₃ and OMe.

49. The compound of any of claims 1-4 where X is restricted phenyl.

50. The compound of any of claims 1-4 where X is selected from a 3,4-disubstituted phenyl, 3-substituted phenyl and 4-substituted phenyl.

51. The compound of any of claims 1-4 where X is 4-substituted phenyl.

52. The compound of any of claims 1-4 where X is 3-substituted phenyl.

53. The compound of any of claims 1-52 where Y is —CH₂O— or —OCH₂ with the rightmost radical connected to the Z substituent.

54. The compound of any of claims 1-52 where Y is —CH₂CH₂— with the rightmost radical connected to the Z substituent.

55. The compound of any of claims 1-52 where Y is —CH₂O— with the rightmost radical connected to the Z substituent.

56. The compound of any of claims 1-52 where Y is —OCH₂— with the rightmost radical connected to the Z substituent.

57. The compound of any of claims 1-56 where Z is selected from heteroaryl consisting of 6 ring atoms and a heterobicyclic ring system

58. The compound of any of claims 1-56 where Z is a heterobicyclic ring system.

59. The compound of any of claims 1-56 where Z is a heterobicyclic ring system where one ring is aromatic.

60. The compound of any of claims 1-56 where Z is a heterobicyclic ring system where both rings are aromatic.

61. The compound of any of claims 1-56 where Z is a heterobicyclic ring system containing exactly 9 ring atoms.

62. The compound of any of claims 1-56 where Z is a heterobicyclic ring system containing exactly 10 ring atoms.

63. The compound of any of claims 1-56 where Z is selected from benzimidazolyl, quinolinyl, tetrahydroquinolyl, imidazo[1,2-c]pyridin-2-yl, tetrahydroisoquinolyl, 5-methylpyridin-2-yl, 3,5-dimethylpyridin-2-yl, 6-fluoroquinolyl and isoquinolinyl, all of which may be optionally substituted with up to 3 substituents independently selected from C₁-C₄ alkyl, C₁-C₄ alkoxy, C₃-C₆ cycloalkyl, C₃-C₆ cycloalkyloxy, C₄-C₈ cycloalkylalkyl, C₄-C₈ cycloalkylalkoxy, halogen, alkylsulfonyl and cyano and nitro.

64. The compound of any of claims 1-56 where Z is 2-quinolinyl substituted with up to 3 substituents independently selected from C₁-C₄ alkyl, C₁-C₄ alkoxy, C₃-C₆ cycloalkyl, C₃-C₆ cycloalkyloxy, C₄-C₈ cycloalkylalkyl, C₄-C₈ cycloalkylalkoxy, halogen, alkylsulfonyl and cyano and nitro.

65. The compound of any of claims 1-56 where Z is 3,5-dimethylpyridin-2-yl substituted with up to 3 substituents independently selected from C₁-C₄ alkyl, C₁-C₄ alkoxy, C₃-C₆ cycloalkyl, C₃-C₆ cycloalkyloxy, C₄-C₈ cycloalkylalkyl, C₄-C₈ cycloalkylalkoxy, halogen, alkylsulfonyl and cyano and nitro.

66. The compound of any of claims 1-56 where Z is 5-methylpyridin-2-yl substituted with up to 3 substituents independently selected from C₁-C₄ alkyl, C₁-C₄ alkoxy, C₃-C₆ cycloalkyl, C₃-C₆ cycloalkyloxy, C₄-C₈ cycloalkylalkyl, C₄-C₈ cycloalkylalkoxy, halogen, alkylsulfonyl and cyano and nitro.

67. The compound of any of claims 1-56 where Z is 2-quinolinyl.

68. The compound of any of claims 1-56 where Z is heteroaryl consisting of 6 ring atoms selected from C and N provided the total number of ring nitrogens is less than or equal to two; said ring is optionally substituted with up to 2 substituents independently selected from C₁-C₄ alkyl, C₁-C₄ alkoxy, C₃-C₆ cycloalkyl, C₃-C₆ cycloalkyloxy, C₄-C₈ cycloalkylalkyl, C₄-C₈ cycloalkylalkoxy, halogen, alkylsulfonyl and cyano and nitro.

69. The compound of any of claims 1-56 where Z is heteroaryl consisting of 6 ring atoms selected from C and N provided the total number of ring nitrogens is less than or equal to two

70. The compound of any of claims 1-56 where Z is pyridinyl optionally substituted with up to 2 substituents independently selected from C₁-C₄ alkyl, C₁-C₄ alkoxy, C₃-C₆ cycloalkyl, C₃-C₆ cycloalkyloxy, C₄-C₈ cycloalkylalkyl, C₄-C₈ cycloalkylalkoxy, halogen, alkylsulfonyl and cyano and nitro.

71. The compound of any of claims 1-70 where R₁ is selected from C₁-C₄ alkyl, CF₃, C₃-C₆ cycloalkyl, C₃-C₆ cycloalkyloxy, C₄-C₈ cycloalkylalkyl, C₄-C₈ cycloalkylalkoxy, alkoxyalkyl, halogen, C₁-C₄ alkoxy, thioalkyl, alkylsulfonyl, cyano, amino, alkylamino, dialkylamino, amido, alkylamido, dialkylamido and nitro.

72. The compound of any of claims 1-70 where R₁ is selected halogen, CF₃, cyano, C₁-C₄ alkoxy, C₃-C₆ cycloalkoxy and alkoxyalkyl

73. The compound of any of claims 1-70 where R₁ is selected from halogen, CF₃, cyano and C₁-C₄ alkoxy.

74. The compound of any of claims 1-70 where R₁ is selected from halogen, CF₃ and cyano.

75. The compound of any of claims 1-70 where R₁ is halogen.

76. The compound of any of claims 1-70 where R₁ is cyano.

77. The compound of any of claims 1-70 where R₁ is methoxy

78. The compound of any of claims 1-70 where R₁ is CF₃.

79. The compound of any of claims 1-78 having Formula:

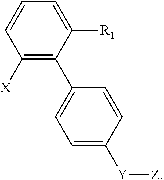

80. The compound of any of claims 1-78 having Formula:

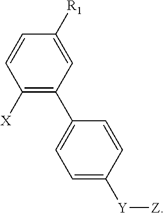

81. The compound of any of claims 1-78 having Formula:

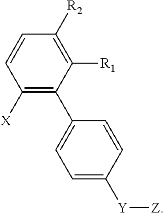

82. The compound of any of claims 1-81 where R₂ is selected from hydrogen, C₁-C₄ alkyl, halogen, C₁-C₄ alkoxy, alkylthio, alkylsulfonyl, cyano or nitro.

83. The compound of any of claims 1-81 where R₂ is selected from hydrogen, C₁-C₄ alkyl, halogen, C₁-C₄ alkoxy and cyano.

84. The compound of any of claims 1-81 where R₂ is selected from hydrogen, halogen, C₁-C₄ alkoxy and cyano.

85. The compound of any of claims 1-81 where R₂ is hydrogen.

86. The compound or pharmaceutically acceptable salt thereof selected from any of Examples 1-1947.

87. A pharmaceutical composition comprising the compound of any of claims 1-86 and a pharmaceutically acceptable carrier or excipient.

88. A method for treating a CNS disorder comprising administering to a human a therapeutically effective amount of the pharmaceutical composition of claim 87.

89. A method for treating eating disorders, obesity, compulsive gambling, sexual disorders, narcolepsy, sleep disorders, diabetes, metabolic syndrome or for use in smoking cessation treatment comprising administering to a human thereof a therapeutically effective amount of the pharmaceutical composition of claim 87.

90. A method for treating obesity, schizophrenia, schizo-affective conditions, Huntington's disease, dystonic conditions and tardive dyskinesia comprising administering to a human thereof a therapeutically effective amount of the pharmaceutical composition of claim 87.

91. A method for treating schizophrenia and schizo-affective conditions comprising comprising administering to a human thereof a therapeutically effective amount of the pharmaceutical composition of claim 87.

92. A method for treating Huntington's disease comprising administering to a human thereof a therapeutically effective amount of the pharmaceutical composition of claim 87.

93. A method for treating obesity and metabolic syndrome comprising administering to a human thereof a therapeutically effective amount of the pharmaceutical composition of claim 87.
